(19) United States
(12) Patent Application Publication
(10) Pub. No.: US 2009/0216068 A1
(43) Pub. Date: Aug. 27, 2009

(54) THERAPEUTIC LOW FREQUENCY PULSED MAGNETIC FIELDS AND DEVICES THEREFOR

(76) Inventors: Alex W. Thomas, London (CA); John Robertson, London (CA); Thas Yuwaraj, Markham (CA); Shyam Mali, Etobicoke (CA)

Correspondence Address: VENABLE LLP, P.O. BOX 34385, WASHINGTON, DC, 20043-9998, US

(21) Appl. No.: 11/917,717

(22) PCT Filed: Jun. 15, 2006
(86) PCT No.: PCT/CA2006/000988

§ 371 Date: Dec. 2, 2008

Related U.S. Application Data
(60) Provisional application No. 60/690,683, filed on Jun. 15, 2005.
(60) Provisional application No. 60/791,426, filed on Apr. 13, 2006.

(51) Int. Cl. A61N 2/04

(52) U.S. Cl. 600/14

(57) ABSTRACT

The present invention is a device for delivering a low frequency magnetic field pulse (Cnp) to affect the physiological and/or neurological conditions of an animal or human. In particular, distinct Cnps are designed to have anti-depression, analgesic, or anti-anxiety effects. These Cnps may be applied in combination in a sequence.

### CROSS-REFERENCE TO RELATED APPLICATIONS

The present application claims priority from U.S. Provisional Patent Application Nos. 60/690,683, filed Jun. 15, 2005; and 60/791,426, filed Apr. 13, 2006.

### FIELD OF THE INVENTION

This invention relates to the use of low frequency pulsed magnetic fields in modifying various physiological and neurological conditions in humans and in animals, and devices therefor.

### BACKGROUND OF THE INVENTION

Diverse studies have shown that the behavioral, cellular and physiological functions of animals can be affected by magnetic stimuli. Weak magnetic fields exert a variety of biological effects ranging from alterations in cellular ion flux to modifications of animal orientation and learning, and therapeutic actions in humans. A number of magnetic field exposures have been shown to reduce exogenous opiate (e.g. morphine) and endogenous opioid peptide (e.g. endorphin) mediated analgesia in various species, including humans (Kavaliers et al., Biol Bull, 180:301-309, 1991; Prato et al., Mag Res Imag, 5:9-14, 1987; Betancur et al., Neurosci Lett, 182:147-150, 1994; Kavaliers et al., On the nature of electromagnetic field interactions with biological systems, Austin: RG Landis Co., p 181-190, 1994; Del Seppia et al., Bioelectromagnetics, 16:290-294 1995; and Papi et al., Bioelectromagnetics, 16:295-300, 1995). As well, extremely low frequency (ELF) magnetic field exposures are reported to modify homing pigeon behavior (Papi et al., J Exp Biol, 160:169-179, 1992) and spatial learning in rodents (Kavaliers et al., J Comp Physiol A, 173:241-248, 1993; and Kavaliers et al., J Comp Physiol A, 179:715-724, 1996) in a manner consistent with alterations in opioid function.

Without committing to any theory, several proposals have been made as to the mechanism behind low frequency magnetic field therapy. For example, low frequency magnetic field exposures have been proposed to exert their effects through the induction of electric currents (Polk, Science, Wash, 247:459-462, 1992; and Weaver et al., Science, Wash, 247:459-462, 1990). Weak magnetic fields have also been proposed to be detected by particles of magnetite in tissue and by virtue of this detection have a physiological effect (Kirschvink et al., Magnetite biomineralization and magnetoreception in organisms: a new biomagnetism, New York: Plenum Press, p 243-256, 1985); however, this magnetite based mechanism is not widely believed (Prato et al., Bioelectromagnetics, 17:123-130, 1996).

Use of low frequency magnetic pulsed fields in treating a physiological, neurological, or behavioral disorder has been described in U.S. Pat. No. 6,234,953, which is hereby incorporated by reference. That patent also discloses that the low frequency pulsed magnetic field may comprise a waveform that has analgesic effects.

Extremely low frequency magnetic fields have little attenuation in tissue, and therefore can be used to alter endogenous processes provided they can be detected in situ and said detection is coupled to a physiological process. It is now shown that magnetic fields may be designed as time varying modulated signals such that they can be used to alter specific targeted physiological and/or neurological processes and in this manner can be used to treat/modify various neurological and physiological conditions and behavioral disorders. Therefore, it is expected that low frequency pulsed magnetic fields would be effective in treating a variety of physiological, neurological, and behavioral disorders, including depression and anxiety.

Depression is a condition which affects the lives of 17 million people in the United States and millions more around the world. It is a serious disease which causes in people a variety of symptoms including lack of sleep, fatigue, loss of appetite, inability to function and suicidal ideation. It is a cause of significant morbidity and mortality in all societies. Depression is currently treated with a variety of pharmacologic agents of which the most widely used are Selective Serotonin Uptake Inhibitors (SSRIs). Although effective to some degree, they are not without side effects including insomnia, appetite disturbance and sexual dysfunction.

Anxiety is a condition which affects the lives of 19 million people in the United States and millions more around the world. It is a serious disease which causes in people a variety of symptoms including fatigue, insomnia and obsessive-compulsive tendencies. It is a cause of significant morbidity in all societies. Anxiety is currently treated with a variety of pharmacologic agents of which the most widely used are SSRIs and benzodiazepines. Although effective to some degree, they are not without side effects including somnolence and the potential for addiction.

It is well known that certain conditions or disease states may be associated with each other. For example, studies have shown that there is an interrelation between chronic pain and depression. Patients with chronic migraine show a high instance of depression (up to 80%), and those patients also having fibromyalgia or fatigue further have a high tendency to have severe depression (30.5% and 29.5%, respectively) (Mercante et al., Arq Neuropsiquiatr, 63:217-220, 2005). Mease (J Rheumatology, 32(S75):6-21) found that fibromyalgia is associated with a high instance of depression (22%) and noted that patients were often prescribed antidepressants as part of their therapy. A similar interrelation between depression and pain was also found in patients with diabetic peripheral neuropathy (Gore et al., J Pain Symptom Management, 30:374-385), with 59.2% of patients having self-reported anxiety/depression. Accordingly, it was suggested that treatment of depression should form a component of therapy for chronic pain.

Chronic pain is known to be associated with not only depression, but anxiety as well. It was found that 35.1% of patients suffering from chronic pain also suffered from an anxiety disorder. This compares to 18.1% incidence of anxiety in the general population (L. A. McWilliams et al., Pain 106 (2003) 127-133). Anxiety is very commonly associated with depression and it was found that approximately 85% of patients with depression experienced symptoms of anxiety while depression occurs in up to 90% of patients with anxiety disorders (Gorman, J M, Depress Anxiety, 1996-97, 4(4), 160-8). Eating disorders are also known to be associated with depression and anxiety. Based on the comorbidity of pain, depression, and anxiety, it is logical to treatment these conditions or disease states together.

The invention described hereinafter offers an alternative therapy which does not require any pharmacological compound or agent to be ingested, and a device for the delivery of said therapy, for the treatment of a variety of physiological, neurological, and behavioral disorders. As such, the use of the present invention can relieve (in whole or in part) the symptoms of these disorders without the associated serious side effects of pharmacologic therapy.

### SUMMARY OF THE INVENTION

The present invention is a device for delivering a low frequency magnetic field pulse (Cnp) to affect the physiological and/or neurological conditions of an animal or human.

One aspect of the present invention is an electrotherapy device comprising a transducer and a controller, the controller causing the transducer to produce a specific low frequency magnetic field pulse (Cnp), said Cnp comprising a plurality of intermittent waveforms, with a latency period between waveforms.

In some embodiments of the device above, the waveform is designed to initially mimic an endogenous electrical activity of target tissue of said subject.

In some embodiments of the device described above, the Cnp is designed to have anti-depression or anti-anxiety effects.

In another embodiment of the device described above, the output encompasses at least 2 Cnps, which are separated by refractory periods to form a Cnp pulse train, said refractory periods being variable.

Preferably, the refractory period between Cnps varies in a predetermined manner over time.

In another embodiment of the device described above, the controller is capable of causing the transducer to produce at least two different pluralities of Cnps in succession.

In another embodiment of the device described above, the transducer is a wire coil.

Preferably, the wire coil has an elliptical shape.

A further aspect of the present invention is a method for treating a disorder selected from the group of physiological, neurological and behavioral disorders, said method comprising applying to a subject a specific low frequency magnetic field pulse (Cnp), said Cnp comprises a plurality of intermittent waveforms.

In another embodiment of the method above, the waveforms have at least one latency period between them.

In another embodiment of the method above, at least two different pluralities of Cnps are applied to the subject in succession.

Another aspect of the present invention is a use of the device described above for treating at least one disorder selected from the group comprising: depression, anxiety, chronic pain.

In another embodiment of the use above, the device is capable of producing at least two different pluralities of Cnps in succession.

Another aspect of the present invention is a low frequency magnetic field pulse (Cnp) comprising a plurality of intermittent waveforms, with a latency period between waveforms, and said waveforms being designed to initially mimic an endogenous electrical activity of target tissue of a subject.

Another aspect of the invention is an electrotherapy device comprising transducers oriented such that optimal deep tissue penetration is achieved.

In another embodiment, the electrotherapy device comprises transducers oriented such that the deep tissue magnetic field is nullified without compromising the magnetic field at surface tissues.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

Certain embodiments of the invention will now be described in relation to the drawings in which:

FIG. 1 shows a schematic representation of a neuro-modulation therapy device.

FIG. 2 illustrates one embodiment of a transducer used in the neuromodulation therapy device, which is a magnetic wire coil.

FIG. 3 shows the wire coil of FIG. 2 worn in a head-set.

FIG. 4 shows a core-less embodiment of the wire coil of FIG. 2.

FIG. 5 shows a solid core embodiment of the wire coil of FIG. 2.

FIG. 6 illustrates a layered printed circuit board (PCB) embodiment of the wire coil of FIG. 2.

FIG. 7 shows several views of the head-set of FIG. 3: front and back view (A), side view (B), top view (C), and bottom view (D).

FIG. 8 shows a systems-level representation of the neuro-modulation therapy device.

FIG. 9 illustrates the data transfer in one embodiment of the neuro-modulation therapy device.

FIG. 10 shows a basic waveform of Cnp-1; Cnp-1 being a specific Cnp designed to have anti-depressive effects.

FIG. 11 shows one Cnp-1 pulse.

FIG. 12 shows a Cnp-1 pulse train.

FIG. 13 shows a basic waveform of Cnp-2; Cnp-2 being a specific Cnp designed to have analgesic effects.

FIG. 14 shows a Cnp-2 pulse train.

FIG. 15 shows a basic waveform of Cnp-3; Cnp-3 being a specific Cnp designed to have anti-anxiety effect.

FIG. 16 shows one Cnp-3 pulse.

FIG. 17 shows a Cnp-3 pulse train.

FIG. 18 shows the head-set of FIG. 3, defining the coordinate axes and indicating the mid-plane of the head-set.

FIG. 19 shows the experimental setup used to measure the magnetic field generated by the head-set of FIG. 3.

FIG. 20 is a 3D spatial profile of peak flux density at mid-plane between the coils in the head-set of FIG. 3.

FIG. 21 is a 3D spatial profile of peak flux density at 2 cm below mid-plane in the head-set of FIG. 3.

FIG. 22 is a 3D spatial profile of peak flux density at 4 cm below mid-plane in the head-set of FIG. 3.

FIG. 23 is a graph showing changes in anxiety levels in different subjects as a result of treatment using one embodiment of the present invention.

FIG. 24 is a graph showing changes in depression levels in different subjects as a result of treatment using one embodiment of the present invention.

### DETAILED DESCRIPTION OF THE PREFERRED EMBODIMENTS

As hereinbefore mentioned, the present invention provides designed and characterized low frequency magnetic field pulses (Cnps), and devices therefor, which have specific effects on physiological, neurological and behavioral conditions in animals and human. The specific low frequency magnetic field pulses are designed for complex neuroelectromagnetic applications and permit the development of therapeutic strategies in order to treat and/or alter various physiological, neurological and behavioral disorders particularly in mammals and more specifically in humans.

Magnetic fields have been demonstrated to have various biological effects in humans, rodents and snails. Such magnetic fields can be detected and this detection can be broadly linked to certain physiological processes. It is now demonstrated that low frequency magnetic field pulses can be designed specifically to alter specific targeted physiological processes and in this manner provide a therapeutic method for treatment and alleviation of certain conditions without the need for pharmacological intervention which is expensive and which poses several problems with respect to side effects of certain drugs.

According to one broad aspect of the invention, the embodiment of the device is a Neuromodulation Therapy (NMT) Device. NMT refers to the system that is used to deliver specific low frequency magnetic field pulses (Cnps). The device comprising a transducer and a controller, the controller being capable of causing the transducer to produce a specific Cnp, said Cnp comprising a series of waveforms.

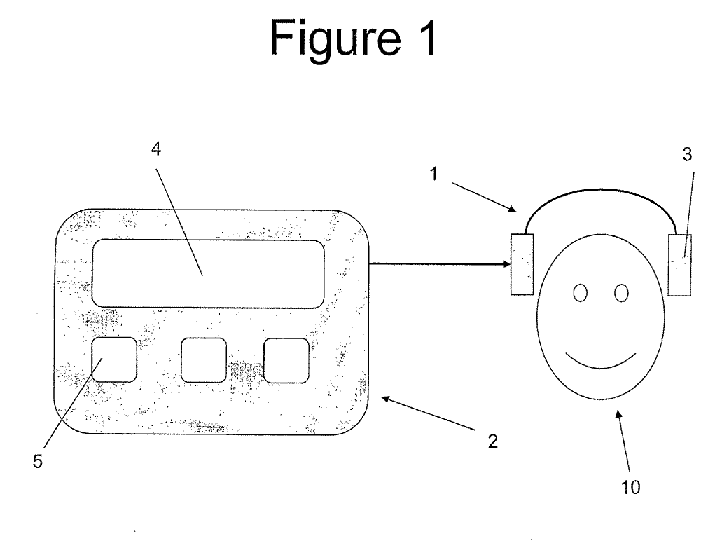

FIG. 1 shows a conceptual representation of one embodiment of a NMT Device illustrating the components. In the embodiment illustrated in FIG. 1, the NMT Device is composed of three components: a headset 1, a hand-held device 2, and firmware (not illustrated).

The headset 1 is comprised of coils of wire that act as transducers 3 to convert electrical signals to specific electromagnetic signals (ie. Cnps), and is worn by a user 10.

The hand-held device 2 is comprised of electronics to store and deliver specifically designed electrical signals to the headsets. The hand held device 2 preferably has a display 4 for showing the operational status of the NMT, and buttons 5 for user control.

The coils of headset 1 are placed in a harness for suitable placement in the head-region. The coil can also be embedded in a head-mounted holster such as a head-band or cap. Alternatively, the wire coils can be assembled in a harness that is mounted to the side arms of an eye-glass or earlobe-mounted clips. This makes the design aesthetically pleasing and conceals the wire coils. It will be understood by a person skilled in the art that although the present device is described with respect to wire coils, any similar structure capable of generating the Cnps of the present invention, such as another type of transducer or a magnetic field generator, could be used.

The wire coils may be wound as a self-supporting core-less structure (without a solid core) in an ellipsoid or square shape. This makes the coil flexible. Alternatively, the coil can be wound on a solid core or etched on a multi-layer printed circuit board (PCB). These will be discussed in greater detail with regard to the elliptical coil configuration below.

Preferably, the transducer is designed to generate maximum magnetic flux density at a target region without significantly increasing peripheral levels. Further preferably, the transducer is a coil capable of generating an electromagnetic field when appropriately energized (ie. via time varying voltage or current signals). More preferably, the coil is a wire coil with a unique coil geometry. More preferably, the geometry of the wire coil is elliptical. Alternative embodiments encompass other electromagnetic sources well know to those skilled in the art.

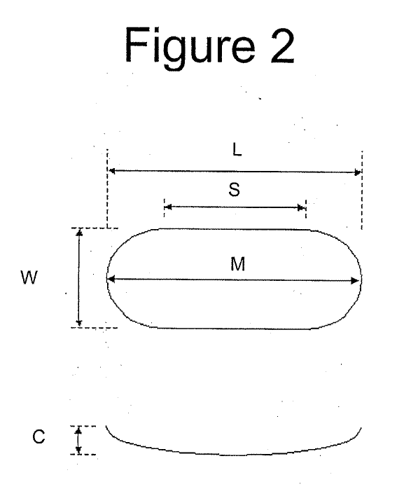

In one embodiment, illustrated in FIG. 2, a desired wire coil size of 9.0 cm by 3.5 cm is chosen to adequately cover the entire cingulate cortex region of a human subject without the necessity of moving or repositioning the wire coils on the subject. A net deep-brain field strength of 1 G (100 μT) is desired for this embodiment. As shown in FIG. 2, the wire coil has a length L of 9.0 cm, a width W of 3.5 cm, and a separation S of 5.5 cm between the rounded ends. The wire coil has a curvature C of 1 cm along its length L, corresponding to the curvature of the head at 2.5 cm above the ears. Preferably, the wire coil comprises 32 AWG wire, 475 turns, with a total resistance of 58 Ohms, and a total inductance of 24 mH.

Use of a large diameter (major axis M of the ellipse, FIG. 2) helps maximize the ratio of flux density at the deep-brain region to that at the surface: B_{deep-brain}/B_{surface}. In other words, maximum possible flux density is achieved at the deep-brain region without significantly elevating the flux density levels at the surface of the brain.

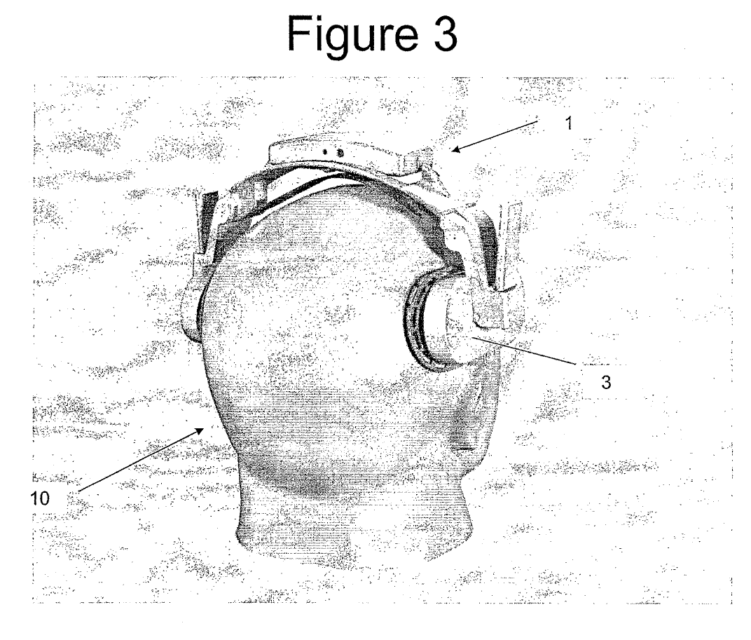

Advantageously, when placed such that the centre of the wire coils are above the ears (see FIG. 3), the wire coils apply a relatively uniform flux density in the mid-line along the sagittal plane (front to back). As shown in FIG. 3, the wire coils can be placed in a custom-designed headset that provides a comfortable and consistent means of positioning the coils on the head.

Additionally, this wire coil geometry provides a field profile that is comparable to a large circular wire coil (with a diameter that is the mean of the major and minor diameter of the ellipse).

The wire coil geometry also provides a field profile that is uniform in a confined volume that is centrally located between the pair of wire coils.

Using this wire coil geometry and wire coil placement, there is the option to either optimize the field strength at the mid-point region between the pair of wire coils (corresponding to deep-brain region) or generate null field at this region. In the latter case, field strength is not compromised at the surface of each of the wire coils (corresponding to brain surface regions). Optimum field strength at mid-point is achieved by orienting the current directions in each of the two wire coils such that the vectors corresponding to the magnetic fields generated by each of the wire coils are oriented in the same direction (parallel vectors). Field strength at mid-point is nullified by orienting the current directions in each of the two wire coils such that the above mentioned vectors are opposite to each other (anti-parallel vectors). Similarly, the phase difference between the signals used to excite each of the wire coils can be deterministically adjusted to achieve optimum field strength at various pre-determined regions between the two wire coils. This feature can be used to deliver energy to selective regions of the brain. In other words, focused delivery of energy is achieved. An embodiment of the invention is an electrotherapy device comprising transducers oriented such that optimal deep tissue penetration is achieved. In an further embodiment of the invention, the electrotherapy device comprises transducers oriented such that the deep tissue magnetic field is nullified without compromising the magnetic field at surface tissues.

This wire coil design is also advantageous for user-friendliness. This wire coil geometry provides the ability to deliver therapy to multiple regions of the brain without the need for repositioning the wire coils over target regions. In other words, the subject/patient can adorn the headset at one consistent position and receive therapeutic levels of specific magnetic fields at all target regions of interest.

The elliptical wire coil can be suitably curved so that a comfortable profile around the head is achieved. This design enhancement avoids the introduction of pressure points that would otherwise cause discomfort over prolonged use. This design enhancement also serves to increase the magnetic field density on the concave side of the wire coil and reduce the same on the convex side.

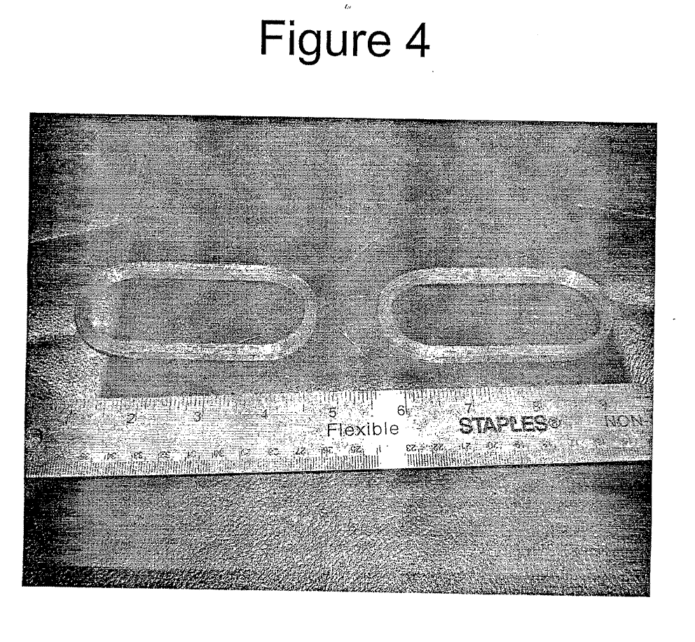

The wire coil can be realized in a number of ways. Self-supporting core-less wire coils are illustrated in FIG. 4. Such wire coils do not require any support framework. Instead, they are wound on a support and then mechanically stabilized using an electrically and magnetically inert material (ie. epoxy). Such a configuration is suitable in scenarios where the resulting weight of the wire coil has to be kept to a minimum. Because the core-less wire coils do not require a core, they are more flexible and hence more adaptable to a variety of head-set configurations.

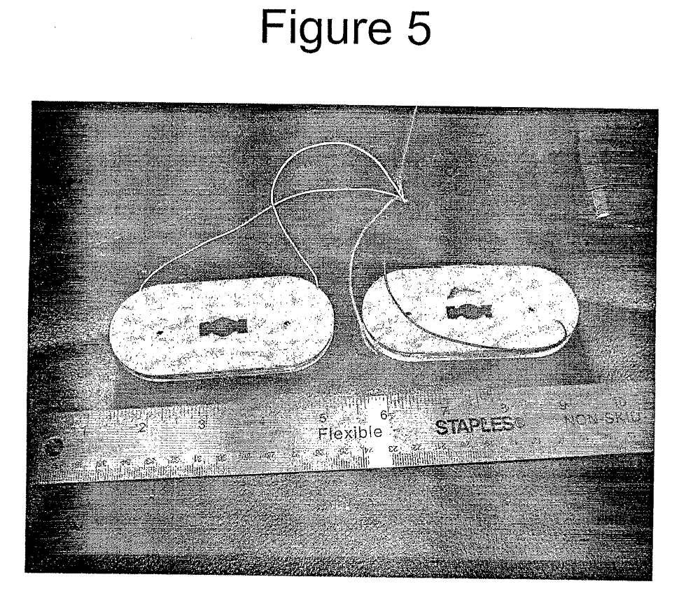

The wire coils can also be formed by winding on a frame (also known as a bobbin) that retains the required elliptical shape. Such an embodiment is suitable in applications where a robust means of constructing the wire coil is essential. Also, such a configuration simplifies the high-level assembly process when the wire coil has to be placed in an enclosure. An example of this design is shown in FIG. 5.

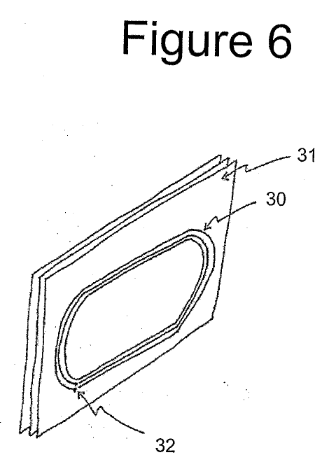

Yet another method of forming the wire coils is by using a printed circuit board (PCB). In this method, the wire coils are etched on to a multi-layer PCB with appropriate number of ‘turns’ in each layer. A schematic representation of this design is shown in FIG. 6. The spacing between the PCB layers and the turns of the wire coil have been exaggerated in the illustration. The wire coil is formed by copper tracks 30 on a multi-layer PCB 31. The copper tracks 30 pass through holes 32 in the PCB 31 to provide one complete conductor with multiple loops that span multiple layers.

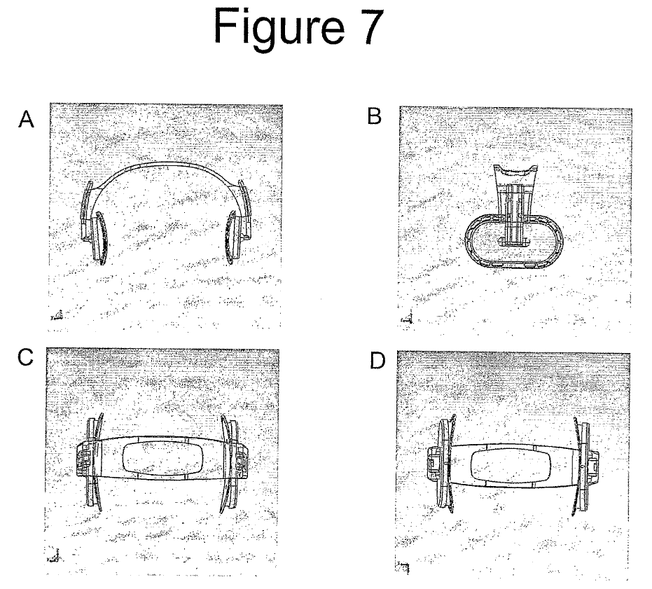

One embodiment of the head-set 1 is illustrated in FIG. 7. FIG. 7 shows the front and back view (7A), side view (7B), top view (7C) and bottom view (7D) of the head-set. The head-set provides a comfortable and consistent fit over the head. The box-like structure at the top of the headset can potentially enclose signal-generator electronics that would energize the wire coils.

The headset 1 is connected to the output of the hand-held NMT device 2 (see FIG. 1). This connection is preferably a physical (i.e. wire) link. A wire-less connection is also included in the design to enhance comfort (and compliance). The wire-less link could be through such technologies as Bluetooth or other high-bandwidth/short-range wireless links.

Even though the head is the main target region, similar wire coils and/or other transducers known in the art can be used to deliver therapy to other relatively shallow tissue targets such as the knee, elbow, wrist, shoulder, ankle and back. The firmware is comprised of the software that is embedded in the hand-held device 2 (FIG. 1) to provide control of the therapy with appropriate user interaction.

The firmware is designed such that it can compare a metric that is derived from the numerical values of the particular waveform of a particular Cnp (also known as a check-sum) against a previously computed value. This provides a means of ensuring that the data transferred to the device is not corrupted in the data transfer process.

The firmware uses the same design to confirm the checksum of the waveform prior to its application in each therapy session. This ensures that the waveform is not corrupted during regular use of the device (after it has been dispensed to a patient). The firmware uses standard encryption methods to ensure that the waveform stored in the device cannot be easily manipulated or extracted by unauthorized personnel.

The day-to-day usage of the device by a patient is stored in the local memory of the device for later retrieval. This usage pattern will provide compliance information to the physician. The same information can be also used for maintenance purpose. The firmware has provisions for automatically detecting if the headset 1 is attached to the device. If a therapy session is initiated without proper connection of the headset 1 (wired or wireless) the device 2 will generate a warning message, which may be shown on the display 4, and not initiate the therapy session until the headset 1 is plugged into the device 2 (FIG. 1).

The Cnp to be delivered in a therapy session can be stored in the device in a parametric form to conserve storage space in the device. In other words, the basic characteristics of the Cnp is stored along with additional parameters (such as play-back rate, time periods between Cnp waveforms) in order to recreate the Cnp during normal use of the device. This facilitates the use of the same basic waveform shape for a number of therapies that only differ in terms of frequency of the pulses and their overall repetition rate.

The device also has provisions for storing the Cnp in ‘raw’ form. In other words, the exact timing parameters of the Cnp can be pre-computed and the exact representation of the Cnp (as a function of time) can be stored in the internal memory of the device. The device supports the storage of a multiplicity of such Cnps.

A specific treatment for a particular, or group of, disorders, can be created by grouping similar and/or different Cnps that will be delivered in a sequential manner. This is comparable to the use of a ‘play list’ in a portable music player.

The hardware contains a real-time clock to time-stamp and track usage of the device. This component facilitates monitoring of compliance with the treatment regiment.

In a preferred embodiment, the entire hardware is contained in a portable enclosure to facilitate ease of use.

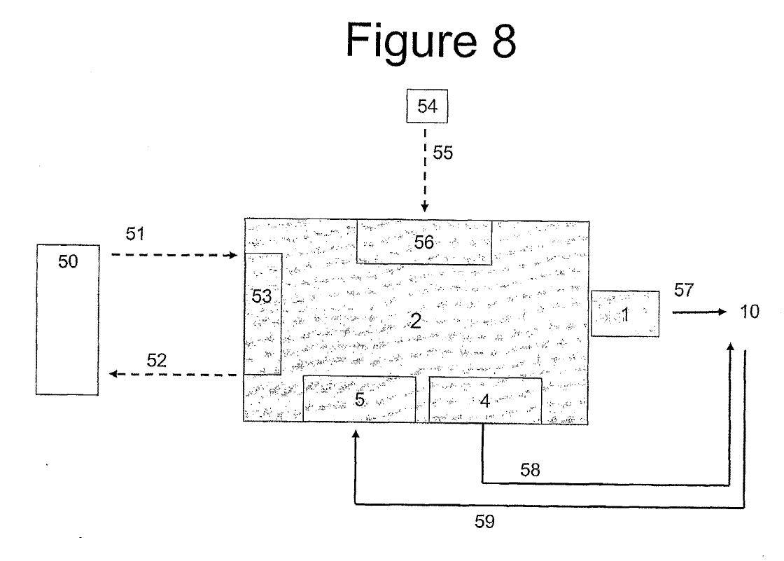

Apart from the above components, the device also preferably has provisions for communicating with an outside computer to perform various maintenance and configuration operations. Interaction between the various system components and the outside world is illustrated in FIG. 8 which shows a system-level representation of an embodiment of the NMT device 2.

Solid lines in FIG. 8 indicate interactions that exist during the delivery of a particular therapy. Dotted lines indicate the interactions that exist either during maintenance or configuration mode (i.e. during the dispensing of the device or during regular maintenance).

The device 2 can be physically connected to a host computer 50 via a communication link such as USB, serial port or other wire-less means. This communication link allows transfer of data, such as the firmware and the Cnp data. The firmware and the Cnp data can be transferred to the device 2 via a removable storage medium (such as compact flash card, flash memory etc.).

FIG. 8 shows how the device 2 may communicate with the host computer 50 via uploads 51 from the host computer 50 to the device 2 of the Cnp data and system diagnostics, and downloads 52 of usage logs and diagnostic results. Such communication is coordinated by the communication unit 53 within the device 2.

Downloading 52 of the usage-logs from the device 2 allows a physician to be able to assess the usage pattern of a particular patient and recommend changes in usage (dosage) as necessary.

Uploading 51 of updates for the firmware resident in the device 2 allows any enhancements or design changes to the software that is resident in the device to be deployed by the physician without necessitating the return of the device to a service facility.

The device 2 can run a specific set of internal diagnostics upon request from the host computer 50 and download 52 the results of the tests to the host computer 50. This functionality will facilitate fault analysis at remote sites and uploading of the results to a central facility for analysis and diagnosis of the device.

The application resident in the host-PC 50 includes a user-authentication stage that ensures that only authorized personnel may establish a communication link with the device 2 (for maintenance or configuration purposes). This ensures that the device's functionality cannot be tampered with or inadvertently modified.

In the case where the device 2 is rechargeable, it must occasionally get an energy charge 55 from an external power supply 54. This can be stored in a battery 56 or other power source within the device 2.

In operation, the device 2 sends an electromagnetic pulse 57 to the user 10, via the headset 1. The user 10 is provided with various status information 58 via the display 4, and is able to provide input 59 via the buttons 5.

Preferably, the device of the present invention is able to deliver an arbitrarily pulsed magnetic field with the following criteria: a) is simple enough for a patient to operate; b) is dependent only on battery or home AC power; c) is capable of reproducing a sophisticated waveform such as that incorporated in a Cnp; d) is contained within a compact case; e) has sufficient power output to deliver without appreciable waveform degradation a minimum of 100 μT (peak) magnetic field to any part of the brain using an appropriate transducer (preferably two 1″ diameter coils) and a minimum of 400 μT (peak) magnetic field 1 cm from an appropriate transducer (preferably a single 3″ diameter coil) for relatively shallow tissue target; f) can monitor patient compliance.

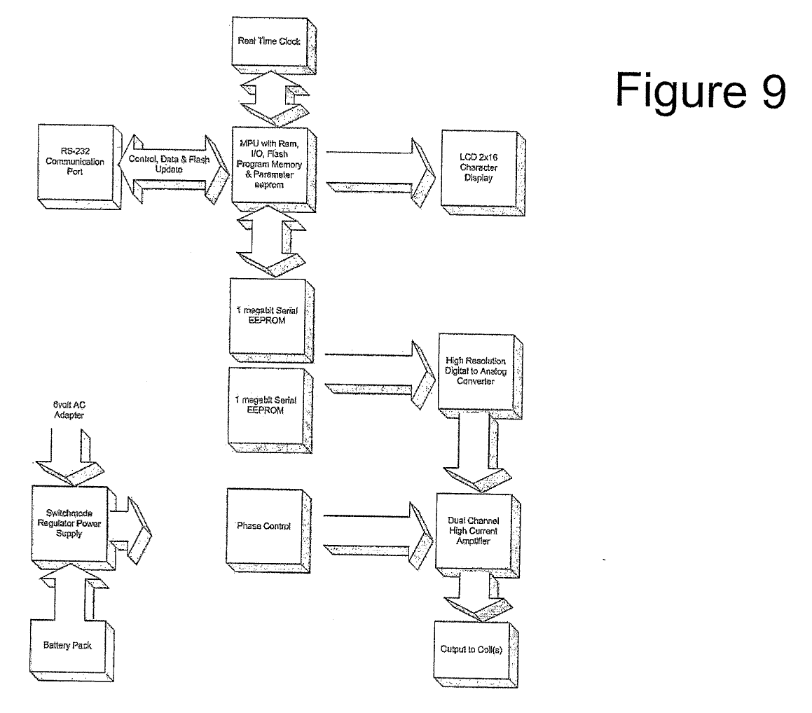

In one embodiment, all functions are controlled by the Micro Processing Unit, but Cnp data passes directly from the selected EEPROM (electronically erasable-programmable read-only memory) to the Digital to Analog converter. This unique feature, illustrated in FIG. 9, permits faster data transfer from memory to the digital to analog converter and maintains complete flexibility and control.

In one preferred embodiment, the device has 2 output channels, with a range of +/−6 Volts, to product a 400 micro Tesla field at the surface of the coil. The power input is 4 ‘AA’ Alkaline or Ni-MH batteries, lasting 12-15 hours or a 6 Volt AC adapter. The Cnp data are stored in 2 1 Mbit EEPROMs, which provide two patterns of 65, 536 points with 12 bit amplitude resolution. It has a LCD. Communication with an outside computer is via a 19.2 Kb RS-232 serial port. With batteries, the device has an approximate weight of 300 g, and its dimensions are 18 cm×9 cm×3.2 cm, making it light and convenient to carry. It also comprises a real time clock and log file, which provides for tracking patient compliance. A 180 degree phase control switch allows for deep or shallow brain exposure. Other embodiments may include a reduction in size, more pattern storage, an audible tone, lower power consumption, alternative communication ports such as USB or wireless links, alternative displays such as LEDs, circuitry to support a rechargeable energy source or FlashCard support.

In a preferred embodiment, the head set further comprises wire coils that can deliver a maximum of 400 micro Tesla (4 Gauss) at the outer edge of the head, to a minimum of 100 micro Tesla (2 Gauss) to the deep brain.

The device of the present invention is designed to deliver one or more Cnps in particular patterns. The characteristics of a Cnp is described in detail below.

### Design of Waveform

The Cnps are comprised of a plurality of intermittent waveforms. The waveform is designed to mimic the corresponding electromagnetic waveform of the target tissue. For example, if the target tissue were a part, or parts, of the brain then the waveform would correspond to the energetic activity of those parts. The waveform is necessarily not sinusoidal as the waveform may be designed to affect critical functions that do not rely on sinusoidal waveforms. The waveform typically has a first rise/fall to prepare for stimulation, followed by an opposite fall/rise to stimulate the firing of axons in the tissue type of interest, and a built in delay to reduce the probability of neuronal excitation as the waveform ends.

### Latency Period

After each waveform or between successive waveforms there is a delay, a latency period. This delay may be progressively set to increase, or decrease, in length with time. This effectively modulates, in time, the frequency of appearance of the waveform. The specific lengths and progression of the Cnp waveforms are related to the target tissue. With respect to the central nervous system (CNS) for example, there are a number of characteristic frequencies which relate to: a) frequencies specific to the area of the brain; b) frequencies associated with communication/connection between different brain regions; and c) frequencies and phase offsets associated with the co-ordination of different brain regions for a specific function. Now, although the waveform has been designed to stimulate neuronal activity for a specific region, electrical activity of a region of the CNS will vary between individuals, and over time, within an individual. Therefore, to target a function the frequency of presentation of the waveform should match the frequency of the target. However, the target is varying within a frequency bandwidth. These CNS frequencies vary between approximately 7 Hz to 300 Hz. (For example: 7 Hz corresponds to alpha rhythm; 10 Hz thalamic activity; 15 Hz autonomic time; 30 Hz intralaminar thalamus and temporal regions associated with memory and consciousness; 40 Hz connection between hippocampal and amygdal temporal regions; 45 Hz hippocampal endogenous frequency; 80 Hz hippocampal-thalamic communication; 300 H z motor control.) These frequencies have upper limits due to neuronal electrical properties, that is: after a neuron “fires” it is left in a hyperpolarized state and cannot fire again until it recovers. Therefore, the latency period: a) allows neurons to recover so that when the waveform is reapplied the neuron can respond; and b) its length is set so that the frequency of presentation of the waveform matches or approximates the frequencies associated with the target. A group of waveforms separated by a series of latency periods forms a Cnp.

### Modulation of Latency Period

To change the electrical activity of the target tissue in the CNS, the Cnp must “latch on” by matching the endogenous frequency of the target tissue, or more appropriately, entrain, to the appropriate frequency and either slow it down or speed it up. The waveform itself does not change substantially, rather, the frequency discussed herein corresponds to the rate at which the waveform is presented and the rate at which electrical spikes occur in the target tissue. Generally, for the CNS, as the frequency of neuronal activity is increased the amount of tissue involved per burst of activity decreases. Conversely, as the frequency is decreased a greater amount of tissue is synchronized and recruited throughout the CNS. For example, a) greater speed of cognitive processing can be associated with increased rates; b) if the rate is decreased significantly in humans or animals with epileptic-type disorders so much tissue can be recruited that seizures will occur. Therefore, the ramping up or ramping down of the rate of presentation of the waveform will: a) ensure that at least at some time the applied and endogenous rates will be matched (provided of course that the initial rate is greater than the endogenous if the purpose is to reduce the endogenous rate or lower if the purpose is to increase the endogenous rate); and b) “pull down” or “push up” the endogenous rate.

### Refractory Period

As a result of the application of the Cnp the synchrony of the electrical activity of the target can be disrupted. Before the application of another Cnp can be effectual the tissue must recover its synchrony. It is allowed to do so by providing a refractory period between application of successive Cnps where the length of the refractory period is determined by the target. For example, if the Cnps are applied to a target in humans which is associated with “awareness”, then the target will recover only after the awareness anticipation time is exceeded (e.g. 1200 ms). Another example would be the application for the same target, but in rodents without significant awareness, in which case the refractory period could be reduced to 400 ms. If the Cnps are to be applied for long periods of time per day, e.g. hours, then the refractory periods should be increased to 10 seconds to avoid possible immunosuppression. Immunosuppression has been show to occur when the CNS is stimulated chronically and this may be minimized if the refractory periods of this stimulation are increased to more than 7 seconds. A group of Cnps separated by a series of refractory periods form a Cnp “pulse train”.

### Variability in Features

It must be pointed out that the Cnp features are related to the underlying physiology and that endogenous frequencies vary between individuals and within an individual. Therefore, there is tolerance on the feature specifications for any Cnp designed for a specific target. The features of the Cnp, as discussed above, can be varied somewhat and the outcome will remain similar due to biological variations in the target. As well, as more and more is learned about biological interactions, the Cnp can be modified to take advantage of the new knowledge to make the Cnp even more specific.

### Pulse Trains

As discussed above, the Cnp waveform are aggregated into specific low frequency magnetic field pulses (ie. Cnp). In order to further counter the homeostatic mechanisms of the target tissue in the brain, arrangement of the Cnp into Cnp pulse trains can be effective. In a preferable arrangement the first three refractory periods are relatively short in duration and progressively increase in duration. The fourth refractory period is of typical length for that Cnp and as such is much longer than the previous three (ie. refractory period 1<refractory period<refractory period 3<refractory period 4). The cycle then begins with the next refractory period being the shortest of the four (ie. refractory period 1). The progressively increasing refractory periods counter homeostasis by allowing the brain to respond to a different level for each pulse (e.g. reaction vs. cognitive processing). This prevents the brain from acclimatizing to a particular pattern of excitation and homeostatically decreasing sensitivity to it.

The Cnps generated by the devices of the present invention will now be described both generally and specifically with respect to Cnps designed to have anti-depressive, analgesic and anti-anxiety effects.

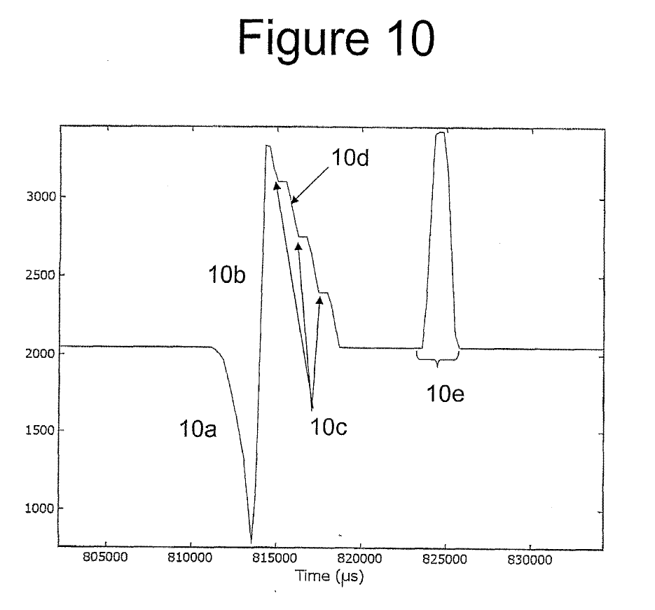

Reference will be made to FIGS. 10-17. The y-axis in these Figures indicate the “normalized value”. The normalized value is a digital representation of the Cnp. These values are stored as binary numbers in the memory of the electrotherapy device. A digital-to-analog converter in the device linearly transforms them to potential differences (expressed in Volts) and then the transducer or coil linearly transforms them to magnetic fields (expressed in micro-Tesla).

A preferred embodiment would translate the numerical values shown in FIGS. 10-17 into voltage levels of +/−12V peak-to-peak. These voltage ranges, in turn, translate to a peak magnetic field of approximately 110 micro-Tesla.

Reference also will be made to Tables 6-8, in which the Amplitude column indicates the voltage produced by the device using normalized values. Preferably, a voltage level of +/−12V peak-to-peak is translated to a peak magnetic field of approximately 110 micro-Tesla.

### Cnp-1-Anti-Depression Cnp:

Cnp-1 can be characterized by its waveform shape and characteristics, latency periods, amplitude modulation, and refractory periods. This Cnp is designed to have anti-depressive effects.

Generally speaking, Cnps are comprised of a plurality of intermittent waveforms. The waveform is designed to mimic the corresponding electromagnetic waveform of the target tissue. In the case of depression, the primary regions of the brain that is to be affected by low frequency magnetic fields is the medial thalamus, prefrontal cortex areas anterior cingulate and frontal cortex of the brain.

The basic waveform is as depicted in FIG. 10. It is important to note that multiple units of this waveform can be arranged in series without separation by a latency period. The duration of the waveform is preferably of 1-30 ms. As seen in FIG. 10, the waveform is not sinusoidal as this waveform was designed to affect critical functions that do not rely on sinusoidal waveforms.

As shown in FIG. 10, the waveform comprises the features of a fall (10a), a rise (10b), delays (10c), a rapid down (10d), and an up and down (10e). During the fall (10a), the waveform prepares for the stimulation of neurons. The rise (10b) stimulates the firing of axons in the tissue type of interest. The built in delay (10c) reduces the probability of neuronal excitation as the waveform ends. The rapid down (10d) causes the firing of neurons in synchrony with their natural frequency. Finally, the up and down (10e) serves as directional noise which prevents cerebral tissue from reaching homeostasis, and prepares for the next sequence of features 10a-10d.

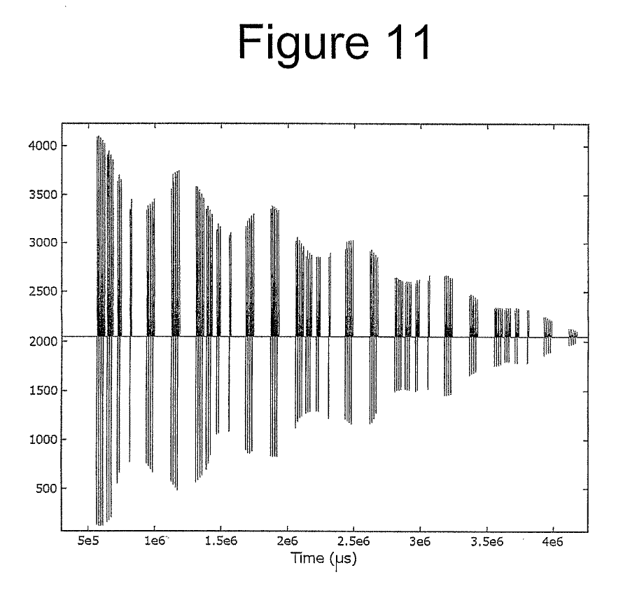

It has been previously described that the waveforms of a Cnp can occur in groups not separated by a latency period. Conversely, multiple waveforms separated by latency periods (of similar or varying lengths) can form a Cnp. The latency period is preferably in the range of 1-300 ms. One embodiment of Cnp-1 is shown in FIG. 11.

The Cnps may be separated by a refractory period. As a result of the application of the Cnps, the neural tissue in the targeted area is stimulated in a pattern that is in accordance with the Cnp. However, it is observed that nervous tissue can accommodate to the stimulus and thereby lose responsiveness. Amplitude modulation serves to progressively upregulate the sensitivity of the nervous tissue as the stimulus gradually wines in amplitude along the Cnp. After the refractory period, the new Cnp then commences at high amplitude which “shocks” the target tissue into awareness and responsiveness of the stimulus.

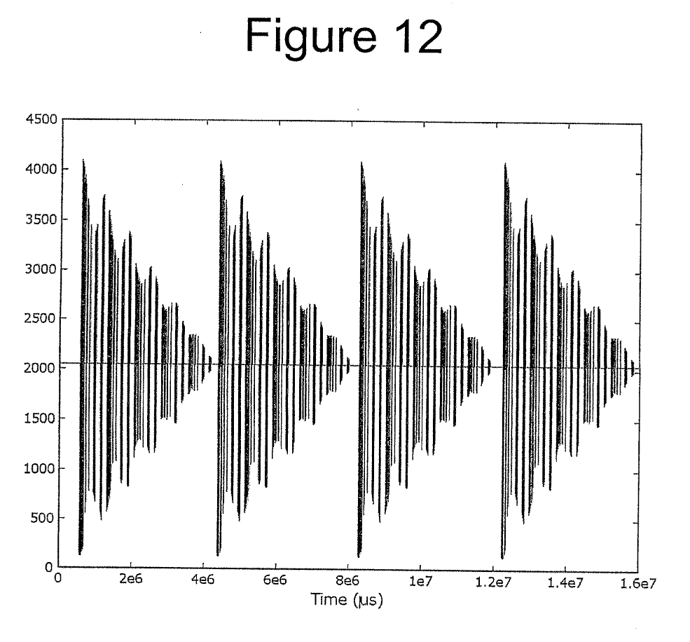

A series of Cnps separated by refractory periods comprise a Cnp pulse train. One embodiment of a Cnp-1 pulse train is shown in FIG. 12. Preferably, there are four refractory periods, the first three shorter refractory periods progressively increasing in duration, and the longer fourth refractory period being typical for the Cnp-1 pulse. The shorter refractory periods are preferably in the range of 100-400 ms, and the longer refractory period is preferably in the range of 3000-4000 ms. One preferable embodiment of the series of refractory periods for the Cnp-1 pulse train have approximate durations as listed in Table 1.

TABLE 1

| Refractory period | Time duration (s) |
|---|---|
| 1 | 0.198765 |
| 2 | 0.273546 |
| 3 | 0.348327 |
| 4 | 3.672194 |

One embodiment of a Cnp-1 is described by the data-points in Table 6. As discussed previously, the Amplitude of the data-points indicate the voltage generated by the device, which then causes the transducer to produce a magnetic field.

The Cnp-1 preferably comprises an amplitude modulation with a negatively sloping, bipolar sinusoidal envelope.

### Cnp-2-Analgesic Cnp:

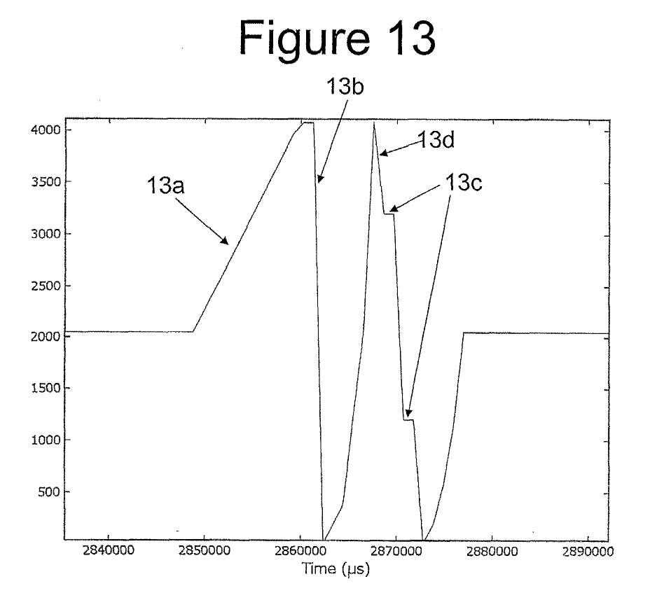

Cnp-2 comprises the analgesic waveform shown in FIG. 13 (which was previously disclosed in U.S. Pat. No. 6,234,953).

As shown in FIG. 13, the analgesic waveform comprises the features of a rise (13a), a fall (13b), delays (13c), and a rapid down (13d). During the rise (13a), the waveform reaches a maximum and prepares for the stimulation of neurons. The fall (13b) stimulates the firing of axons in the tissue type of interest. The built in delay (13c) serves to reduce the probability of neuronal excitation as the waveform ends. Finally, the rapid down (13d) causes the firing of neurons in synchrony with their natural frequency.

One embodiment of a single analgesic waveform is described by the data-points in Table 2. As discussed previously, the Amplitude of the data-points indicate the voltage generated by the device, which then causes the transducer to produce a magnetic field.

Preferably, the Cnp-2 comprises a series of Cnp-2 waveforms separated by varying latency periods. The latency periods are preferably in the range of 1-50 ms. One preferable embodiment of the series of latency periods comprise 17 such periods, with respective approximate durations as listed in Table 7.

TABLE 2

| Latency period | Time duration (s) |
|---|---|
| 1 | 0.001037 |
| 2 | 0.001037 |
| 3 | 0.001037 |
| 4 | 0.001037 |
| 5 | 0.013557 |
| 6 | 0.013637 |
| 7 | 0.013557 |
| 8 | 0.013637 |
| 9 | 0.027195 |
| 10 | 0.028232 |
| 11 | 0.028232 |
| 12 | 0.028232 |
| 13 | 0.040752 |
| 14 | 0.041869 |
| 15 | 0.041789 |
| 16 | 0.041789 |
| 17 | 0.041869 |

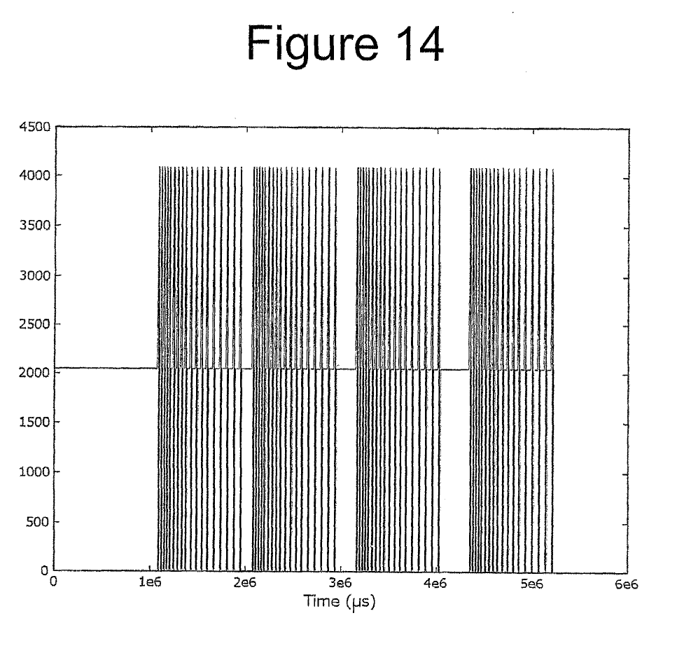

One embodiment of a Cnp-2 pulse train is shown in FIG. 14. Preferably, the Cnp-2 pulse train comprises a series of Cnp-2 separated by varying refractory periods. The series of varying refractory periods preferably comprise three shorter refractory periods and a fourth longer refractory period, the fourth refractory period having a duration suitable to Cnp-2. The shorter refractory periods are preferably in the range of 50-350 ms, and the longer refractory period is preferably in the range of 1600-1900 ms. One preferable embodiment of the series of refractory periods comprise 4 such periods, with respective approximate durations as listed in Table 3.

TABLE 3

| Refractory period | Time duration (s) |
|---|---|
| 1 | 0.103356 |
| 2 | 0.201449 |
| 3 | 0.299541 |
| 4 | 1.780325 |

### Cnp-3-Anti-Anxiety Cnp:

The Cnp-3 field can be characterized by its waveform shape, latency period, and refractory period. This Cnp is designed to have anti-anxiety properties. This Cnp is designed to preferably target the insular cortex, Papez circuit, and anterior cingulate areas of the brain.

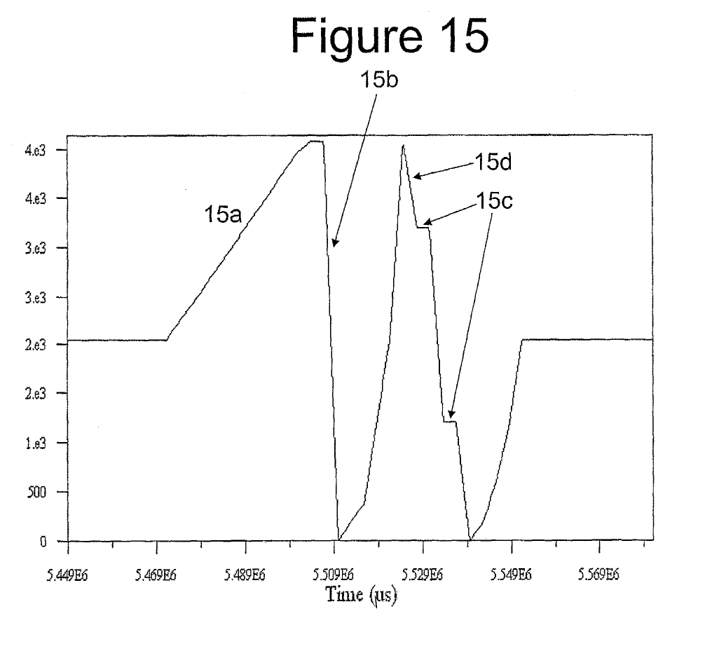

One embodiment of the basic waveform of Cnp-3 is depicted in FIG. 15. The duration of the waveform is preferably 50-100 ms. As shown in FIG. 15, the waveform comprises the features of a rise (15a), a fall (15b), delays (15c), and a rapid down (15d). Daring the rise (15a), the waveform reaches a maximum and prepares for the stimulation of neurons. The fall (15b) stimulates the firing of axons in the tissue type of interest. The built in delay (15c) serves to reduce the probability of neuronal excitation as the waveform ends. Finally, the rapid down (15d) causes the firing of neurons in synchrony with their natural frequency.

One embodiment of a single anti-anxiety waveform is described by the data-points in Table 8.

The anti-anxiety Cnp pulse has a latency period. This latency is progressively set to increase, or decrease, in length with time, modulating the frequency of appearance of the waveform. As in Cnp-1 and Cnp-2, the specific lengths and progression of the waveforms are related to the target tissue. In order to match or approximate the frequency of the target tissue, the latency period between waveforms in each pulse of the anti-anxiety Cnp is preferably in the range of 1-250 ms. One preferable embodiment of the Cnp-3 pulse has a series of 17 latency periods, with respective approximate durations as listed in Table 4.

TABLE 4

| Latency | |
|---|---|
| period | Time duration (s) |
| 1 | 0.002941 |
| 2 | 0.002941 |
| 3 | 0.002941 |
| 4 | 0.002941 |
| 5 | 0.038458 |
| 6 | 0.038684 |
| 7 | 0.038458 |
| 8 | 0.038684 |
| 9 | 0.077143 |
| 10 | 0.080084 |
| 11 | 0.080084 |
| 12 | 0.080084 |
| 13 | 0.115601 |
| 14 | 0.118768 |
| 15 | 0.118542 |
| 16 | 0.118542 |
| 17 | 0.118768 |

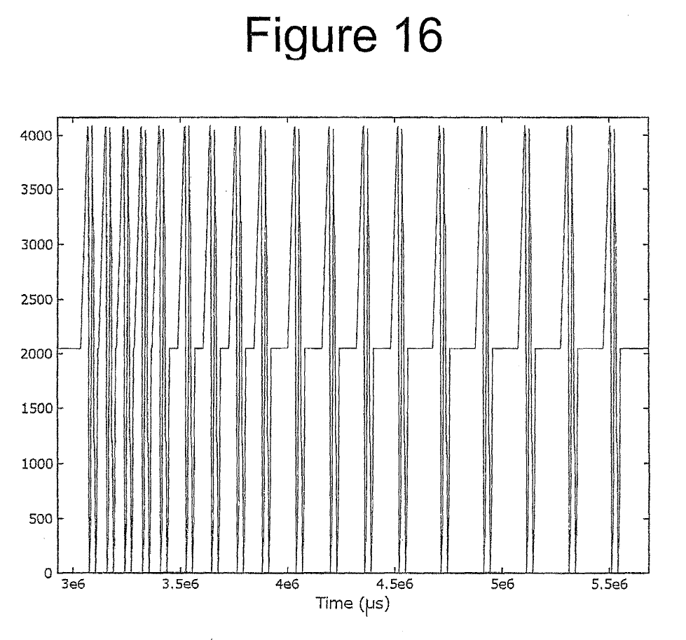

A Cnp-3 is comprised of several Cnp-3 waveforms, one embodiment of which is shown in FIG. 16. Unlike the anti-depression Cnp, the Cnp-3 does not exhibit significant amplitude modulation of its constituent waveforms.

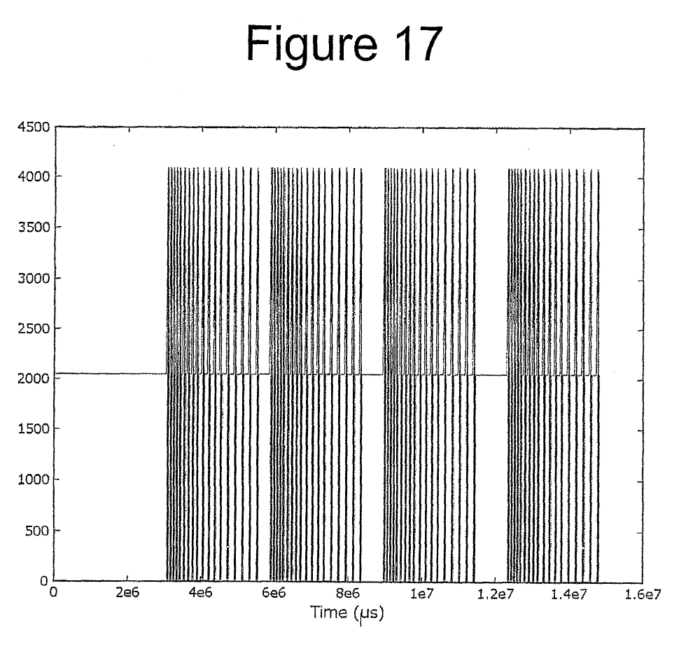

One embodiment of a Cnp-3 pulse train is shown in FIG. 17. Preferably, there are 4 varying refractory periods, with three shorter refractory periods followed by a fourth longer refractory period. The three shorter refractory periods preferably are in the range of 200-900 ms, and the longer refractory period is preferable in the range of 5000-7000 ms. One embodiment of the Cnp-3 pulse has refractory periods with respective approximate durations as listed in Table 5.

TABLE 5

| Refractory period | Time duration (s) |
|---|---|
| 1 | 0.293188 |
| 2 | 0.571444 |
| 3 | 0.849701 |
| 4 | 6.046166 |

### Treatment Cocktail

According to another embodiment of the invention, the device delivers different Cnps (of varying numbers for varying lengths of time-namely, varying numbers of Cnp pulse trains) in succession. This sequence of different Cnps is referred to as a “treatment cocktail”.

It is well known that certain conditions or disease states may be associated with each other. To treat such conditions using a treatment cocktail, the first step is to identify the Cnps corresponding to these components. Then, the Cnps are applied in sequence. While the order of Cnps may be interchangeable, certain preferred embodiments occur.

Based on studies showing that chronic pain is associated with depression, it is predicted that a treatment cocktail comprising an anti-depression Cnp pulse train followed by an analgesic Cnp pulse train followed by an anti-anxiety Cnp pulse train would be desirable. In particular, the preferred sequence is Cnp-1 then Cnp-2 then Cnp-3; the Cnp pulse trains each encompassing a typical duration of 5 min, 30 min, and 5 min respectively.

Eating disorders are known to be associated with depression and anxiety. A treatment cocktail comprising an anti-depression Cnp pulse train followed by an anti-anxiety Cnp pulse train shows promise for treating eating disorders (see Experiment 3, below). In particular, the preferred sequence is Cnp-1 then Cnp-3; the Cnp pulse trains each having a typical duration of 20-30 min each.

The advantages of the present invention are further illustrated by the following examples. The examples and their particular details set forth herein are presented for illustration only and should not be construed as a limitation on the claims of the present invention.

### Experiment 1

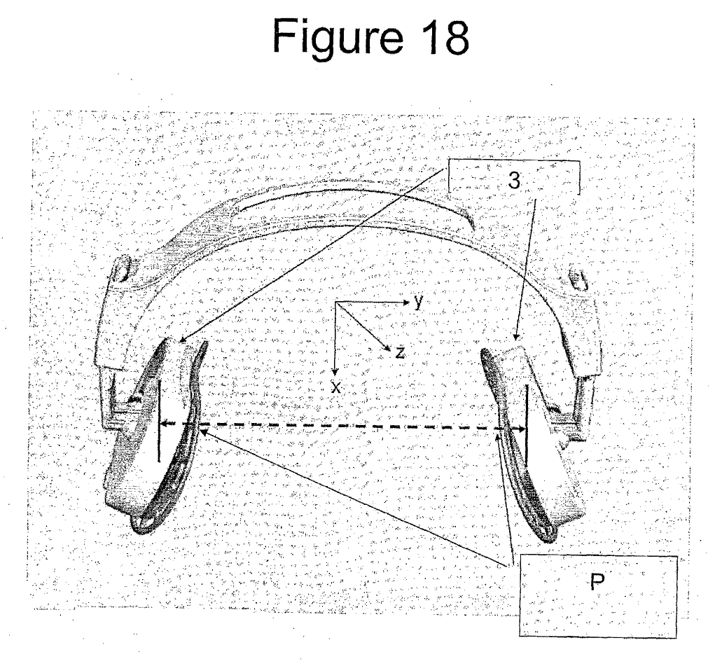

This experiment demonstrates the focusing of the magnetic field by the embodiment of the head-set with elliptical coils, as shown in FIG. 18. The dotted line in FIG. 18 indicates the mid-plane of the head-set, which is between the centers of the two coils 3. Also indicated is the orientation of coordinate axes relative to the coils, and the positions where magnetic flux density is the highest (P), at the centre of the coils on the coil enclosure surfaces.

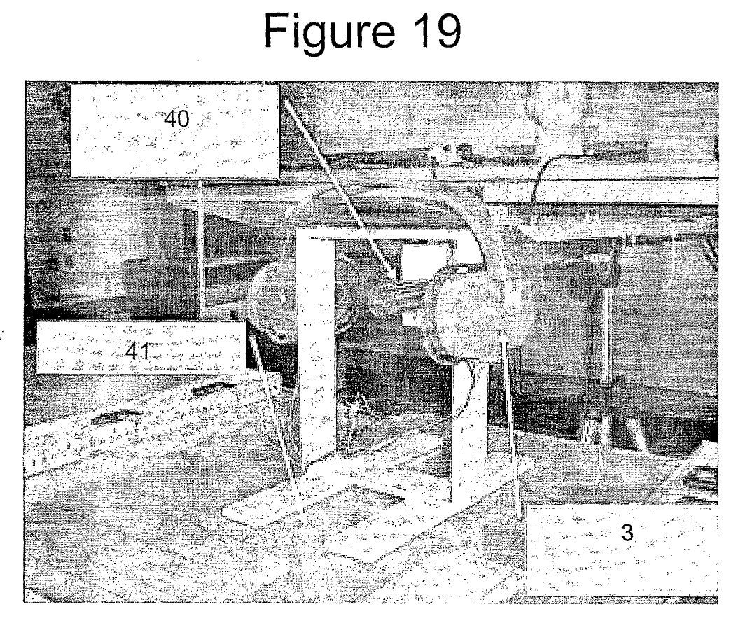

The measurement setup is illustrated in FIG. 19. Spatial profile of the magnetic field generated by the pair of coils 3 in the headset was measured using a sensitive fluxgate magnetometer 40. A spatial grid 41 was generated using an acrylic sheet to ensure repeatability and consistency of measurements. The magnetometer 40 was mounted on a tripod that was stabilized to remove vibrations. The headset was placed on a frame that could be easily moved along the grid.

At each position, peak flux density in all three orthogonal directions (Bx, By and Bz) were measured. Magnitude was then computed using the formula:

B=\sqrt{Bₓ²+B_{y}²+B_{z}²}

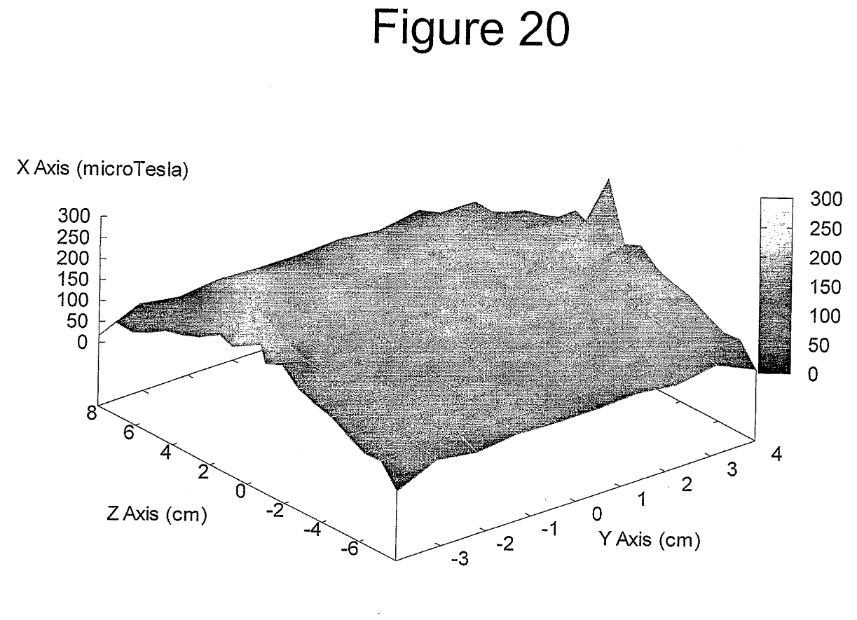

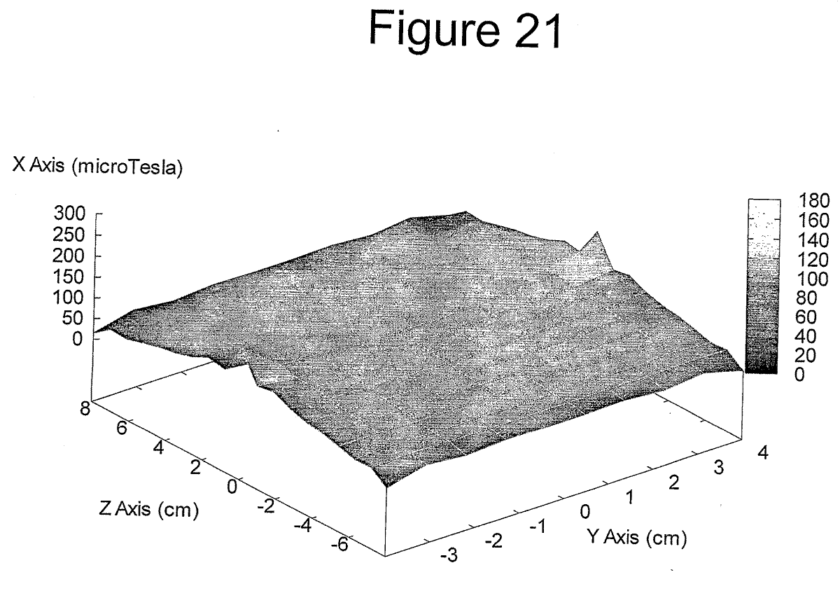

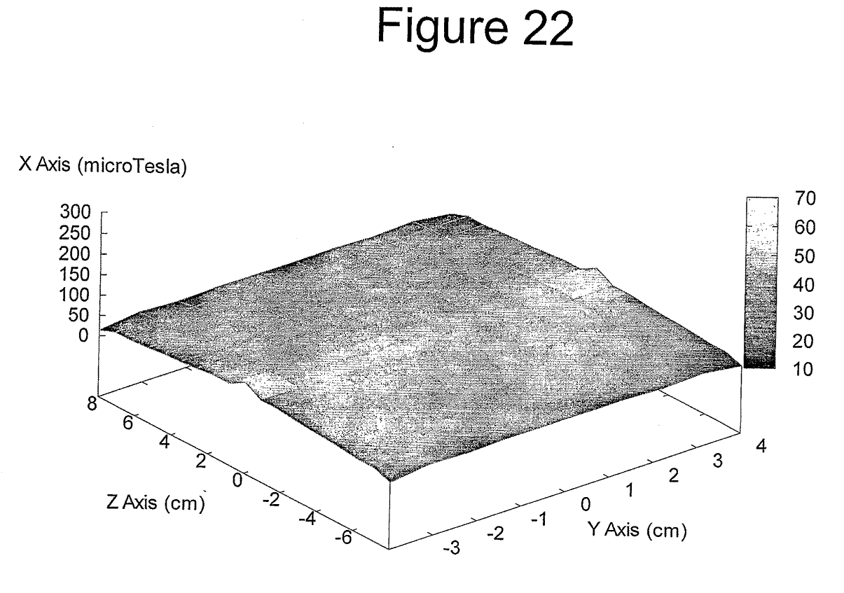

A three-dimensional approximation to the field profile was generated by sampling a horizontal 2D plane at three heights. First, the scanning plane was aligned with the mid-plane that dissected the coils into equal top and bottom halves (horizontal plane of symmetry). Results of this scan is presented in FIG. 20. A grayscale-bar is used to facilitate visual evaluation of the field profile and numerical values of this grayscale-scheme correspond to flux density measured in micro-Tesla. The same procedure was repeated for two parallel planes that are 2 cm and 4 cm below the mid-plane, and the results are presented in FIGS. 21 and 22, respectively. It is reasonable to deduce that the 2D profile will be identical above the horizontal mid-plane.

As evident from the above field profile figures, the flux density (B) decreases gradually along the vertical plane perpendicular to the Y axis that passes through Y=0. This is in sharp contrast with the rate of decay closer to the outer regions of the brain (vertical planes at Y=−4 and Y=4). Hence, the flux density is more uniform near the centre (deep-brain region), as desired.

### Experiment 2

This experiment illustrates use of the Cnp-2 pulse train in the treatment of pain. Relatively weak (100 μT to 400 μT) low frequency (<1000 Hz) specific pulsed magnetic fields (Cnps) have been shown to have an antinociceptive (analgesic) effect in the land snail, mice, and in healthy human volunteers measuring thermal sensory and pain thresholds. The analgesic effect of Cnp-2 therapy in patients with chronic pain from musculoskeletal causes was found to be comparable to that achieved in randomized controlled trials involving common prescription opioid analgesics.

### Method

The analgesic efficacy of a (±1400 μT head surface to ±35 μT deep brain) low frequency specific pulsed magnetic field (Cnp-2) was compared to that of common prescription opioid analgesics on similar patient populations. Cnp-2 was applied as a pulse train (FIG. 14). Data from randomized clinical trials involving morphine formulations, CR Codeine and CR Oxycodone were used. All three studies included patients with chronic pain from osteoarthritis. Data on analgesia achieved by Cnp-2 was gathered through St. Joseph's Health Care (London) Outpatient Pain Clinic in a double-blinded randomized placebo-controlled trial of patients with chronic musculoskeletal pain. In this study, subjects were loaned a Cnp-2 unit (either active or placebo) for 7 days, to be used at least twice per day for 40 minutes (compliance was monitored through a hidden data logger). Pain diaries were completed after each treatment, and during each day of a washout week following the trial (after the patients returned the Cnp-2 unit). In all studies, pain was measured before and after treatment using a visual analog scale (VAS) with higher numbers indicating greater pain. The analgesic effect of treatment and placebo was calculated as:

(VAS baseline−VAS post intervention)/VAS baseline×100%

The difference in analgesic effect between treatment and placebo is the net analgesic effect (NAE) for each modality and represents the amount of pain relief affected over and above placebo. For each opioid analgesic, the dosage used in the study was converted into an equivalent oral morphine dose using standard conversion ratios.

### Results

Comparison of Cnp-2 and Prescription Opioid Analgesia in Humans.

| | | Morphine | Net | | | |
|---|---|---|---|---|---|---|
| | | daily | analgesic | Number of | | |
| | | equivalent | effect | patients | | Follow- |
| | Dosage | dose | (NAE) | Rx | Placebo | up |
| Avinza | 30 mg | 30 | 12% | 46 | 50 | 28 d |
| | qam | | | | | |
| Avinza | 30 mg | 30 | 8% | 40 | 50 | 28 d |
| | qpm | | | | | |
| MS Contin | 15 mg | 30 | 8% | 48 | 50 | 28 d |
| | bid | | | | | |
| CR Oxycodone | 10 mg | 40 | 16% | 20 | 18 | 14 d |
| | bid | | | | | |
| CR Oxycodone | 20 mg | 80 | 23% | 25 | 18 | 14 d |
| | bid | | | | | |
| CR Oxycodone | 40 mg/ | 80 | 23% | 34 | 36 | 28 d |
| | day | | | | | |
| IR Oxycodone | 40 mg/ | 80 | 21% | 37 | 36 | 28 d |
| | day | | | | | |
| Cnp-2 (VAS > | 40 min | NA | 21% | 10 | 16 | 7 d |
| 4/10) | bid | | | | | |

### Discussion

Table 1 presents the analgesic efficacy of seven preparations of opioid analgesia. The results of treatment with Cnp-2 are presented on the bottom line of Table 1. Similar data analysis techniques (last observation carried forward) were used in all studies and the data presented for Cnp-2 represents those patients who had an intake VAS pain score of greater than or equal to 4 out of 10. This criterion for entry is common to the other studies. Comparison of the data reveals that the net analgesic effect of Cnp-2 is approximately equal to or greater than that achieved by opioid analgesia. Interestingly, the data also shows a growing cumulative effect of pain relief over the treatment week, and a significant retained effect more than 1 day after entering the ‘washout’ phase (the patients had turned in their Cnp-2 units) [data not shown].

### Experiment 3

Eating disorders are known to be associated with high levels of depression and anxiety. This experiment investigated the effect of a cocktail of the anti-depression and anti-anxiety Cnps (Cnp-1 and Cnp-3, respectively) on patients suffering from anxiety and eating disorders. A small pilot study of 8 patients with eating disorders, 5 patients with generalized anxiety and 5 control patients were prescreened with an initial interview, a psychological test battery, and routine lab work. They were each given Neuromodulation Therapy (NMT) two times per week for a one hour period for a total of 8 one hour sessions in a four week period. A follow-up interview, psychological test battery and lab work were repeated following the therapy. A portable source of pulsed magnetic field of 35 micro Tesla was used.

### Method

To evaluate effects of NMT, 8 patients with severe anorexia (average BMI of 16.8, SD=3.2), 5 patients suffering from severe anxiety (average BMI of 21.3, SD=4.2), and 5 normal controls (average BMI of 20.6, SD=2.4), were exposed to repeated semi-weekly sessions of NMT over a one month period. The NMT comprised sessions of Cnp-1 for 30 minutes followed by Cnp-3 for 30 minutes, for a total of 1 hour, twice weekly. At the onset of the study, the anorexia group significantly differed from normal controls on the majority of scales of the Eating Disorder Inventory, Version 2 (e.g., with respect to measures of body dissatisfaction, feeling ineffective, drive for thinness, social insecurity, and interpersonal mistrust). Both the anorexia and the anxiety patients had significantly higher scores than the control group on the Hamilton Anxiety Scale, Hamilton Depression Scale and Yale-Brown Obsessive Compulsive Scale. The three groups did not significantly differ with respect to their age and body height (p>0.05). All were females.

### Results

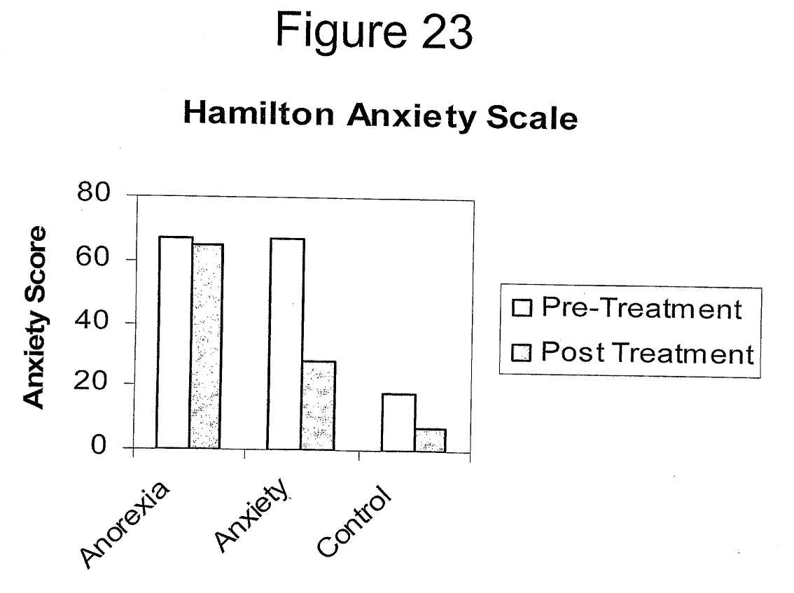

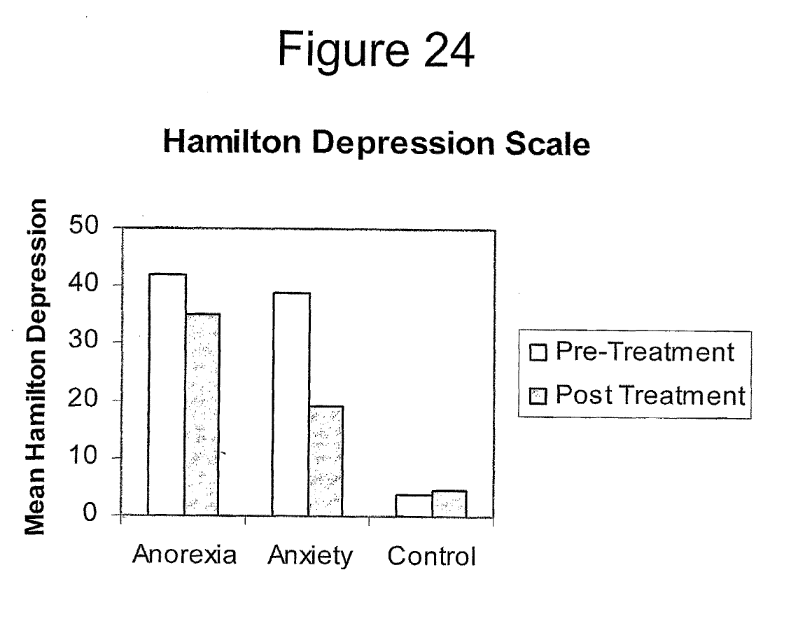

Following one month of NMT, there was a significant decrease, in the Hamilton Anxiety Scale and Hamilton Depression Scale, in the anxiety group (ANOVA, two-tailed, p<0.05), as shown in FIGS. 23 and 24. The anorexia group demonstrated a slight trend toward improvement on both scales.

There were no other statistically significant changes in any of the 3 groups. However, it should be noted that with very few exceptions, the trends in the anorexia group were in the expected direction (improvement in the score level).

### Discussion

There was a statistically significant improvement following one month of biweekly NMT in patients who suffered from anxiety: their anxiety and depression scores significantly decreased. Although the scores showed the same trend in a group of anorexia patients, the trend did not reach statistical significance possibly due to the small sample size and the short duration of NMT (one month only).

Although preferred embodiments of the invention have been described herein, it will be understood by those skilled in the art that variations may be made thereto without departing from the spirit of the invention or the scope of the appended claims.

TABLE 6

| Time (sec) | Amplitude |
|---|---|
| 0.560001915 | 0 |
| 0.5602455 | 0 |
| 0.560489085 | 0 |
| 0.56073267 | 0 |
| 0.560976255 | 0 |
| 0.56121984 | 0 |
| 0.561463425 | 0 |
| 0.56170701 | 0 |
| 0.561950595 | 0 |
| 0.56219418 | −0.173 |
| 0.562437765 | −0.368 |
| 0.56268135 | −0.754 |
| 0.562924935 | −1.536 |
| 0.56316852 | −2.317 |
| 0.563412105 | −3.248 |
| 0.56365569 | −4.224 |
| 0.563899275 | −5.405 |
| 0.56414286 | −7.358 |
| 0.564386445 | −9.31 |
| 0.56463003 | −4.897 |
| 0.564873615 | 2.912 |
| 0.5651172 | 9.895 |
| 0.565360785 | 8.919 |
| 0.56560437 | 7.942 |
| 0.565847955 | 7.488 |
| 0.56609154 | 7.488 |
| 0.566335125 | 7.487 |
| 0.56657871 | 6.533 |
| 0.566822295 | 5.556 |
| 0.56706588 | 4.99 |
| 0.567309465 | 4.99 |
| 0.56755305 | 4.989 |
| 0.567796635 | 4.148 |
| 0.56804022 | 3.173 |
| 0.568283805 | 2.494 |
| 0.56852739 | 2.494 |
| 0.568770975 | 2.494 |
| 0.56901456 | 1.766 |
| 0.569258145 | 0.792 |
| 0.56950173 | 0 |
| 0.569745315 | 0 |
| 0.5699889 | 0 |
| 0.570232485 | 0 |
| 0.57047607 | 0 |
| 0.570719655 | 0 |
| 0.57096324 | 0 |
| 0.571206825 | 0 |
| 0.57145041 | 0 |
| 0.571693995 | 0 |
| 0.57193758 | 0 |
| 0.572181165 | 0 |
| 0.57242475 | 0 |
| 0.572668335 | 0 |
| 0.57291192 | 0 |
| 0.573155505 | 0 |
| 0.57339909 | 0 |
| 0.573642675 | 0 |
| 0.57388626 | 0 |
| 0.574129845 | 0 |
| 0.57437343 | 0 |
| 0.574617015 | 2.816 |
| 0.5748606 | 6.704 |
| 0.575104185 | 9.948 |
| 0.57534777 | 9.946 |
| 0.575591355 | 9.945 |
| 0.57583494 | 7.581 |
| 0.576078525 | 3.693 |
| 0.57632211 | 0 |
| 0.576565695 | 0 |
| 0.57680928 | 0 |
| 0.577052865 | 0 |
| 0.57729645 | 0 |
| 0.577540035 | 0 |
| 0.57778362 | −0.181 |
| 0.578027205 | −0.375 |
| 0.57827079 | −0.789 |
| 0.578514375 | −1.565 |
| 0.57875796 | −2.341 |
| 0.579001545 | −3.276 |
| 0.57924513 | −4.246 |
| 0.579488715 | −5.467 |
| 0.5797323 | −7.406 |
| 0.579975885 | −9.345 |
| 0.58021947 | −4.478 |
| 0.580463055 | 3.28 |
| 0.58070664 | 9.78 |
| 0.580950225 | 8.81 |
| 0.58119381 | 7.839 |
| 0.581437395 | 7.437 |
| 0.58168098 | 7.436 |
| 0.581924565 | 7.408 |
| 0.58216815 | 6.438 |
| 0.582411735 | 5.469 |
| 0.58265532 | 4.955 |
| 0.582898905 | 4.954 |
| 0.58314249 | 4.953 |
| 0.583386075 | 4.07 |
| 0.58362966 | 3.101 |
| 0.583873245 | 2.476 |
| 0.58411683 | 2.475 |
| 0.584360415 | 2.475 |
| 0.584604 | 1.705 |
| 0.584847585 | 0.737 |
| 0.58509117 | 0 |
| 0.585334755 | 0 |
| 0.58557834 | 0 |
| 0.585821925 | 0 |
| 0.58606551 | 0 |
| 0.586309095 | 0 |
| 0.58655268 | 0 |
| 0.586796265 | 0 |
| 0.58703985 | 0 |
| 0.587283435 | 0 |
| 0.58752702 | 0 |
| 0.587770605 | 0 |
| 0.58801419 | 0 |
| 0.588257775 | 0 |
| 0.58850136 | 0 |
| 0.588744945 | 0 |
| 0.58898853 | 0 |
| 0.589232115 | 0 |
| 0.5894757 | 0 |
| 0.589719285 | 0 |
| 0.58996287 | 0 |
| 0.590206455 | 2.983 |
| 0.59045004 | 6.838 |
| 0.590693625 | 9.86 |
| 0.59093721 | 9.858 |
| 0.591180795 | 9.857 |
| 0.59142438 | 7.321 |
| 0.591667965 | 3.468 |
| 0.59191155 | 0 |
| 0.592155135 | 0 |
| 0.59239872 | 0 |
| 0.592642305 | 0 |
| 0.59288589 | 0 |
| 0.593129475 | 0 |
| 0.59337306 | −0.189 |
| 0.593616645 | −0.382 |
| 0.59386023 | −0.819 |
| 0.594103815 | −1.588 |
| 0.5943474 | −2.357 |
| 0.594590985 | −3.292 |
| 0.59483457 | −4.252 |
| 0.595078155 | −5.51 |
| 0.59532174 | −7.43 |
| 0.595565325 | −9.349 |
| 0.59580891 | −4.05 |
| 0.596052495 | 3.631 |
| 0.59629608 | 9.634 |
| 0.596539665 | 8.673 |
| 0.59678325 | 7.712 |
| 0.597026835 | 7.361 |
| 0.59727042 | 7.36 |
| 0.597514005 | 7.284 |
| 0.59775759 | 6.324 |
| 0.598001175 | 5.364 |
| 0.59824476 | 4.903 |
| 0.598488345 | 4.902 |
| 0.59873193 | 4.901 |
| 0.598975515 | 3.979 |
| 0.5992191 | 3.021 |
| 0.599462685 | 2.449 |
| 0.59970627 | 2.449 |
| 0.599949855 | 2.448 |
| 0.60019344 | 1.638 |
| 0.600437025 | 0.681 |
| 0.60068061 | 0 |
| 0.600924195 | 0 |
| 0.60116778 | 0 |
| 0.601411365 | 0 |
| 0.60165495 | 0 |
| 0.601898535 | 0 |
| 0.60214212 | 0 |
| 0.602385705 | 0 |
| 0.60262929 | 0 |
| 0.602872875 | 0 |
| 0.60311646 | 0 |
| 0.603360045 | 0 |
| 0.60360363 | 0 |
| 0.603847215 | 0 |
| 0.6040908 | 0 |
| 0.604334385 | 0 |
| 0.60457797 | 0 |
| 0.604821555 | 0 |
| 0.60506514 | 0 |
| 0.605308725 | 0 |
| 0.60555231 | 0 |
| 0.605795895 | 3.138 |
| 0.60603948 | 6.946 |
| 0.606283065 | 9.741 |
| 0.60652665 | 9.739 |
| 0.606770235 | 9.737 |
| 0.60701382 | 7.041 |
| 0.607257405 | 3.235 |
| 0.60750099 | 0 |
| 0.607744575 | 0 |
| 0.60798816 | 0 |
| 0.608231745 | 0 |
| 0.60847533 | 0 |
| 0.608718915 | −0.006 |
| 0.6089625 | −0.196 |
| 0.609206085 | −0.386 |
| 0.60944967 | −0.847 |
| 0.609693255 | −1.606 |
| 0.60993684 | −2.365 |
| 0.610180425 | −3.297 |
| 0.61042401 | −4.245 |
| 0.610667595 | −5.534 |
| 0.61091118 | −7.428 |
| 0.611154765 | −9.322 |
| 0.61139835 | −3.618 |
| 0.611641935 | 3.961 |
| 0.61188552 | 9.46 |
| 0.612129105 | 8.511 |
| 0.61237269 | 7.562 |
| 0.612616275 | 7.263 |
| 0.61285986 | 7.262 |
| 0.613103445 | 7.139 |
| 0.61334703 | 6.192 |
| 0.613590615 | 5.244 |
| 0.6138342 | 4.836 |
| 0.614077785 | 4.835 |
| 0.61432137 | 4.823 |
| 0.614564955 | 3.877 |
| 0.61480854 | 2.932 |
| 0.615052125 | 2.415 |
| 0.61529571 | 2.415 |
| 0.615539295 | 2.414 |
| 0.61578288 | 1.569 |
| 0.616026465 | 0.625 |
| 0.61627005 | 0 |
| 0.616513635 | 0 |
| 0.61675722 | 0 |
| 0.617000805 | 0 |
| 0.61724439 | 0 |
| 0.617487975 | 0 |
| 0.61773156 | 0 |
| 0.617975145 | 0 |
| 0.61821873 | 0 |
| 0.618462315 | 0 |
| 0.6187059 | 0 |
| 0.618949485 | 0 |
| 0.61919307 | 0 |
| 0.619436655 | 0 |
| 0.61968024 | 0 |
| 0.619923825 | 0 |
| 0.62016741 | 0 |
| 0.620410995 | 0 |
| 0.62065458 | 0 |
| 0.620898165 | 0 |
| 0.62114175 | 0 |
| 0.621385335 | 3.278 |
| 0.62162892 | 7.029 |
| 0.621872505 | 9.593 |
| 0.62211609 | 9.591 |
| 0.622359675 | 9.588 |
| 0.62260326 | 6.747 |
| 0.622846845 | 2.999 |
| 0.62309043 | 0 |
| 0.623334015 | 0 |
| 0.6235776 | 0 |
| 0.623821185 | 0 |
| 0.62406477 | 0 |
| 0.624308355 | 0 |
| 0.62455194 | 0 |
| 0.624795525 | 0 |
| 0.62503911 | 0 |
| 0.625282695 | 0 |
| 0.62552628 | 0 |
| 0.625769865 | 0 |
| 0.62601345 | 0 |
| 0.626257035 | 0 |
| 0.62650062 | 0 |
| 0.626744205 | 0 |
| 0.62698779 | 0 |
| 0.627231375 | 0 |
| 0.62747496 | 0 |
| 0.627718545 | 0 |
| 0.62796213 | 0 |
| 0.628205715 | 0 |
| 0.6284493 | 0 |
| 0.628692885 | 0 |
| 0.62893647 | 0 |
| 0.629180055 | 0 |
| 0.62942364 | 0 |
| 0.629667225 | 0 |
| 0.62991081 | 0 |
| 0.630154395 | 0 |
| 0.63039798 | 0 |
| 0.630641565 | 0 |
| 0.63088515 | 0 |
| 0.631128735 | 0 |
| 0.63137232 | 0 |
| 0.631615905 | 0 |
| 0.63185949 | 0 |
| 0.632103075 | 0 |
| 0.63234666 | 0 |
| 0.632590245 | 0 |
| 0.63283383 | 0 |
| 0.633077415 | 0 |
| 0.633321 | 0 |
| 0.633564585 | 0 |
| 0.63380817 | 0 |
| 0.634051755 | 0 |
| 0.63429534 | 0 |
| 0.634538925 | 0 |
| 0.63478251 | 0 |
| 0.635026095 | 0 |
| 0.63526968 | 0 |
| 0.635513265 | 0 |
| 0.63575685 | 0 |
| 0.636000435 | 0 |
| 0.63624402 | 0 |
| 0.636487605 | 0 |
| 0.63673119 | 0 |
| 0.636974775 | 0 |
| 0.63721836 | 0 |
| 0.637461945 | 0 |
| 0.63770553 | 0 |
| 0.637949115 | 0 |
| 0.6381927 | 0 |
| 0.638436285 | 0 |
| 0.63867987 | 0 |
| 0.638923455 | 0 |
| 0.63916704 | 0 |
| 0.639410625 | 0 |
| 0.63965421 | 0 |
| 0.639897795 | −0.025 |
| 0.64014138 | −0.208 |
| 0.640384965 | −0.391 |
| 0.64062855 | −0.891 |
| 0.640872135 | −1.624 |
| 0.64111572 | −2.36 |
| 0.641359305 | −3.275 |
| 0.64160289 | −4.19 |
| 0.641846475 | −5.525 |
| 0.64209006 | −7.353 |
| 0.642333645 | −9.181 |
| 0.64257723 | −2.762 |
| 0.642820815 | 4.554 |
| 0.6430644 | 9.037 |
| 0.643307985 | 8.121 |
| 0.64355157 | 7.205 |
| 0.643795155 | 7.007 |
| 0.64403874 | 7.005 |
| 0.644282325 | 6.794 |
| 0.64452591 | 5.88 |
| 0.644769495 | 4.966 |
| 0.64501308 | 4.664 |
| 0.645256665 | 4.662 |
| 0.64550025 | 4.559 |
| 0.645743835 | 3.647 |
| 0.64598742 | 2.735 |
| 0.646231005 | 2.328 |
| 0.64647459 | 2.327 |
| 0.646718175 | 2.326 |
| 0.64696176 | 1.421 |
| 0.647205345 | 0.511 |
| 0.64744893 | 0 |
| 0.647692515 | 0 |
| 0.6479361 | 0 |
| 0.648179685 | 0 |
| 0.64842327 | 0 |
| 0.648666855 | 0 |
| 0.64891044 | 0 |
| 0.649154025 | 0 |
| 0.64939761 | 0 |
| 0.649641195 | 0 |
| 0.64988478 | 0 |
| 0.650128365 | 0 |
| 0.65037195 | 0 |
| 0.650615535 | 0 |
| 0.65085912 | 0 |
| 0.651102705 | 0 |
| 0.65134629 | 0 |
| 0.651589875 | 0 |
| 0.65183346 | 0 |
| 0.652077045 | 0 |
| 0.65232063 | 0 |
| 0.652564215 | 3.512 |
| 0.6528078 | 7.119 |
| 0.653051385 | 9.223 |
| 0.65329497 | 9.22 |
| 0.653538555 | 9.217 |
| 0.65378214 | 6.125 |
| 0.654025725 | 2.522 |
| 0.65426931 | 0 |
| 0.654512895 | 0 |
| 0.65475648 | 0 |
| 0.655000065 | 0 |
| 0.65524365 | 0 |
| 0.655487235 | −0.033 |
| 0.65573082 | −0.213 |
| 0.655974405 | −0.392 |
| 0.65621799 | −0.908 |
| 0.656461575 | −1.625 |
| 0.65670516 | −2.355 |
| 0.656948745 | −3.25 |
| 0.65719233 | −4.145 |
| 0.657435915 | −5.495 |
| 0.6576795 | −7.283 |
| 0.657923085 | −9.072 |
| 0.65816667 | −2.344 |
| 0.658410255 | 4.812 |
| 0.65865384 | 8.796 |
| 0.658897425 | 7.899 |
| 0.65914101 | 7.003 |
| 0.659384595 | 6.853 |
| 0.65962818 | 6.851 |
| 0.659871765 | 6.6 |
| 0.66011535 | 5.706 |
| 0.660358935 | 4.812 |
| 0.66060252 | 4.561 |
| 0.660846105 | 4.559 |
| 0.66108969 | 4.413 |
| 0.661333275 | 3.521 |
| 0.66157686 | 2.63 |
| 0.661820445 | 2.276 |
| 0.66206403 | 2.275 |
| 0.662307615 | 2.233 |
| 0.6625512 | 1.344 |
| 0.662794785 | 0.455 |
| 0.66303837 | 0 |
| 0.663281955 | 0 |
| 0.66352554 | 0 |
| 0.663769125 | 0 |
| 0.66401271 | 0 |
| 0.664256295 | 0 |
| 0.66449988 | 0 |
| 0.664743465 | 0 |
| 0.66498705 | 0 |
| 0.665230635 | 0 |
| 0.66547422 | 0 |
| 0.665717805 | 0 |
| 0.66596139 | 0 |
| 0.666204975 | 0 |
| 0.66644856 | 0 |
| 0.666692145 | 0 |
| 0.66693573 | 0 |
| 0.667179315 | 0 |
| 0.6674229 | 0 |
| 0.667666485 | 0 |
| 0.66791007 | 0.085 |
| 0.668153655 | 3.607 |
| 0.66839724 | 7.129 |
| 0.668640825 | 9.008 |
| 0.66888441 | 9.004 |
| 0.669127995 | 9.001 |
| 0.66937158 | 5.806 |
| 0.669615165 | 2.287 |
| 0.66985875 | 0 |
| 0.670102335 | 0 |
| 0.67034592 | 0 |
| 0.670589505 | 0 |
| 0.67083309 | 0 |
| 0.671076675 | −0.041 |
| 0.67132026 | −0.216 |
| 0.671563845 | −0.391 |
| 0.67180743 | −0.921 |
| 0.672051015 | −1.621 |
| 0.6722946 | −2.342 |
| 0.672538185 | −3.215 |
| 0.67278177 | −4.089 |
| 0.673025355 | −5.45 |
| 0.67326894 | −7.195 |
| 0.673512525 | −8.923 |
| 0.67375611 | −1.939 |
| 0.673999695 | 5.045 |
| 0.67424328 | 8.539 |
| 0.674486865 | 7.664 |
| 0.67473045 | 6.789 |
| 0.674974035 | 6.687 |
| 0.67521762 | 6.684 |
| 0.675461205 | 6.396 |
| 0.67570479 | 5.523 |
| 0.675948375 | 4.651 |
| 0.67619196 | 4.449 |
| 0.676435545 | 4.447 |
| 0.67667913 | 4.261 |
| 0.676922715 | 3.391 |
| 0.6771663 | 2.521 |
| 0.677409885 | 2.22 |
| 0.67765347 | 2.219 |
| 0.677897055 | 2.135 |
| 0.67814064 | 1.268 |
| 0.678384225 | 0.401 |
| 0.67862781 | 0 |
| 0.678871395 | 0 |
| 0.67911498 | 0 |
| 0.679358565 | 0 |
| 0.67960215 | 0 |
| 0.679845735 | 0 |
| 0.68008932 | 0 |
| 0.680332905 | 0 |
| 0.68057649 | 0 |
| 0.680820075 | 0 |
| 0.68106366 | 0 |
| 0.681307245 | 0 |
| 0.68155083 | 0 |
| 0.681794415 | 0 |
| 0.682038 | 0 |
| 0.682281585 | 0 |
| 0.68252517 | 0 |
| 0.682768755 | 0 |
| 0.68301234 | 0 |
| 0.683255925 | 0 |
| 0.68349951 | 0.254 |
| 0.683743095 | 3.686 |
| 0.68398668 | 7.118 |
| 0.684230265 | 8.777 |
| 0.68447385 | 8.773 |
| 0.684717435 | 8.77 |
| 0.68496102 | 5.485 |
| 0.685204605 | 2.057 |
| 0.68544819 | 0 |
| 0.685691775 | 0 |
| 0.68593536 | 0 |
| 0.686178945 | 0 |
| 0.68642253 | 0 |
| 0.686666115 | 0 |
| 0.6869097 | 0 |
| 0.687153285 | 0 |
| 0.68739687 | 0 |
| 0.687640455 | 0 |
| 0.68788404 | 0 |
| 0.688127625 | 0 |
| 0.68837121 | 0 |
| 0.688614795 | 0 |
| 0.68885838 | 0 |
| 0.689101965 | 0 |
| 0.68934555 | 0 |
| 0.689589135 | 0 |
| 0.68983272 | 0 |
| 0.690076305 | 0 |
| 0.69031989 | 0 |
| 0.690563475 | 0 |
| 0.69080706 | 0 |
| 0.691050645 | 0 |
| 0.69129423 | 0 |
| 0.691537815 | 0 |
| 0.6917814 | 0 |
| 0.692024985 | 0 |
| 0.69226857 | 0 |
| 0.692512155 | 0 |
| 0.69275574 | 0 |
| 0.692999325 | 0 |
| 0.69324291 | 0 |
| 0.693486495 | 0 |
| 0.69373008 | 0 |
| 0.693973665 | 0 |
| 0.69421725 | 0 |
| 0.694460835 | 0 |
| 0.69470442 | 0 |
| 0.694948005 | 0 |
| 0.69519159 | 0 |
| 0.695435175 | 0 |
| 0.69567876 | 0 |
| 0.695922345 | 0 |
| 0.69616593 | 0 |
| 0.696409515 | 0 |
| 0.6966531 | 0 |
| 0.696896685 | 0 |
| 0.69714027 | 0 |
| 0.697383855 | 0 |
| 0.69762744 | 0 |
| 0.697871025 | 0 |
| 0.69811461 | 0 |
| 0.698358195 | 0 |
| 0.69860178 | 0 |
| 0.698845365 | 0 |
| 0.69908895 | 0 |
| 0.699332535 | 0 |
| 0.69957612 | 0 |
| 0.699819705 | 0 |
| 0.70006329 | 0 |
| 0.700306875 | 0 |
| 0.70055046 | 0 |
| 0.700794045 | 0 |
| 0.70103763 | 0 |
| 0.701281215 | 0 |
| 0.7015248 | 0 |
| 0.701768385 | 0 |
| 0.70201197 | 0 |
| 0.702255555 | 0 |
| 0.70249914 | 0 |
| 0.702742725 | 0 |
| 0.70298631 | 0 |
| 0.703229895 | 0 |
| 0.70347348 | 0 |
| 0.703717065 | 0 |
| 0.70396065 | 0 |
| 0.704204235 | 0 |
| 0.70444782 | 0 |
| 0.704691405 | 0 |
| 0.70493499 | 0 |
| 0.705178575 | 0 |
| 0.70542216 | 0 |
| 0.705665745 | 0 |
| 0.70590933 | 0 |
| 0.706152915 | 0 |
| 0.7063965 | 0 |
| 0.706640085 | 0 |
| 0.70688367 | 0 |
| 0.707127255 | 0 |
| 0.70737084 | 0 |
| 0.707614425 | 0 |
| 0.70785801 | 0 |
| 0.708101595 | 0 |
| 0.70834518 | 0 |
| 0.708588765 | 0 |
| 0.70883235 | 0 |
| 0.709075935 | 0 |
| 0.70931952 | 0 |
| 0.709563105 | 0 |
| 0.70980669 | 0 |
| 0.710050275 | 0 |
| 0.71029386 | 0 |
| 0.710537445 | 0 |
| 0.71078103 | 0 |
| 0.711024615 | 0 |
| 0.7112682 | 0 |
| 0.711511785 | 0 |
| 0.71175537 | 0 |
| 0.711998955 | 0 |
| 0.71224254 | 0 |
| 0.712486125 | 0 |
| 0.71272971 | 0 |
| 0.712973295 | 0 |
| 0.71321688 | 0 |
| 0.713460465 | 0 |
| 0.71370405 | 0 |
| 0.713947635 | 0 |
| 0.71419122 | 0 |
| 0.714434805 | 0 |
| 0.71467839 | 0 |
| 0.714921975 | 0 |
| 0.71516556 | 0 |
| 0.715409145 | 0 |
| 0.71565273 | 0 |
| 0.715896315 | 0 |
| 0.7361399 | 0 |
| 0.716383485 | 0 |
| 0.71662707 | 0 |
| 0.716870655 | 0 |
| 0.71711424 | 0 |
| 0.717357825 | 0 |
| 0.71760141 | 0 |
| 0.717844995 | −0.062 |
| 0.71808858 | −0.223 |
| 0.718332165 | −0.384 |
| 0.71857575 | −0.944 |
| 0.718819335 | −1.587 |
| 0.71906292 | −2.273 |
| 0.719306505 | −3.076 |
| 0.71955009 | −3.878 |
| 0.719793675 | −5.249 |
| 0.72003726 | −6.852 |
| 0.720280845 | −7.236 |
| 0.72052443 | −0.82 |
| 0.720768015 | 5.597 |
| 0.7210116 | 7.724 |
| 0.721255185 | 6.919 |
| 0.72149877 | 6.144 |
| 0.721742355 | 6.141 |
| 0.72198594 | 6.138 |
| 0.722229525 | 5.753 |
| 0.72247311 | 4.952 |
| 0.722716695 | 4.15 |
| 0.72296028 | 4.084 |
| 0.723203865 | 4.082 |
| 0.72344745 | 3.792 |
| 0.723691035 | 2.993 |
| 0.72393462 | 2.195 |
| 0.724178205 | 2.037 |
| 0.72442179 | 2.036 |
| 0.724665375 | 1.84 |
| 0.72490896 | 1.044 |
| 0.725152545 | 0.248 |
| 0.72539613 | 0 |
| 0.725639715 | 0 |
| 0.7258833 | 0 |
| 0.726126885 | 0 |
| 0.72637047 | 0 |
| 0.726614055 | 0 |
| 0.72685764 | 0 |
| 0.727101225 | 0 |
| 0.72734481 | 0 |
| 0.727588395 | 0 |
| 0.72783198 | 0 |
| 0.728075565 | 0 |
| 0.72831915 | 0 |
| 0.728562735 | 0 |
| 0.72880632 | 0 |
| 0.729049905 | 0 |
| 0.72929349 | 0 |
| 0.729537075 | 0 |
| 0.72978066 | 0 |
| 0.730024245 | 0 |
| 0.73026783 | 0.704 |
| 0.730511415 | 3.849 |
| 0.730755 | 6.993 |
| 0.730998585 | 8.04 |
| 0.73124217 | 8.037 |
| 0.731485755 | 7.694 |
| 0.73172934 | 4.554 |
| 0.731972925 | 1.413 |
| 0.73221651 | 0 |
| 0.732460095 | 0 |
| 0.73270368 | 0 |
| 0.732947265 | 0 |
| 0.73319085 | 0 |
| 0.733434435 | −0.068 |
| 0.73367802 | −0.224 |
| 0.733921605 | −0.38 |
| 0.73416519 | −0.946 |
| 0.734408775 | −1.57 |
| 0.73465236 | −2.243 |
| 0.734895945 | −3.022 |
| 0.73513953 | −3.8 |
| 0.735383115 | −5.169 |
| 0.7356267 | −6.723 |
| 0.735870285 | −6.707 |
| 0.73611387 | −0.484 |
| 0.736357455 | 5.739 |
| 0.73660104 | 7.452 |
| 0.736844625 | 6.672 |
| 0.73708821 | 5.958 |
| 0.737331795 | 5.955 |
| 0.73757538 | 5.952 |
| 0.737818965 | 5.541 |
| 0.73806255 | 4.763 |
| 0.738306135 | 3.986 |
| 0.73854972 | 3.961 |
| 0.738793305 | 3.959 |
| 0.73903689 | 3.638 |
| 0.739280475 | 2.864 |
| 0.73952406 | 2.089 |
| 0.739767645 | 1.975 |
| 0.74001123 | 1.975 |
| 0.740254815 | 1.745 |
| 0.7404984 | 0.974 |
| 0.740741985 | 0.202 |
| 0.74098557 | 0 |
| 0.741229155 | 0 |
| 0.74147274 | 0 |
| 0.741716325 | 0 |
| 0.74195991 | 0 |
| 0.742203495 | 0 |
| 0.74244708 | 0 |
| 0.742690665 | 0 |
| 0.74293425 | 0 |
| 0.743177835 | 0 |
| 0.74342142 | 0 |
| 0.743665005 | 0 |
| 0.74390859 | 0 |
| 0.744152175 | 0 |
| 0.74439576 | 0 |
| 0.744639345 | 0 |
| 0.74488293 | 0 |
| 0.745126515 | 0 |
| 0.7453701 | 0 |
| 0.745613685 | 0 |
| 0.74585727 | 0.835 |
| 0.746100855 | 3.884 |
| 0.74634444 | 6.932 |
| 0.746588025 | 7.796 |
| 0.74683161 | 7.792 |
| 0.747075195 | 7.308 |
| 0.74731878 | 4.263 |
| 0.747562365 | 1.218 |
| 0.74780595 | 0 |
| 0.748049535 | 0 |
| 0.74829312 | 0 |
| 0.748536705 | 0 |
| 0.74878029 | 0 |
| 0.749023875 | 0 |
| 0.74926746 | 0 |
| 0.749511045 | 0 |
| 0.74975463 | 0 |
| 0.749998215 | 0 |
| 0.7502418 | 0 |
| 0.750485385 | 0 |
| 0.75072897 | 0 |
| 0.750972555 | 0 |
| 0.75121614 | 0 |
| 0.751459725 | 0 |
| 0.75170331 | 0 |
| 0.751946895 | 0 |
| 0.75219048 | 0 |
| 0.752434065 | 0 |
| 0.75267765 | 0 |
| 0.752921235 | 0 |
| 0.75316482 | 0 |
| 0.753408405 | 0 |
| 0.75365199 | 0 |
| 0.753895575 | 0 |
| 0.75413916 | 0 |
| 0.754382745 | 0 |
| 0.75462633 | 0 |
| 0.754869915 | 0 |
| 0.7551135 | 0 |
| 0.755357085 | 0 |
| 0.75560067 | 0 |
| 0.755844255 | 0 |
| 0.75608784 | 0 |
| 0.756331425 | 0 |
| 0.75657501 | 0 |
| 0.756818595 | 0 |
| 0.75706218 | 0 |
| 0.757305765 | 0 |
| 0.75754935 | 0 |
| 0.757792935 | 0 |
| 0.75803652 | 0 |
| 0.758280105 | 0 |
| 0.75852369 | 0 |
| 0.758767275 | 0 |
| 0.75901086 | 0 |
| 0.759254445 | 0 |
| 0.75949803 | 0 |
| 0.759741615 | 0 |
| 0.7599852 | 0 |
| 0.760228785 | 0 |
| 0.76047237 | 0 |
| 0.760715955 | 0 |
| 0.76095954 | 0 |
| 0.761203125 | 0 |
| 0.76144671 | 0 |
| 0.761690295 | 0 |
| 0.76193388 | 0 |
| 0.762177465 | 0 |
| 0.76242105 | 0 |
| 0.762664635 | 0 |
| 0.76290822 | 0 |
| 0.763151805 | 0 |
| 0.76339539 | 0 |
| 0.763638975 | 0 |
| 0.76388256 | 0 |
| 0.764126145 | 0 |
| 0.76436973 | 0 |
| 0.764613315 | 0 |
| 0.7648569 | 0 |
| 0.765100485 | 0 |
| 0.76534407 | 0 |
| 0.765587655 | 0 |
| 0.76583124 | 0 |
| 0.766074825 | 0 |
| 0.76631841 | 0 |
| 0.766561995 | 0 |
| 0.76680558 | 0 |
| 0.767049165 | 0 |
| 0.76729275 | 0 |
| 0.767536335 | 0 |
| 0.76777992 | 0 |
| 0.768023505 | 0 |
| 0.76826709 | 0 |
| 0.768510675 | 0 |
| 0.76875426 | 0 |
| 0.768997845 | 0 |
| 0.76924143 | 0 |
| 0.769485015 | 0 |
| 0.7697286 | 0 |
| 0.769972185 | 0 |
| 0.77021577 | 0 |
| 0.770459355 | 0 |
| 0.77070294 | 0 |
| 0.770946525 | 0 |
| 0.77119011 | 0 |
| 0.771433695 | 0 |
| 0.77167728 | 0 |
| 0.771920865 | 0 |
| 0.77216445 | 0 |
| 0.772408035 | 0 |
| 0.77265162 | 0 |
| 0.772895205 | 0 |
| 0.77313879 | 0 |
| 0.773382375 | 0 |
| 0.77362596 | 0 |
| 0.773869545 | 0 |
| 0.77411313 | 0 |
| 0.774356715 | 0 |
| 0.7746003 | 0 |
| 0.774843885 | 0 |
| 0.77508747 | 0 |
| 0.775331055 | 0 |
| 0.77557464 | 0 |
| 0.775818225 | 0 |
| 0.77606181 | 0 |
| 0.776305395 | 0 |
| 0.77654898 | 0 |
| 0.776792565 | 0 |
| 0.77703615 | 0 |
| 0.777279735 | 0 |
| 0.77752332 | 0 |
| 0.777766905 | 0 |
| 0.77801049 | 0 |
| 0.778254075 | 0 |
| 0.77849766 | 0 |
| 0.778741245 | 0 |
| 0.77898483 | 0 |
| 0.779228415 | 0 |
| 0.779472 | 0 |
| 0.779715585 | 0 |
| 0.77995917 | 0 |
| 0.780202755 | 0 |
| 0.78044634 | 0 |
| 0.780689925 | 0 |
| 0.78093351 | 0 |
| 0.781177095 | 0 |
| 0.78142068 | 0 |
| 0.781664265 | 0 |
| 0.78190785 | 0 |
| 0.782151435 | 0 |
| 0.78239502 | 0 |
| 0.782638605 | 0 |
| 0.78288219 | 0 |
| 0.783125775 | 0 |
| 0.78336936 | 0 |
| 0.783612945 | 0 |
| 0.78385653 | 0 |
| 0.784100115 | 0 |
| 0.7843437 | 0 |
| 0.784587285 | 0 |
| 0.78483087 | 0 |
| 0.785074455 | 0 |
| 0.78531804 | 0 |
| 0.785561625 | 0 |
| 0.78580521 | 0 |
| 0.786048795 | 0 |
| 0.78629238 | 0 |
| 0.786535965 | 0 |
| 0.78677955 | 0 |
| 0.787023135 | 0 |
| 0.78726672 | 0 |
| 0.787510305 | 0 |
| 0.78775389 | 0 |
| 0.787997475 | 0 |
| 0.78824106 | 0 |
| 0.788484645 | 0 |
| 0.78872823 | 0 |
| 0.788971815 | 0 |
| 0.7892154 | 0 |
| 0.789458985 | 0 |
| 0.78970257 | 0 |
| 0.789946155 | 0 |
| 0.79018974 | 0 |
| 0.790433325 | 0 |
| 0.79067691 | 0 |
| 0.790920495 | 0 |
| 0.79116408 | 0 |
| 0.791407665 | 0 |
| 0.79165125 | 0 |
| 0.791894835 | 0 |
| 0.79213842 | 0 |
| 0.792382005 | 0 |
| 0.79262559 | 0 |
| 0.792869175 | 0 |
| 0.79311276 | 0 |
| 0.793356345 | 0 |
| 0.79359993 | 0 |
| 0.793843515 | 0 |
| 0.7940871 | 0 |
| 0.794330685 | 0 |
| 0.79457427 | 0 |
| 0.794817855 | 0 |
| 0.79506144 | 0 |
| 0.795305025 | 0 |
| 0.79554861 | 0 |
| 0.795792195 | 0 |
| 0.79603578 | 0 |
| 0.796279365 | 0 |
| 0.79652295 | 0 |
| 0.796766535 | 0 |
| 0.79701012 | 0 |
| 0.797253705 | 0 |
| 0.79749729 | 0 |
| 0.797740875 | 0 |
| 0.79798446 | 0 |
| 0.798228045 | 0 |
| 0.79847163 | 0 |
| 0.798715215 | 0 |
| 0.7989588 | 0 |
| 0.799202385 | 0 |
| 0.79944597 | 0 |
| 0.799689555 | 0 |
| 0.79993314 | 0 |
| 0.800176725 | 0 |
| 0.80042031 | 0 |
| 0.800663895 | 0 |
| 0.80090748 | 0 |
| 0.801151065 | 0 |
| 0.80139465 | 0 |
| 0.801638235 | 0 |
| 0.80188182 | 0 |
| 0.802125405 | 0 |
| 0.80236899 | 0 |
| 0.802612575 | 0 |
| 0.80285616 | 0 |
| 0.803099745 | 0 |
| 0.80334333 | 0 |
| 0.803586915 | 0 |
| 0.8038305 | 0 |
| 0.804074085 | 0 |
| 0.80431767 | 0 |
| 0.804561255 | 0 |
| 0.80480484 | 0 |
| 0.805048425 | 0 |
| 0.80529201 | 0 |
| 0.805535595 | 0 |
| 0.80577918 | 0 |
| 0.806022765 | 0 |
| 0.80626635 | 0 |
| 0.806509935 | 0 |
| 0.80675352 | 0 |
| 0.806997105 | 0 |
| 0.80724069 | 0 |
| 0.807484275 | 0 |
| 0.80772786 | 0 |
| 0.807971445 | 0 |
| 0.80821503 | 0 |
| 0.808458615 | 0 |
| 0.8087022 | 0 |
| 0.808945785 | 0 |
| 0.80918937 | 0 |
| 0.809432955 | 0 |
| 0.80967654 | 0 |
| 0.809920125 | 0 |
| 0.81016371 | 0 |
| 0.810407295 | 0 |
| 0.81065088 | 0 |
| 0.810894465 | 0 |
| 0.81113805 | 0 |
| 0.811381635 | −0.092 |
| 0.81162522 | −0.227 |
| 0.811868805 | −0.413 |
| 0.81211239 | −0.952 |
| 0.812355975 | −1.492 |
| 0.81259956 | −2.107 |
| 0.812843145 | −2.78 |
| 0.81308673 | −3.459 |
| 0.813330315 | −4.804 |
| 0.8135739 | −6.148 |
| 0.813817485 | −4.453 |
| 0.81406107 | 0.927 |
| 0.814304655 | 6.308 |
| 0.81454824 | 6.276 |
| 0.814791825 | 5.601 |
| 0.81503541 | 5.153 |
| 0.815278995 | 5.151 |
| 0.81552258 | 5.149 |
| 0.815766165 | 4.626 |
| 0.81600975 | 3.953 |
| 0.816253335 | 3.429 |
| 0.81649692 | 3.428 |
| 0.816740505 | 3.426 |
| 0.81698409 | 2.982 |
| 0.817227675 | 2.312 |
| 0.81747126 | 1.711 |
| 0.817714845 | 1.711 |
| 0.81795843 | 1.71 |
| 0.818202015 | 1.345 |
| 0.8184456 | 0.676 |
| 0.818689185 | 0.008 |
| 0.81893277 | 0 |
| 0.819176355 | 0 |
| 0.81941994 | 0 |
| 0.819663525 | 0 |
| 0.81990711 | 0 |
| 0.820150695 | 0 |
| 0.82039428 | 0 |
| 0.820637865 | 0 |
| 0.82088145 | 0 |
| 0.821125035 | 0 |
| 0.82136862 | 0 |
| 0.821612205 | 0 |
| 0.82185579 | 0 |
| 0.822099375 | 0 |
| 0.82234296 | 0 |
| 0.822586545 | 0 |
| 0.82283013 | 0 |
| 0.823073715 | 0 |
| 0.8233173 | 0 |
| 0.823560885 | 0 |
| 0.82380447 | 1.388 |
| 0.824048055 | 4.037 |
| 0.82429164 | 6.685 |
| 0.824535225 | 6.773 |
| 0.82477881 | 6.771 |
| 0.825022395 | 5.69 |
| 0.82526598 | 3.043 |
| 0.825509565 | 0.397 |
| 0.82575315 | 0 |
| 0.825996735 | 0 |
| 0.82624032 | 0 |
| 0.826483905 | 0 |
| 0.82672749 | 0 |
| 0.826971075 | 0 |
| 0.82721466 | 0 |
| 0.827458245 | 0 |
| 0.82770183 | 0 |
| 0.827945415 | 0 |
| 0.828189 | 0 |
| 0.828432585 | 0 |
| 0.82867617 | 0 |
| 0.828919755 | 0 |
| 0.82916334 | 0 |
| 0.829406925 | 0 |
| 0.82965051 | 0 |
| 0.829894095 | 0 |
| 0.83013768 | 0 |
| 0.830381265 | 0 |
| 0.83062485 | 0 |
| 0.830868435 | 0 |
| 0.83111202 | 0 |
| 0.831355605 | 0 |
| 0.83159919 | 0 |
| 0.831842775 | 0 |
| 0.83208636 | 0 |
| 0.832329945 | 0 |
| 0.83257353 | 0 |
| 0.832817115 | 0 |
| 0.8330607 | 0 |
| 0.833304285 | 0 |
| 0.83354787 | 0 |
| 0.833791455 | 0 |
| 0.83403504 | 0 |
| 0.834278625 | 0 |
| 0.83452221 | 0 |
| 0.834765795 | 0 |
| 0.83500938 | 0 |
| 0.835252965 | 0 |
| 0.83549655 | 0 |
| 0.835740135 | 0 |
| 0.83598372 | 0 |
| 0.836227305 | 0 |
| 0.83647089 | 0 |
| 0.836714475 | 0 |
| 0.83695806 | 0 |
| 0.837201645 | 0 |
| 0.83744523 | 0 |
| 0.837688815 | 0 |
| 0.8379324 | 0 |
| 0.838175985 | 0 |
| 0.83841957 | 0 |
| 0.838663155 | 0 |
| 0.83890674 | 0 |
| 0.839150325 | 0 |
| 0.83939391 | 0 |
| 0.839637495 | 0 |
| 0.83988108 | 0 |
| 0.840124665 | 0 |
| 0.84036825 | 0 |
| 0.840611835 | 0 |
| 0.84085542 | 0 |
| 0.841099005 | 0 |
| 0.84134259 | 0 |
| 0.841586175 | 0 |
| 0.84182976 | 0 |
| 0.842073345 | 0 |
| 0.84231693 | 0 |
| 0.842560515 | 0 |
| 0.8428041 | 0 |
| 0.843047685 | 0 |
| 0.84329127 | 0 |
| 0.843534855 | 0 |
| 0.84377844 | 0 |
| 0.844022025 | 0 |
| 0.84426561 | 0 |
| 0.844509195 | 0 |
| 0.84475278 | 0 |
| 0.844996365 | 0 |
| 0.84523995 | 0 |
| 0.845483535 | 0 |
| 0.84572712 | 0 |
| 0.845970705 | 0 |
| 0.84621429 | 0 |
| 0.846457875 | 0 |
| 0.84670146 | 0 |
| 0.846945045 | 0 |
| 0.84718863 | 0 |
| 0.847432215 | 0 |
| 0.8476758 | 0 |
| 0.847919385 | 0 |
| 0.84816297 | 0 |
| 0.848406555 | 0 |
| 0.84865014 | 0 |
| 0.848893725 | 0 |
| 0.84913731 | 0 |
| 0.849380895 | 0 |
| 0.84962448 | 0 |
| 0.849868065 | 0 |
| 0.85011165 | 0 |
| 0.850355235 | 0 |
| 0.85059882 | 0 |
| 0.850842405 | 0 |
| 0.85108599 | 0 |
| 0.851329575 | 0 |
| 0.85157316 | 0 |
| 0.851816745 | 0 |
| 0.85206033 | 0 |
| 0.852303915 | 0 |
| 0.8525475 | 0 |
| 0.852791085 | 0 |
| 0.85303467 | 0 |
| 0.853278255 | 0 |
| 0.85352184 | 0 |
| 0.853765425 | 0 |
| 0.85400901 | 0 |
| 0.854252595 | 0 |
| 0.85449618 | 0 |
| 0.854739765 | 0 |
| 0.85498335 | 0 |
| 0.855226935 | 0 |
| 0.85547052 | 0 |
| 0.855714105 | 0 |
| 0.85595769 | 0 |
| 0.856201275 | 0 |
| 0.85644486 | 0 |
| 0.856688445 | 0 |
| 0.85693203 | 0 |
| 0.857175615 | 0 |
| 0.8574192 | 0 |
| 0.857662785 | 0 |
| 0.85790637 | 0 |
| 0.858149955 | 0 |
| 0.85839354 | 0 |
| 0.858637125 | 0 |
| 0.85888071 | 0 |
| 0.859124295 | 0 |
| 0.85936788 | 0 |
| 0.859611465 | 0 |
| 0.85985505 | 0 |
| 0.860098635 | 0 |
| 0.86034222 | 0 |
| 0.860585805 | 0 |
| 0.86082939 | 0 |
| 0.861072975 | 0 |
| 0.86131656 | 0 |
| 0.861560145 | 0 |
| 0.86180373 | 0 |
| 0.862047315 | 0 |
| 0.8622909 | 0 |
| 0.862534485 | 0 |
| 0.86277807 | 0 |
| 0.863021655 | 0 |
| 0.86326524 | 0 |
| 0.863508825 | 0 |
| 0.86375241 | 0 |
| 0.863995995 | 0 |
| 0.86423958 | 0 |
| 0.864483165 | 0 |
| 0.86472675 | 0 |
| 0.864970335 | 0 |
| 0.86521392 | 0 |
| 0.865457505 | 0 |
| 0.86570109 | 0 |
| 0.865944675 | 0 |
| 0.86618826 | 0 |
| 0.866431845 | 0 |
| 0.86667543 | 0 |
| 0.866919015 | 0 |
| 0.8671626 | 0 |
| 0.867406185 | 0 |
| 0.86764977 | 0 |
| 0.867893355 | 0 |
| 0.86813694 | 0 |
| 0.868380525 | 0 |
| 0.86862411 | 0 |
| 0.868867695 | 0 |
| 0.86911128 | 0 |
| 0.869354865 | 0 |
| 0.86959845 | 0 |
| 0.869842035 | 0 |
| 0.87008562 | 0 |
| 0.870329205 | 0 |
| 0.87057279 | 0 |
| 0.870816375 | 0 |
| 0.87105996 | 0 |
| 0.871303545 | 0 |
| 0.87154713 | 0 |
| 0.871790715 | 0 |
| 0.8720343 | 0 |
| 0.872277885 | 0 |
| 0.87252147 | 0 |
| 0.872765055 | 0 |
| 0.87300864 | 0 |
| 0.873252225 | 0 |
| 0.87349581 | 0 |
| 0.873739395 | 0 |
| 0.87398298 | 0 |
| 0.874226565 | 0 |
| 0.87447015 | 0 |
| 0.874713735 | 0 |
| 0.87495732 | 0 |
| 0.875200905 | 0 |
| 0.87544449 | 0 |
| 0.875688075 | 0 |
| 0.87593166 | 0 |
| 0.876175245 | 0 |
| 0.87641883 | 0 |
| 0.876662415 | 0 |
| 0.876906 | 0 |
| 0.877149585 | 0 |
| 0.87739317 | 0 |
| 0.877636755 | 0 |
| 0.87788034 | 0 |
| 0.878123925 | 0 |
| 0.87836751 | 0 |
| 0.878611095 | 0 |
| 0.87885468 | 0 |
| 0.879098265 | 0 |
| 0.87934185 | 0 |
| 0.879585435 | 0 |
| 0.87982902 | 0 |
| 0.880072605 | 0 |
| 0.88031619 | 0 |
| 0.880559775 | 0 |
| 0.88080336 | 0 |
| 0.881046945 | 0 |
| 0.88129053 | 0 |
| 0.881534115 | 0 |
| 0.8817777 | 0 |
| 0.882021285 | 0 |
| 0.88226487 | 0 |
| 0.882508455 | 0 |
| 0.88275204 | 0 |
| 0.882995625 | 0 |
| 0.88323921 | 0 |
| 0.883482795 | 0 |
| 0.88372638 | 0 |
| 0.883969965 | 0 |
| 0.88421355 | 0 |
| 0.884457135 | 0 |
| 0.88470072 | 0 |
| 0.884944305 | 0 |
| 0.88518789 | 0 |
| 0.885431475 | 0 |
| 0.88567506 | 0 |
| 0.885918645 | 0 |
| 0.88616223 | 0 |
| 0.886405815 | 0 |
| 0.8866494 | 0 |
| 0.886892985 | 0 |
| 0.88713657 | 0 |
| 0.887380155 | 0 |
| 0.88762374 | 0 |
| 0.887867325 | 0 |
| 0.88811091 | 0 |
| 0.888354495 | 0 |
| 0.88859808 | 0 |
| 0.888841665 | 0 |
| 0.88908525 | 0 |
| 0.889328835 | 0 |
| 0.88957242 | 0 |
| 0.889816005 | 0 |
| 0.89005959 | 0 |
| 0.890303175 | 0 |
| 0.89054676 | 0 |
| 0.890790345 | 0 |
| 0.89103393 | 0 |
| 0.891277515 | 0 |
| 0.8915211 | 0 |
| 0.891764685 | 0 |
| 0.89200827 | 0 |
| 0.892251855 | 0 |
| 0.89249544 | 0 |
| 0.892739025 | 0 |
| 0.89298261 | 0 |
| 0.893226195 | 0 |
| 0.89346978 | 0 |
| 0.893713365 | 0 |
| 0.89395695 | 0 |
| 0.894200535 | 0 |
| 0.89444412 | 0 |
| 0.894687705 | 0 |
| 0.89493129 | 0 |
| 0.895174875 | 0 |
| 0.89541846 | 0 |
| 0.895662045 | 0 |
| 0.89590563 | 0 |
| 0.896149215 | 0 |
| 0.8963928 | 0 |
| 0.896636385 | 0 |
| 0.89687997 | 0 |
| 0.897123555 | 0 |
| 0.89736714 | 0 |
| 0.897610725 | 0 |
| 0.89785431 | 0 |
| 0.898097895 | 0 |
| 0.89834148 | 0 |
| 0.898585065 | 0 |
| 0.89882865 | 0 |
| 0.899072235 | 0 |
| 0.89931582 | 0 |
| 0.899559405 | 0 |
| 0.89980299 | 0 |
| 0.900046575 | 0 |
| 0.90029016 | 0 |
| 0.900533745 | 0 |
| 0.90077733 | 0 |
| 0.901020915 | 0 |
| 0.9012645 | 0 |
| 0.901508085 | 0 |
| 0.90175167 | 0 |
| 0.901995255 | 0 |
| 0.90223884 | 0 |
| 0.902482425 | 0 |
| 0.90272601 | 0 |
| 0.902969595 | 0 |
| 0.90321318 | 0 |
| 0.903456765 | 0 |
| 0.90370035 | 0 |
| 0.903943935 | 0 |
| 0.90418752 | 0 |
| 0.904431105 | 0 |
| 0.90467469 | 0 |
| 0.904918275 | 0 |
| 0.90516186 | 0 |
| 0.905405445 | 0 |
| 0.90564903 | 0 |
| 0.905892615 | 0 |
| 0.9061362 | 0 |
| 0.906379785 | 0 |
| 0.90662337 | 0 |
| 0.906866955 | 0 |
| 0.90711054 | 0 |
| 0.907354125 | 0 |
| 0.90759771 | 0 |
| 0.907841295 | 0 |
| 0.90808488 | 0 |
| 0.908328465 | 0 |
| 0.90857205 | 0 |
| 0.908815635 | 0 |
| 0.90905922 | 0 |
| 0.909302805 | 0 |
| 0.90954639 | 0 |
| 0.909789975 | 0 |
| 0.91003356 | 0 |
| 0.910277145 | 0 |
| 0.91052073 | 0 |
| 0.910764315 | 0 |
| 0.9110079 | 0 |
| 0.911251485 | 0 |
| 0.91149507 | 0 |
| 0.911738655 | 0 |
| 0.91198224 | 0 |
| 0.912225825 | 0 |
| 0.91246941 | 0 |
| 0.912712995 | 0 |
| 0.91295658 | 0 |
| 0.913200165 | 0 |
| 0.91344375 | 0 |
| 0.913687335 | 0 |
| 0.91393092 | 0 |
| 0.914174505 | 0 |
| 0.91441809 | 0 |
| 0.914661675 | 0 |
| 0.91490526 | 0 |
| 0.915148845 | 0 |
| 0.91539243 | 0 |
| 0.915636015 | 0 |
| 0.9158796 | 0 |
| 0.916123185 | 0 |
| 0.91636677 | 0 |
| 0.916610355 | 0 |
| 0.91685394 | 0 |
| 0.917097525 | 0 |
| 0.91734111 | 0 |
| 0.917584695 | 0 |
| 0.91782828 | 0 |
| 0.918071865 | 0 |
| 0.91831545 | 0 |
| 0.918559035 | 0 |
| 0.91880262 | 0 |
| 0.919046205 | 0 |
| 0.91928979 | 0 |
| 0.919533375 | 0 |
| 0.91977696 | 0 |
| 0.920020545 | 0 |
| 0.92026413 | 0 |
| 0.920507715 | 0 |
| 0.9207513 | 0 |
| 0.920994885 | 0 |
| 0.92123847 | 0 |
| 0.921482055 | 0 |
| 0.92172564 | 0 |
| 0.921969225 | 0 |
| 0.92221281 | 0 |
| 0.922456395 | 0 |
| 0.92269998 | 0 |
| 0.922943565 | 0 |
| 0.92318715 | 0 |
| 0.923430735 | 0 |
| 0.92367432 | 0 |
| 0.923917905 | 0 |
| 0.92416149 | 0 |
| 0.924405075 | 0 |
| 0.92464866 | 0 |
| 0.924892245 | 0 |
| 0.92513583 | 0 |
| 0.925379415 | 0 |
| 0.925623 | 0 |
| 0.925866585 | 0 |
| 0.92611017 | 0 |
| 0.926353755 | 0 |
| 0.92659734 | 0 |
| 0.926840925 | 0 |
| 0.92708451 | 0 |
| 0.927328095 | 0 |
| 0.92757168 | 0 |
| 0.927815265 | 0 |
| 0.92805885 | 0 |
| 0.928302435 | 0 |
| 0.92854602 | 0 |
| 0.928789605 | 0 |
| 0.92903319 | 0 |
| 0.929276775 | 0 |
| 0.92952036 | 0 |
| 0.929763945 | 0 |
| 0.93000753 | 0 |
| 0.930251115 | 0 |
| 0.9304947 | 0 |
| 0.930738285 | 0 |
| 0.93098187 | 0 |
| 0.931225455 | 0 |
| 0.93146904 | 0 |
| 0.931712625 | 0 |
| 0.93195621 | 0 |
| 0.932199795 | 0 |
| 0.93244338 | 0 |
| 0.932686965 | 0 |
| 0.93293055 | 0 |
| 0.933174135 | 0 |
| 0.93341772 | 0 |
| 0.933661305 | 0 |
| 0.93390489 | 0 |
| 0.934148475 | 0 |
| 0.93439206 | 0 |
| 0.934635645 | 0 |
| 0.93487923 | 0 |
| 0.935122815 | 0 |
| 0.9353664 | 0 |
| 0.935609985 | 0 |
| 0.93585357 | 0.011 |
| 0.936097155 | −0.136 |
| 0.93634074 | −0.262 |
| 0.936584325 | −0.585 |
| 0.93682791 | −1.088 |
| 0.937071495 | −1.59 |
| 0.93731508 | −2.215 |
| 0.937558665 | −2.843 |
| 0.93780225 | −3.729 |
| 0.938045835 | −4.986 |
| 0.93828942 | −6.244 |
| 0.938533005 | −2.146 |
| 0.93877659 | 2.882 |
| 0.939020175 | 6.248 |
| 0.93926376 | 5.62 |
| 0.939507345 | 4.992 |
| 0.93975093 | 4.826 |
| 0.939994515 | 4.827 |
| 0.9402381 | 4.716 |
| 0.940481685 | 4.087 |
| 0.94072527 | 3.459 |
| 0.940968855 | 3.22 |
| 0.94121244 | 3.22 |
| 0.941456025 | 3.182 |
| 0.94169961 | 2.553 |
| 0.941943195 | 1.923 |
| 0.94218678 | 1.611 |
| 0.942430365 | 1.611 |
| 0.94267395 | 1.612 |
| 0.942917535 | 1.016 |
| 0.94316112 | 0.386 |
| 0.943404705 | 0 |
| 0.94364829 | 0 |
| 0.943891875 | 0 |
| 0.94413546 | 0 |
| 0.944379045 | 0 |
| 0.94462263 | 0 |
| 0.944866215 | 0 |
| 0.9451098 | 0 |
| 0.945353385 | 0 |
| 0.94559697 | 0 |
| 0.945840555 | 0 |
| 0.94608414 | 0 |
| 0.946327725 | 0 |
| 0.94657131 | 0 |
| 0.946814895 | 0 |
| 0.94705848 | 0 |
| 0.947302065 | 0 |
| 0.94754565 | 0 |
| 0.947789235 | 0 |
| 0.94803282 | 0 |
| 0.948276405 | 0 |
| 0.94851999 | 2.339 |
| 0.948763575 | 4.87 |
| 0.94900716 | 6.476 |
| 0.949250745 | 6.477 |
| 0.94949433 | 6.478 |
| 0.949737915 | 4.432 |
| 0.9499815 | 1.899 |
| 0.950225085 | 0 |
| 0.95046867 | 0 |
| 0.950712255 | 0 |
| 0.95095584 | 0 |
| 0.951199425 | 0 |
| 0.95144301 | −0.017 |
| 0.951686595 | −0.144 |
| 0.95193018 | −0.271 |
| 0.952173765 | −0.617 |
| 0.95241735 | −1.125 |
| 0.952660935 | −1.635 |
| 0.95290452 | −2.27 |
| 0.953148105 | −2.905 |
| 0.95339169 | −3.833 |
| 0.953635275 | −5.104 |
| 0.95387886 | −6.376 |
| 0.954122445 | −1.915 |
| 0.95436603 | 3.169 |
| 0.954609615 | 6.285 |
| 0.9548532 | 5.651 |
| 0.955096785 | 5.016 |
| 0.95534037 | 4.881 |
| 0.955583955 | 4.882 |
| 0.95582754 | 4.738 |
| 0.956071125 | 4.102 |
| 0.95631471 | 3.467 |
| 0.956558295 | 3.257 |
| 0.95680188 | 3.258 |
| 0.957045465 | 3.187 |
| 0.95728905 | 2.551 |
| 0.957532635 | 1.914 |
| 0.95777622 | 1.63 |
| 0.958019805 | 1.631 |
| 0.95826339 | 1.631 |
| 0.958506975 | 0.996 |
| 0.95875056 | 0.358 |
| 0.958994145 | 0 |
| 0.95923773 | 0 |
| 0.959481315 | 0 |
| 0.9597249 | 0 |
| 0.959968485 | 0 |
| 0.96021207 | 0 |
| 0.960455655 | 0 |
| 0.96069924 | 0 |
| 0.960942825 | 0 |
| 0.96118641 | 0 |
| 0.961429995 | 0 |
| 0.96167358 | 0 |
| 0.961917165 | 0 |
| 0.96216075 | 0 |
| 0.962404335 | 0 |
| 0.96264792 | 0 |
| 0.962891505 | 0 |
| 0.96313509 | 0 |
| 0.963378675 | 0 |
| 0.96362226 | 0 |
| 0.963865845 | 0 |
| 0.96410943 | 2.498 |
| 0.964353015 | 5.064 |
| 0.9645966 | 6.562 |
| 0.964840185 | 6.564 |
| 0.96508377 | 6.565 |
| 0.965327355 | 4.363 |
| 0.96557094 | 1.797 |
| 0.965814525 | 0 |
| 0.96605811 | 0 |
| 0.966301695 | 0 |
| 0.96654528 | 0 |
| 0.966788865 | 0 |
| 0.96703245 | −0.024 |
| 0.967276035 | −0.152 |
| 0.96751962 | −0.281 |
| 0.967763205 | −0.651 |
| 0.96800679 | −1.166 |
| 0.968250375 | −1.69 |
| 0.96849396 | −2.335 |
| 0.968737545 | −2.979 |
| 0.96898113 | −3.953 |
| 0.969224715 | −5.242 |
| 0.9694683 | −6.532 |
| 0.969711885 | −1.684 |
| 0.96995547 | 3.473 |
| 0.970199055 | 6.345 |
| 0.97044264 | 5.701 |
| 0.970686225 | 5.057 |
| 0.97092981 | 4.953 |
| 0.971173395 | 4.954 |
| 0.97141698 | 4.776 |
| 0.971660565 | 4.131 |
| 0.97190415 | 3.486 |
| 0.972147735 | 3.306 |
| 0.97239132 | 3.307 |
| 0.972634905 | 3.203 |
| 0.97287849 | 2.557 |
| 0.973122075 | 1.911 |
| 0.97336566 | 1.655 |
| 0.973609245 | 1.656 |
| 0.97385283 | 1.626 |
| 0.974096415 | 0.979 |
| 0.97434 | 0.331 |
| 0.974583585 | 0 |
| 0.97482717 | 0 |
| 0.975070755 | 0 |
| 0.97531434 | 0 |
| 0.975557925 | 0 |
| 0.97580151 | 0 |
| 0.976045095 | 0 |
| 0.97628868 | 0 |
| 0.976532265 | 0 |
| 0.97677585 | 0 |
| 0.977019435 | 0 |
| 0.97726302 | 0 |
| 0.977506605 | 0 |
| 0.97775019 | 0 |
| 0.977993775 | 0 |
| 0.97823736 | 0 |
| 0.978480945 | 0 |
| 0.97872453 | 0 |
| 0.978968115 | 0 |
| 0.9792117 | 0 |
| 0.979455285 | 0.063 |
| 0.97969887 | 2.67 |
| 0.979942455 | 5.277 |
| 0.98018604 | 6.67 |
| 0.980429625 | 6.672 |
| 0.98067321 | 6.674 |
| 0.980916795 | 4.305 |
| 0.98116038 | 1.696 |
| 0.981403965 | 0 |
| 0.98164755 | 0 |
| 0.981891135 | 0 |
| 0.98213472 | 0 |
| 0.982378305 | 0 |
| 0.98262189 | −0.031 |
| 0.982865475 | −0.161 |
| 0.98310906 | −0.292 |
| 0.983352645 | −0.689 |
| 0.98359623 | −1.213 |
| 0.983839815 | −1.752 |
| 0.9840834 | −2.408 |
| 0.984326985 | −3.063 |
| 0.98457057 | −4.086 |
| 0.984814155 | −5.398 |
| 0.98505774 | −6.698 |
| 0.985301325 | −1.451 |
| 0.98554491 | 3.796 |
| 0.985788495 | 6.422 |
| 0.98603208 | 5.768 |
| 0.986275665 | 5.113 |
| 0.98651925 | 5.04 |
| 0.986762835 | 5.041 |
| 0.98700642 | 4.827 |
| 0.987250005 | 4.171 |
| 0.98749359 | 3.515 |
| 0.987737175 | 3.365 |
| 0.98798076 | 3.366 |
| 0.988224345 | 3.227 |
| 0.98846793 | 2.57 |
| 0.988711515 | 1.912 |
| 0.9889551 | 1.685 |
| 0.989198685 | 1.685 |
| 0.98944227 | 1.623 |
| 0.989685855 | 0.963 |
| 0.98992944 | 0.304 |
| 0.990173025 | 0 |
| 0.99041661 | 0 |
| 0.990660195 | 0 |
| 0.99090378 | 0 |
| 0.991147365 | 0 |
| 0.99139095 | 0 |
| 0.991634535 | 0 |
| 0.99187812 | 0 |
| 0.992121705 | 0 |
| 0.99236529 | 0 |
| 0.992608875 | 0 |
| 0.99285246 | 0 |
| 0.993096045 | 0 |
| 0.99333963 | 0 |
| 0.993583215 | 0 |
| 0.9938268 | 0 |
| 0.994070385 | 0 |
| 0.99431397 | 0 |
| 0.994557555 | 0 |
| 0.99480114 | 0 |
| 0.995044725 | 0.197 |
| 0.99528831 | 2.853 |
| 0.995531895 | 5.51 |
| 0.99577548 | 6.796 |
| 0.996019065 | 6.798 |
| 0.99626265 | 6.801 |
| 0.996506235 | 4.254 |
| 0.99674982 | 1.595 |
| 0.996993405 | 0 |
| 0.99723699 | 0 |
| 0.997480575 | 0 |
| 0.99772416 | 0 |
| 0.997967745 | 0 |
| 0.99821133 | 0 |
| 0.998454915 | 0 |
| 0.9986985 | 0 |
| 0.998942085 | 0 |
| 0.99918567 | 0 |
| 0.999429255 | 0 |
| 0.99967284 | 0 |
| 0.999916425 | 0 |
| 1.00016001 | 0 |
| 1.000403595 | 0 |
| 1.00064718 | 0 |
| 1.000890765 | 0 |
| 1.00113435 | 0 |
| 1.001377935 | 0 |
| 1.00162152 | 0 |
| 1.001865105 | 0 |
| 1.00210869 | 0 |
| 1.002352275 | 0 |
| 1.00259586 | 0 |
| 1.002839445 | 0 |
| 1.00308303 | 0 |
| 1.003326615 | 0 |
| 1.0035702 | 0 |
| 1.003813785 | 0 |
| 1.00405737 | 0 |
| 1.004300955 | 0 |
| 1.00454454 | 0 |
| 1.004788125 | 0 |
| 1.00503171 | 0 |
| 1.005275295 | 0 |
| 1.00551888 | 0 |
| 1.005762465 | 0 |
| 1.00600605 | 0 |
| 1.006249635 | 0 |
| 1.00649322 | 0 |
| 1.006736805 | 0 |
| 1.00698039 | 0 |
| 1.007223975 | 0 |
| 1.00746756 | 0 |
| 1.007711145 | 0 |
| 1.00795473 | 0 |
| 1.008198315 | 0 |
| 1.0084419 | 0 |
| 1.008685485 | 0 |
| 1.00892907 | 0 |
| 1.009172655 | 0 |
| 1.00941624 | 0 |
| 1.009659825 | 0 |
| 1.00990341 | 0 |
| 1.010146995 | 0 |
| 1.01039058 | 0 |
| 1.010634165 | 0 |
| 1.01087775 | 0 |
| 1.011121335 | 0 |
| 1.01136492 | 0 |
| 1.011608505 | 0 |
| 1.01185209 | 0 |
| 1.012095675 | 0 |
| 1.01233926 | 0 |
| 1.012582845 | 0 |
| 1.01282643 | 0 |
| 1.013070015 | 0 |
| 1.0133136 | 0 |
| 1.013557185 | 0 |
| 1.01380077 | 0 |
| 1.014044355 | 0 |
| 1.01428794 | 0 |
| 1.014531525 | 0 |
| 1.01477511 | 0 |
| 1.015018695 | 0 |
| 1.01526228 | 0 |
| 1.015505865 | 0 |
| 1.01574945 | 0 |
| 1.015993035 | 0 |
| 1.01623662 | 0 |
| 1.016480205 | 0 |
| 1.01672379 | 0 |
| 1.016967375 | 0 |
| 1.01721096 | 0 |
| 1.017454545 | 0 |
| 1.01769813 | 0 |
| 1.017941715 | 0 |
| 1.0181853 | 0 |
| 1.018428885 | 0 |
| 1.01867247 | 0 |
| 1.018916055 | 0 |
| 1.01915964 | 0 |
| 1.019403225 | 0 |
| 1.01964681 | 0 |
| 1.019890395 | 0 |
| 1.02013398 | 0 |
| 1.020377565 | 0 |
| 1.02062115 | 0 |
| 1.020864735 | 0 |
| 1.02110832 | 0 |
| 1.021351905 | 0 |
| 1.02159549 | 0 |
| 1.021839075 | 0 |
| 1.02208266 | 0 |
| 1.022326245 | 0 |
| 1.02256983 | 0 |
| 1.022813415 | 0 |
| 1.023057 | 0 |
| 1.023300585 | 0 |
| 1.02354417 | 0 |
| 1.023787755 | 0 |
| 1.02403134 | 0 |
| 1.024274925 | 0 |
| 1.02451851 | 0 |
| 1.024762095 | 0 |
| 1.02500568 | 0 |
| 1.025249265 | 0 |
| 1.02549285 | 0 |
| 1.025736435 | 0 |
| 1.02598002 | 0 |
| 1.026223605 | 0 |
| 1.02646719 | 0 |
| 1.026710775 | 0 |
| 1.02695436 | 0 |
| 1.027197945 | 0 |
| 1.02744153 | 0 |
| 1.027685115 | 0 |
| 1.0279287 | 0 |
| 1.028172285 | 0 |
| 1.02841587 | 0 |
| 1.028659455 | 0 |
| 1.02890304 | 0 |
| 1.029146625 | 0 |
| 1.02939021 | 0 |
| 1.029633795 | 0 |
| 1.02987738 | 0 |
| 1.030120965 | 0 |
| 1.03036455 | 0 |
| 1.030608135 | 0 |
| 1.03085172 | 0 |
| 1.031095305 | 0 |
| 1.03133889 | 0 |
| 1.031582475 | 0 |
| 1.03182606 | 0 |
| 1.032069645 | 0 |
| 1.03231323 | 0 |
| 1.032556815 | 0 |
| 1.0328004 | 0 |
| 1.033043985 | 0 |
| 1.03328757 | 0 |
| 1.033531155 | 0 |
| 1.03377474 | 0 |
| 1.034018325 | 0 |
| 1.03426191 | 0 |
| 1.034505495 | 0 |
| 1.03474908 | 0 |
| 1.034992665 | 0 |
| 1.03523625 | 0 |
| 1.035479835 | 0 |
| 1.03572342 | 0 |
| 1.035967005 | 0 |
| 1.03621059 | 0 |
| 1.036454175 | 0 |
| 1.03669776 | 0 |
| 1.036941345 | 0 |
| 1.03718493 | 0 |
| 1.037428515 | 0 |
| 1.0376721 | 0 |
| 1.037915685 | 0 |
| 1.03815927 | 0 |
| 1.038402855 | 0 |
| 1.03864644 | 0 |
| 1.038890025 | 0 |
| 1.03913361 | 0 |
| 1.039377195 | 0 |
| 1.03962078 | 0 |
| 1.039864365 | 0 |
| 1.04010795 | 0 |
| 1.040351535 | 0 |
| 1.04059512 | 0 |
| 1.040838705 | 0 |
| 1.04108229 | 0 |
| 1.041325875 | 0 |
| 1.04156946 | 0 |
| 1.041813045 | 0 |
| 1.04205663 | 0 |
| 1.042300215 | 0 |
| 1.0425438 | 0 |
| 1.042787385 | 0 |
| 1.04303097 | 0 |
| 1.043274555 | 0 |
| 1.04351814 | 0 |
| 1.043761725 | 0 |
| 1.04400531 | 0 |
| 1.044248895 | 0 |
| 1.04449248 | 0 |
| 1.044736065 | 0 |
| 1.04497965 | 0 |
| 1.045223235 | 0 |
| 1.04546682 | 0 |
| 1.045710405 | 0 |
| 1.04595399 | 0 |
| 1.046197575 | 0 |
| 1.04644116 | 0 |
| 1.046684745 | 0 |
| 1.04692833 | 0 |
| 1.047171915 | 0 |
| 1.0474155 | 0 |
| 1.047659085 | 0 |
| 1.04790267 | 0 |
| 1.048146255 | 0 |
| 1.04838984 | 0 |
| 1.048633425 | 0 |
| 1.04887701 | 0 |
| 1.049120595 | 0 |
| 1.04936418 | 0 |
| 1.049607765 | 0 |
| 1.04985135 | 0 |
| 1.050094935 | 0 |
| 1.05033852 | 0 |
| 1.050582105 | 0 |
| 1.05082569 | 0 |
| 1.051069275 | 0 |
| 1.05131286 | 0 |
| 1.051556445 | 0 |
| 1.05180003 | 0 |
| 1.052043615 | 0 |
| 1.0522872 | 0 |
| 1.052530785 | 0 |
| 1.05277437 | 0 |
| 1.053017955 | 0 |
| 1.05326154 | 0 |
| 1.053505125 | 0 |
| 1.05374871 | 0 |
| 1.053992295 | 0 |
| 1.05423588 | 0 |
| 1.054479465 | 0 |
| 1.05472305 | 0 |
| 1.054966635 | 0 |
| 1.05521022 | 0 |
| 1.055453805 | 0 |
| 1.05569739 | 0 |
| 1.055940975 | 0 |
| 1.05618456 | 0 |
| 1.056428145 | 0 |
| 1.05667173 | 0 |
| 1.056915315 | 0 |
| 1.0571589 | 0 |
| 1.057402485 | 0 |
| 1.05764607 | 0 |
| 1.057889655 | 0 |
| 1.05813324 | 0 |
| 1.058376825 | 0 |
| 1.05862041 | 0 |
| 1.058863995 | 0 |
| 1.05910758 | 0 |
| 1.059351165 | 0 |
| 1.05959475 | 0 |
| 1.059838335 | 0 |
| 1.06008192 | 0 |
| 1.060325505 | 0 |
| 1.06056909 | 0 |
| 1.060812675 | 0 |
| 1.06105626 | 0 |
| 1.061299845 | 0 |
| 1.06154343 | 0 |
| 1.061787015 | 0 |
| 1.0620306 | 0 |
| 1.062274185 | 0 |
| 1.06251777 | 0 |
| 1.062761355 | 0 |
| 1.06300494 | 0 |
| 1.063248525 | 0 |
| 1.06349211 | 0 |
| 1.063735695 | 0 |
| 1.06397928 | 0 |
| 1.064222865 | 0 |
| 1.06446645 | 0 |
| 1.064710035 | 0 |
| 1.06495362 | 0 |
| 1.065197205 | 0 |
| 1.06544079 | 0 |
| 1.065684375 | 0 |
| 1.06592796 | 0 |
| 1.066171545 | 0 |
| 1.06641513 | 0 |
| 1.066658715 | 0 |
| 1.0669023 | 0 |
| 1.067145885 | 0 |
| 1.06738947 | 0 |
| 1.067633055 | 0 |
| 1.06787664 | 0 |
| 1.068120225 | 0 |
| 1.06836381 | 0 |
| 1.068607395 | 0 |
| 1.06885098 | 0 |
| 1.069094565 | 0 |
| 1.06933815 | 0 |
| 1.069581735 | 0 |
| 1.06982532 | 0 |
| 1.070068905 | 0 |
| 1.07031249 | 0 |
| 1.070556075 | 0 |
| 1.07079966 | 0 |
| 1.071043245 | 0 |
| 1.07128683 | 0 |
| 1.071530415 | 0 |
| 1.071774 | 0 |
| 1.072017585 | 0 |
| 1.07226117 | 0 |
| 1.072504755 | 0 |
| 1.07274834 | 0 |
| 1.072991925 | 0 |
| 1.07323551 | 0 |
| 1.073479095 | 0 |
| 1.07372268 | 0 |
| 1.073966265 | 0 |
| 1.07420985 | 0 |
| 1.074453435 | 0 |
| 1.07469702 | 0 |
| 1.074940605 | 0 |
| 1.07518419 | 0 |
| 1.075427775 | 0 |
| 1.07567136 | 0 |
| 1.075914945 | 0 |
| 1.07615853 | 0 |
| 1.076402115 | 0 |
| 1.0766457 | 0 |
| 1.076889285 | 0 |
| 1.07713287 | 0 |
| 1.077376455 | 0 |
| 1.07762004 | 0 |
| 1.077863625 | 0 |
| 1.07810721 | 0 |
| 1.078350795 | 0 |
| 1.07859438 | 0 |
| 1.078837965 | 0 |
| 1.07908155 | 0 |
| 1.079325135 | 0 |
| 1.07956872 | 0 |
| 1.079812305 | 0 |
| 1.08005589 | 0 |
| 1.080299475 | 0 |
| 1.08054306 | 0 |
| 1.080786645 | 0 |
| 1.08103023 | 0 |
| 1.081273815 | 0 |
| 1.0815174 | 0 |
| 1.081760985 | 0 |
| 1.08200457 | 0 |
| 1.082248155 | 0 |
| 1.08249174 | 0 |
| 1.082735325 | 0 |
| 1.08297891 | 0 |
| 1.083222495 | 0 |
| 1.08346608 | 0 |
| 1.083709665 | 0 |
| 1.08395325 | 0 |
| 1.084196835 | 0 |
| 1.08444042 | 0 |
| 1.084684005 | 0 |
| 1.08492759 | 0 |
| 1.085171175 | 0 |
| 1.08541476 | 0 |
| 1.085658345 | 0 |
| 1.08590193 | 0 |
| 1.086145515 | 0 |
| 1.0863891 | 0 |
| 1.086632685 | 0 |
| 1.08687627 | 0 |
| 1.087119855 | 0 |
| 1.08736344 | 0 |
| 1.087607025 | 0 |
| 1.08785061 | 0 |
| 1.088094195 | 0 |
| 1.08833778 | 0 |
| 1.088581365 | 0 |
| 1.08882495 | 0 |
| 1.089068535 | 0 |
| 1.08931212 | 0 |
| 1.089555705 | 0 |
| 1.08979929 | 0 |
| 1.090042875 | 0 |
| 1.09028646 | 0 |
| 1.090530045 | 0 |
| 1.09077363 | 0 |
| 1.091017215 | 0 |
| 1.0912608 | 0 |
| 1.091504385 | 0 |
| 1.09174797 | 0 |
| 1.091991555 | 0 |
| 1.09223514 | 0 |
| 1.092478725 | 0 |
| 1.09272231 | 0 |
| 1.092965895 | 0 |
| 1.09320948 | 0 |
| 1.093453065 | 0 |
| 1.09369665 | 0 |
| 1.093940235 | 0 |
| 1.09418382 | 0 |
| 1.094427405 | 0 |
| 1.09467099 | 0 |
| 1.094914575 | 0 |
| 1.09515816 | 0 |
| 1.095401745 | 0 |
| 1.09564533 | 0 |
| 1.095888915 | 0 |
| 1.0961325 | 0 |
| 1.096376085 | 0 |
| 1.09661967 | 0 |
| 1.096863255 | 0 |
| 1.09710684 | 0 |
| 1.097350425 | 0 |
| 1.09759401 | 0 |
| 1.097837595 | 0 |
| 1.09808118 | 0 |
| 1.098324765 | 0 |
| 1.09856835 | 0 |
| 1.098811935 | 0 |
| 1.09905552 | 0 |
| 1.099299105 | 0 |
| 1.09954269 | 0 |
| 1.099786275 | 0 |
| 1.10002986 | 0 |
| 1.100273445 | 0 |
| 1.10051703 | 0 |
| 1.100760615 | 0 |
| 1.1010042 | 0 |
| 1.101247785 | 0 |
| 1.10149137 | 0 |
| 1.101734955 | 0 |
| 1.10197854 | 0 |
| 1.102222125 | 0 |
| 1.10246571 | 0 |
| 1.102709295 | 0 |
| 1.10295288 | 0 |
| 1.103196465 | 0 |
| 1.10344005 | 0 |
| 1.103683635 | 0 |
| 1.10392722 | 0 |
| 1.104170805 | 0 |
| 1.10441439 | 0 |
| 1.104657975 | 0 |
| 1.10490156 | 0 |
| 1.105145145 | 0 |
| 1.10538873 | 0 |
| 1.105632315 | 0 |
| 1.1058759 | 0 |
| 1.106119485 | 0 |
| 1.10636307 | 0 |
| 1.106606655 | 0 |
| 1.10685024 | 0 |
| 1.107093825 | 0 |
| 1.10733741 | 0 |
| 1.107580995 | 0 |
| 1.10782458 | 0 |
| 1.108068165 | 0 |
| 1.10831175 | 0 |
| 1.108555335 | 0 |
| 1.10879892 | 0 |
| 1.109042505 | 0 |
| 1.10928609 | 0 |
| 1.109529675 | 0 |
| 1.10977326 | 0 |
| 1.110016845 | 0 |
| 1.11026043 | 0 |
| 1.110504015 | 0 |
| 1.1107476 | 0 |
| 1.110991185 | 0 |
| 1.11123477 | 0 |
| 1.111478355 | 0 |
| 1.11172194 | 0 |
| 1.111965525 | 0 |
| 1.11220911 | 0 |
| 1.112452695 | 0 |
| 1.11269628 | 0 |
| 1.112939865 | 0 |
| 1.11318345 | 0 |
| 1.113427035 | 0 |
| 1.11367062 | 0 |
| 1.113914205 | 0 |
| 1.11415779 | 0 |
| 1.114401375 | 0 |
| 1.11464496 | 0 |
| 1.114888545 | 0 |
| 1.11513213 | 0 |
| 1.115375715 | 0 |
| 1.1156193 | 0 |
| 1.115862885 | 0 |
| 1.11610647 | 0 |
| 1.116350055 | 0 |
| 1.11659364 | 0 |
| 1.116837225 | 0 |
| 1.11708081 | 0 |
| 1.117324395 | 0 |
| 1.11756798 | 0 |
| 1.117811565 | 0 |
| 1.11805515 | 0 |
| 1.118298735 | 0 |
| 1.11854232 | 0 |
| 1.118785905 | 0 |
| 1.11902949 | 0 |
| 1.119273075 | 0 |
| 1.11951666 | 0 |
| 1.119760245 | 0 |
| 1.12000383 | 0 |
| 1.120247415 | 0 |
| 1.120491 | 0 |
| 1.120734585 | 0 |
| 1.12097817 | 0 |
| 1.121221755 | 0 |
| 1.12146534 | 0 |
| 1.121708925 | 0 |
| 1.12195251 | 0 |
| 1.122196095 | 0 |
| 1.12243968 | 0 |
| 1.122683265 | 0 |
| 1.12292685 | −0.107 |
| 1.123170435 | −0.263 |
| 1.12341402 | −0.478 |
| 1.123657605 | −1.103 |
| 1.12390119 | −1.728 |
| 1.124144775 | −2.442 |
| 1.12438836 | −3.224 |
| 1.124631945 | −4.014 |
| 1.12487553 | −5.579 |
| 1.125119115 | −7.144 |
| 1.1253627 | −5.173 |
| 1.125606285 | 1.085 |
| 1.12584987 | 7.342 |
| 1.126093455 | 7.307 |
| 1.12633704 | 6.526 |
| 1.126580625 | 6.008 |
| 1.12682421 | 6.009 |
| 1.127067795 | 6.01 |
| 1.12731138 | 5.402 |
| 1.127554965 | 4.619 |
| 1.12779855 | 4.009 |
| 1.128042135 | 4.01 |
| 1.12828572 | 4.011 |
| 1.128529305 | 3.493 |
| 1.12877289 | 2.709 |
| 1.129016475 | 2.007 |
| 1.12926006 | 2.007 |
| 1.129503645 | 2.008 |
| 1.12974723 | 1.579 |
| 1.129990815 | 0.794 |
| 1.1302344 | 0.009 |
| 1.130477985 | 0 |
| 1.13072157 | 0 |
| 1.130965155 | 0 |
| 1.13120874 | 0 |
| 1.131452325 | 0 |
| 1.13169591 | 0 |
| 1.131939495 | 0 |
| 1.13218308 | 0 |
| 1.132426665 | 0 |
| 1.13267025 | 0 |
| 1.132913835 | 0 |
| 1.13315742 | 0 |
| 1.133401005 | 0 |
| 1.13364459 | 0 |
| 1.133888175 | 0 |
| 1.13413176 | 0 |
| 1.134375345 | 0 |
| 1.13461893 | 0 |
| 1.134862515 | 0 |
| 1.1351061 | 0 |
| 1.135349685 | 1.653 |
| 1.13559327 | 4.809 |
| 1.135836855 | 7.964 |
| 1.13608044 | 8.073 |
| 1.136324025 | 8.074 |
| 1.13656761 | 6.787 |
| 1.136811195 | 3.63 |
| 1.13705478 | 0.474 |
| 1.137298365 | 0 |
| 1.13754195 | 0 |
| 1.137785535 | 0 |
| 1.13802912 | 0 |
| 1.138272705 | 0 |
| 1.13851629 | −0.116 |
| 1.138759875 | −0.274 |
| 1.13900346 | −0.516 |
| 1.139247045 | −1.149 |
| 1.13949063 | −1.782 |
| 1.139734215 | −2.513 |
| 1.1399778 | −3.304 |
| 1.140221385 | −4.144 |
| 1.14046497 | −5.728 |
| 1.140708555 | −7.312 |
| 1.14095214 | −4.921 |
| 1.141195725 | 1.414 |
| 1.14143931 | 7.75 |
| 1.141682895 | 7.358 |
| 1.14192648 | 6.567 |
| 1.142170065 | 6.081 |
| 1.14241365 | 6.082 |
| 1.142657235 | 6.083 |
| 1.14290082 | 5.428 |
| 1.143144405 | 4.636 |
| 1.14338799 | 4.058 |
| 1.143631575 | 4.058 |
| 1.14387516 | 4.059 |
| 1.144118745 | 3.494 |
| 1.14436233 | 2.701 |
| 1.144605915 | 2.03 |
| 1.1448495 | 2.031 |
| 1.145093085 | 2.031 |
| 1.14533667 | 1.558 |
| 1.145580255 | 0.764 |
| 1.14582384 | 0 |
| 1.146067425 | 0 |
| 1.14631101 | 0 |
| 1.146554595 | 0 |
| 1.14679818 | 0 |
| 1.147041765 | 0 |
| 1.14728535 | 0 |
| 1.147528935 | 0 |
| 1.14777252 | 0 |
| 1.148016105 | 0 |
| 1.14825969 | 0 |
| 1.148503275 | 0 |
| 1.14874686 | 0 |
| 1.148990445 | 0 |
| 1.14923403 | 0 |
| 1.149477615 | 0 |
| 1.1497212 | 0 |
| 1.149964785 | 0 |
| 1.15020837 | 0 |
| 1.150451955 | 0 |
| 1.15069554 | 0 |
| 1.150939125 | 1.83 |
| 1.15118271 | 5.019 |
| 1.151426295 | 8.156 |
| 1.15166988 | 8.158 |
| 1.151913465 | 8.159 |
| 1.15215705 | 6.698 |
| 1.152400635 | 3.509 |
| 1.15264422 | 0.319 |
| 1.152887805 | 0 |
| 1.15313139 | 0 |
| 1.153374975 | 0 |
| 1.15361856 | 0 |
| 1.153862145 | 0 |
| 1.15410573 | −0.125 |
| 1.154349315 | −0.285 |
| 1.1545929 | −0.553 |
| 1.154836485 | −1.192 |
| 1.15508007 | −1.831 |
| 1.155323655 | −2.577 |
| 1.15556724 | −3.377 |
| 1.155810825 | −4.264 |
| 1.15605441 | −5.863 |
| 1.156297995 | −7.463 |
| 1.15654158 | −4.648 |
| 1.156785165 | 1.747 |
| 1.15702875 | 8.143 |
| 1.157272335 | 7.388 |
| 1.15751592 | 6.589 |
| 1.157759505 | 6.139 |
| 1.15800309 | 6.139 |
| 1.158246675 | 6.14 |
| 1.15849026 | 5.438 |
| 1.158733845 | 4.638 |
| 1.15897743 | 4.095 |
| 1.159221015 | 4.095 |
| 1.1594646 | 4.096 |
| 1.159708185 | 3.486 |
| 1.15995177 | 2.686 |
| 1.160195355 | 2.049 |
| 1.16043894 | 2.049 |
| 1.160682525 | 2.049 |
| 1.16092611 | 1.532 |
| 1.161169695 | 0.731 |
| 1.16141328 | 0 |
| 1.161656865 | 0 |
| 1.16190045 | 0 |
| 1.162144035 | 0 |
| 1.16238762 | 0 |
| 1.162631205 | 0 |
| 1.16287479 | 0 |
| 1.163118375 | 0 |
| 1.16336196 | 0 |
| 1.163605545 | 0 |
| 1.16384913 | 0 |
| 1.164092715 | 0 |
| 1.1643363 | 0 |
| 1.164579885 | 0 |
| 1.16482347 | 0 |
| 1.165067055 | 0 |
| 1.16531064 | 0 |
| 1.165554225 | 0 |
| 1.16579781 | 0 |
| 1.166041395 | 0 |
| 1.16628498 | 0 |
| 1.166528565 | 2.005 |
| 1.16677215 | 5.218 |
| 1.167015735 | 8.219 |
| 1.16725932 | 8.22 |
| 1.167502905 | 8.22 |
| 1.16774649 | 6.588 |
| 1.167990075 | 3.374 |
| 1.16823366 | 0.161 |
| 1.168477245 | 0 |
| 1.16872083 | 0 |
| 1.168964415 | 0 |
| 1.169208 | 0 |
| 1.169451585 | 0 |
| 1.16969517 | −0.134 |
| 1.169938755 | −0.295 |
| 1.17018234 | −0.589 |
| 1.170425925 | −1.233 |
| 1.17066951 | −1.876 |
| 1.170913095 | −2.635 |
| 1.17115668 | −3.44 |
| 1.171400265 | −4.373 |
| 1.17164385 | −5.983 |
| 1.171887435 | −7.593 |
| 1.17213102 | −4.358 |
| 1.172374605 | 2.08 |
| 1.17261819 | 8.2 |
| 1.172861775 | 7.395 |
| 1.17310536 | 6.591 |
| 1.173348945 | 6.178 |
| 1.17359253 | 6.178 |
| 1.173836115 | 6.179 |
| 1.1740797 | 5.432 |
| 1.174323285 | 4.627 |
| 1.17456687 | 4.12 |
| 1.174810455 | 4.12 |
| 1.17505404 | 4.121 |
| 1.175297625 | 3.467 |
| 1.17554121 | 2.661 |
| 1.175784795 | 2.061 |
| 1.17602838 | 2.061 |
| 1.176271965 | 2.061 |
| 1.17651555 | 1.5 |
| 1.176759135 | 0.695 |
| 1.17700272 | 0 |
| 1.177246305 | 0 |
| 1.17748989 | 0 |
| 1.177733475 | 0 |
| 1.17797706 | 0 |
| 1.178220645 | 0 |
| 1.17846423 | 0 |
| 1.178707815 | 0 |
| 1.1789514 | 0 |
| 1.179194985 | 0 |
| 1.17943857 | 0 |
| 1.179682155 | 0 |
| 1.17992574 | 0 |
| 1.180169325 | 0 |
| 1.18041291 | 0 |
| 1.180656495 | 0 |
| 1.18090008 | 0 |
| 1.181143665 | 0 |
| 1.18138725 | 0 |
| 1.181630835 | 0 |
| 1.18187442 | 0 |
| 1.182118005 | 2.175 |
| 1.18236159 | 5.403 |
| 1.182605175 | 8.256 |
| 1.18284876 | 8.256 |
| 1.183092345 | 8.257 |
| 1.18333593 | 7.807 |
| 1.183579515 | 7 |
| 1.1838231 | 6.193 |
| 1.184066685 | 6.194 |
| 1.18431027 | 6.194 |
| 1.184553855 | 5.837 |
| 1.18479744 | 5.03 |
| 1.185041025 | 4.223 |
| 1.18528461 | 4.13 |
| 1.185528195 | 4.13 |
| 1.18577178 | 3.867 |
| 1.186015365 | 3.06 |
| 1.18625895 | 2.253 |
| 1.186502535 | 2.065 |
| 1.18674612 | 2.065 |
| 1.186989705 | 1.896 |
| 1.18723329 | 1.088 |
| 1.187476875 | 0.281 |
| 1.18772046 | 0 |
| 1.187964045 | 0 |
| 1.18820763 | 0 |
| 1.188451215 | 0 |
| 1.1886948 | 0 |
| 1.188938385 | 0 |
| 1.18918197 | 0 |
| 1.189425555 | 0 |
| 1.18966914 | 0 |
| 1.189912725 | 0 |
| 1.19015631 | 0 |
| 1.190399895 | 0 |
| 1.19064348 | 0 |
| 1.190887065 | 0 |
| 1.19113065 | 0 |
| 1.191374235 | 0 |
| 1.19161782 | 0 |
| 1.191861405 | 0 |
| 1.19210499 | 0 |
| 1.192348575 | 0 |
| 1.19259216 | 0 |
| 1.192835745 | 0 |
| 1.19307933 | 0 |
| 1.193322915 | 0 |
| 1.1935665 | 0 |
| 1.193810085 | 0 |
| 1.19405367 | 0 |
| 1.194297255 | 0 |
| 1.19454084 | 0 |
| 1.194784425 | 0 |
| 1.19502801 | 0 |
| 1.195271595 | 0 |
| 1.19551518 | 0 |
| 1.195758765 | 0 |
| 1.19600235 | 0 |
| 1.196245935 | 0 |
| 1.19648952 | 0 |
| 1.196733105 | 0 |
| 1.19697669 | 0 |
| 1.197220275 | 0 |
| 1.19746386 | 0 |
| 1.197707445 | 0 |
| 1.19795103 | 0 |
| 1.198194615 | 0 |
| 1.1984382 | 0 |
| 1.198681785 | 0 |
| 1.19892537 | 0 |
| 1.199168955 | 0 |
| 1.19941254 | 0 |
| 1.199656125 | 0 |
| 1.19989971 | 0 |
| 1.200143295 | 0 |
| 1.20038688 | 0 |
| 1.200630465 | 0 |
| 1.20087405 | 0 |
| 1.201117635 | 0 |
| 1.20136122 | 0 |
| 1.201604805 | 0 |
| 1.20184839 | 0 |
| 1.202091975 | 0 |
| 1.20233556 | 0 |
| 1.202579145 | 0 |
| 1.20282273 | 0 |
| 1.203066315 | 0 |
| 1.2033099 | 0 |
| 1.203553485 | 0 |
| 1.20379707 | 0 |
| 1.204040655 | 0 |
| 1.20428424 | 0 |
| 1.204527825 | 0 |
| 1.20477141 | 0 |
| 1.205014995 | 0 |
| 1.20525858 | 0 |
| 1.205502165 | 0 |
| 1.20574575 | 0 |
| 1.205989335 | 0 |
| 1.20623292 | 0 |
| 1.206476505 | 0 |
| 1.20672009 | 0 |
| 1.206963675 | 0 |
| 1.20720726 | 0 |
| 1.207450845 | 0 |
| 1.20769443 | 0 |
| 1.207938015 | 0 |
| 1.2081816 | 0 |
| 1.208425185 | 0 |
| 1.20866877 | 0 |
| 1.208912355 | 0 |
| 1.20915594 | 0 |
| 1.209399525 | 0 |
| 1.20964311 | 0 |
| 1.209886695 | 0 |
| 1.21013028 | 0 |
| 1.210373865 | 0 |
| 1.21061745 | 0 |
| 1.210861035 | 0 |
| 1.21110462 | 0 |
| 1.211348205 | 0 |
| 1.21159179 | 0 |
| 1.211835375 | 0 |
| 1.21207896 | 0 |
| 1.212322545 | 0 |
| 1.21256613 | 0 |
| 1.212809715 | 0 |
| 1.2130533 | 0 |
| 1.213296885 | 0 |
| 1.21354047 | 0 |
| 1.213784055 | 0 |
| 1.21402764 | 0 |
| 1.214271225 | 0 |
| 1.21451481 | 0 |
| 1.214758395 | 0 |
| 1.21500198 | 0 |
| 1.215245565 | 0 |
| 1.21548915 | 0 |
| 1.215732735 | 0 |
| 1.21597632 | 0 |
| 1.216219905 | 0 |
| 1.21646349 | 0 |
| 1.216707075 | 0 |
| 1.21695066 | 0 |
| 1.217194245 | 0 |
| 1.21743783 | 0 |
| 1.217681415 | 0 |
| 1.217925 | 0 |
| 1.218168585 | 0 |
| 1.21841217 | 0 |
| 1.218655755 | 0 |
| 1.21889934 | 0 |
| 1.219142925 | 0 |
| 1.21938651 | 0 |
| 1.219630095 | 0 |
| 1.21987368 | 0 |
| 1.220117265 | 0 |
| 1.22036085 | 0 |
| 1.220604435 | 0 |
| 1.22084802 | 0 |
| 1.221091605 | 0 |
| 1.22133519 | 0 |
| 1.221578775 | 0 |
| 1.22182236 | 0 |
| 1.222065945 | 0 |
| 1.22230953 | 0 |
| 1.222553115 | 0 |
| 1.2227967 | 0 |
| 1.223040285 | 0 |
| 1.22328387 | 0 |
| 1.223527455 | 0 |
| 1.22377104 | 0 |
| 1.224014625 | 0 |
| 1.22425821 | 0 |
| 1.224501795 | 0 |
| 1.22474538 | 0 |
| 1.224988965 | 0 |
| 1.22523255 | 0 |
| 1.225476135 | 0 |
| 1.22571972 | 0 |
| 1.225963305 | 0 |
| 1.22620689 | 0 |
| 1.226450475 | 0 |
| 1.22669406 | 0 |
| 1.226937645 | 0 |
| 1.22718123 | 0 |
| 1.227424815 | 0 |
| 1.2276684 | 0 |
| 1.227911985 | 0 |
| 1.22815557 | 0 |
| 1.228399155 | 0 |
| 1.22864274 | 0 |
| 1.228886325 | 0 |
| 1.22912991 | 0 |
| 1.229373495 | 0 |
| 1.22961708 | 0 |
| 1.229860665 | 0 |
| 1.23010425 | 0 |
| 1.230347835 | 0 |
| 1.23059142 | 0 |
| 1.230835005 | 0 |
| 1.23107859 | 0 |
| 1.231322175 | 0 |
| 1.23156576 | 0 |
| 1.231809345 | 0 |
| 1.23205293 | 0 |
| 1.232296515 | 0 |
| 1.2325401 | 0 |
| 1.232783685 | 0 |
| 1.23302727 | 0 |
| 1.233270855 | 0 |
| 1.23351444 | 0 |
| 1.233758025 | 0 |
| 1.23400161 | 0 |
| 1.234245195 | 0 |
| 1.23448878 | 0 |
| 1.234732365 | 0 |
| 1.23497595 | 0 |
| 1.235219535 | 0 |
| 1.23546312 | 0 |
| 1.235706705 | 0 |
| 1.23595029 | 0 |
| 1.236193875 | 0 |
| 1.23643746 | 0 |
| 1.236681045 | 0 |
| 1.23692463 | 0 |
| 1.237168215 | 0 |
| 1.2374118 | 0 |
| 1.237655385 | 0 |
| 1.23789897 | 0 |
| 1.238142555 | 0 |
| 1.23838614 | 0 |
| 1.238629725 | 0 |
| 1.23887331 | 0 |
| 1.239116895 | 0 |
| 1.23936048 | 0 |
| 1.239604065 | 0 |
| 1.23984765 | 0 |
| 1.240091235 | 0 |
| 1.24033482 | 0 |
| 1.240578405 | 0 |
| 1.24082199 | 0 |
| 1.241065575 | 0 |
| 1.24130916 | 0 |
| 1.241552745 | 0 |
| 1.24179633 | 0 |
| 1.242039915 | 0 |
| 1.2422835 | 0 |
| 1.242527085 | 0 |
| 1.24277067 | 0 |
| 1.243014255 | 0 |
| 1.24325784 | 0 |
| 1.243501425 | 0 |
| 1.24374501 | 0 |
| 1.243988595 | 0 |
| 1.24423218 | 0 |
| 1.244475765 | 0 |
| 1.24471935 | 0 |
| 1.244962935 | 0 |
| 1.24520652 | 0 |
| 1.245450105 | 0 |
| 1.24569369 | 0 |
| 1.245937275 | 0 |
| 1.24618086 | 0 |
| 1.246424445 | 0 |
| 1.24666803 | 0 |
| 1.246911615 | 0 |
| 1.2471552 | 0 |
| 1.247398785 | 0 |
| 1.24764237 | 0 |
| 1.247885955 | 0 |
| 1.24812954 | 0 |
| 1.248373125 | 0 |
| 1.24861671 | 0 |
| 1.248860295 | 0 |
| 1.24910388 | 0 |
| 1.249347465 | 0 |
| 1.24959105 | 0 |
| 1.249834635 | 0 |
| 1.25007822 | 0 |
| 1.250321805 | 0 |
| 1.25056539 | 0 |
| 1.250808975 | 0 |
| 1.25105256 | 0 |
| 1.251296145 | 0 |
| 1.25153973 | 0 |
| 1.251783315 | 0 |
| 1.2520269 | 0 |
| 1.252270485 | 0 |
| 1.25251407 | 0 |
| 1.252757655 | 0 |
| 1.25300124 | 0 |
| 1.253244825 | 0 |
| 1.25348841 | 0 |
| 1.253731995 | 0 |
| 1.25397558 | 0 |
| 1.254219165 | 0 |
| 1.25446275 | 0 |
| 1.254706335 | 0 |
| 1.25494992 | 0 |
| 1.255193505 | 0 |
| 1.25543709 | 0 |
| 1.255680675 | 0 |
| 1.25592426 | 0 |
| 1.256167845 | 0 |
| 1.25641143 | 0 |
| 1.256655015 | 0 |
| 1.2568986 | 0 |
| 1.257142185 | 0 |
| 1.25738577 | 0 |
| 1.257629355 | 0 |
| 1.25787294 | 0 |
| 1.258116525 | 0 |
| 1.25836011 | 0 |
| 1.258603695 | 0 |
| 1.25884728 | 0 |
| 1.259090865 | 0 |
| 1.25933445 | 0 |
| 1.259578035 | 0 |
| 1.25982162 | 0 |
| 1.260065205 | 0 |
| 1.26030879 | 0 |
| 1.260552375 | 0 |
| 1.26079596 | 0 |
| 1.261039545 | 0 |
| 1.26128313 | 0 |
| 1.261526715 | 0 |
| 1.2617703 | 0 |
| 1.262013885 | 0 |
| 1.26225747 | 0 |
| 1.262501055 | 0 |
| 1.26274464 | 0 |
| 1.262988225 | 0 |
| 1.26323181 | 0 |
| 1.263475395 | 0 |
| 1.26371898 | 0 |
| 1.263962565 | 0 |
| 1.26420615 | 0 |
| 1.264449735 | 0 |
| 1.26469332 | 0 |
| 1.264936905 | 0 |
| 1.26518049 | 0 |
| 1.265424075 | 0 |
| 1.26566766 | 0 |
| 1.265911245 | 0 |
| 1.26615483 | 0 |
| 1.266398415 | 0 |
| 1.266642 | 0 |
| 1.266885585 | 0 |
| 1.26712917 | 0 |
| 1.267372755 | 0 |
| 1.26761634 | 0 |
| 1.267859925 | 0 |
| 1.26810351 | 0 |
| 1.268347095 | 0 |
| 1.26859068 | 0 |
| 1.268834265 | 0 |
| 1.26907785 | 0 |
| 1.269321435 | 0 |
| 1.26956502 | 0 |
| 1.269808605 | 0 |
| 1.27005219 | 0 |
| 1.270295775 | 0 |
| 1.27053936 | 0 |
| 1.270782945 | 0 |
| 1.27102653 | 0 |
| 1.271270115 | 0 |
| 1.2715137 | 0 |
| 1.271757285 | 0 |
| 1.27200087 | 0 |
| 1.272244455 | 0 |
| 1.27248804 | 0 |
| 1.272731625 | 0 |
| 1.27297521 | 0 |
| 1.273218795 | 0 |
| 1.27346238 | 0 |
| 1.273705965 | 0 |
| 1.27394955 | 0 |
| 1.274193135 | 0 |
| 1.27443672 | 0 |
| 1.274680305 | 0 |
| 1.27492389 | 0 |
| 1.275167475 | 0 |
| 1.27541106 | 0 |
| 1.275654645 | 0 |
| 1.27589823 | 0 |
| 1.276141815 | 0 |
| 1.2763854 | 0 |
| 1.276628985 | 0 |
| 1.27687257 | 0 |
| 1.277116155 | 0 |
| 1.27735974 | 0 |
| 1.277603325 | 0 |
| 1.27784691 | 0 |
| 1.278090495 | 0 |
| 1.27833408 | 0 |
| 1.278577665 | 0 |
| 1.27882125 | 0 |
| 1.279064835 | 0 |
| 1.27930842 | 0 |
| 1.279552005 | 0 |
| 1.27979559 | 0 |
| 1.280039175 | 0 |
| 1.28028276 | 0 |
| 1.280526345 | 0 |
| 1.28076993 | 0 |
| 1.281013515 | 0 |
| 1.2812571 | 0 |
| 1.281500685 | 0 |
| 1.28174427 | 0 |
| 1.281987855 | 0 |
| 1.28223144 | 0 |
| 1.282475025 | 0 |
| 1.28271861 | 0 |
| 1.282962195 | 0 |
| 1.28320578 | 0 |
| 1.283449365 | 0 |
| 1.28369295 | 0 |
| 1.283936535 | 0 |
| 1.28418012 | 0 |
| 1.284423705 | 0 |
| 1.28466729 | 0 |
| 1.284910875 | 0 |
| 1.28515446 | 0 |
| 1.285398045 | 0 |
| 1.28564163 | 0 |
| 1.285885215 | 0 |
| 1.2861288 | 0 |
| 1.286372385 | 0 |
| 1.28661597 | 0 |
| 1.286859555 | 0 |
| 1.28710314 | 0 |
| 1.287346725 | 0 |
| 1.28759031 | 0 |
| 1.287833895 | 0 |
| 1.28807748 | 0 |
| 1.288321065 | 0 |
| 1.28856465 | 0 |
| 1.288808235 | 0 |
| 1.28905182 | 0 |
| 1.289295405 | 0 |
| 1.28953899 | 0 |
| 1.289782575 | 0 |
| 1.29002616 | 0 |
| 1.290269745 | 0 |
| 1.29051333 | 0 |
| 1.290756915 | 0 |
| 1.2910005 | 0 |
| 1.291244085 | 0 |
| 1.29148767 | 0 |
| 1.291731255 | 0 |
| 1.29197484 | 0 |
| 1.292218425 | 0 |
| 1.29246201 | 0 |
| 1.292705595 | 0 |
| 1.29294918 | 0 |
| 1.293192765 | 0 |
| 1.29343635 | 0 |
| 1.293679935 | 0 |
| 1.29392352 | 0 |
| 1.294167105 | 0 |
| 1.29441069 | 0 |
| 1.294654275 | 0 |
| 1.29489786 | 0 |
| 1.295141445 | 0 |
| 1.29538503 | 0 |
| 1.295628615 | 0 |
| 1.2958722 | 0 |
| 1.296115785 | 0 |
| 1.29635937 | 0 |
| 1.296602955 | 0 |
| 1.29684654 | 0 |
| 1.297090125 | 0 |
| 1.29733371 | 0 |
| 1.297577295 | 0 |
| 1.29782088 | 0 |
| 1.298064465 | 0 |
| 1.29830805 | 0 |
| 1.298551635 | 0 |
| 1.29879522 | 0 |
| 1.299038805 | 0 |
| 1.29928239 | 0 |
| 1.299525975 | 0 |
| 1.29976956 | 0 |
| 1.300013145 | 0 |
| 1.30025673 | 0 |
| 1.300500315 | 0 |
| 1.3007439 | 0 |
| 1.300987485 | 0 |
| 1.30123107 | 0 |
| 1.301474655 | 0 |
| 1.30171824 | 0 |
| 1.301961825 | 0 |
| 1.30220541 | 0 |
| 1.302448995 | 0 |
| 1.30269258 | 0 |
| 1.302936165 | 0 |
| 1.30317975 | 0 |
| 1.303423335 | 0 |
| 1.30366692 | 0 |
| 1.303910505 | 0 |
| 1.30415409 | 0 |
| 1.304397675 | 0 |
| 1.30464126 | 0 |
| 1.304884845 | 0 |
| 1.30512843 | 0 |
| 1.305372015 | 0 |
| 1.3056156 | 0 |
| 1.305859185 | 0 |
| 1.30610277 | 0 |
| 1.306346355 | 0 |
| 1.30658994 | 0 |
| 1.306833525 | 0 |
| 1.30707711 | 0 |
| 1.307320695 | 0 |
| 1.30756428 | 0 |
| 1.307807865 | 0 |
| 1.30805145 | −0.142 |
| 1.308295035 | −0.291 |
| 1.30853862 | −0.618 |
| 1.308782205 | −1.211 |
| 1.30902579 | −1.805 |
| 1.309269375 | −2.523 |
| 1.30951296 | −3.264 |
| 1.309756545 | −4.215 |
| 1.31000013 | −5.696 |
| 1.310243715 | −7.177 |
| 1.3104873 | −3.28 |
| 1.310730885 | 2.645 |
| 1.31097447 | 7.451 |
| 1.311218055 | 6.708 |
| 1.31146164 | 5.966 |
| 1.311705225 | 5.675 |
| 1.31194881 | 5.673 |
| 1.312192395 | 5.633 |
| 1.31243598 | 4.892 |
| 1.312679565 | 4.152 |
| 1.31292315 | 3.776 |
| 1.313166735 | 3.775 |
| 1.31341032 | 3.774 |
| 1.313653905 | 3.083 |
| 1.31389749 | 2.344 |
| 1.314141075 | 1.885 |
| 1.31438466 | 1.884 |
| 1.314628245 | 1.883 |
| 1.31487183 | 1.28 |
| 1.315115415 | 0.543 |
| 1.315359 | 0 |
| 1.315602585 | 0 |
| 1.31584617 | 0 |
| 1.316089755 | 0 |
| 1.31633334 | 0 |
| 1.316576925 | 0 |
| 1.31682051 | 0 |
| 1.317064095 | 0 |
| 1.31730768 | 0 |
| 1.317551265 | 0 |
| 1.31779485 | 0 |
| 1.318038435 | 0 |
| 1.31828202 | 0 |
| 1.318525605 | 0 |
| 1.31876919 | 0 |
| 1.319012775 | 0 |
| 1.31925636 | 0 |
| 1.319499945 | 0 |
| 1.31974353 | 0 |
| 1.319987115 | 0 |
| 1.3202307 | 0 |
| 1.320474285 | 2.327 |
| 1.32071787 | 5.244 |
| 1.320961455 | 7.46 |
| 1.32120504 | 7.457 |
| 1.321448625 | 7.454 |
| 1.32169221 | 5.466 |
| 1.321935795 | 2.552 |
| 1.32217938 | 0 |
| 1.322422965 | 0 |
| 1.32266655 | 0 |
| 1.322910135 | 0 |
| 1.32315372 | 0 |
| 1.323397305 | −0.001 |
| 1.32364089 | −0.146 |
| 1.323884475 | −0.291 |
| 1.32412806 | −0.632 |
| 1.324371645 | −1.212 |
| 1.32461523 | −1.791 |
| 1.324858815 | −2.499 |
| 1.3251024 | −3.223 |
| 1.325345985 | −4.187 |
| 1.32558957 | −5.632 |
| 1.325833155 | −7.077 |
| 1.32607674 | −2.913 |
| 1.326320325 | 2.871 |
| 1.32656391 | 7.236 |
| 1.326807495 | 6.511 |
| 1.32705108 | 5.786 |
| 1.327294665 | 5.538 |
| 1.32753825 | 5.536 |
| 1.327781835 | 5.46 |
| 1.32802542 | 4.738 |
| 1.328269005 | 4.015 |
| 1.32851259 | 3.685 |
| 1.328756175 | 3.683 |
| 1.32899976 | 3.682 |
| 1.329243345 | 2.971 |
| 1.32948693 | 2.251 |
| 1.329730515 | 1.839 |
| 1.3299741 | 1.838 |
| 1.330217685 | 1.837 |
| 1.33046127 | 1.212 |
| 1.330704855 | 0.494 |
| 1.33094844 | 0 |
| 1.331192025 | 0 |
| 1.33143561 | 0 |
| 1.331679195 | 0 |
| 1.33192278 | 0 |
| 1.332166365 | 0 |
| 1.33240995 | 0 |
| 1.332653535 | 0 |
| 1.33289712 | 0 |
| 1.333140705 | 0 |
| 1.33338429 | 0 |
| 1.333627875 | 0 |
| 1.33387146 | 0 |
| 1.334115045 | 0 |
| 1.33435863 | 0 |
| 1.334602215 | 0 |
| 1.3348458 | 0 |
| 1.335089385 | 0 |
| 1.33533297 | 0 |
| 1.335576555 | 0 |
| 1.33582014 | 0 |
| 1.336063725 | 2.41 |
| 1.33630731 | 5.253 |
| 1.336550895 | 7.27 |
| 1.33679448 | 7.267 |
| 1.337038065 | 7.264 |
| 1.33728165 | 5.184 |
| 1.337525235 | 2.345 |
| 1.33776882 | 0 |
| 1.338012405 | 0 |
| 1.33825599 | 0 |
| 1.338499575 | 0 |
| 1.33874316 | 0 |
| 1.338986745 | −0.008 |
| 1.33923033 | −0.15 |
| 1.339473915 | −0.291 |
| 1.3397175 | −0.644 |
| 1.339961085 | −1.208 |
| 1.34020467 | −1.773 |
| 1.340448255 | −2.469 |
| 1.34069184 | −3.174 |
| 1.340935425 | −4.148 |
| 1.34117901 | −5.555 |
| 1.341422595 | −6.963 |
| 1.34166618 | −2.555 |
| 1.341909765 | 3.077 |
| 1.34215335 | 7.01 |
| 1.342396935 | 6.304 |
| 1.34264052 | 5.598 |
| 1.342884105 | 5.392 |
| 1.34312769 | 5.39 |
| 1.343371275 | 5.281 |
| 1.34361486 | 4.577 |
| 1.343858445 | 3.873 |
| 1.34410203 | 3.587 |
| 1.344345615 | 3.585 |
| 1.3445892 | 3.558 |
| 1.344832785 | 2.857 |
| 1.34507637 | 2.156 |
| 1.345319955 | 1.789 |
| 1.34556354 | 1.789 |
| 1.345807125 | 1.788 |
| 1.34605071 | 1.145 |
| 1.346294295 | 0.446 |
| 1.34653788 | 0 |
| 1.346781465 | 0 |
| 1.34702505 | 0 |
| 1.347268635 | 0 |
| 1.34751222 | 0 |
| 1.347755805 | 0 |
| 1.34799939 | 0 |
| 1.348242975 | 0 |
| 1.34848656 | 0 |
| 1.348730145 | 0 |
| 1.34897373 | 0 |
| 1.349217315 | 0 |
| 1.3494609 | 0 |
| 1.349704485 | 0 |
| 1.34994807 | 0 |
| 1.350191655 | 0 |
| 1.35043524 | 0 |
| 1.350678825 | 0 |
| 1.35092241 | 0 |
| 1.351165995 | 0 |
| 1.35140958 | 0 |
| 1.351653165 | 2.482 |
| 1.35189675 | 5.246 |
| 1.352140335 | 7.069 |
| 1.35238392 | 7.066 |
| 1.352627505 | 7.063 |
| 1.35287109 | 4.903 |
| 1.353114675 | 2.142 |
| 1.35335826 | 0 |
| 1.353601845 | 0 |
| 1.35384543 | 0 |
| 1.354089015 | 0 |
| 1.3543326 | 0 |
| 1.354576185 | −0.015 |
| 1.35481977 | −0.152 |
| 1.355063355 | −0.29 |
| 1.35530694 | −0.653 |
| 1.355550525 | −1.202 |
| 1.35579411 | −1.751 |
| 1.356037695 | −2.434 |
| 1.35628128 | −3.119 |
| 1.356524865 | −4.1 |
| 1.35676845 | −5.468 |
| 1.357012035 | −6.835 |
| 1.35725562 | −2.21 |
| 1.357499205 | 3.264 |
| 1.35774279 | 6.778 |
| 1.357986375 | 6.092 |
| 1.35822996 | 5.406 |
| 1.358473545 | 5.239 |
| 1.35871713 | 5.237 |
| 1.358960715 | 5.097 |
| 1.3592043 | 4.413 |
| 1.359447885 | 3.729 |
| 1.35969147 | 3.485 |
| 1.359935055 | 3.483 |
| 1.36017864 | 3.423 |
| 1.360422225 | 2.742 |
| 1.36066581 | 2.06 |
| 1.360909395 | 1.738 |
| 1.36115298 | 1.738 |
| 1.361396565 | 1.737 |
| 1.36164015 | 1.078 |
| 1.361883735 | 0.399 |
| 1.36212732 | 0 |
| 1.362370905 | 0 |
| 1.36261449 | 0 |
| 1.362858075 | 0 |
| 1.36310166 | 0 |
| 1.363345245 | 0 |
| 1.36358883 | 0 |
| 1.363832415 | 0 |
| 1.364076 | 0 |
| 1.364319585 | 0 |
| 1.36456317 | 0 |
| 1.364806755 | 0 |
| 1.36505034 | 0 |
| 1.365293925 | 0 |
| 1.36553751 | 0 |
| 1.365781095 | 0 |
| 1.36602468 | 0 |
| 1.366268265 | 0 |
| 1.36651185 | 0 |
| 1.366755435 | 0 |
| 1.36699902 | 0 |
| 1.367242605 | 2.544 |
| 1.36748619 | 5.227 |
| 1.367729775 | 6.863 |
| 1.36797336 | 6.859 |
| 1.368216945 | 6.856 |
| 1.36846053 | 4.626 |
| 1.368704115 | 1.946 |
| 1.3689477 | 0 |
| 1.369191285 | 0 |
| 1.36943487 | 0 |
| 1.369678455 | 0 |
| 1.36992204 | 0 |
| 1.370165625 | 0 |
| 1.37040921 | 0 |
| 1.370652795 | 0 |
| 1.37089638 | 0 |
| 1.371139965 | 0 |
| 1.37138355 | 0 |
| 1.371627135 | 0 |
| 1.37187072 | 0 |
| 1.372114305 | 0 |
| 1.37235789 | 0 |
| 1.372601475 | 0 |
| 1.37284506 | 0 |
| 1.373088645 | 0 |
| 1.37333223 | 0 |
| 1.373575815 | 0 |
| 1.3738194 | 0 |
| 1.374062985 | 0 |
| 1.37430657 | 0 |
| 1.374550155 | 0 |
| 1.37479374 | 0 |
| 1.375037325 | 0 |
| 1.37528091 | 0 |
| 1.375524495 | 0 |
| 1.37576808 | 0 |
| 1.376011665 | 0 |
| 1.37625525 | 0 |
| 1.376498835 | 0 |
| 1.37674242 | 0 |
| 1.376986005 | 0 |
| 1.37722959 | 0 |
| 1.377473175 | 0 |
| 1.37771676 | 0 |
| 1.377960345 | 0 |
| 1.37820393 | 0 |
| 1.378447515 | 0 |
| 1.3786911 | 0 |
| 1.378934685 | 0 |
| 1.37917827 | 0 |
| 1.379421855 | 0 |
| 1.37966544 | 0 |
| 1.379909025 | 0 |
| 1.38015261 | 0 |
| 1.380396195 | 0 |
| 1.38063978 | 0 |
| 1.380883365 | 0 |
| 1.38112695 | 0 |
| 1.381370535 | 0 |
| 1.38161412 | 0 |
| 1.381857705 | 0 |
| 1.38210129 | 0 |
| 1.382344875 | 0 |
| 1.38258846 | 0 |
| 1.382832045 | 0 |
| 1.38307563 | 0 |
| 1.383319215 | 0 |
| 1.3835628 | 0 |
| 1.383806385 | 0 |
| 1.38404997 | 0 |
| 1.384293555 | 0 |
| 1.38453714 | 0 |
| 1.384780725 | 0 |
| 1.38502431 | 0 |
| 1.385267895 | 0 |
| 1.38551148 | 0 |
| 1.385755065 | −0.027 |
| 1.38599865 | −0.156 |
| 1.386242235 | −0.285 |
| 1.38648582 | −0.666 |
| 1.386729405 | −1.182 |
| 1.38697299 | −1.71 |
| 1.387216575 | −2.354 |
| 1.38746016 | −2.998 |
| 1.387703745 | −3.985 |
| 1.38794733 | −5.271 |
| 1.388190915 | −6.557 |
| 1.3884345 | −1.564 |
| 1.388678085 | 3.584 |
| 1.38892167 | 6.31 |
| 1.389165255 | 5.664 |
| 1.38940884 | 5.019 |
| 1.389652425 | 4.926 |
| 1.38989601 | 4.924 |
| 1.390139595 | 4.728 |
| 1.39038318 | 4.085 |
| 1.390626765 | 3.442 |
| 1.39087035 | 3.276 |
| 1.391113935 | 3.274 |
| 1.39135752 | 3.154 |
| 1.391601105 | 2.513 |
| 1.39184469 | 1.873 |
| 1.392088275 | 1.634 |
| 1.39233186 | 1.633 |
| 1.392575445 | 1.588 |
| 1.39281903 | 0.95 |
| 1.393062615 | 0.311 |
| 1.3933062 | 0 |
| 1.393549785 | 0 |
| 1.39379337 | 0 |
| 1.394036955 | 0 |
| 1.39428054 | 0 |
| 1.394524125 | 0 |
| 1.39476771 | 0 |
| 1.395011295 | 0 |
| 1.39525488 | 0 |
| 1.395498465 | 0 |
| 1.39574205 | 0 |
| 1.395985635 | 0 |
| 1.39622922 | 0 |
| 1.396472805 | 0 |
| 1.39671639 | 0 |
| 1.396959975 | 0 |
| 1.39720356 | 0 |
| 1.397447145 | 0 |
| 1.39769073 | 0 |
| 1.397934315 | 0 |
| 1.3981779 | 0.121 |
| 1.398421485 | 2.641 |
| 1.39866507 | 5.162 |
| 1.398908655 | 6.445 |
| 1.39915224 | 6.442 |
| 1.399395825 | 6.439 |
| 1.39963941 | 4.093 |
| 1.399882995 | 1.576 |
| 1.40012658 | 0 |
| 1.400370165 | 0 |
| 1.40061375 | 0 |
| 1.400857335 | 0 |
| 1.40110092 | 0 |
| 1.401344505 | −0.032 |
| 1.40158809 | −0.158 |
| 1.401831675 | −0.283 |
| 1.40207526 | −0.67 |
| 1.402318845 | −1.17 |
| 1.40256243 | −1.688 |
| 1.402806015 | −2.312 |
| 1.4030496 | −2.935 |
| 1.403293185 | −3.922 |
| 1.40353677 | −5.168 |
| 1.403780355 | −6.252 |
| 1.40402394 | −1.266 |
| 1.404267525 | 3.721 |
| 1.40451111 | 6.081 |
| 1.404754695 | 5.455 |
| 1.40499828 | 4.83 |
| 1.405241865 | 4.772 |
| 1.40548545 | 4.769 |
| 1.405729035 | 4.548 |
| 1.40597262 | 3.926 |
| 1.406216205 | 3.303 |
| 1.40645979 | 3.173 |
| 1.406703375 | 3.172 |
| 1.40694696 | 3.024 |
| 1.407190545 | 2.403 |
| 1.40743413 | 1.783 |
| 1.407677715 | 1.583 |
| 1.4079213 | 1.582 |
| 1.408164885 | 1.507 |
| 1.40840847 | 0.889 |
| 1.408652055 | 0.271 |
| 1.40889564 | 0 |
| 1.409139225 | 0 |
| 1.40938281 | 0 |
| 1.409626395 | 0 |
| 1.40986998 | 0 |
| 1.410113565 | 0 |
| 1.41035715 | 0 |
| 1.410600735 | 0 |
| 1.41084432 | 0 |
| 1.411087905 | 0 |
| 1.41133149 | 0 |
| 1.411575075 | 0 |
| 1.41181866 | 0 |
| 1.412062245 | 0 |
| 1.41230583 | 0 |
| 1.412549415 | 0 |
| 1.412793 | 0 |
| 1.413036585 | 0 |
| 1.41328017 | 0 |
| 1.413523755 | 0 |
| 1.41376734 | 0.239 |
| 1.414010925 | 2.68 |
| 1.41425451 | 5.121 |
| 1.414498095 | 6.243 |
| 1.41474168 | 6.239 |
| 1.414985265 | 6.236 |
| 1.41522885 | 3.842 |
| 1.415472435 | 1.404 |
| 1.41571602 | 0 |
| 1.415959605 | 0 |
| 1.41620319 | 0 |
| 1.416446775 | 0 |
| 1.41669036 | 0 |
| 1.416933945 | −0.037 |
| 1.41717753 | −0.159 |
| 1.417421115 | −0.28 |
| 1.4176647 | −0.673 |
| 1.417908285 | −1.158 |
| 1.41815187 | −1.665 |
| 1.418395455 | −2.269 |
| 1.41863904 | −2.873 |
| 1.418882625 | −3.859 |
| 1.41912621 | −5.066 |
| 1.419369795 | −5.814 |
| 1.41961338 | −0.984 |
| 1.419856965 | 3.845 |
| 1.42010055 | 5.859 |
| 1.420344135 | 5.254 |
| 1.42058772 | 4.648 |
| 1.420831305 | 4.622 |
| 1.42107489 | 4.62 |
| 1.421318475 | 4.376 |
| 1.42156206 | 3.772 |
| 1.421805645 | 3.169 |
| 1.42204923 | 3.074 |
| 1.422292815 | 3.072 |
| 1.4225364 | 2.899 |
| 1.422779985 | 2.298 |
| 1.42302357 | 1.697 |
| 1.423267155 | 1.533 |
| 1.42351074 | 1.532 |
| 1.423754325 | 1.43 |
| 1.42399791 | 0.831 |
| 1.424241495 | 0.232 |
| 1.42448508 | 0 |
| 1.424728665 | 0 |
| 1.42497225 | 0 |
| 1.425215835 | 0 |
| 1.42545942 | 0 |
| 1.425703005 | 0 |
| 1.42594659 | 0 |
| 1.426190175 | 0 |
| 1.42643376 | 0 |
| 1.426677345 | 0 |
| 1.42692093 | 0 |
| 1.427164515 | 0 |
| 1.4274081 | 0 |
| 1.427651685 | 0 |
| 1.42789527 | 0 |
| 1.428138855 | 0 |
| 1.42838244 | 0 |
| 1.428626025 | 0 |
| 1.42886961 | 0 |
| 1.429113195 | 0 |
| 1.42935678 | 0.35 |
| 1.429600365 | 2.715 |
| 1.42984395 | 5.08 |
| 1.430087535 | 6.048 |
| 1.43033112 | 6.045 |
| 1.430574705 | 5.967 |
| 1.43081829 | 3.605 |
| 1.431061875 | 1.243 |
| 1.43130546 | 0 |
| 1.431549045 | 0 |
| 1.43179263 | 0 |
| 1.432036215 | 0 |
| 1.4322798 | 0 |
| 1.432523385 | 0 |
| 1.43276697 | 0 |
| 1.433010555 | 0 |
| 1.43325414 | 0 |
| 1.433497725 | 0 |
| 1.43374131 | 0 |
| 1.433984895 | 0 |
| 1.43422848 | 0 |
| 1.434472065 | 0 |
| 1.43471565 | 0 |
| 1.434959235 | 0 |
| 1.43520282 | 0 |
| 1.435446405 | 0 |
| 1.43568999 | 0 |
| 1.435933575 | 0 |
| 1.43617716 | 0 |
| 1.436420745 | 0 |
| 1.43666433 | 0 |
| 1.436907915 | 0 |
| 1.4371515 | 0 |
| 1.437395085 | 0 |
| 1.43763867 | 0 |
| 1.437882255 | 0 |
| 1.43812584 | 0 |
| 1.438369425 | 0 |
| 1.43861301 | 0 |
| 1.438856595 | 0 |
| 1.43910018 | 0 |
| 1.439343765 | 0 |
| 1.43958735 | 0 |
| 1.439830935 | 0 |
| 1.44007452 | 0 |
| 1.440318105 | 0 |
| 1.44056169 | 0 |
| 1.440805275 | 0 |
| 1.44104886 | 0 |
| 1.441292445 | 0 |
| 1.44153603 | 0 |
| 1.441779615 | 0 |
| 1.4420232 | 0 |
| 1.442266785 | 0 |
| 1.44251037 | 0 |
| 1.442753955 | 0 |
| 1.44299754 | 0 |
| 1.443241125 | 0 |
| 1.44348471 | 0 |
| 1.443728295 | 0 |
| 1.44397188 | 0 |
| 1.444215465 | 0 |
| 1.44445905 | 0 |
| 1.444702635 | 0 |
| 1.44494622 | 0 |
| 1.445189805 | 0 |
| 1.44543339 | 0 |
| 1.445676975 | 0 |
| 1.44592056 | 0 |
| 1.446164145 | 0 |
| 1.44640773 | 0 |
| 1.446651315 | 0 |
| 1.4468949 | 0 |
| 1.447138485 | 0 |
| 1.44738207 | 0 |
| 1.447625655 | 0 |
| 1.44786924 | 0 |
| 1.448112825 | 0 |
| 1.44835641 | 0 |
| 1.448599995 | 0 |
| 1.44884358 | 0 |
| 1.449087165 | 0 |
| 1.44933075 | 0 |
| 1.449574335 | 0 |
| 1.44981792 | 0 |
| 1.450061505 | 0 |
| 1.45030509 | 0 |
| 1.450548675 | 0 |
| 1.45079226 | 0 |
| 1.451035845 | 0 |
| 1.45127943 | 0 |
| 1.451523015 | 0 |
| 1.4517666 | 0 |
| 1.452010185 | 0 |
| 1.45225377 | 0 |
| 1.452497355 | 0 |
| 1.45274094 | 0 |
| 1.452984525 | 0 |
| 1.45322811 | 0 |
| 1.453471695 | 0 |
| 1.45371528 | 0 |
| 1.453958865 | 0 |
| 1.45420245 | 0 |
| 1.454446035 | 0 |
| 1.45468962 | 0 |
| 1.454933205 | 0 |
| 1.45517679 | 0 |
| 1.455420375 | 0 |
| 1.45566396 | 0 |
| 1.455907545 | 0 |
| 1.45615113 | 0 |
| 1.456394715 | 0 |
| 1.4566383 | 0 |
| 1.456881885 | 0 |
| 1.45712547 | 0 |
| 1.457369055 | 0 |
| 1.45761264 | 0 |
| 1.457856225 | 0 |
| 1.45809981 | 0 |
| 1.458343395 | 0 |
| 1.45858698 | 0 |
| 1.458830565 | 0 |
| 1.45907415 | 0 |
| 1.459317735 | 0 |
| 1.45956132 | 0 |
| 1.459804905 | 0 |
| 1.46004849 | 0 |
| 1.460292075 | 0 |
| 1.46053566 | 0 |
| 1.460779245 | 0 |
| 1.46102283 | 0 |
| 1.461266415 | 0 |
| 1.46151 | 0 |
| 1.461753585 | 0 |
| 1.46199717 | 0 |
| 1.462240755 | 0 |
| 1.46248434 | 0 |
| 1.462727925 | 0 |
| 1.46297151 | 0 |
| 1.463215095 | 0 |
| 1.46345868 | 0 |
| 1.463702265 | −0.051 |
| 1.46394585 | −0.162 |
| 1.464189435 | −0.272 |
| 1.46443302 | −0.682 |
| 1.464676605 | −1.125 |
| 1.46492019 | −1.605 |
| 1.465163775 | −2.157 |
| 1.46540736 | −2.71 |
| 1.465650945 | −3.694 |
| 1.46589453 | −4.798 |
| 1.466138115 | −4.654 |
| 1.4663817 | −0.237 |
| 1.466625285 | 4.179 |
| 1.46686887 | 5.276 |
| 1.467112455 | 4.722 |
| 1.46735604 | 4.23 |
| 1.467599625 | 4.228 |
| 1.46784321 | 4.226 |
| 1.468086795 | 3.921 |
| 1.46833038 | 3.369 |
| 1.468573965 | 2.817 |
| 1.46881755 | 2.813 |
| 1.469061135 | 2.812 |
| 1.46930472 | 2.571 |
| 1.469548305 | 2.021 |
| 1.46979189 | 1.471 |
| 1.470035475 | 1.403 |
| 1.47027906 | 1.403 |
| 1.470522645 | 1.227 |
| 1.47076623 | 0.679 |
| 1.471009815 | 0.13 |
| 1.4712534 | 0 |
| 1.471496985 | 0 |
| 1.47174057 | 0 |
| 1.471984155 | 0 |
| 1.47222774 | 0 |
| 1.472471325 | 0 |
| 1.47271491 | 0 |
| 1.472958495 | 0 |
| 1.47320208 | 0 |
| 1.473445665 | 0 |
| 1.47368925 | 0 |
| 1.473932835 | 0 |
| 1.47417642 | 0 |
| 1.474420005 | 0 |
| 1.47466359 | 0 |
| 1.474907175 | 0 |
| 1.47515076 | 0 |
| 1.475394345 | 0 |
| 1.47563793 | 0 |
| 1.475881515 | 0 |
| 1.4761251 | 0.647 |
| 1.476368685 | 2.818 |
| 1.47661227 | 4.988 |
| 1.476855855 | 5.551 |
| 1.47709944 | 5.549 |
| 1.477343025 | 5.152 |
| 1.47758661 | 2.984 |
| 1.477830195 | 0.815 |
| 1.47807378 | 0 |
| 1.478317365 | 0 |
| 1.47856095 | 0 |
| 1.478804535 | 0 |
| 1.47904812 | 0 |
| 1.479291705 | −0.055 |
| 1.47953529 | −0.163 |
| 1.479778875 | −0.271 |
| 1.48002246 | −0.686 |
| 1.480266045 | −1.118 |
| 1.48050963 | −1.591 |
| 1.480753215 | −2.129 |
| 1.4809968 | −2.668 |
| 1.481240385 | −3.654 |
| 1.48148397 | −4.73 |
| 1.481727555 | −4.321 |
| 1.48197114 | −0.016 |
| 1.482214725 | 4.29 |
| 1.48245831 | 5.117 |
| 1.482701895 | 4.577 |
| 1.48294548 | 4.124 |
| 1.483189065 | 4.122 |
| 1.48343265 | 4.121 |
| 1.483676235 | 3.796 |
| 1.48391982 | 3.258 |
| 1.484163405 | 2.744 |
| 1.48440699 | 2.743 |
| 1.484650575 | 2.742 |
| 1.48489416 | 2.481 |
| 1.485137745 | 1.944 |
| 1.48538133 | 1.408 |
| 1.485624915 | 1.369 |
| 1.4858685 | 1.369 |
| 1.486112085 | 1.17 |
| 1.48635567 | 0.635 |
| 1.486599255 | 0.101 |
| 1.48684284 | 0 |
| 1.487086425 | 0 |
| 1.48733001 | 0 |
| 1.487573595 | 0 |
| 1.48781718 | 0 |
| 1.488060765 | 0 |
| 1.48830435 | 0 |
| 1.488547935 | 0 |
| 1.48879152 | 0 |
| 1.489035105 | 0 |
| 1.48927869 | 0 |
| 1.489522275 | 0 |
| 1.48976586 | 0 |
| 1.490009445 | 0 |
| 1.49025303 | 0 |
| 1.490496615 | 0 |
| 1.4907402 | 0 |
| 1.490983785 | 0 |
| 1.49122737 | 0 |
| 1.491470955 | 0 |
| 1.49171454 | 0.738 |
| 1.491958125 | 2.858 |
| 1.49220171 | 4.979 |
| 1.492445295 | 5.423 |
| 1.49268888 | 5.421 |
| 1.492932465 | 4.928 |
| 1.49317605 | 2.809 |
| 1.493419635 | 0.691 |
| 1.49366322 | 0 |
| 1.493906805 | 0 |
| 1.49415039 | 0 |
| 1.494393975 | 0 |
| 1.49463756 | 0 |
| 1.494881145 | 0 |
| 1.49512473 | 0 |
| 1.495368315 | 0 |
| 1.4956119 | 0 |
| 1.495855485 | 0 |
| 1.49609907 | 0 |
| 1.496342655 | 0 |
| 1.49658624 | 0 |
| 1.496829825 | 0 |
| 1.49707341 | 0 |
| 1.497316995 | 0 |
| 1.49756058 | 0 |
| 1.497804165 | 0 |
| 1.49804775 | 0 |
| 1.498291335 | 0 |
| 1.49853492 | 0 |
| 1.498778505 | 0 |
| 1.49902209 | 0 |
| 1.499265675 | 0 |
| 1.49950926 | 0 |
| 1.499752845 | 0 |
| 1.49999643 | 0 |
| 1.500240015 | 0 |
| 1.5004836 | 0 |
| 1.500727185 | 0 |
| 1.50097077 | 0 |
| 1.501214355 | 0 |
| 1.50145794 | 0 |
| 1.501701525 | 0 |
| 1.50194511 | 0 |
| 1.502188695 | 0 |
| 1.50243228 | 0 |
| 1.502675865 | 0 |
| 1.50291945 | 0 |
| 1.503163035 | 0 |
| 1.50340662 | 0 |
| 1.503650205 | 0 |
| 1.50389379 | 0 |
| 1.504137375 | 0 |
| 1.50438096 | 0 |
| 1.504624545 | 0 |
| 1.50486813 | 0 |
| 1.505111715 | 0 |
| 1.5053553 | 0 |
| 1.505598885 | 0 |
| 1.50584247 | 0 |
| 1.506086055 | 0 |
| 1.50632964 | 0 |
| 1.506573225 | 0 |
| 1.50681681 | 0 |
| 1.507060395 | 0 |
| 1.50730398 | 0 |
| 1.507547565 | 0 |
| 1.50779115 | 0 |
| 1.508034735 | 0 |
| 1.50827832 | 0 |
| 1.508521905 | 0 |
| 1.50876549 | 0 |
| 1.509009075 | 0 |
| 1.50925266 | 0 |
| 1.509496245 | 0 |
| 1.50973983 | 0 |
| 1.509983415 | 0 |
| 1.510227 | 0 |
| 1.510470585 | 0 |
| 1.51071417 | 0 |
| 1.510957755 | 0 |
| 1.51120134 | 0 |
| 1.511444925 | 0 |
| 1.51168851 | 0 |
| 1.511932095 | 0 |
| 1.51217568 | 0 |
| 1.512419265 | 0 |
| 1.51266285 | 0 |
| 1.512906435 | 0 |
| 1.51315002 | 0 |
| 1.513393605 | 0 |
| 1.51363719 | 0 |
| 1.513880775 | 0 |
| 1.51412436 | 0 |
| 1.514367945 | 0 |
| 1.51461153 | 0 |
| 1.514855115 | 0 |
| 1.5150987 | 0 |
| 1.515342285 | 0 |
| 1.51558587 | 0 |
| 1.515829455 | 0 |
| 1.51607304 | 0 |
| 1.516316625 | 0 |
| 1.51656021 | 0 |
| 1.516803795 | 0 |
| 1.51704738 | 0 |
| 1.517290965 | 0 |
| 1.51753455 | 0 |
| 1.517778135 | 0 |
| 1.51802172 | 0 |
| 1.518265305 | 0 |
| 1.51850889 | 0 |
| 1.518752475 | 0 |
| 1.51899606 | 0 |
| 1.519239645 | 0 |
| 1.51948323 | 0 |
| 1.519726815 | 0 |
| 1.5199704 | 0 |
| 1.520213985 | 0 |
| 1.52045757 | 0 |
| 1.520701155 | 0 |
| 1.52094474 | 0 |
| 1.521188325 | 0 |
| 1.52143191 | 0 |
| 1.521675495 | 0 |
| 1.52191908 | 0 |
| 1.522162665 | 0 |
| 1.52240625 | 0 |
| 1.522649835 | 0 |
| 1.52289342 | 0 |
| 1.523137005 | 0 |
| 1.52338059 | 0 |
| 1.523624175 | 0 |
| 1.52386776 | 0 |
| 1.524111345 | 0 |
| 1.52435493 | 0 |
| 1.524598515 | 0 |
| 1.5248421 | 0 |
| 1.525085685 | 0 |
| 1.52532927 | 0 |
| 1.525572855 | 0 |
| 1.52581644 | 0 |
| 1.526060025 | 0 |
| 1.52630361 | 0 |
| 1.526547195 | 0 |
| 1.52679078 | 0 |
| 1.527034365 | 0 |
| 1.52727795 | 0 |
| 1.527521535 | 0 |
| 1.52776512 | 0 |
| 1.528008705 | 0 |
| 1.52825229 | 0 |
| 1.528495875 | 0 |
| 1.52873946 | 0 |
| 1.528983045 | 0 |
| 1.52922663 | 0 |
| 1.529470215 | 0 |
| 1.5297138 | 0 |
| 1.529957385 | 0 |
| 1.53020097 | 0 |
| 1.530444555 | 0 |
| 1.53068814 | 0 |
| 1.530931725 | 0 |
| 1.53117531 | 0 |
| 1.531418895 | 0 |
| 1.53166248 | 0 |
| 1.531906065 | 0 |
| 1.53214965 | 0 |
| 1.532393235 | 0 |
| 1.53263682 | 0 |
| 1.532880405 | 0 |
| 1.53312399 | 0 |
| 1.533367575 | 0 |
| 1.53361116 | 0 |
| 1.533854745 | 0 |
| 1.53409833 | 0 |
| 1.534341915 | 0 |
| 1.5345855 | 0 |
| 1.534829085 | 0 |
| 1.53507267 | 0 |
| 1.535316255 | 0 |
| 1.53555984 | 0 |
| 1.535803425 | 0 |
| 1.53604701 | 0 |
| 1.536290595 | 0 |
| 1.53653418 | 0 |
| 1.536777765 | 0 |
| 1.53702135 | 0 |
| 1.537264935 | 0 |
| 1.53750852 | 0 |
| 1.537752105 | 0 |
| 1.53799569 | 0 |
| 1.538239275 | 0 |
| 1.53848286 | 0 |
| 1.538726445 | 0 |
| 1.53897003 | 0 |
| 1.539213615 | 0 |
| 1.5394572 | 0 |
| 1.539700785 | 0 |
| 1.53994437 | 0 |
| 1.540187955 | 0 |
| 1.54043154 | 0 |
| 1.540675125 | 0 |
| 1.54091871 | 0 |
| 1.541162295 | 0 |
| 1.54140588 | 0 |
| 1.541649465 | 0 |
| 1.54189305 | 0 |
| 1.542136635 | 0 |
| 1.54238022 | 0 |
| 1.542623805 | 0 |
| 1.54286739 | 0 |
| 1.543110975 | 0 |
| 1.54335456 | 0 |
| 1.543598145 | 0 |
| 1.54384173 | 0 |
| 1.544085315 | 0 |
| 1.5443289 | 0 |
| 1.544572485 | 0 |
| 1.54481607 | 0 |
| 1.545059655 | 0 |
| 1.54530324 | 0 |
| 1.545546825 | 0 |
| 1.54579041 | 0 |
| 1.546033995 | 0 |
| 1.54627758 | 0 |
| 1.546521165 | 0 |
| 1.54676475 | 0 |
| 1.547008335 | 0 |
| 1.54725192 | 0 |
| 1.547495505 | 0 |
| 1.54773909 | 0 |
| 1.547982675 | 0 |
| 1.54822626 | 0 |
| 1.548469845 | 0 |
| 1.54871343 | 0 |
| 1.548957015 | 0 |
| 1.5492006 | 0 |
| 1.549444185 | 0 |
| 1.54968777 | 0 |
| 1.549931355 | 0 |
| 1.55017494 | 0 |
| 1.550418525 | 0 |
| 1.55066211 | 0 |
| 1.550905695 | 0 |
| 1.55114928 | 0 |
| 1.551392865 | 0 |
| 1.55163645 | 0 |
| 1.551880035 | 0 |
| 1.55212362 | 0 |
| 1.552367205 | 0 |
| 1.55261079 | 0 |
| 1.552854375 | 0 |
| 1.55309796 | 0 |
| 1.553341545 | 0 |
| 1.55358513 | 0 |
| 1.553828715 | 0 |
| 1.5540723 | 0 |
| 1.554315885 | 0 |
| 1.55455947 | 0 |
| 1.554803055 | 0 |
| 1.55504664 | 0 |
| 1.555290225 | 0 |
| 1.55553381 | 0 |
| 1.555777395 | 0 |
| 1.55602098 | 0 |
| 1.556264565 | 0 |
| 1.55650815 | 0 |
| 1.556751735 | 0 |
| 1.55699532 | 0 |
| 1.557238905 | −0.076 |
| 1.55748249 | −0.176 |
| 1.557726075 | −0.337 |
| 1.55796966 | −0.739 |
| 1.558213245 | −1.14 |
| 1.55845683 | −1.606 |
| 1.558700415 | −2.108 |
| 1.558944 | −2.652 |
| 1.559187585 | −3.655 |
| 1.55943117 | −4.658 |
| 1.559674755 | −3.023 |
| 1.55991834 | 0.991 |
| 1.560161925 | 5.005 |
| 1.56040551 | 4.648 |
| 1.560649095 | 4.146 |
| 1.56089268 | 3.85 |
| 1.561136265 | 3.849 |
| 1.56137985 | 3.849 |
| 1.561623435 | 3.422 |
| 1.56186702 | 2.92 |
| 1.562110605 | 2.566 |
| 1.56235419 | 2.566 |
| 1.562597775 | 2.566 |
| 1.56284136 | 2.197 |
| 1.563084945 | 1.695 |
| 1.56332853 | 1.283 |
| 1.563572115 | 1.283 |
| 1.5638157 | 1.283 |
| 1.564059285 | 0.972 |
| 1.56430287 | 0.47 |
| 1.564546455 | 0 |
| 1.56479004 | 0 |
| 1.565033625 | 0 |
| 1.56527721 | 0 |
| 1.565520795 | 0 |
| 1.56576438 | 0 |
| 1.566007965 | 0 |
| 1.56625155 | 0 |
| 1.566495135 | 0 |
| 1.56673872 | 0 |
| 1.566982305 | 0 |
| 1.56722589 | 0 |
| 1.567469475 | 0 |
| 1.56771306 | 0 |
| 1.567956645 | 0 |
| 1.56820023 | 0 |
| 1.568443815 | 0 |
| 1.5686874 | 0 |
| 1.568930985 | 0 |
| 1.56917457 | 0 |
| 1.569418155 | 0 |
| 1.56966174 | 1.199 |
| 1.569905325 | 3.204 |
| 1.57014891 | 5.129 |
| 1.570392495 | 5.129 |
| 1.57063608 | 5.129 |
| 1.570879665 | 4.163 |
| 1.57112325 | 2.158 |
| 1.571366835 | 0.152 |
| 1.57161042 | 0 |
| 1.571854005 | 0 |
| 1.57209759 | 0 |
| 1.572341175 | 0 |
| 1.57258476 | 0 |
| 1.572828345 | 0 |
| 1.57307193 | 0 |
| 1.573315515 | 0 |
| 1.5735591 | 0 |
| 1.573802685 | 0 |
| 1.57404627 | 0 |
| 1.574289855 | 0 |
| 1.57453344 | 0 |
| 1.574777025 | 0 |
| 1.57502061 | 0 |
| 1.575264195 | 0 |
| 1.57550778 | 0 |
| 1.575751365 | 0 |
| 1.57599495 | 0 |
| 1.576238535 | 0 |
| 1.57648212 | 0 |
| 1.576725705 | 0 |
| 1.57696929 | 0 |
| 1.577212875 | 0 |
| 1.57745646 | 0 |
| 1.577700045 | 0 |
| 1.57794363 | 0 |
| 1.578187215 | 0 |
| 1.5784308 | 0 |
| 1.578674385 | 0 |
| 1.57891797 | 0 |
| 1.579161555 | 0 |
| 1.57940514 | 0 |
| 1.579648725 | 0 |
| 1.57989231 | 0 |
| 1.580135895 | 0 |
| 1.58037948 | 0 |
| 1.580623065 | 0 |
| 1.58086665 | 0 |
| 1.581110235 | 0 |
| 1.58135382 | 0 |
| 1.581597405 | 0 |
| 1.58184099 | 0 |
| 1.582084575 | 0 |
| 1.58232816 | 0 |
| 1.582571745 | 0 |
| 1.58281533 | 0 |
| 1.583058915 | 0 |
| 1.5833025 | 0 |
| 1.583546085 | 0 |
| 1.58378967 | 0 |
| 1.584033253 | 0 |
| 1.58427684 | 0 |
| 1.584520425 | 0 |
| 1.58476401 | 0 |
| 1.585007595 | 0 |
| 1.58525118 | 0 |
| 1.585494765 | 0 |
| 1.58573835 | 0 |
| 1.585981935 | 0 |
| 1.58622552 | 0 |
| 1.586469105 | 0 |
| 1.58671269 | 0 |
| 1.586956275 | 0 |
| 1.58719986 | 0 |
| 1.587443445 | 0 |
| 1.58768703 | 0 |
| 1.587930615 | 0 |
| 1.5881742 | 0 |
| 1.588417785 | 0 |
| 1.58866137 | 0 |
| 1.588904955 | 0 |
| 1.58914854 | 0 |
| 1.589392125 | 0 |
| 1.58963571 | 0 |
| 1.589879295 | 0 |
| 1.59012288 | 0 |
| 1.590366465 | 0 |
| 1.59061005 | 0 |
| 1.590853635 | 0 |
| 1.59109722 | 0 |
| 1.591340805 | 0 |
| 1.59158439 | 0 |
| 1.591827975 | 0 |
| 1.59207156 | 0 |
| 1.592315145 | 0 |
| 1.59255873 | 0 |
| 1.592802315 | 0 |
| 1.5930459 | 0 |
| 1.593289485 | 0 |
| 1.59353307 | 0 |
| 1.593776655 | 0 |
| 1.59402024 | 0 |
| 1.594263825 | 0 |
| 1.59450741 | 0 |
| 1.594750995 | 0 |
| 1.59499458 | 0 |
| 1.595238165 | 0 |
| 1.59548175 | 0 |
| 1.595725335 | 0 |
| 1.59596892 | 0 |
| 1.596212505 | 0 |
| 1.59645609 | 0 |
| 1.596699675 | 0 |
| 1.59694326 | 0 |
| 1.597186845 | 0 |
| 1.59743043 | 0 |
| 1.597674015 | 0 |
| 1.5979176 | 0 |
| 1.598161185 | 0 |
| 1.59840477 | 0 |
| 1.598648355 | 0 |
| 1.59889194 | 0 |
| 1.599135525 | 0 |
| 1.59937911 | 0 |
| 1.599622695 | 0 |
| 1.59986628 | 0 |
| 1.600109865 | 0 |
| 1.60035345 | 0 |
| 1.600597035 | 0 |
| 1.60084062 | 0 |
| 1.601084205 | 0 |
| 1.60132779 | 0 |
| 1.601571375 | 0 |
| 1.60181496 | 0 |
| 1.602058545 | 0 |
| 1.60230213 | 0 |
| 1.602545715 | 0 |
| 1.6027893 | 0 |
| 1.603032885 | 0 |
| 1.60327647 | 0 |
| 1.603520055 | 0 |
| 1.60376364 | 0 |
| 1.604007225 | 0 |
| 1.60425081 | 0 |
| 1.604494395 | 0 |
| 1.60473798 | 0 |
| 1.604981565 | 0 |
| 1.60522515 | 0 |
| 1.605468735 | 0 |
| 1.60571232 | 0 |
| 1.605955905 | 0 |
| 1.60619949 | 0 |
| 1.606443075 | 0 |
| 1.60668666 | 0 |
| 1.606930245 | 0 |
| 1.60717383 | 0 |
| 1.607417415 | 0 |
| 1.607661 | 0 |
| 1.607904585 | 0 |
| 1.60814817 | 0 |
| 1.608391755 | 0 |
| 1.60863534 | 0 |
| 1.608878925 | 0 |
| 1.60912251 | 0 |
| 1.609366095 | 0 |
| 1.60960968 | 0 |
| 1.609853265 | 0 |
| 1.61009685 | 0 |
| 1.610340435 | 0 |
| 1.61058402 | 0 |
| 1.610827605 | 0 |
| 1.61107119 | 0 |
| 1.611314775 | 0 |
| 1.61155836 | 0 |
| 1.611801945 | 0 |
| 1.61204553 | 0 |
| 1.612289115 | 0 |
| 1.6125327 | 0 |
| 1.612776285 | 0 |
| 1.61301987 | 0 |
| 1.613263455 | 0 |
| 1.61350704 | 0 |
| 1.613750625 | 0 |
| 1.61399421 | 0 |
| 1.614237795 | 0 |
| 1.61448138 | 0 |
| 1.614724965 | 0 |
| 1.61496855 | 0 |
| 1.615212135 | 0 |
| 1.61545572 | 0 |
| 1.615699305 | 0 |
| 1.61594289 | 0 |
| 1.616186475 | 0 |
| 1.61643006 | 0 |
| 1.616673645 | 0 |
| 1.61691723 | 0 |
| 1.617160815 | 0 |
| 1.6174044 | 0 |
| 1.617647985 | 0 |
| 1.61789157 | 0 |
| 1.618135155 | 0 |
| 1.61837874 | 0 |
| 1.618622325 | 0 |
| 1.61886591 | 0 |
| 1.619109495 | 0 |
| 1.61935308 | 0 |
| 1.619596665 | 0 |
| 1.61984025 | 0 |
| 1.620083835 | 0 |
| 1.62032742 | 0 |
| 1.620571005 | 0 |
| 1.62081459 | 0 |
| 1.621058175 | 0 |
| 1.62130176 | 0 |
| 1.621545345 | 0 |
| 1.62178893 | 0 |
| 1.622032515 | 0 |
| 1.6222761 | 0 |
| 1.622519685 | 0 |
| 1.62276327 | 0 |
| 1.623006855 | 0 |
| 1.62325044 | 0 |
| 1.623494025 | 0 |
| 1.62373761 | 0 |
| 1.623981195 | 0 |
| 1.62422478 | 0 |
| 1.624468365 | 0 |
| 1.62471195 | 0 |
| 1.624955535 | 0 |
| 1.62519912 | 0 |
| 1.625442705 | 0 |
| 1.62568629 | 0 |
| 1.625929875 | 0 |
| 1.62617346 | 0 |
| 1.626417045 | 0 |
| 1.62666063 | 0 |
| 1.626904215 | 0 |
| 1.6271478 | 0 |
| 1.627391385 | 0 |
| 1.62763497 | 0 |
| 1.627878555 | 0 |
| 1.62812214 | 0 |
| 1.628365725 | 0 |
| 1.62860931 | 0 |
| 1.628852895 | 0 |
| 1.62909648 | 0 |
| 1.629340065 | 0 |
| 1.62958365 | 0 |
| 1.629827235 | 0 |
| 1.63007082 | 0 |
| 1.630314405 | 0 |
| 1.63055799 | 0 |
| 1.630801575 | 0 |
| 1.63104516 | 0 |
| 1.631288745 | 0 |
| 1.63153233 | 0 |
| 1.631775915 | 0 |
| 1.6320195 | 0 |
| 1.632263085 | 0 |
| 1.63250667 | 0 |
| 1.632750255 | 0 |
| 1.63299384 | 0 |
| 1.633237425 | 0 |
| 1.63348101 | 0 |
| 1.633724595 | 0 |
| 1.63396818 | 0 |
| 1.634211765 | 0 |
| 1.63445535 | 0 |
| 1.634698935 | 0 |
| 1.63494252 | 0 |
| 1.635186105 | 0 |
| 1.63542969 | 0 |
| 1.635673275 | 0 |
| 1.63591686 | 0 |
| 1.636160445 | 0 |
| 1.63640403 | 0 |
| 1.636647615 | 0 |
| 1.6368912 | 0 |
| 1.637134785 | 0 |
| 1.63737837 | 0 |
| 1.637621955 | 0 |
| 1.63786554 | 0 |
| 1.638109125 | 0 |
| 1.63835271 | 0 |
| 1.638596295 | 0 |
| 1.63883988 | 0 |
| 1.639083465 | 0 |
| 1.63932705 | 0 |
| 1.639570635 | 0 |
| 1.63981422 | 0 |
| 1.640057805 | 0 |
| 1.64030139 | 0 |
| 1.640544975 | 0 |
| 1.64078856 | 0 |
| 1.641032145 | 0 |
| 1.64127573 | 0 |
| 1.641519315 | 0 |
| 1.6417629 | 0 |
| 1.642006485 | 0 |
| 1.64225007 | 0 |
| 1.642493655 | 0 |
| 1.64273724 | 0 |
| 1.642980825 | 0 |
| 1.64322441 | 0 |
| 1.643467995 | 0 |
| 1.64371158 | 0 |
| 1.643955165 | 0 |
| 1.64419875 | 0 |
| 1.644442335 | 0 |
| 1.64468592 | 0 |
| 1.644929505 | 0 |
| 1.64517309 | 0 |
| 1.645416675 | 0 |
| 1.64566026 | 0 |
| 1.645903845 | 0 |
| 1.64614743 | 0 |
| 1.646391015 | 0 |
| 1.6466346 | 0 |
| 1.646878185 | 0 |
| 1.64712177 | 0 |
| 1.647365355 | 0 |
| 1.64760894 | 0 |
| 1.647852525 | 0 |
| 1.64809611 | 0 |
| 1.648339695 | 0 |
| 1.64858328 | 0 |
| 1.648826865 | 0 |
| 1.64907045 | 0 |
| 1.649314035 | 0 |
| 1.64955762 | 0 |
| 1.649801205 | 0 |
| 1.65004479 | 0 |
| 1.650288375 | 0 |
| 1.65053196 | 0 |
| 1.650775545 | 0 |
| 1.65101913 | 0 |
| 1.651262715 | 0 |
| 1.6515063 | 0 |
| 1.651749885 | 0 |
| 1.65199347 | 0 |
| 1.652237055 | 0 |
| 1.65248064 | 0 |
| 1.652724225 | 0 |
| 1.65296781 | 0 |
| 1.653211395 | 0 |
| 1.65345498 | 0 |
| 1.653698565 | 0 |
| 1.65394215 | 0 |
| 1.654185735 | 0 |
| 1.65442932 | 0 |
| 1.654672905 | 0 |
| 1.65491649 | 0 |
| 1.655160075 | 0 |
| 1.65540366 | 0 |
| 1.655647245 | 0 |
| 1.65589083 | 0 |
| 1.656134415 | 0 |
| 1.656378 | 0 |
| 1.656621585 | 0 |
| 1.65686517 | 0 |
| 1.657108755 | 0 |
| 1.65735234 | 0 |
| 1.657595925 | 0 |
| 1.65783951 | 0 |
| 1.658083095 | 0 |
| 1.65832668 | 0 |
| 1.658570265 | 0 |
| 1.65881385 | 0 |
| 1.659057435 | 0 |
| 1.65930102 | 0 |
| 1.659544605 | 0 |
| 1.65978819 | 0 |
| 1.660031775 | 0 |
| 1.66027536 | 0 |
| 1.660518945 | 0 |
| 1.66076253 | 0 |
| 1.661006115 | 0 |
| 1.6612497 | 0 |
| 1.661493285 | 0 |
| 1.66173687 | 0 |
| 1.661980455 | 0 |
| 1.66222404 | 0 |
| 1.662467625 | 0 |
| 1.66271121 | 0 |
| 1.662954795 | 0 |
| 1.66319838 | 0 |
| 1.663441965 | 0 |
| 1.66368555 | 0 |
| 1.663929135 | 0 |
| 1.66417272 | 0 |
| 1.664416305 | 0 |
| 1.66465989 | 0 |
| 1.664903475 | 0 |
| 1.66514706 | 0 |
| 1.665390645 | 0 |
| 1.66563423 | 0 |
| 1.665877815 | 0 |
| 1.6661214 | 0 |
| 1.666364985 | 0 |
| 1.66660857 | 0 |
| 1.666852155 | 0 |
| 1.66709574 | 0 |
| 1.667339325 | 0 |
| 1.66758291 | 0 |
| 1.667826495 | 0 |
| 1.66807008 | 0 |
| 1.668313665 | 0 |
| 1.66855725 | 0 |
| 1.668800835 | 0 |
| 1.66904442 | 0 |
| 1.669288005 | 0 |
| 1.66953159 | 0 |
| 1.669775175 | 0 |
| 1.67001876 | 0 |
| 1.670262345 | 0 |
| 1.67050593 | 0 |
| 1.670749515 | 0 |
| 1.6709931 | 0 |
| 1.671236685 | 0 |
| 1.67148027 | 0 |
| 1.671723855 | 0 |
| 1.67196744 | 0 |
| 1.672211025 | 0 |
| 1.67245461 | 0 |
| 1.672698195 | 0 |
| 1.67294178 | 0 |
| 1.673185365 | 0 |
| 1.67342895 | 0 |
| 1.673672535 | 0 |
| 1.67391612 | 0 |
| 1.674159705 | 0 |
| 1.67440329 | 0 |
| 1.674646875 | 0 |
| 1.67489046 | 0 |
| 1.675134045 | 0 |
| 1.67537763 | 0 |
| 1.675621215 | 0 |
| 1.6758648 | 0 |
| 1.676108385 | 0 |
| 1.67635197 | 0 |
| 1.676595555 | 0 |
| 1.67683914 | 0 |
| 1.677082725 | 0 |
| 1.67732631 | 0 |
| 1.677569895 | 0 |
| 1.67781348 | 0 |
| 1.678057065 | 0 |
| 1.67830065 | 0 |
| 1.678544235 | 0 |
| 1.67878782 | 0 |
| 1.679031405 | 0 |
| 1.67927499 | 0 |
| 1.679518575 | 0 |
| 1.67976216 | 0 |
| 1.680005745 | 0 |
| 1.68024933 | 0 |
| 1.680492915 | 0 |
| 1.6807365 | 0 |
| 1.680980085 | 0 |
| 1.68122367 | 0 |
| 1.681467255 | 0 |
| 1.68171084 | −0.017 |
| 1.681954425 | −0.127 |
| 1.68219801 | −0.237 |
| 1.682441595 | −0.545 |
| 1.68268518 | −0.986 |
| 1.682928765 | −1.431 |
| 1.68317235 | −1.982 |
| 1.683415935 | −2.533 |
| 1.68365952 | −3.351 |
| 1.683903105 | −4.454 |
| 1.68414669 | −5.558 |
| 1.684390275 | −1.555 |
| 1.68463386 | 2.856 |
| 1.684877445 | 5.441 |
| 1.68512103 | 4.891 |
| 1.685364615 | 4.341 |
| 1.6856082 | 4.237 |
| 1.685851785 | 4.239 |
| 1.68609537 | 4.101 |
| 1.686338955 | 3.549 |
| 1.68658254 | 2.998 |
| 1.686826125 | 2.829 |
| 1.68706971 | 2.83 |
| 1.687313295 | 2.756 |
| 1.68755688 | 2.203 |
| 1.687800465 | 1.65 |
| 1.68804405 | 1.417 |
| 1.688287635 | 1.417 |
| 1.68853122 | 1.407 |
| 1.688774805 | 0.853 |
| 1.68901839 | 0.298 |
| 1.689261975 | 0 |
| 1.68950556 | 0 |
| 1.689749145 | 0 |
| 1.68999273 | 0 |
| 1.690236315 | 0 |
| 1.6904799 | 0 |
| 1.690723485 | 0 |
| 1.69096707 | 0 |
| 1.691210655 | 0 |
| 1.69145424 | 0 |
| 1.691697825 | 0 |
| 1.69194141 | 0 |
| 1.692184995 | 0 |
| 1.69242858 | 0 |
| 1.692672165 | 0 |
| 1.69291575 | 0 |
| 1.693159335 | 0 |
| 1.69340292 | 0 |
| 1.693646505 | 0 |
| 1.69389009 | 0 |
| 1.694133675 | 0 |
| 1.69437726 | 2.231 |
| 1.694620845 | 4.467 |
| 1.69486443 | 5.719 |
| 1.695108015 | 5.721 |
| 1.6953516 | 5.723 |
| 1.695595185 | 3.75 |
| 1.69583877 | 1.512 |
| 1.696082355 | 0 |
| 1.69632594 | 0 |
| 1.696569525 | 0 |
| 1.69681311 | 0 |
| 1.697056695 | 0 |
| 1.69730028 | −0.023 |
| 1.697543865 | −0.136 |
| 1.69778745 | −0.248 |
| 1.698031035 | −0.579 |
| 1.69827462 | −1.029 |
| 1.698518205 | −1.488 |
| 1.69876179 | −2.051 |
| 1.699005375 | −2.614 |
| 1.69924896 | −3.477 |
| 1.699492545 | −4.603 |
| 1.69973613 | −5.729 |
| 1.699979715 | −1.362 |
| 1.7002233 | 3.141 |
| 1.700466885 | 5.526 |
| 1.70071047 | 4.965 |
| 1.700954055 | 4.403 |
| 1.70119764 | 4.326 |
| 1.701441225 | 4.327 |
| 1.70168481 | 4.158 |
| 1.701928395 | 3.595 |
| 1.70217198 | 3.032 |
| 1.702415565 | 2.888 |
| 1.70265915 | 2.889 |
| 1.702902735 | 2.785 |
| 1.70314632 | 2.221 |
| 1.703389905 | 1.656 |
| 1.70363349 | 1.447 |
| 1.703877075 | 1.447 |
| 1.70412066 | 1.408 |
| 1.704364245 | 0.842 |
| 1.70460783 | 0.276 |
| 1.704851415 | 0 |
| 1.705095 | 0 |
| 1.705338585 | 0 |
| 1.70558217 | 0 |
| 1.705825755 | 0 |
| 1.70606934 | 0 |
| 1.706312925 | 0 |
| 1.70655651 | 0 |
| 1.706800095 | 0 |
| 1.70704368 | 0 |
| 1.707287265 | 0 |
| 1.70753085 | 0 |
| 1.707774435 | 0 |
| 1.70801802 | 0 |
| 1.708261605 | 0 |
| 1.70850519 | 0 |
| 1.708748775 | 0 |
| 1.70899236 | 0 |
| 1.709235945 | 0 |
| 1.70947953 | 0 |
| 1.709723115 | 0.11 |
| 1.7099667 | 2.391 |
| 1.710210285 | 4.673 |
| 1.71045387 | 5.838 |
| 1.710697455 | 5.84 |
| 1.71094104 | 5.842 |
| 1.711184625 | 3.713 |
| 1.71142821 | 1.43 |
| 1.711671795 | 0 |
| 1.71191538 | 0 |
| 1.712158965 | 0 |
| 1.71240255 | 0 |
| 1.712646135 | 0 |
| 1.71288972 | −0.03 |
| 1.713133305 | −0.144 |
| 1.71337689 | −0.259 |
| 1.713620475 | −0.614 |
| 1.71386406 | −1.073 |
| 1.714107645 | −1.548 |
| 1.71435123 | −2.122 |
| 1.714594815 | −2.696 |
| 1.7148384 | −3.606 |
| 1.715081985 | −4.756 |
| 1.71532557 | −5.756 |
| 1.715569155 | −1.16 |
| 1.71581274 | 3.435 |
| 1.716056325 | 5.612 |
| 1.71629991 | 5.038 |
| 1.716543495 | 4.465 |
| 1.71678708 | 4.415 |
| 1.717030665 | 4.416 |
| 1.71727425 | 4.215 |
| 1.717517835 | 3.64 |
| 1.71776142 | 3.065 |
| 1.718005005 | 2.948 |
| 1.71824859 | 2.949 |
| 1.718492175 | 2.813 |
| 1.71873576 | 2.237 |
| 1.718979345 | 1.661 |
| 1.71922293 | 1.476 |
| 1.719466515 | 1.477 |
| 1.7197101 | 1.408 |
| 1.719953685 | 0.83 |
| 1.72019727 | 0.253 |
| 1.720440855 | 0 |
| 1.72068444 | 0 |
| 1.720928025 | 0 |
| 1.72117161 | 0 |
| 1.721415195 | 0 |
| 1.72165878 | 0 |
| 1.721902365 | 0 |
| 1.72214595 | 0 |
| 1.722389535 | 0 |
| 1.72263312 | 0 |
| 1.722876705 | 0 |
| 1.72312029 | 0 |
| 1.723363875 | 0 |
| 1.72360746 | 0 |
| 1.723851045 | 0 |
| 1.72409463 | 0 |
| 1.724338215 | 0 |
| 1.7245818 | 0 |
| 1.724825385 | 0 |
| 1.72506897 | 0 |
| 1.725312555 | 0.228 |
| 1.72555614 | 2.556 |
| 1.725799725 | 4.884 |
| 1.72604331 | 5.956 |
| 1.726286895 | 5.958 |
| 1.72653048 | 5.96 |
| 1.726774065 | 3.672 |
| 1.72701765 | 1.342 |
| 1.727261235 | 0 |
| 1.72750482 | 0 |
| 1.727748405 | 0 |
| 1.72799199 | 0 |
| 1.728235575 | 0 |
| 1.72847916 | −0.036 |
| 1.728722745 | −0.153 |
| 1.72896633 | −0.27 |
| 1.729209915 | −0.65 |
| 1.7294535 | −1.118 |
| 1.729697085 | −1.608 |
| 1.72994067 | −2.194 |
| 1.730184255 | −2.779 |
| 1.73042784 | −3.737 |
| 1.730671425 | −4.909 |
| 1.73091501 | −5.636 |
| 1.731158595 | −0.949 |
| 1.73140218 | 3.738 |
| 1.731645765 | 5.694 |
| 1.73188935 | 5.109 |
| 1.732132935 | 4.524 |
| 1.73237652 | 4.502 |
| 1.732620105 | 4.504 |
| 1.73286369 | 4.269 |
| 1.733107275 | 3.683 |
| 1.73335086 | 3.097 |
| 1.733594445 | 3.006 |
| 1.73383803 | 3.007 |
| 1.734081615 | 2.839 |
| 1.7343252 | 2.252 |
| 1.734568785 | 1.665 |
| 1.73481237 | 1.505 |
| 1.735055955 | 1.506 |
| 1.73529954 | 1.406 |
| 1.735543125 | 0.817 |
| 1.73578671 | 0.228 |
| 1.736030295 | 0 |
| 1.73627388 | 0 |
| 1.736517465 | 0 |
| 1.73676105 | 0 |
| 1.737004635 | 0 |
| 1.73724822 | 0 |
| 1.737491805 | 0 |
| 1.73773539 | 0 |
| 1.737978975 | 0 |
| 1.73822256 | 0 |
| 1.738466145 | 0 |
| 1.73870973 | 0 |
| 1.738953315 | 0 |
| 1.7391969 | 0 |
| 1.739440485 | 0 |
| 1.73968407 | 0 |
| 1.739927655 | 0 |
| 1.74017124 | 0 |
| 1.740414825 | 0 |
| 1.74065841 | 0 |
| 1.740901995 | 0.351 |
| 1.74114558 | 2.724 |
| 1.741389165 | 5.096 |
| 1.74163275 | 6.07 |
| 1.741876335 | 6.071 |
| 1.74211992 | 5.997 |
| 1.742363505 | 3.623 |
| 1.74260709 | 1.249 |
| 1.742850675 | 0 |
| 1.74309426 | 0 |
| 1.743337845 | 0 |
| 1.74358143 | 0 |
| 1.743825015 | 0 |
| 1.7440686 | 0 |
| 1.744312185 | 0 |
| 1.74455577 | 0 |
| 1.744799355 | 0 |
| 1.74504294 | 0 |
| 1.745286525 | 0 |
| 1.74553011 | 0 |
| 1.745773695 | 0 |
| 1.74601728 | 0 |
| 1.746260865 | 0 |
| 1.74650445 | 0 |
| 1.746748035 | 0 |
| 1.74699162 | 0 |
| 1.747235205 | 0 |
| 1.74747879 | 0 |
| 1.747722375 | 0 |
| 1.74796596 | 0 |
| 1.748209545 | 0 |
| 1.74845313 | 0 |
| 1.748696715 | 0 |
| 1.7489403 | 0 |
| 1.749183885 | 0 |
| 1.74942747 | 0 |
| 1.749671055 | 0 |
| 1.74991464 | 0 |
| 1.750158225 | 0 |
| 1.75040181 | 0 |
| 1.750645395 | 0 |
| 1.75088898 | 0 |
| 1.751132565 | 0 |
| 1.75137615 | 0 |
| 1.751619735 | 0 |
| 1.75186332 | 0 |
| 1.752106905 | 0 |
| 1.75235049 | 0 |
| 1.752594075 | 0 |
| 1.75283766 | 0 |
| 1.753081245 | 0 |
| 1.75332483 | 0 |
| 1.753568415 | 0 |
| 1.753812 | 0 |
| 1.754055585 | 0 |
| 1.75429917 | 0 |
| 1.754542755 | 0 |
| 1.75478634 | 0 |
| 1.755029925 | 0 |
| 1.75527351 | 0 |
| 1.755517095 | 0 |
| 1.75576068 | 0 |
| 1.756004265 | 0 |
| 1.75624785 | 0 |
| 1.756491435 | 0 |
| 1.75673502 | 0 |
| 1.756978605 | 0 |
| 1.75722219 | 0 |
| 1.757465775 | 0 |
| 1.75770936 | 0 |
| 1.757952945 | 0 |
| 1.75819653 | 0 |
| 1.758440115 | 0 |
| 1.7586837 | 0 |
| 1.758927285 | 0 |
| 1.75917087 | 0 |
| 1.759414455 | 0 |
| 1.75965804 | 0 |
| 1.759901625 | 0 |
| 1.76014521 | 0 |
| 1.760388795 | 0 |
| 1.76063238 | 0 |
| 1.760875965 | 0 |
| 1.76111955 | 0 |
| 1.761363135 | 0 |
| 1.76160672 | 0 |
| 1.761850305 | 0 |
| 1.76209389 | 0 |
| 1.762337475 | 0 |
| 1.76258106 | 0 |
| 1.762824645 | 0 |
| 1.76306823 | 0 |
| 1.763311815 | 0 |
| 1.7635554 | 0 |
| 1.763798985 | 0 |
| 1.76404257 | 0 |
| 1.764286155 | 0 |
| 1.76452974 | 0 |
| 1.764773325 | 0 |
| 1.76501691 | 0 |
| 1.765260495 | 0 |
| 1.76550408 | 0 |
| 1.765747665 | 0 |
| 1.76599125 | 0 |
| 1.766234835 | 0 |
| 1.76647842 | 0 |
| 1.766722005 | 0 |
| 1.76696559 | 0 |
| 1.767209175 | 0 |
| 1.76745276 | 0 |
| 1.767696345 | 0 |
| 1.76793993 | 0 |
| 1.768183515 | 0 |
| 1.7684271 | 0 |
| 1.768670685 | 0 |
| 1.76891427 | 0 |
| 1.769157855 | 0 |
| 1.76940144 | 0 |
| 1.769645025 | 0 |
| 1.76988861 | 0 |
| 1.770132195 | 0 |
| 1.77037578 | 0 |
| 1.770619365 | 0 |
| 1.77086295 | 0 |
| 1.771106535 | 0 |
| 1.77135012 | 0 |
| 1.771593705 | 0 |
| 1.77183729 | 0 |
| 1.772080875 | 0 |
| 1.77232446 | 0 |
| 1.772568045 | 0 |
| 1.77281163 | 0 |
| 1.773055215 | 0 |
| 1.7732988 | 0 |
| 1.773542385 | 0 |
| 1.77378597 | 0 |
| 1.774029555 | 0 |
| 1.77427314 | 0 |
| 1.774516725 | 0 |
| 1.77476031 | 0 |
| 1.775003895 | 0 |
| 1.77524748 | 0 |
| 1.775491065 | 0 |
| 1.77573465 | 0 |
| 1.775978235 | 0 |
| 1.77622182 | 0 |
| 1.776465405 | 0 |
| 1.77670899 | 0 |
| 1.776952575 | 0 |
| 1.77719616 | 0 |
| 1.777439745 | 0 |
| 1.77768333 | 0 |
| 1.777926915 | 0 |
| 1.7781705 | 0 |
| 1.778414085 | 0 |
| 1.77865767 | 0 |
| 1.778901255 | 0 |
| 1.77914484 | 0 |
| 1.779388425 | 0 |
| 1.77963201 | 0 |
| 1.779875595 | 0 |
| 1.78011918 | 0 |
| 1.780362765 | 0 |
| 1.78060635 | 0 |
| 1.780849935 | 0 |
| 1.78109352 | 0 |
| 1.781337105 | 0 |
| 1.78158069 | 0 |
| 1.781824275 | 0 |
| 1.78206786 | 0 |
| 1.782311445 | 0 |
| 1.78255503 | 0 |
| 1.782798615 | 0 |
| 1.7830422 | 0 |
| 1.783285785 | 0 |
| 1.78352937 | 0 |
| 1.783772955 | 0 |
| 1.78401654 | 0 |
| 1.784260125 | 0 |
| 1.78450371 | 0 |
| 1.784747295 | 0 |
| 1.78499088 | 0 |
| 1.785234465 | 0 |
| 1.78547805 | 0 |
| 1.785721635 | 0 |
| 1.78596522 | 0 |
| 1.786208805 | 0 |
| 1.78645239 | 0 |
| 1.786695975 | 0 |
| 1.78693956 | 0 |
| 1.787183145 | 0 |
| 1.78742673 | 0 |
| 1.787670315 | 0 |
| 1.7879139 | 0 |
| 1.788157485 | 0 |
| 1.78840107 | 0 |
| 1.788644655 | 0 |
| 1.78888824 | 0 |
| 1.789131825 | 0 |
| 1.78937541 | 0 |
| 1.789618995 | 0 |
| 1.78986258 | 0 |
| 1.790106165 | 0 |
| 1.79034975 | 0 |
| 1.790593335 | 0 |
| 1.79083692 | 0 |
| 1.791080505 | 0 |
| 1.79132409 | 0 |
| 1.791567675 | 0 |
| 1.79181126 | 0 |
| 1.792054845 | 0 |
| 1.79229843 | 0 |
| 1.792542015 | 0 |
| 1.7927856 | 0 |
| 1.793029185 | 0 |
| 1.79327277 | 0 |
| 1.793516355 | 0 |
| 1.79375994 | 0 |
| 1.794003525 | 0 |
| 1.79424711 | 0 |
| 1.794490695 | 0 |
| 1.79473428 | 0 |
| 1.794977865 | 0 |
| 1.79522145 | 0 |
| 1.795465035 | 0 |
| 1.79570862 | 0 |
| 1.795952205 | 0 |
| 1.79619579 | 0 |
| 1.796439375 | 0 |
| 1.79668296 | 0 |
| 1.796926545 | 0 |
| 1.79717013 | 0 |
| 1.797413715 | 0 |
| 1.7976573 | 0 |
| 1.797900885 | 0 |
| 1.79814447 | 0 |
| 1.798388055 | 0 |
| 1.79863164 | 0 |
| 1.798875225 | 0 |
| 1.79911881 | 0 |
| 1.799362395 | 0 |
| 1.79960598 | 0 |
| 1.799849565 | 0 |
| 1.80009315 | 0 |
| 1.800336735 | 0 |
| 1.80058032 | 0 |
| 1.800823905 | 0 |
| 1.80106749 | 0 |
| 1.801311075 | 0 |
| 1.80155466 | 0 |
| 1.801798245 | 0 |
| 1.80204183 | 0 |
| 1.802285415 | 0 |
| 1.802529 | 0 |
| 1.802772585 | 0 |
| 1.80301617 | 0 |
| 1.803259755 | 0 |
| 1.80350334 | 0 |
| 1.803746925 | 0 |
| 1.80399051 | 0 |
| 1.804234095 | 0 |
| 1.80447768 | 0 |
| 1.804721265 | 0 |
| 1.80496485 | 0 |
| 1.805208435 | 0 |
| 1.80545202 | 0 |
| 1.805695605 | 0 |
| 1.80593919 | 0 |
| 1.806182775 | 0 |
| 1.80642636 | 0 |
| 1.806669945 | 0 |
| 1.80691353 | 0 |
| 1.807157115 | 0 |
| 1.8074007 | 0 |
| 1.807644285 | 0 |
| 1.80788787 | 0 |
| 1.808131455 | 0 |
| 1.80837504 | 0 |
| 1.808618625 | 0 |
| 1.80886221 | 0 |
| 1.809105795 | 0 |
| 1.80934938 | 0 |
| 1.809592965 | 0 |
| 1.80983655 | 0 |
| 1.810080135 | 0 |
| 1.81032372 | 0 |
| 1.810567305 | 0 |
| 1.81081089 | 0 |
| 1.811054475 | 0 |
| 1.81129806 | 0 |
| 1.811541645 | 0 |
| 1.81178523 | 0 |
| 1.812028815 | 0 |
| 1.8122724 | 0 |
| 1.812515985 | 0 |
| 1.81275957 | 0 |
| 1.813003155 | 0 |
| 1.81324674 | 0 |
| 1.813490325 | 0 |
| 1.81373391 | 0 |
| 1.813977495 | 0 |
| 1.81422108 | 0 |
| 1.814464665 | 0 |
| 1.81470825 | 0 |
| 1.814951835 | 0 |
| 1.81519542 | 0 |
| 1.815439005 | 0 |
| 1.81568259 | 0 |
| 1.815926175 | 0 |
| 1.81616976 | 0 |
| 1.816413345 | 0 |
| 1.81665693 | 0 |
| 1.816900515 | 0 |
| 1.8171441 | 0 |
| 1.817387685 | 0 |
| 1.81763127 | 0 |
| 1.817874855 | 0 |
| 1.81811844 | 0 |
| 1.818362025 | 0 |
| 1.81860561 | 0 |
| 1.818849195 | 0 |
| 1.81909278 | 0 |
| 1.819336365 | 0 |
| 1.81957995 | 0 |
| 1.819823535 | 0 |
| 1.82006712 | 0 |
| 1.820310705 | 0 |
| 1.82055429 | 0 |
| 1.820797875 | 0 |
| 1.82104146 | 0 |
| 1.821285045 | 0 |
| 1.82152863 | 0 |
| 1.821772215 | 0 |
| 1.8220158 | 0 |
| 1.822259385 | 0 |
| 1.82250297 | 0 |
| 1.822746555 | 0 |
| 1.82299014 | 0 |
| 1.823233725 | 0 |
| 1.82347731 | 0 |
| 1.823720895 | 0 |
| 1.82396448 | 0 |
| 1.824208065 | 0 |
| 1.82445165 | 0 |
| 1.824695235 | 0 |
| 1.82493882 | 0 |
| 1.825182405 | 0 |
| 1.82542599 | 0 |
| 1.825669575 | 0 |
| 1.82591316 | 0 |
| 1.826156745 | 0 |
| 1.82640033 | 0 |
| 1.826643915 | 0 |
| 1.8268875 | 0 |
| 1.827131085 | 0 |
| 1.82737467 | 0 |
| 1.827618255 | 0 |
| 1.82786184 | 0 |
| 1.828105425 | 0 |
| 1.82834901 | 0 |
| 1.828592595 | 0 |
| 1.82883618 | 0 |
| 1.829079765 | 0 |
| 1.82932335 | 0 |
| 1.829566935 | 0 |
| 1.82981052 | 0 |
| 1.830054105 | 0 |
| 1.83029769 | 0 |
| 1.830541275 | 0 |
| 1.83078486 | 0 |
| 1.831028445 | 0 |
| 1.83127203 | 0 |
| 1.831515615 | 0 |
| 1.8317592 | 0 |
| 1.832002785 | 0 |
| 1.83224637 | 0 |
| 1.832489955 | 0 |
| 1.83273354 | 0 |
| 1.832977125 | 0 |
| 1.83322071 | 0 |
| 1.833464295 | 0 |
| 1.83370788 | 0 |
| 1.833951465 | 0 |
| 1.83419505 | 0 |
| 1.834438635 | 0 |
| 1.83468222 | 0 |
| 1.834925805 | 0 |
| 1.83516939 | 0 |
| 1.835412975 | 0 |
| 1.83565656 | 0 |
| 1.835900145 | 0 |
| 1.83614373 | 0 |
| 1.836387315 | 0 |
| 1.8366309 | 0 |
| 1.836874485 | 0 |
| 1.83711807 | 0 |
| 1.837361655 | 0 |
| 1.83760524 | 0 |
| 1.837848825 | 0 |
| 1.83809241 | 0 |
| 1.838335995 | 0 |
| 1.83857958 | 0 |
| 1.838823165 | 0 |
| 1.83906675 | 0 |
| 1.839310335 | 0 |
| 1.83955392 | 0 |
| 1.839797505 | 0 |
| 1.84004109 | 0 |
| 1.840284675 | 0 |
| 1.84052826 | 0 |
| 1.840771845 | 0 |
| 1.84101543 | 0 |
| 1.841259015 | 0 |
| 1.8415026 | 0 |
| 1.841746185 | 0 |
| 1.84198977 | 0 |
| 1.842233355 | 0 |
| 1.84247694 | 0 |
| 1.842720525 | 0 |
| 1.84296411 | 0 |
| 1.843207695 | 0 |
| 1.84345128 | 0 |
| 1.843694865 | 0 |
| 1.84393845 | 0 |
| 1.844182035 | 0 |
| 1.84442562 | 0 |
| 1.844669205 | 0 |
| 1.84491279 | 0 |
| 1.845156375 | 0 |
| 1.84539996 | 0 |
| 1.845643545 | 0 |
| 1.84588713 | 0 |
| 1.846130715 | 0 |
| 1.8463743 | 0 |
| 1.846617885 | 0 |
| 1.84686147 | 0 |
| 1.847105055 | 0 |
| 1.84734864 | 0 |
| 1.847592225 | 0 |
| 1.84783581 | 0 |
| 1.848079395 | 0 |
| 1.84832298 | 0 |
| 1.848566565 | 0 |
| 1.84881015 | 0 |
| 1.849053735 | 0 |
| 1.84929732 | 0 |
| 1.849540905 | 0 |
| 1.84978449 | 0 |
| 1.850028075 | 0 |
| 1.85027166 | 0 |
| 1.850515245 | 0 |
| 1.85075883 | 0 |
| 1.851002415 | 0 |
| 1.851246 | 0 |
| 1.851489585 | 0 |
| 1.85173317 | 0 |
| 1.851976755 | 0 |
| 1.85222034 | 0 |
| 1.852463925 | 0 |
| 1.85270751 | 0 |
| 1.852951095 | 0 |
| 1.85319468 | 0 |
| 1.853438265 | 0 |
| 1.85368185 | 0 |
| 1.853925435 | 0 |
| 1.85416902 | 0 |
| 1.854412605 | 0 |
| 1.85465619 | 0 |
| 1.854899775 | 0 |
| 1.85514336 | 0 |
| 1.855386945 | 0 |
| 1.85563053 | 0 |
| 1.855874115 | 0 |
| 1.8561177 | 0 |
| 1.856361285 | 0 |
| 1.85660487 | 0 |
| 1.856848455 | 0 |
| 1.85709204 | 0 |
| 1.857335625 | 0 |
| 1.85757921 | 0 |
| 1.857822795 | 0 |
| 1.85806638 | 0 |
| 1.858309965 | 0 |
| 1.85855355 | 0 |
| 1.858797135 | 0 |
| 1.85904072 | 0 |
| 1.859284305 | 0 |
| 1.85952789 | 0 |
| 1.859771475 | 0 |
| 1.86001506 | 0 |
| 1.860258645 | 0 |
| 1.86050223 | 0 |
| 1.860745815 | 0 |
| 1.8609894 | 0 |
| 1.861232985 | 0 |
| 1.86147657 | 0 |
| 1.861720155 | 0 |
| 1.86196374 | 0 |
| 1.862207325 | 0 |
| 1.86245091 | 0 |
| 1.862694495 | 0 |
| 1.86293808 | 0 |
| 1.863181665 | 0 |
| 1.86342525 | 0 |
| 1.863668835 | 0 |
| 1.86391242 | 0 |
| 1.864156005 | 0 |
| 1.86439959 | 0 |
| 1.864643175 | 0 |
| 1.86488676 | 0 |
| 1.865130345 | 0 |
| 1.86537393 | 0 |
| 1.865617515 | 0 |
| 1.8658611 | 0 |
| 1.866104685 | 0 |
| 1.86634827 | 0 |
| 1.866591855 | 0 |
| 1.86683544 | 0 |
| 1.867079025 | 0 |
| 1.86732261 | 0 |
| 1.867566195 | 0 |
| 1.86780978 | 0 |
| 1.868053365 | 0 |
| 1.86829695 | 0 |
| 1.868540535 | 0 |
| 1.86878412 | −0.096 |
| 1.869027705 | −0.223 |
| 1.86927129 | −0.426 |
| 1.869514875 | −0.934 |
| 1.86975846 | −1.442 |
| 1.870002045 | −2.031 |
| 1.87024563 | −2.665 |
| 1.870489215 | −3.353 |
| 1.8707328 | −4.621 |
| 1.870976385 | −5.89 |
| 1.87121997 | −3.822 |
| 1.871463555 | 1.253 |
| 1.87170714 | 6.328 |
| 1.871950725 | 5.876 |
| 1.87219431 | 5.241 |
| 1.872437895 | 4.867 |
| 1.87268148 | 4.866 |
| 1.872925065 | 4.866 |
| 1.87316865 | 4.325 |
| 1.873412235 | 3.691 |
| 1.87365582 | 3.243 |
| 1.873899405 | 3.243 |
| 1.87414299 | 3.243 |
| 1.874386575 | 2.776 |
| 1.87463016 | 2.142 |
| 1.874873745 | 1.621 |
| 1.87511733 | 1.621 |
| 1.875360915 | 1.621 |
| 1.8756045 | 1.228 |
| 1.875848085 | 0.594 |
| 1.87609167 | 0 |
| 1.876335255 | 0 |
| 1.87657884 | 0 |
| 1.876822425 | 0 |
| 1.87706601 | 0 |
| 1.877309595 | 0 |
| 1.87755318 | 0 |
| 1.877796765 | 0 |
| 1.87804035 | 0 |
| 1.878283935 | 0 |
| 1.87852752 | 0 |
| 1.878771105 | 0 |
| 1.87901469 | 0 |
| 1.879258275 | 0 |
| 1.87950186 | 0 |
| 1.879745445 | 0 |
| 1.87998903 | 0 |
| 1.880232615 | 0 |
| 1.8804762 | 0 |
| 1.880719785 | 0 |
| 1.88096337 | 0 |
| 1.881206955 | 1.512 |
| 1.88145054 | 4.04 |
| 1.881694125 | 6.467 |
| 1.88193771 | 6.466 |
| 1.882181295 | 6.465 |
| 1.88242488 | 5.247 |
| 1.882668465 | 2.719 |
| 1.88291205 | 0.192 |
| 1.883155635 | 0 |
| 1.88339922 | 0 |
| 1.883642805 | 0 |
| 1.88388639 | 0 |
| 1.884129975 | 0 |
| 1.88437356 | −0.102 |
| 1.884617145 | −0.228 |
| 1.88486073 | −0.449 |
| 1.885104315 | −0.954 |
| 1.8853479 | −1.458 |
| 1.885591485 | −2.05 |
| 1.88583507 | −2.681 |
| 1.886078655 | −3.396 |
| 1.88632224 | −4.656 |
| 1.886565825 | −5.917 |
| 1.88680941 | −3.547 |
| 1.887052995 | 1.497 |
| 1.88729658 | 6.438 |
| 1.887540165 | 5.807 |
| 1.88778375 | 5.176 |
| 1.888027335 | 4.835 |
| 1.88827092 | 4.835 |
| 1.888514505 | 4.834 |
| 1.88875809 | 4.265 |
| 1.889001675 | 3.635 |
| 1.88924526 | 3.221 |
| 1.889488845 | 3.221 |
| 1.88973243 | 3.221 |
| 1.889976015 | 2.726 |
| 1.8902196 | 2.096 |
| 1.890463185 | 1.61 |
| 1.89070677 | 1.609 |
| 1.890950355 | 1.609 |
| 1.89119394 | 1.188 |
| 1.891437525 | 0.559 |
| 1.89168111 | 0 |
| 1.891924695 | 0 |
| 1.89216828 | 0 |
| 1.892411865 | 0 |
| 1.89265545 | 0 |
| 1.892899035 | 0 |
| 1.89314262 | 0 |
| 1.893386205 | 0 |
| 1.89362979 | 0 |
| 1.893873375 | 0 |
| 1.89411696 | 0 |
| 1.894360545 | 0 |
| 1.89460413 | 0 |
| 1.894847715 | 0 |
| 1.8950913 | 0 |
| 1.895334885 | 0 |
| 1.89557847 | 0 |
| 1.895822055 | 0 |
| 1.89606564 | 0 |
| 1.896309225 | 0 |
| 1.89655281 | 0 |
| 1.896796395 | 1.624 |
| 1.89703998 | 4.131 |
| 1.897283565 | 6.412 |
| 1.89752715 | 6.411 |
| 1.897770735 | 6.41 |
| 1.89801432 | 5.077 |
| 1.898257905 | 2.571 |
| 1.89850149 | 0.065 |
| 1.898745075 | 0 |
| 1.89898866 | 0 |
| 1.899232245 | 0 |
| 1.89947583 | 0 |
| 1.899719415 | 0 |
| 1.899963 | −0.107 |
| 1.900206585 | −0.232 |
| 1.90045017 | −0.47 |
| 1.900693755 | −0.97 |
| 1.90093734 | −1.47 |
| 1.901180925 | −2.062 |
| 1.90142451 | −2.687 |
| 1.901668095 | −3.426 |
| 1.90191168 | −4.675 |
| 1.902155265 | −5.924 |
| 1.90239885 | −3.263 |
| 1.902642435 | 1.732 |
| 1.90288602 | 6.345 |
| 1.903129605 | 5.72 |
| 1.90337319 | 5.095 |
| 1.903616775 | 4.788 |
| 1.90386036 | 4.787 |
| 1.904103945 | 4.786 |
| 1.90434753 | 4.192 |
| 1.904591115 | 3.568 |
| 1.9048347 | 3.189 |
| 1.905078285 | 3.189 |
| 1.90532187 | 3.188 |
| 1.905565455 | 2.667 |
| 1.90580904 | 2.043 |
| 1.906052625 | 1.593 |
| 1.90629621 | 1.593 |
| 1.906539795 | 1.593 |
| 1.90678338 | 1.144 |
| 1.907026965 | 0.522 |
| 1.90727055 | 0 |
| 1.907514135 | 0 |
| 1.90775772 | 0 |
| 1.908001305 | 0 |
| 1.90824489 | 0 |
| 1.908488475 | 0 |
| 1.90873206 | 0 |
| 1.908975645 | 0 |
| 1.90921923 | 0 |
| 1.909462815 | 0 |
| 1.9097064 | 0 |
| 1.909949985 | 0 |
| 1.91019357 | 0 |
| 1.910437155 | 0 |
| 1.91068074 | 0 |
| 1.910924325 | 0 |
| 1.91116791 | 0 |
| 1.911411495 | 0 |
| 1.91165508 | 0 |
| 1.911898665 | 0 |
| 1.91214225 | 0 |
| 1.912385835 | 1.729 |
| 1.91262942 | 4.207 |
| 1.912873005 | 6.337 |
| 1.91311659 | 6.336 |
| 1.913360175 | 6.335 |
| 1.91360376 | 4.893 |
| 1.913847345 | 2.417 |
| 1.91409093 | 0 |
| 1.914334515 | 0 |
| 1.9145781 | 0 |
| 1.914821685 | 0 |
| 1.91506527 | 0 |
| 1.915308855 | 0 |
| 1.91555244 | −0.112 |
| 1.915796025 | −0.236 |
| 1.91603961 | −0.489 |
| 1.916283195 | −0.983 |
| 1.91652678 | −1.476 |
| 1.916770365 | −2.068 |
| 1.91701395 | −2.684 |
| 1.917257535 | −3.445 |
| 1.91750112 | −4.678 |
| 1.917744705 | −5.91 |
| 1.91798829 | −2.976 |
| 1.918231875 | 1.956 |
| 1.91847546 | 6.233 |
| 1.918719045 | 5.616 |
| 1.91896263 | 4.998 |
| 1.919206215 | 4.726 |
| 1.9194498 | 4.725 |
| 1.919693385 | 4.723 |
| 1.91993697 | 4.106 |
| 1.920180555 | 3.49 |
| 1.92042414 | 3.147 |
| 1.920667725 | 3.146 |
| 1.92091131 | 3.146 |
| 1.921154895 | 2.6 |
| 1.92139848 | 1.985 |
| 1.921642065 | 1.572 |
| 1.92188565 | 1.571 |
| 1.922129235 | 1.571 |
| 1.92237282 | 1.098 |
| 1.922616405 | 0.484 |
| 1.92285999 | 0 |
| 1.923103575 | 0 |
| 1.92334716 | 0 |
| 1.923590745 | 0 |
| 1.92383433 | 0 |
| 1.924077915 | 0 |
| 1.9243215 | 0 |
| 1.924565085 | 0 |
| 1.92480867 | 0 |
| 1.925052255 | 0 |
| 1.92529584 | 0 |
| 1.925539425 | 0 |
| 1.92578301 | 0 |
| 1.926026595 | 0 |
| 1.92627018 | 0 |
| 1.926513765 | 0 |
| 1.92675735 | 0 |
| 1.927000935 | 0 |
| 1.92724452 | 0 |
| 1.927488105 | 0 |
| 1.92773169 | 0 |
| 1.927975275 | 1.826 |
| 1.92821886 | 4.266 |
| 1.928462445 | 6.243 |
| 1.92870603 | 6.241 |
| 1.928949615 | 6.24 |
| 1.9291932 | 5.853 |
| 1.929436785 | 5.242 |
| 1.92968037 | 4.676 |
| 1.929923955 | 4.675 |
| 1.93016754 | 4.674 |
| 1.930411125 | 4.358 |
| 1.93065471 | 3.749 |
| 1.930898295 | 3.139 |
| 1.93114188 | 3.112 |
| 1.931385465 | 3.112 |
| 1.93162905 | 2.868 |
| 1.931872635 | 2.259 |
| 1.93211622 | 1.651 |
| 1.932359805 | 1.554 |
| 1.93260339 | 1.554 |
| 1.932846975 | 1.381 |
| 1.93309056 | 0.774 |
| 1.933334145 | 0.166 |
| 1.93357773 | 0 |
| 1.933821315 | 0 |
| 1.9340649 | 0 |
| 1.934308485 | 0 |
| 1.93455207 | 0 |
| 1.934795655 | 0 |
| 1.93503924 | 0 |
| 1.935282825 | 0 |
| 1.93552641 | 0 |
| 1.935769995 | 0 |
| 1.93601358 | 0 |
| 1.936257165 | 0 |
| 1.93650075 | 0 |
| 1.936744335 | 0 |
| 1.93698792 | 0 |
| 1.937231505 | 0 |
| 1.93747509 | 0 |
| 1.937718675 | 0 |
| 1.93796226 | 0 |
| 1.938205845 | 0 |
| 1.93844943 | 0 |
| 1.938693015 | 0 |
| 1.9389366 | 0 |
| 1.939180185 | 0 |
| 1.93942377 | 0 |
| 1.939667355 | 0 |
| 1.93991094 | 0 |
| 1.940154525 | 0 |
| 1.94039811 | 0 |
| 1.940641695 | 0 |
| 1.94088528 | 0 |
| 1.941128865 | 0 |
| 1.94137245 | 0 |
| 1.941616035 | 0 |
| 1.94185962 | 0 |
| 1.942103205 | 0 |
| 1.94234679 | 0 |
| 1.942590375 | 0 |
| 1.94283396 | 0 |
| 1.943077545 | 0 |
| 1.94332113 | 0 |
| 1.943564715 | 0 |
| 1.9438083 | 0 |
| 1.944051885 | 0 |
| 1.94429547 | 0 |
| 1.944539055 | 0 |
| 1.94478264 | 0 |
| 1.945026225 | 0 |
| 1.94526981 | 0 |
| 1.945513395 | 0 |
| 1.94575698 | 0 |
| 1.946000565 | 0 |
| 1.94624415 | 0 |
| 1.946487735 | 0 |
| 1.94673132 | 0 |
| 1.946974905 | 0 |
| 1.94721849 | 0 |
| 1.947462075 | 0 |
| 1.94770566 | 0 |
| 1.947949245 | 0 |
| 1.94819283 | 0 |
| 1.948436415 | 0 |
| 1.94868 | 0 |
| 1.948923585 | 0 |
| 1.94916717 | 0 |
| 1.949410755 | 0 |
| 1.94965434 | 0 |
| 1.949897925 | 0 |
| 1.95014151 | 0 |
| 1.950385095 | 0 |
| 1.95062868 | 0 |
| 1.950872265 | 0 |
| 1.95111585 | 0 |
| 1.951359435 | 0 |
| 1.95160302 | 0 |
| 1.951846605 | 0 |
| 1.95209019 | 0 |
| 1.952333775 | 0 |
| 1.95257736 | 0 |
| 1.952820945 | 0 |
| 1.95306453 | 0 |
| 1.953308115 | 0 |
| 1.9535517 | 0 |
| 1.953795285 | 0 |
| 1.95403887 | 0 |
| 1.954282455 | 0 |
| 1.95452604 | 0 |
| 1.954769625 | 0 |
| 1.95501321 | 0 |
| 1.955256795 | 0 |
| 1.95550038 | 0 |
| 1.955743965 | 0 |
| 1.95598755 | 0 |
| 1.956231135 | 0 |
| 1.95647472 | 0 |
| 1.956718305 | 0 |
| 1.95696189 | 0 |
| 1.957205475 | 0 |
| 1.95744906 | 0 |
| 1.957692645 | 0 |
| 1.95793623 | 0 |
| 1.958179815 | 0 |
| 1.9584234 | 0 |
| 1.958666985 | 0 |
| 1.95891057 | 0 |
| 1.959154155 | 0 |
| 1.95939774 | 0 |
| 1.959641325 | 0 |
| 1.95988491 | 0 |
| 1.960128495 | 0 |
| 1.96037208 | 0 |
| 1.960615665 | 0 |
| 1.96085925 | 0 |
| 1.961102835 | 0 |
| 1.96134642 | 0 |
| 1.961590005 | 0 |
| 1.96183359 | 0 |
| 1.962077175 | 0 |
| 1.96232076 | 0 |
| 1.962564345 | 0 |
| 1.96280793 | 0 |
| 1.963051515 | 0 |
| 1.9632951 | 0 |
| 1.963538685 | 0 |
| 1.96378227 | 0 |
| 1.964025855 | 0 |
| 1.96426944 | 0 |
| 1.964513025 | 0 |
| 1.96475661 | 0 |
| 1.965000195 | 0 |
| 1.96524378 | 0 |
| 1.965487365 | 0 |
| 1.96573095 | 0 |
| 1.965974535 | 0 |
| 1.96621812 | 0 |
| 1.966461705 | 0 |
| 1.96670529 | 0 |
| 1.966948875 | 0 |
| 1.96719246 | 0 |
| 1.967436045 | 0 |
| 1.96767963 | 0 |
| 1.967923215 | 0 |
| 1.9681668 | 0 |
| 1.968410385 | 0 |
| 1.96865397 | 0 |
| 1.968897555 | 0 |
| 1.96914114 | 0 |
| 1.969384725 | 0 |
| 1.96962831 | 0 |
| 1.969871895 | 0 |
| 1.97011548 | 0 |
| 1.970359065 | 0 |
| 1.97060265 | 0 |
| 1.970846235 | 0 |
| 1.97108982 | 0 |
| 1.971333405 | 0 |
| 1.97157699 | 0 |
| 1.971820575 | 0 |
| 1.97206416 | 0 |
| 1.972307745 | 0 |
| 1.97255133 | 0 |
| 1.972794915 | 0 |
| 1.9730385 | 0 |
| 1.973282085 | 0 |
| 1.97352567 | 0 |
| 1.973769255 | 0 |
| 1.97401284 | 0 |
| 1.974256425 | 0 |
| 1.97450001 | 0 |
| 1.974743595 | 0 |
| 1.97498718 | 0 |
| 1.975230765 | 0 |
| 1.97547435 | 0 |
| 1.975717935 | 0 |
| 1.97596152 | 0 |
| 1.976205105 | 0 |
| 1.97644869 | 0 |
| 1.976692275 | 0 |
| 1.97693586 | 0 |
| 1.977179445 | 0 |
| 1.97742303 | 0 |
| 1.977666615 | 0 |
| 1.9779102 | 0 |
| 1.978153785 | 0 |
| 1.97839737 | 0 |
| 1.978640955 | 0 |
| 1.97888454 | 0 |
| 1.979128125 | 0 |
| 1.97937171 | 0 |
| 1.979615295 | 0 |
| 1.97985888 | 0 |
| 1.980102465 | 0 |
| 1.98034605 | 0 |
| 1.980589635 | 0 |
| 1.98083322 | 0 |
| 1.981076805 | 0 |
| 1.98132039 | 0 |
| 1.981563975 | 0 |
| 1.98180756 | 0 |
| 1.982051145 | 0 |
| 1.98229473 | 0 |
| 1.982538315 | 0 |
| 1.9827819 | 0 |
| 1.983025485 | 0 |
| 1.98326907 | 0 |
| 1.983512655 | 0 |
| 1.98375624 | 0 |
| 1.983999825 | 0 |
| 1.98424341 | 0 |
| 1.984486995 | 0 |
| 1.98473058 | 0 |
| 1.984974165 | 0 |
| 1.98521775 | 0 |
| 1.985461335 | 0 |
| 1.98570492 | 0 |
| 1.985948505 | 0 |
| 1.98619209 | 0 |
| 1.986435675 | 0 |
| 1.98667926 | 0 |
| 1.986922845 | 0 |
| 1.98716643 | 0 |
| 1.987410015 | 0 |
| 1.9876536 | 0 |
| 1.987897185 | 0 |
| 1.98814077 | 0 |
| 1.988384355 | 0 |
| 1.98862794 | 0 |
| 1.988871525 | 0 |
| 1.98911511 | 0 |
| 1.989358695 | 0 |
| 1.98960228 | 0 |
| 1.989845865 | 0 |
| 1.99008945 | 0 |
| 1.990333035 | 0 |
| 1.99057662 | 0 |
| 1.990820205 | 0 |
| 1.99106379 | 0 |
| 1.991307375 | 0 |
| 1.99155096 | 0 |
| 1.991794545 | 0 |
| 1.99203813 | 0 |
| 1.992281715 | 0 |
| 1.9925253 | 0 |
| 1.992768885 | 0 |
| 1.99301247 | 0 |
| 1.993256055 | 0 |
| 1.99349964 | 0 |
| 1.993743225 | 0 |
| 1.99398681 | 0 |
| 1.994230395 | 0 |
| 1.99447398 | 0 |
| 1.994717565 | 0 |
| 1.99496115 | 0 |
| 1.995204735 | 0 |
| 1.99544832 | 0 |
| 1.995691905 | 0 |
| 1.99593549 | 0 |
| 1.996179075 | 0 |
| 1.99642266 | 0 |
| 1.996666245 | 0 |
| 1.99690983 | 0 |
| 1.997153415 | 0 |
| 1.997397 | 0 |
| 1.997640585 | 0 |
| 1.99788417 | 0 |
| 1.998127755 | 0 |
| 1.99837134 | 0 |
| 1.998614925 | 0 |
| 1.99885851 | 0 |
| 1.999102095 | 0 |
| 1.99934568 | 0 |
| 1.999589265 | 0 |
| 1.99983285 | 0 |
| 2.000076435 | 0 |
| 2.00032002 | 0 |
| 2.000563605 | 0 |
| 2.00080719 | 0 |
| 2.001050775 | 0 |
| 2.00129436 | 0 |
| 2.001537945 | 0 |
| 2.00178153 | 0 |
| 2.002025115 | 0 |
| 2.0022687 | 0 |
| 2.002512285 | 0 |
| 2.00275587 | 0 |
| 2.002999455 | 0 |
| 2.00324304 | 0 |
| 2.003486625 | 0 |
| 2.00373021 | 0 |
| 2.003973795 | 0 |
| 2.00421738 | 0 |
| 2.004460965 | 0 |
| 2.00470455 | 0 |
| 2.004948135 | 0 |
| 2.00519172 | 0 |
| 2.005435305 | 0 |
| 2.00567889 | 0 |
| 2.005922475 | 0 |
| 2.00616606 | 0 |
| 2.006409645 | 0 |
| 2.00665323 | 0 |
| 2.006896815 | 0 |
| 2.0071404 | 0 |
| 2.007383985 | 0 |
| 2.00762757 | 0 |
| 2.007871155 | 0 |
| 2.00811474 | 0 |
| 2.008358325 | 0 |
| 2.00860191 | 0 |
| 2.008845495 | 0 |
| 2.00908908 | 0 |
| 2.009332665 | 0 |
| 2.00957625 | 0 |
| 2.009819835 | 0 |
| 2.01006342 | 0 |
| 2.010307005 | 0 |
| 2.01055059 | 0 |
| 2.010794175 | 0 |
| 2.01103776 | 0 |
| 2.011281345 | 0 |
| 2.01152493 | 0 |
| 2.011768515 | 0 |
| 2.0120121 | 0 |
| 2.012255685 | 0 |
| 2.01249927 | 0 |
| 2.012742855 | 0 |
| 2.01298644 | 0 |
| 2.013230025 | 0 |
| 2.01347361 | 0 |
| 2.013717195 | 0 |
| 2.01396078 | 0 |
| 2.014204365 | 0 |
| 2.01444795 | 0 |
| 2.014691535 | 0 |
| 2.01493512 | 0 |
| 2.015178705 | 0 |
| 2.01542229 | 0 |
| 2.015665875 | 0 |
| 2.01590946 | 0 |
| 2.016153045 | 0 |
| 2.01639663 | 0 |
| 2.016640215 | 0 |
| 2.0168838 | 0 |
| 2.017127385 | 0 |
| 2.01737097 | 0 |
| 2.017614555 | 0 |
| 2.01785814 | 0 |
| 2.018101725 | 0 |
| 2.01834531 | 0 |
| 2.018588895 | 0 |
| 2.01883248 | 0 |
| 2.019076065 | 0 |
| 2.01931965 | 0 |
| 2.019563235 | 0 |
| 2.01980682 | 0 |
| 2.020050405 | 0 |
| 2.02029399 | 0 |
| 2.020537575 | 0 |
| 2.02078116 | 0 |
| 2.021024745 | 0 |
| 2.02126833 | 0 |
| 2.021511915 | 0 |
| 2.0217555 | 0 |
| 2.021999085 | 0 |
| 2.02224267 | 0 |
| 2.022486255 | 0 |
| 2.02272984 | 0 |
| 2.022973425 | 0 |
| 2.02321701 | 0 |
| 2.023460595 | 0 |
| 2.02370418 | 0 |
| 2.023947765 | 0 |
| 2.02419135 | 0 |
| 2.024434935 | 0 |
| 2.02467852 | 0 |
| 2.024922105 | 0 |
| 2.02516569 | 0 |
| 2.025409275 | 0 |
| 2.02565286 | 0 |
| 2.025896445 | 0 |
| 2.02614003 | 0 |
| 2.026383615 | 0 |
| 2.0266272 | 0 |
| 2.026870785 | 0 |
| 2.02711437 | 0 |
| 2.027357955 | 0 |
| 2.02760154 | 0 |
| 2.027845125 | 0 |
| 2.02808871 | 0 |
| 2.028332295 | 0 |
| 2.02857588 | 0 |
| 2.028819465 | 0 |
| 2.02906305 | 0 |
| 2.029306635 | 0 |
| 2.02955022 | 0 |
| 2.029793805 | 0 |
| 2.03003739 | 0 |
| 2.030280975 | 0 |
| 2.03052456 | 0 |
| 2.030768145 | 0 |
| 2.03101173 | 0 |
| 2.031255315 | 0 |
| 2.0314989 | 0 |
| 2.031742485 | 0 |
| 2.03191607 | 0 |
| 2.032229655 | 0 |
| 2.03247324 | 0 |
| 2.032716825 | 0 |
| 2.03296041 | 0 |
| 2.033203995 | 0 |
| 2.03344758 | 0 |
| 2.033691165 | 0 |
| 2.03393475 | 0 |
| 2.034178335 | 0 |
| 2.03442192 | 0 |
| 2.034665505 | 0 |
| 2.03490909 | 0 |
| 2.035152675 | 0 |
| 2.03539626 | 0 |
| 2.035639845 | 0 |
| 2.03588343 | 0 |
| 2.036127015 | 0 |
| 2.0363706 | 0 |
| 2.036614185 | 0 |
| 2.03685777 | 0 |
| 2.037101355 | 0 |
| 2.03734494 | 0 |
| 2.037588525 | 0 |
| 2.03783211 | 0 |
| 2.038075695 | 0 |
| 2.03831928 | 0 |
| 2.038562865 | 0 |
| 2.03880645 | 0 |
| 2.039050035 | 0 |
| 2.03929362 | 0 |
| 2.039537205 | 0 |
| 2.03978079 | 0 |
| 2.040024375 | 0 |
| 2.04026796 | 0 |
| 2.040511545 | 0 |
| 2.04075513 | 0 |
| 2.040998715 | 0 |
| 2.0412423 | 0 |
| 2.041485885 | 0 |
| 2.04172947 | 0 |
| 2.041973055 | 0 |
| 2.04221664 | 0 |
| 2.042460225 | 0 |
| 2.04270381 | 0 |
| 2.042947395 | 0 |
| 2.04319098 | 0 |
| 2.043434565 | 0 |
| 2.04367815 | 0 |
| 2.043921735 | 0 |
| 2.04416532 | 0 |
| 2.044408905 | 0 |
| 2.04465249 | 0 |
| 2.044896075 | 0 |
| 2.04513966 | 0 |
| 2.045383245 | 0 |
| 2.04562683 | 0 |
| 2.045870415 | 0 |
| 2.046114 | 0 |
| 2.046357585 | 0 |
| 2.04660117 | 0 |
| 2.046844755 | 0 |
| 2.04708834 | 0 |
| 2.047331925 | 0 |
| 2.04757551 | 0 |
| 2.047819095 | 0 |
| 2.04806268 | 0 |
| 2.048306265 | 0 |
| 2.04854985 | 0 |
| 2.048793435 | 0 |
| 2.04903702 | 0 |
| 2.049280605 | 0 |
| 2.04952419 | 0 |
| 2.049767775 | 0 |
| 2.05001136 | 0 |
| 2.050254945 | 0 |
| 2.05049853 | 0 |
| 2.050742115 | 0 |
| 2.0509857 | 0 |
| 2.051229285 | 0 |
| 2.05147287 | 0 |
| 2.051716455 | 0 |
| 2.05196004 | 0 |
| 2.052203625 | 0 |
| 2.05244721 | 0 |
| 2.052690795 | 0 |
| 2.05293438 | 0 |
| 2.053177965 | 0 |
| 2.05342155 | 0 |
| 2.053665135 | 0 |
| 2.05390872 | 0 |
| 2.054152305 | 0 |
| 2.05439589 | 0 |
| 2.054639475 | 0 |
| 2.05488306 | 0 |
| 2.055126645 | 0 |
| 2.05537023 | 0 |
| 2.055613815 | −0.035 |
| 2.0558574 | −0.133 |
| 2.056100985 | −0.231 |
| 2.05634457 | −0.563 |
| 2.056588155 | −0.954 |
| 2.05683174 | −1.368 |
| 2.057075325 | −1.855 |
| 2.05731891 | −2.342 |
| 2.057562495 | −3.162 |
| 2.05780608 | −4.135 |
| 2.058049665 | −4.495 |
| 2.05829325 | −0.599 |
| 2.058536835 | 3.296 |
| 2.05878042 | 4.702 |
| 2.059024005 | 4.213 |
| 2.05926759 | 3.729 |
| 2.059511175 | 3.728 |
| 2.05975476 | 3.726 |
| 2.059998345 | 3.505 |
| 2.06024193 | 3.018 |
| 2.060485515 | 2.531 |
| 2.0607291 | 2.479 |
| 2.060972685 | 2.477 |
| 2.06121627 | 2.313 |
| 2.061459855 | 1.829 |
| 2.06170344 | 1.344 |
| 2.061947025 | 1.236 |
| 2.06219061 | 1.235 |
| 2.062434195 | 1.129 |
| 2.06267778 | 0.646 |
| 2.062921365 | 0.163 |
| 2.06316495 | 0 |
| 2.063408535 | 0 |
| 2.06365212 | 0 |
| 2.063895705 | 0 |
| 2.06413929 | 0 |
| 2.064382875 | 0 |
| 2.06462646 | 0 |
| 2.064870045 | 0 |
| 2.06511363 | 0 |
| 2.065357215 | 0 |
| 2.0656008 | 0 |
| 2.065844385 | 0 |
| 2.06608797 | 0 |
| 2.066331555 | 0 |
| 2.06657514 | 0 |
| 2.066818725 | 0 |
| 2.06706231 | 0 |
| 2.067305895 | 0 |
| 2.06754948 | 0 |
| 2.067793065 | 0 |
| 2.06803665 | 0.377 |
| 2.068280235 | 2.282 |
| 2.06852382 | 4.188 |
| 2.068767405 | 4.872 |
| 2.06901099 | 4.869 |
| 2.069254575 | 4.711 |
| 2.06949816 | 2.808 |
| 2.069741745 | 0.906 |
| 2.06998533 | 0 |
| 2.070228915 | 0 |
| 2.0704725 | 0 |
| 2.070716085 | 0 |
| 2.07095967 | 0 |
| 2.071203255 | −0.039 |
| 2.07144684 | −0.133 |
| 2.071690425 | −0.228 |
| 2.07193401 | −0.563 |
| 2.072177595 | −0.941 |
| 2.07242118 | −1.346 |
| 2.072664765 | −1.817 |
| 2.07290835 | −2.289 |
| 2.073151935 | −3.105 |
| 2.07339552 | −4.046 |
| 2.073639105 | −4.159 |
| 2.07388269 | −0.391 |
| 2.074126275 | 3.376 |
| 2.07436986 | 4.523 |
| 2.074613445 | 4.051 |
| 2.07485703 | 3.606 |
| 2.075100615 | 3.604 |
| 2.0753442 | 3.603 |
| 2.075587785 | 3.365 |
| 2.07583137 | 2.895 |
| 2.076074955 | 2.424 |
| 2.07631854 | 2.397 |
| 2.076562125 | 2.395 |
| 2.07680571 | 2.214 |
| 2.077049295 | 1.745 |
| 2.07729288 | 1.276 |
| 2.077536465 | 1.195 |
| 2.07778005 | 1.195 |
| 2.078023635 | 1.068 |
| 2.07826722 | 0.601 |
| 2.078510805 | 0.134 |
| 2.07875439 | 0 |
| 2.078997975 | 0 |
| 2.07924156 | 0 |
| 2.079485145 | 0 |
| 2.07972873 | 0 |
| 2.079972315 | 0 |
| 2.0802159 | 0 |
| 2.080459485 | 0 |
| 2.08070307 | 0 |
| 2.080946655 | 0 |
| 2.08119024 | 0 |
| 2.081433825 | 0 |
| 2.08167741 | 0 |
| 2.081920995 | 0 |
| 2.08216458 | 0 |
| 2.082408165 | 0 |
| 2.08265175 | 0 |
| 2.082895335 | 0 |
| 2.08313892 | 0 |
| 2.083382505 | 0 |
| 2.08362609 | 0.457 |
| 2.083869675 | 2.3 |
| 2.08411326 | 4.142 |
| 2.084356845 | 4.712 |
| 2.08460043 | 4.709 |
| 2.084844015 | 4.464 |
| 2.0850876 | 2.624 |
| 2.085331185 | 0.784 |
| 2.08557477 | 0 |
| 2.085818355 | 0 |
| 2.08606194 | 0 |
| 2.086305525 | 0 |
| 2.08654911 | 0 |
| 2.086792695 | −0.042 |
| 2.08703628 | −0.133 |
| 2.087279865 | −0.225 |
| 2.08752345 | −0.563 |
| 2.087767035 | −0.929 |
| 2.08801062 | −1.325 |
| 2.088254205 | −1.781 |
| 2.08849779 | −2.237 |
| 2.088741375 | −3.049 |
| 2.08898496 | −3.959 |
| 2.089228545 | −3.84 |
| 2.08947213 | −0.196 |
| 2.089715715 | 3.448 |
| 2.0899593 | 4.353 |
| 2.090202885 | 3.896 |
| 2.09044647 | 3.489 |
| 2.090690055 | 3.487 |
| 2.09093364 | 3.485 |
| 2.091177225 | 3.233 |
| 2.09142081 | 2.778 |
| 2.091664395 | 2.323 |
| 2.09190798 | 2.319 |
| 2.092151565 | 2.318 |
| 2.09239515 | 2.119 |
| 2.092638735 | 1.666 |
| 2.09288232 | 1.212 |
| 2.093125905 | 1.156 |
| 2.09336949 | 1.156 |
| 2.093613075 | 1.011 |
| 2.09385666 | 0.559 |
| 2.094100245 | 0.108 |
| 2.09434383 | 0 |
| 2.094587415 | 0 |
| 2.094831 | 0 |
| 2.095074585 | 0 |
| 2.09531817 | 0 |
| 2.095561755 | 0 |
| 2.09580534 | 0 |
| 2.096048925 | 0 |
| 2.09629251 | 0 |
| 2.096536095 | 0 |
| 2.09677968 | 0 |
| 2.097023265 | 0 |
| 2.09726685 | 0 |
| 2.097510435 | 0 |
| 2.09775402 | 0 |
| 2.097997605 | 0 |
| 2.09824119 | 0 |
| 2.098484775 | 0 |
| 2.09872836 | 0 |
| 2.098971945 | 0 |
| 2.09921553 | 0.532 |
| 2.099459115 | 2.315 |
| 2.0997027 | 4.099 |
| 2.099946285 | 4.561 |
| 2.10018987 | 4.558 |
| 2.100433455 | 4.232 |
| 2.10067704 | 2.451 |
| 2.100920625 | 0.67 |
| 2.10116421 | 0 |
| 2.101407795 | 0 |
| 2.10165138 | 0 |
| 2.101894965 | 0 |
| 2.10213855 | 0 |
| 2.102382135 | −0.045 |
| 2.10262572 | −0.134 |
| 2.102869305 | −0.222 |
| 2.10311289 | −0.563 |
| 2.103356475 | −0.917 |
| 2.10360006 | −1.305 |
| 2.103843645 | −1.746 |
| 2.10408723 | −2.188 |
| 2.104330815 | −2.996 |
| 2.1045744 | −3.878 |
| 2.104817985 | −3.542 |
| 2.10506157 | −0.013 |
| 2.105305155 | 3.515 |
| 2.10554874 | 4.193 |
| 2.105792325 | 3.75 |
| 2.10603591 | 3.378 |
| 2.106279495 | 3.377 |
| 2.10652308 | 3.375 |
| 2.106766665 | 3.109 |
| 2.10701025 | 2.668 |
| 2.107253835 | 2.247 |
| 2.10749742 | 2.246 |
| 2.107741005 | 2.245 |
| 2.10798459 | 2.031 |
| 2.108228175 | 1.591 |
| 2.10847176 | 1.152 |
| 2.108715345 | 1.12 |
| 2.10895893 | 1.12 |
| 2.109202515 | 0.957 |
| 2.1094461 | 0.52 |
| 2.109689685 | 0.082 |
| 2.10993327 | 0 |
| 2.110176855 | 0 |
| 2.11042044 | 0 |
| 2.110664025 | 0 |
| 2.11090761 | 0 |
| 2.111151195 | 0 |
| 2.11139478 | 0 |
| 2.111638365 | 0 |
| 2.11188195 | 0 |
| 2.112125535 | 0 |
| 2.11236912 | 0 |
| 2.112612705 | 0 |
| 2.11285629 | 0 |
| 2.113099875 | 0 |
| 2.11334346 | 0 |
| 2.113587045 | 0 |
| 2.11383063 | 0 |
| 2.114074215 | 0 |
| 2.1143178 | 0 |
| 2.114561385 | 0 |
| 2.11480497 | 0.602 |
| 2.115048555 | 2.331 |
| 2.11529214 | 4.059 |
| 2.115535725 | 4.421 |
| 2.11577931 | 4.419 |
| 2.116022895 | 4.017 |
| 2.11626648 | 2.29 |
| 2.116510065 | 0.563 |
| 2.11675365 | 0 |
| 2.116997235 | 0 |
| 2.11724082 | 0 |
| 2.117484405 | 0 |
| 2.11772799 | 0 |
| 2.117971575 | 0 |
| 2.11821516 | 0 |
| 2.118458745 | 0 |
| 2.11870233 | 0 |
| 2.118945915 | 0 |
| 2.1191895 | 0 |
| 2.119433085 | 0 |
| 2.11967667 | 0 |
| 2.119920255 | 0 |
| 2.12016384 | 0 |
| 2.120407425 | 0 |
| 2.12065101 | 0 |
| 2.120894595 | 0 |
| 2.12113818 | 0 |
| 2.121381765 | 0 |
| 2.12162535 | 0 |
| 2.121868935 | 0 |
| 2.12211252 | 0 |
| 2.122356105 | 0 |
| 2.12259969 | 0 |
| 2.122843275 | 0 |
| 2.12308686 | 0 |
| 2.123330445 | 0 |
| 2.12357403 | 0 |
| 2.123817615 | 0 |
| 2.1240612 | 0 |
| 2.124304785 | 0 |
| 2.12454837 | 0 |
| 2.124791955 | 0 |
| 2.12503554 | 0 |
| 2.125279125 | 0 |
| 2.12552271 | 0 |
| 2.125766295 | 0 |
| 2.12600988 | 0 |
| 2.126253465 | 0 |
| 2.12649705 | 0 |
| 2.126740635 | 0 |
| 2.12698422 | 0 |
| 2.127227805 | 0 |
| 2.12747139 | 0 |
| 2.127714975 | 0 |
| 2.12795856 | 0 |
| 2.128202145 | 0 |
| 2.12844573 | 0 |
| 2.128689315 | 0 |
| 2.1289329 | 0 |
| 2.129176485 | 0 |
| 2.12942007 | 0 |
| 2.129663655 | 0 |
| 2.12990724 | 0 |
| 2.130150825 | 0 |
| 2.13039441 | 0 |
| 2.130637995 | 0 |
| 2.13088158 | 0 |
| 2.131125165 | 0 |
| 2.13136875 | 0 |
| 2.131612335 | 0 |
| 2.13185592 | 0 |
| 2.132099505 | 0 |
| 2.13234309 | 0 |
| 2.132586675 | 0 |
| 2.13283026 | 0 |
| 2.133073845 | 0 |
| 2.13331743 | 0 |
| 2.133561015 | −0.051 |
| 2.1338046 | −0.134 |
| 2.134048185 | −0.23 |
| 2.13429177 | −0.564 |
| 2.134535355 | −0.897 |
| 2.13477894 | −1.272 |
| 2.135022525 | −1.688 |
| 2.13526611 | −2.104 |
| 2.135509695 | −2.908 |
| 2.13575328 | −3.74 |
| 2.135996865 | −3.007 |
| 2.13624045 | 0.321 |
| 2.136484035 | 3.648 |
| 2.13672762 | 3.913 |
| 2.136971205 | 3.496 |
| 2.13721479 | 3.187 |
| 2.137458375 | 3.185 |
| 2.13770196 | 3.184 |
| 2.137945545 | 2.892 |
| 2.13818913 | 2.476 |
| 2.138432715 | 2.12 |
| 2.1386763 | 2.119 |
| 2.138919885 | 2.118 |
| 2.13916347 | 1.875 |
| 2.139407055 | 1.461 |
| 2.13965064 | 1.058 |
| 2.139894225 | 1.057 |
| 2.14013781 | 1.057 |
| 2.140381395 | 0.863 |
| 2.14062498 | 0.449 |
| 2.140868565 | 0.036 |
| 2.14111215 | 0 |
| 2.141355735 | 0 |
| 2.14159932 | 0 |
| 2.141842905 | 0 |
| 2.14208649 | 0 |
| 2.142330075 | 0 |
| 2.14257366 | 0 |
| 2.142817245 | 0 |
| 2.14306083 | 0 |
| 2.143304415 | 0 |
| 2.143548 | 0 |
| 2.143791585 | 0 |
| 2.14403517 | 0 |
| 2.144278755 | 0 |
| 2.14452234 | 0 |
| 2.144765925 | 0 |
| 2.14500951 | 0 |
| 2.145253095 | 0 |
| 2.14549668 | 0 |
| 2.145740265 | 0 |
| 2.14598385 | 0.733 |
| 2.146227435 | 2.369 |
| 2.14647102 | 4.005 |
| 2.146714605 | 4.183 |
| 2.14695819 | 4.182 |
| 2.147201775 | 3.638 |
| 2.14744536 | 2.004 |
| 2.147688945 | 0.369 |
| 2.14793253 | 0 |
| 2.148176115 | 0 |
| 2.1484197 | 0 |
| 2.148663285 | 0 |
| 2.14890687 | 0 |
| 2.149150455 | −0.054 |
| 2.14939404 | −0.135 |
| 2.149637625 | −0.241 |
| 2.14988121 | −0.566 |
| 2.150124795 | −0.891 |
| 2.15036838 | −1.26 |
| 2.150611965 | −1.666 |
| 2.15085555 | −2.072 |
| 2.151099135 | −2.876 |
| 2.15134272 | −3.687 |
| 2.151586305 | −2.77 |
| 2.15182989 | 0.475 |
| 2.152073475 | 3.72 |
| 2.15231706 | 3.796 |
| 2.152560645 | 3.389 |
| 2.15280423 | 3.108 |
| 2.153047815 | 3.107 |
| 2.1532914 | 3.106 |
| 2.153534985 | 2.801 |
| 2.15377857 | 2.395 |
| 2.154022155 | 2.068 |
| 2.15426574 | 2.068 |
| 2.154509325 | 2.067 |
| 2.15475291 | 1.809 |
| 2.154996495 | 1.405 |
| 2.15524008 | 1.032 |
| 2.155483665 | 1.032 |
| 2.15572725 | 1.032 |
| 2.155970835 | 0.822 |
| 2.15621442 | 0.419 |
| 2.156458005 | 0.015 |
| 2.15670159 | 0 |
| 2.156945175 | 0 |
| 2.15718876 | 0 |
| 2.157432345 | 0 |
| 2.15767593 | 0 |
| 2.157919515 | 0 |
| 2.1581631 | 0 |
| 2.158406685 | 0 |
| 2.15865027 | 0 |
| 2.158893855 | 0 |
| 2.15913744 | 0 |
| 2.159381025 | 0 |
| 2.15962461 | 0 |
| 2.159868195 | 0 |
| 2.16011178 | 0 |
| 2.160355365 | 0 |
| 2.16059895 | 0 |
| 2.160842535 | 0 |
| 2.16108612 | 0 |
| 2.161329705 | 0 |
| 2.16157329 | 0.796 |
| 2.161816875 | 2.395 |
| 2.16206046 | 3.994 |
| 2.162304045 | 4.089 |
| 2.16254763 | 4.087 |
| 2.162791215 | 3.476 |
| 2.1630348 | 1.879 |
| 2.163278385 | 0.281 |
| 2.16352197 | 0 |
| 2.163765555 | 0 |
| 2.16400914 | 0 |
| 2.164252725 | 0 |
| 2.16449631 | 0 |
| 2.164739895 | −0.056 |
| 2.16498348 | −0.136 |
| 2.165227065 | −0.251 |
| 2.16547065 | −0.569 |
| 2.165714235 | −0.888 |
| 2.16595782 | −1.253 |
| 2.166201405 | −1.65 |
| 2.16644499 | −2.06 |
| 2.166688575 | −2.854 |
| 2.16693216 | −3.647 |
| 2.167175745 | −2.552 |
| 2.16741933 | 0.624 |
| 2.167662915 | 3.8 |
| 2.1679065 | 3.695 |
| 2.168150085 | 3.297 |
| 2.16839367 | 3.042 |
| 2.168637255 | 3.041 |
| 2.16888084 | 3.04 |
| 2.169124425 | 2.722 |
| 2.16936801 | 2.325 |
| 2.169611595 | 2.025 |
| 2.16985518 | 2.025 |
| 2.170098765 | 2.024 |
| 2.17034235 | 1.752 |
| 2.170585935 | 1.356 |
| 2.17082952 | 1.011 |
| 2.171073105 | 1.011 |
| 2.17131669 | 1.01 |
| 2.171560275 | 0.785 |
| 2.17180386 | 0.39 |
| 2.172047445 | 0 |
| 2.17229103 | 0 |
| 2.172534615 | 0 |
| 2.1727782 | 0 |
| 2.173021785 | 0 |
| 2.17326537 | 0 |
| 2.173508955 | 0 |
| 2.17375254 | 0 |
| 2.173996125 | 0 |
| 2.17423971 | 0 |
| 2.174483295 | 0 |
| 2.17472688 | 0 |
| 2.174970465 | 0 |
| 2.17521405 | 0 |
| 2.175457635 | 0 |
| 2.17570122 | 0 |
| 2.175944805 | 0 |
| 2.17618839 | 0 |
| 2.176431975 | 0 |
| 2.17667556 | 0 |
| 2.176919145 | 0 |
| 2.17716273 | 0.86 |
| 2.177406315 | 2.428 |
| 2.1776499 | 3.997 |
| 2.177893485 | 4.012 |
| 2.17813707 | 4.011 |
| 2.178380655 | 3.333 |
| 2.17862424 | 1.765 |
| 2.178867825 | 0.198 |
| 2.17911141 | 0 |
| 2.179354995 | 0 |
| 2.17959858 | 0 |
| 2.179842165 | 0 |
| 2.18008575 | 0 |
| 2.180329335 | 0 |
| 2.18057292 | 0 |
| 2.180816505 | 0 |
| 2.18106009 | 0 |
| 2.181303675 | 0 |
| 2.18154726 | 0 |
| 2.181790845 | 0 |
| 2.18203443 | 0 |
| 2.182278015 | 0 |
| 2.1825216 | 0 |
| 2.182765185 | 0 |
| 2.18300877 | 0 |
| 2.183252355 | 0 |
| 2.18349594 | 0 |
| 2.183739525 | 0 |
| 2.18398311 | 0 |
| 2.184226695 | 0 |
| 2.18447028 | 0 |
| 2.184713865 | 0 |
| 2.18495745 | 0 |
| 2.185201035 | 0 |
| 2.18544462 | 0 |
| 2.185688205 | 0 |
| 2.18593179 | 0 |
| 2.186175375 | 0 |
| 2.18641896 | 0 |
| 2.186662545 | 0 |
| 2.18690613 | 0 |
| 2.187149715 | 0 |
| 2.1873933 | 0 |
| 2.187636885 | 0 |
| 2.18788047 | 0 |
| 2.188124055 | 0 |
| 2.18836764 | 0 |
| 2.188611225 | 0 |
| 2.18885481 | 0 |
| 2.189098395 | 0 |
| 2.18934198 | 0 |
| 2.189585565 | 0 |
| 2.18982915 | 0 |
| 2.190072735 | 0 |
| 2.19031632 | 0 |
| 2.190559905 | 0 |
| 2.19080349 | 0 |
| 2.191047075 | 0 |
| 2.19129066 | 0 |
| 2.191534245 | 0 |
| 2.19177783 | 0 |
| 2.192021415 | 0 |
| 2.192265 | 0 |
| 2.192508585 | 0 |
| 2.19275217 | 0 |
| 2.192995755 | 0 |
| 2.19323934 | 0 |
| 2.193482925 | 0 |
| 2.19372651 | 0 |
| 2.193970095 | 0 |
| 2.19421368 | 0 |
| 2.194457265 | 0 |
| 2.19470085 | 0 |
| 2.194944435 | 0 |
| 2.19518802 | 0 |
| 2.195431605 | 0 |
| 2.19567519 | 0 |
| 2.195918775 | 0 |
| 2.19616236 | 0 |
| 2.196405945 | 0 |
| 2.19664953 | 0 |
| 2.196893115 | 0 |
| 2.1971367 | 0 |
| 2.197380285 | 0 |
| 2.19762387 | 0 |
| 2.197867455 | 0 |
| 2.19811104 | 0 |
| 2.198354625 | 0 |
| 2.19859821 | 0 |
| 2.198841795 | 0 |
| 2.19908538 | 0 |
| 2.199328965 | 0 |
| 2.19957255 | 0 |
| 2.199816135 | 0 |
| 2.20005972 | 0 |
| 2.200303305 | 0 |
| 2.20054689 | 0 |
| 2.200790475 | 0 |
| 2.20103406 | 0 |
| 2.201277645 | 0 |
| 2.20152123 | 0 |
| 2.201764815 | 0 |
| 2.2020084 | 0 |
| 2.202251985 | 0 |
| 2.20249557 | 0 |
| 2.202739155 | 0 |
| 2.20298274 | 0 |
| 2.203226325 | 0 |
| 2.20346991 | 0 |
| 2.203713495 | 0 |
| 2.20395708 | 0 |
| 2.204200665 | 0 |
| 2.20444425 | 0 |
| 2.204687835 | 0 |
| 2.20493142 | 0 |
| 2.205175005 | 0 |
| 2.20541859 | 0 |
| 2.205662175 | 0 |
| 2.20590576 | 0 |
| 2.206149345 | 0 |
| 2.20639293 | 0 |
| 2.206636515 | 0 |
| 2.2068801 | 0 |
| 2.207123685 | 0 |
| 2.20736727 | 0 |
| 2.207610855 | 0 |
| 2.20785444 | 0 |
| 2.208098025 | 0 |
| 2.20834161 | 0 |
| 2.208585195 | 0 |
| 2.20882878 | 0 |
| 2.209072365 | 0 |
| 2.20931595 | 0 |
| 2.209559535 | 0 |
| 2.20980312 | 0 |
| 2.210046705 | 0 |
| 2.21029029 | 0 |
| 2.210533875 | 0 |
| 2.21077746 | 0 |
| 2.211021045 | 0 |
| 2.21126463 | 0 |
| 2.211508215 | −0.066 |
| 2.2117518 | −0.142 |
| 2.211995385 | −0.287 |
| 2.21223897 | −0.593 |
| 2.212482555 | −0.898 |
| 2.21272614 | −1.26 |
| 2.212969725 | −1.642 |
| 2.21321331 | −2.094 |
| 2.213456895 | −2.857 |
| 2.21370048 | −3.62 |
| 2.213944065 | −1.994 |
| 2.21418765 | 1.059 |
| 2.214431235 | 3.878 |
| 2.21467482 | 3.497 |
| 2.214918405 | 3.115 |
| 2.21516199 | 2.927 |
| 2.215405575 | 2.927 |
| 2.21564916 | 2.927 |
| 2.215892745 | 2.564 |
| 2.21613633 | 2.182 |
| 2.216379915 | 1.951 |
| 2.2166235 | 1.951 |
| 2.216867085 | 1.95 |
| 2.21711067 | 1.632 |
| 2.217354255 | 1.25 |
| 2.21759784 | 0.975 |
| 2.217841425 | 0.975 |
| 2.21808501 | 0.975 |
| 2.218328595 | 0.7 |
| 2.21857218 | 0.319 |
| 2.218815765 | 0 |
| 2.21905935 | 0 |
| 2.219302935 | 0 |
| 2.21954652 | 0 |
| 2.219790105 | 0 |
| 2.22003369 | 0 |
| 2.220277275 | 0 |
| 2.22052086 | 0 |
| 2.220764445 | 0 |
| 2.22100803 | 0 |
| 2.221251615 | 0 |
| 2.2214952 | 0 |
| 2.221738785 | 0 |
| 2.22198237 | 0 |
| 2.222225955 | 0 |
| 2.22246954 | 0 |
| 2.222713125 | 0 |
| 2.22295671 | 0 |
| 2.223200295 | 0 |
| 2.22344388 | 0 |
| 2.223687465 | 0 |
| 2.22393105 | 1.062 |
| 2.224174635 | 2.584 |
| 2.22441822 | 3.893 |
| 2.224661805 | 3.892 |
| 2.22490539 | 3.892 |
| 2.225148975 | 3.007 |
| 2.22539256 | 1.485 |
| 2.225636145 | 0 |
| 2.22587973 | 0 |
| 2.226123315 | 0 |
| 2.2263669 | 0 |
| 2.226610485 | 0 |
| 2.22685407 | 0 |
| 2.227097655 | −0.069 |
| 2.22734124 | −0.145 |
| 2.227584825 | −0.301 |
| 2.22782841 | −0.605 |
| 2.228071995 | −0.909 |
| 2.22831558 | −1.274 |
| 2.228559165 | −1.654 |
| 2.22880275 | −2.123 |
| 2.229046335 | −2.883 |
| 2.22928992 | −3.643 |
| 2.229533505 | −1.834 |
| 2.22977709 | 1.207 |
| 2.230020675 | 3.844 |
| 2.23026426 | 3.464 |
| 2.230507845 | 3.084 |
| 2.23075143 | 2.917 |
| 2.230995015 | 2.917 |
| 2.2312386 | 2.916 |
| 2.231482185 | 2.536 |
| 2.23172577 | 2.156 |
| 2.231969355 | 1.944 |
| 2.23221294 | 1.944 |
| 2.232456525 | 1.944 |
| 2.23270011 | 1.608 |
| 2.232943695 | 1.228 |
| 2.23318728 | 0.972 |
| 2.233430865 | 0.972 |
| 2.23367445 | 0.972 |
| 2.233918035 | 0.679 |
| 2.23416162 | 0.299 |
| 2.234405205 | 0 |
| 2.23464879 | 0 |
| 2.234892375 | 0 |
| 2.23513596 | 0 |
| 2.235379545 | 0 |
| 2.23562313 | 0 |
| 2.235866715 | 0 |
| 2.2361103 | 0 |
| 2.236353885 | 0 |
| 2.23659747 | 0 |
| 2.236841055 | 0 |
| 2.23708464 | 0 |
| 2.237328225 | 0 |
| 2.23757181 | 0 |
| 2.237815395 | 0 |
| 2.23805898 | 0 |
| 2.238302565 | 0 |
| 2.23854615 | 0 |
| 2.238789735 | 0 |
| 2.23903332 | 0 |
| 2.239276905 | 0 |
| 2.23952049 | 1.137 |
| 2.239764075 | 2.658 |
| 2.24000766 | 3.889 |
| 2.240251245 | 3.889 |
| 2.24049483 | 3.889 |
| 2.240738415 | 2.928 |
| 2.240982 | 1.408 |
| 2.241225585 | 0 |
| 2.24146917 | 0 |
| 2.241712755 | 0 |
| 2.24195634 | 0 |
| 2.242199925 | 0 |
| 2.24244351 | 0 |
| 2.242687095 | 0 |
| 2.24293068 | 0 |
| 2.243174265 | 0 |
| 2.24341785 | 0 |
| 2.243661435 | 0 |
| 2.24390502 | 0 |
| 2.244148605 | 0 |
| 2.24439219 | 0 |
| 2.244635775 | 0 |
| 2.24487936 | 0 |
| 2.245122945 | 0 |
| 2.24536653 | 0 |
| 2.245610115 | 0 |
| 2.2458537 | 0 |
| 2.246097285 | 0 |
| 2.24634087 | 0 |
| 2.246584455 | 0 |
| 2.24682804 | 0 |
| 2.247071625 | 0 |
| 2.24731521 | 0 |
| 2.247558795 | 0 |
| 2.24780238 | 0 |
| 2.248045965 | 0 |
| 2.24828955 | 0 |
| 2.248533135 | 0 |
| 2.24877672 | 0 |
| 2.249020305 | 0 |
| 2.24926389 | 0 |
| 2.249507475 | 0 |
| 2.24975106 | 0 |
| 2.249994645 | 0 |
| 2.25023823 | 0 |
| 2.250481815 | 0 |
| 2.2507254 | 0 |
| 2.250968985 | 0 |
| 2.25121257 | 0 |
| 2.251456155 | 0 |
| 2.25169974 | 0 |
| 2.251943325 | 0 |
| 2.25218691 | 0 |
| 2.252430495 | 0 |
| 2.25267408 | 0 |
| 2.252917665 | 0 |
| 2.25316125 | 0 |
| 2.253404835 | 0 |
| 2.25364842 | 0 |
| 2.253892005 | 0 |
| 2.25413559 | 0 |
| 2.254379175 | 0 |
| 2.25462276 | 0 |
| 2.254866345 | 0 |
| 2.25510993 | 0 |
| 2.255353515 | 0 |
| 2.2555971 | 0 |
| 2.255840685 | 0 |
| 2.25608427 | 0 |
| 2.256327855 | 0 |
| 2.25657144 | 0 |
| 2.256815025 | 0 |
| 2.25705861 | 0 |
| 2.257302195 | 0 |
| 2.25754578 | 0 |
| 2.257789365 | 0 |
| 2.25803295 | 0 |
| 2.258276535 | 0 |
| 2.25852012 | 0 |
| 2.258763705 | 0 |
| 2.25900729 | 0 |
| 2.259250875 | 0 |
| 2.25949446 | 0 |
| 2.259738045 | 0 |
| 2.25998163 | 0 |
| 2.260225215 | 0 |
| 2.2604688 | 0 |
| 2.260712385 | 0 |
| 2.26095597 | 0 |
| 2.261199555 | 0 |
| 2.26144314 | 0 |
| 2.261686725 | 0 |
| 2.26193031 | 0 |
| 2.262173895 | 0 |
| 2.26241748 | 0 |
| 2.262661065 | 0 |
| 2.26290465 | 0 |
| 2.263148235 | 0 |
| 2.26339182 | 0 |
| 2.263635405 | 0 |
| 2.26387899 | 0 |
| 2.264122575 | 0 |
| 2.26436616 | 0 |
| 2.264609745 | 0 |
| 2.26485333 | 0 |
| 2.265096915 | 0 |
| 2.2653405 | 0 |
| 2.265584085 | 0 |
| 2.26582767 | 0 |
| 2.266071255 | 0 |
| 2.26631484 | 0 |
| 2.266558425 | 0 |
| 2.26680201 | 0 |
| 2.267045595 | 0 |
| 2.26728918 | 0 |
| 2.267532765 | 0 |
| 2.26777635 | 0 |
| 2.268019935 | 0 |
| 2.26826352 | 0 |
| 2.268507105 | 0 |
| 2.26875069 | 0 |
| 2.268994275 | 0 |
| 2.26923786 | 0 |
| 2.269481445 | 0 |
| 2.26972503 | 0 |
| 2.269968615 | 0 |
| 2.2702122 | 0 |
| 2.270455785 | 0 |
| 2.27069937 | 0 |
| 2.270942955 | 0 |
| 2.27118654 | 0 |
| 2.271430125 | 0 |
| 2.27167371 | 0 |
| 2.271917295 | 0 |
| 2.27216088 | 0 |
| 2.272404465 | 0 |
| 2.27264805 | 0 |
| 2.272891635 | 0 |
| 2.27313522 | 0 |
| 2.273378805 | 0 |
| 2.27362239 | 0 |
| 2.273865975 | 0 |
| 2.27410956 | 0 |
| 2.274353145 | 0 |
| 2.27459673 | 0 |
| 2.274840315 | 0 |
| 2.2750839 | 0 |
| 2.275327485 | 0 |
| 2.27557107 | 0 |
| 2.275814655 | 0 |
| 2.27605824 | 0 |
| 2.276301825 | 0 |
| 2.27654541 | 0 |
| 2.276788995 | 0 |
| 2.27703258 | 0 |
| 2.277276165 | 0 |
| 2.27751975 | 0 |
| 2.277763335 | 0 |
| 2.27800692 | 0 |
| 2.278250505 | 0 |
| 2.27849409 | 0 |
| 2.278737675 | 0 |
| 2.27898126 | 0 |
| 2.279224845 | 0 |
| 2.27946843 | 0 |
| 2.279712015 | 0 |
| 2.2799556 | 0 |
| 2.280199185 | 0 |
| 2.28044277 | 0 |
| 2.280686355 | 0 |
| 2.28092994 | 0 |
| 2.281173525 | 0 |
| 2.28141711 | 0 |
| 2.281660695 | 0 |
| 2.28190428 | 0 |
| 2.282147865 | 0 |
| 2.28239145 | 0 |
| 2.282635035 | 0 |
| 2.28287862 | 0 |
| 2.283122205 | 0 |
| 2.28336579 | 0 |
| 2.283609375 | 0 |
| 2.28385296 | 0 |
| 2.284096545 | 0 |
| 2.28434013 | 0 |
| 2.284583715 | 0 |
| 2.2848273 | 0 |
| 2.285070885 | 0 |
| 2.28531447 | 0 |
| 2.285558055 | 0 |
| 2.28580164 | 0 |
| 2.286045225 | 0 |
| 2.28628881 | 0 |
| 2.286532395 | 0 |
| 2.28677598 | 0 |
| 2.287019565 | 0 |
| 2.28726315 | 0 |
| 2.287506735 | 0 |
| 2.28775032 | 0 |
| 2.287993905 | 0 |
| 2.28823749 | 0 |
| 2.288481075 | 0 |
| 2.28872466 | 0 |
| 2.288968245 | 0 |
| 2.28921183 | 0 |
| 2.289455415 | 0 |
| 2.289699 | 0 |
| 2.289942585 | 0 |
| 2.29018617 | 0 |
| 2.290429755 | 0 |
| 2.29067334 | 0 |
| 2.290916925 | 0 |
| 2.29116051 | 0 |
| 2.291404095 | 0 |
| 2.29164768 | 0 |
| 2.291891265 | 0 |
| 2.29213485 | 0 |
| 2.292378435 | 0 |
| 2.29262202 | 0 |
| 2.292865605 | 0 |
| 2.29310919 | 0 |
| 2.293352775 | 0 |
| 2.29359636 | 0 |
| 2.293839945 | 0 |
| 2.29408353 | 0 |
| 2.294327115 | 0 |
| 2.2945707 | 0 |
| 2.294814285 | 0 |
| 2.29505787 | 0 |
| 2.295301455 | 0 |
| 2.29554504 | 0 |
| 2.295788625 | 0 |
| 2.29603221 | 0 |
| 2.296275795 | 0 |
| 2.29651938 | 0 |
| 2.296762965 | 0 |
| 2.29700655 | 0 |
| 2.297250135 | 0 |
| 2.29749372 | 0 |
| 2.297737305 | 0 |
| 2.29798089 | 0 |
| 2.298224475 | 0 |
| 2.29846806 | 0 |
| 2.298711645 | 0 |
| 2.29895523 | 0 |
| 2.299198815 | 0 |
| 2.2994424 | 0 |
| 2.299685985 | 0 |
| 2.29992957 | 0 |
| 2.300173155 | 0 |
| 2.30041674 | 0 |
| 2.300660325 | 0 |
| 2.30090391 | 0 |
| 2.301147495 | 0 |
| 2.30139108 | 0 |
| 2.301634665 | 0 |
| 2.30187825 | 0 |
| 2.302121835 | 0 |
| 2.30236542 | 0 |
| 2.302609005 | 0 |
| 2.30285259 | 0 |
| 2.303096175 | 0 |
| 2.30333976 | 0 |
| 2.303583345 | 0 |
| 2.30382693 | 0 |
| 2.304070515 | 0 |
| 2.3043141 | 0 |
| 2.304557685 | 0 |
| 2.30480127 | −0.012 |
| 2.305044855 | −0.091 |
| 2.30528844 | −0.17 |
| 2.305532025 | −0.392 |
| 2.30577561 | −0.708 |
| 2.306019195 | −1.027 |
| 2.30626278 | −1.423 |
| 2.306506365 | −1.819 |
| 2.30674995 | −2.406 |
| 2.306993535 | −3.198 |
| 2.30723712 | −3.99 |
| 2.307480705 | −1.116 |
| 2.30772429 | 2.05 |
| 2.307967875 | 3.905 |
| 2.30821146 | 3.51 |
| 2.308455045 | 3.115 |
| 2.30869863 | 3.041 |
| 2.308942215 | 3.041 |
| 2.3091858 | 2.942 |
| 2.309429385 | 2.546 |
| 2.30967297 | 2.15 |
| 2.309916555 | 2.03 |
| 2.31016014 | 2.03 |
| 2.310403725 | 1.977 |
| 2.31064731 | 1.58 |
| 2.310890895 | 1.183 |
| 2.31113448 | 1.016 |
| 2.311378065 | 1.016 |
| 2.31162165 | 1.009 |
| 2.311865235 | 0.611 |
| 2.31210882 | 0.214 |
| 2.312352405 | 0 |
| 2.31259599 | 0 |
| 2.312839575 | 0 |
| 2.31308316 | 0 |
| 2.313326745 | 0 |
| 2.31357033 | 0 |
| 2.313813915 | 0 |
| 2.3140575 | 0 |
| 2.314301085 | 0 |
| 2.31454467 | 0 |
| 2.314788255 | 0 |
| 2.31503184 | 0 |
| 2.315275425 | 0 |
| 2.31551901 | 0 |
| 2.315762595 | 0 |
| 2.31600618 | 0 |
| 2.316249765 | 0 |
| 2.31649335 | 0 |
| 2.316736935 | 0 |
| 2.31698052 | 0 |
| 2.317224105 | 0 |
| 2.31746769 | 1.597 |
| 2.317711275 | 3.197 |
| 2.31795486 | 4.094 |
| 2.318198445 | 4.095 |
| 2.31844203 | 4.096 |
| 2.318685615 | 2.684 |
| 2.3189292 | 1.082 |
| 2.319172785 | 0 |
| 2.31941637 | 0 |
| 2.319659955 | 0 |
| 2.31990354 | 0 |
| 2.320147125 | 0 |
| 2.32039071 | 0 |
| 2.320634295 | 0 |
| 2.32087788 | 0 |
| 2.321121465 | 0 |
| 2.32136505 | 0 |
| 2.321608635 | 0 |
| 2.32185222 | 0 |
| 2.322095805 | 0 |
| 2.32233939 | 0 |
| 2.322582975 | 0 |
| 2.32282656 | 0 |
| 2.323070145 | 0 |
| 2.32331373 | 0 |
| 2.323557315 | 0 |
| 2.3238009 | 0 |
| 2.324044485 | 0 |
| 2.32428807 | 0 |
| 2.324531655 | 0 |
| 2.32477524 | 0 |
| 2.325018825 | 0 |
| 2.32526241 | 0 |
| 2.325505995 | 0 |
| 2.32574958 | 0 |
| 2.325993165 | 0 |
| 2.32623675 | 0 |
| 2.326480335 | 0 |
| 2.32672392 | 0 |
| 2.326967505 | 0 |
| 2.32721109 | 0 |
| 2.327454675 | 0 |
| 2.32769826 | 0 |
| 2.327941845 | 0 |
| 2.32818543 | 0 |
| 2.328429015 | 0 |
| 2.3286726 | 0 |
| 2.328916185 | 0 |
| 2.32915977 | 0 |
| 2.329403355 | 0 |
| 2.32964694 | 0 |
| 2.329890525 | 0 |
| 2.33013411 | 0 |
| 2.330377695 | 0 |
| 2.33062128 | 0 |
| 2.330864865 | 0 |
| 2.33110845 | 0 |
| 2.331352035 | 0 |
| 2.33159562 | 0 |
| 2.331839205 | 0 |
| 2.33208279 | 0 |
| 2.332326375 | 0 |
| 2.33256996 | 0 |
| 2.332813545 | 0 |
| 2.33305713 | 0 |
| 2.333300715 | 0 |
| 2.3335443 | 0 |
| 2.333787885 | 0 |
| 2.33403147 | 0 |
| 2.334275055 | 0 |
| 2.33451864 | 0 |
| 2.334762225 | 0 |
| 2.33500581 | 0 |
| 2.335249395 | 0 |
| 2.33549298 | 0 |
| 2.335736565 | 0 |
| 2.33598015 | 0 |
| 2.336223735 | 0 |
| 2.33646732 | 0 |
| 2.336710905 | 0 |
| 2.33695449 | 0 |
| 2.337198075 | 0 |
| 2.33744166 | 0 |
| 2.337685245 | 0 |
| 2.33792883 | 0 |
| 2.338172415 | 0 |
| 2.338416 | 0 |
| 2.338659585 | 0 |
| 2.33890317 | 0 |
| 2.339146755 | 0 |
| 2.33939034 | 0 |
| 2.339633925 | 0 |
| 2.33987751 | 0 |
| 2.340121095 | 0 |
| 2.34036468 | 0 |
| 2.340608265 | 0 |
| 2.34085185 | 0 |
| 2.341095435 | 0 |
| 2.34133902 | 0 |
| 2.341582605 | 0 |
| 2.34182619 | 0 |
| 2.342069775 | 0 |
| 2.34231336 | 0 |
| 2.342556945 | 0 |
| 2.34280053 | 0 |
| 2.343044115 | 0 |
| 2.3432877 | 0 |
| 2.343531285 | 0 |
| 2.34377487 | 0 |
| 2.344018455 | 0 |
| 2.34426204 | 0 |
| 2.344505625 | 0 |
| 2.34474921 | 0 |
| 2.344992795 | 0 |
| 2.34523638 | 0 |
| 2.345479965 | 0 |
| 2.34572355 | 0 |
| 2.345967135 | 0 |
| 2.34621072 | 0 |
| 2.346454305 | 0 |
| 2.34669789 | 0 |
| 2.346941475 | 0 |
| 2.34718506 | 0 |
| 2.347428645 | 0 |
| 2.34767223 | 0 |
| 2.347915815 | 0 |
| 2.3481594 | 0 |
| 2.348402985 | 0 |
| 2.34864657 | 0 |
| 2.348890155 | 0 |
| 2.34913374 | 0 |
| 2.349377325 | 0 |
| 2.34962091 | 0 |
| 2.349864495 | 0 |
| 2.35010808 | 0 |
| 2.350351665 | 0 |
| 2.35059525 | 0 |
| 2.350838835 | 0 |
| 2.35108242 | 0 |
| 2.351326005 | 0 |
| 2.35156959 | 0 |
| 2.351813175 | 0 |
| 2.35205676 | 0 |
| 2.352300345 | 0 |
| 2.35254393 | 0 |
| 2.352787515 | 0 |
| 2.3530311 | 0 |
| 2.353274685 | 0 |
| 2.35351827 | 0 |
| 2.353761855 | 0 |
| 2.35400544 | 0 |
| 2.354249025 | 0 |
| 2.35449261 | 0 |
| 2.354736195 | 0 |
| 2.35497978 | 0 |
| 2.355223365 | 0 |
| 2.35546695 | 0 |
| 2.355710535 | 0 |
| 2.35595412 | 0 |
| 2.356197705 | 0 |
| 2.35644129 | 0 |
| 2.356684875 | 0 |
| 2.35692846 | 0 |
| 2.357172045 | 0 |
| 2.35741563 | 0 |
| 2.357659215 | 0 |
| 2.3579028 | 0 |
| 2.358146385 | 0 |
| 2.35838997 | 0 |
| 2.358633555 | 0 |
| 2.35887714 | 0 |
| 2.359120725 | 0 |
| 2.35936431 | 0 |
| 2.359607895 | 0 |
| 2.35985148 | 0 |
| 2.360095065 | 0 |
| 2.36033865 | 0 |
| 2.360582235 | 0 |
| 2.36082582 | 0 |
| 2.361069405 | 0 |
| 2.36131299 | 0 |
| 2.361556575 | 0 |
| 2.36180016 | 0 |
| 2.362043745 | 0 |
| 2.36228733 | 0 |
| 2.362530915 | 0 |
| 2.3627745 | 0 |
| 2.363018085 | 0 |
| 2.36326167 | 0 |
| 2.363505255 | 0 |
| 2.36374884 | 0 |
| 2.363992425 | 0 |
| 2.36423601 | 0 |
| 2.364479595 | 0 |
| 2.36472318 | 0 |
| 2.364966765 | 0 |
| 2.36521035 | 0 |
| 2.365453935 | 0 |
| 2.36569752 | 0 |
| 2.365941105 | 0 |
| 2.36618469 | 0 |
| 2.366428275 | 0 |
| 2.36667186 | 0 |
| 2.366915445 | 0 |
| 2.36715903 | 0 |
| 2.367402615 | 0 |
| 2.3676462 | 0 |
| 2.367889785 | 0 |
| 2.36813337 | 0 |
| 2.368376955 | 0 |
| 2.36862054 | 0 |
| 2.368864125 | 0 |
| 2.36910771 | 0 |
| 2.369351295 | 0 |
| 2.36959488 | 0 |
| 2.369838465 | 0 |
| 2.37008205 | 0 |
| 2.370325635 | 0 |
| 2.37056922 | 0 |
| 2.370812805 | 0 |
| 2.37105639 | 0 |
| 2.371299975 | 0 |
| 2.37154356 | 0 |
| 2.371787145 | 0 |
| 2.37203073 | 0 |
| 2.372274315 | 0 |
| 2.3725179 | 0 |
| 2.372761485 | 0 |
| 2.37300507 | 0 |
| 2.373248655 | 0 |
| 2.37349224 | 0 |
| 2.373735825 | 0 |
| 2.37397941 | 0 |
| 2.374222995 | 0 |
| 2.37446658 | 0 |
| 2.374710165 | 0 |
| 2.37495375 | 0 |
| 2.375197335 | 0 |
| 2.37544092 | 0 |
| 2.375684505 | 0 |
| 2.37592809 | 0 |
| 2.376171675 | 0 |
| 2.37641526 | 0 |
| 2.376658845 | 0 |
| 2.37690243 | 0 |
| 2.377146015 | 0 |
| 2.3773896 | 0 |
| 2.377633185 | 0 |
| 2.37787677 | 0 |
| 2.378120355 | 0 |
| 2.37836394 | 0 |
| 2.378607525 | 0 |
| 2.37885111 | 0 |
| 2.379094695 | 0 |
| 2.37933828 | 0 |
| 2.379581865 | 0 |
| 2.37982545 | 0 |
| 2.380069035 | 0 |
| 2.38031262 | 0 |
| 2.380556205 | 0 |
| 2.38079979 | 0 |
| 2.381043375 | 0 |
| 2.38128696 | 0 |
| 2.381530545 | 0 |
| 2.38177413 | 0 |
| 2.382017715 | 0 |
| 2.3822613 | 0 |
| 2.382504885 | 0 |
| 2.38274847 | 0 |
| 2.382992055 | 0 |
| 2.38323564 | 0 |
| 2.383479225 | 0 |
| 2.38372281 | 0 |
| 2.383966395 | 0 |
| 2.38420998 | 0 |
| 2.384453565 | 0 |
| 2.38469715 | 0 |
| 2.384940735 | 0 |
| 2.38518432 | 0 |
| 2.385427905 | 0 |
| 2.38567149 | 0 |
| 2.385915075 | 0 |
| 2.38615866 | 0 |
| 2.386402245 | 0 |
| 2.38664583 | 0 |
| 2.386889415 | 0 |
| 2.387133 | 0 |
| 2.387376585 | 0 |
| 2.38762017 | 0 |
| 2.387863755 | 0 |
| 2.38810734 | 0 |
| 2.388350925 | 0 |
| 2.38859451 | 0 |
| 2.388838095 | 0 |
| 2.38908168 | 0 |
| 2.389325265 | 0 |
| 2.38956885 | 0 |
| 2.389812435 | 0 |
| 2.39005602 | 0 |
| 2.390299605 | 0 |
| 2.39054319 | 0 |
| 2.390786775 | 0 |
| 2.39103036 | 0 |
| 2.391273945 | 0 |
| 2.39151753 | 0 |
| 2.391761115 | 0 |
| 2.3920047 | 0 |
| 2.392248285 | 0 |
| 2.39249187 | 0 |
| 2.392735455 | 0 |
| 2.39297904 | 0 |
| 2.393222625 | 0 |
| 2.39346621 | 0 |
| 2.393709795 | 0 |
| 2.39395338 | 0 |
| 2.394196965 | 0 |
| 2.39444055 | 0 |
| 2.394684135 | 0 |
| 2.39492772 | 0 |
| 2.395171305 | 0 |
| 2.39541489 | 0 |
| 2.395658475 | 0 |
| 2.39590206 | 0 |
| 2.396145645 | 0 |
| 2.39638923 | 0 |
| 2.396632815 | 0 |
| 2.3968764 | 0 |
| 2.397119985 | 0 |
| 2.39736357 | 0 |
| 2.397607155 | 0 |
| 2.39785074 | 0 |
| 2.398094325 | 0 |
| 2.39833791 | 0 |
| 2.398581495 | 0 |
| 2.39882508 | 0 |
| 2.399068665 | 0 |
| 2.39931225 | 0 |
| 2.399555835 | 0 |
| 2.39979942 | 0 |
| 2.400043005 | 0 |
| 2.40028659 | 0 |
| 2.400530175 | 0 |
| 2.40077376 | 0 |
| 2.401017345 | 0 |
| 2.40126093 | 0 |
| 2.401504515 | 0 |
| 2.4017481 | 0 |
| 2.401991685 | 0 |
| 2.40223527 | 0 |
| 2.402478855 | 0 |
| 2.40272244 | 0 |
| 2.402966025 | 0 |
| 2.40320961 | 0 |
| 2.403453195 | 0 |
| 2.40369678 | 0 |
| 2.403940365 | 0 |
| 2.40418395 | 0 |
| 2.404427535 | 0 |
| 2.40467112 | 0 |
| 2.404914705 | 0 |
| 2.40515829 | 0 |
| 2.405401875 | 0 |
| 2.40564546 | 0 |
| 2.405889045 | 0 |
| 2.40613263 | 0 |
| 2.406376215 | 0 |
| 2.4066198 | 0 |
| 2.406863385 | 0 |
| 2.40710697 | 0 |
| 2.407350555 | 0 |
| 2.40759414 | 0 |
| 2.407837725 | 0 |
| 2.40808131 | 0 |
| 2.408324895 | 0 |
| 2.40856848 | 0 |
| 2.408812065 | 0 |
| 2.40905565 | 0 |
| 2.409299235 | 0 |
| 2.40954282 | 0 |
| 2.409786405 | 0 |
| 2.41002999 | 0 |
| 2.410273575 | 0 |
| 2.41051716 | 0 |
| 2.410760745 | 0 |
| 2.41100433 | 0 |
| 2.411247915 | 0 |
| 2.4114915 | 0 |
| 2.411735085 | 0 |
| 2.41197867 | 0 |
| 2.412222255 | 0 |
| 2.41246584 | 0 |
| 2.412709425 | 0 |
| 2.41295301 | 0 |
| 2.413196595 | 0 |
| 2.41344018 | 0 |
| 2.413683765 | 0 |
| 2.41392735 | 0 |
| 2.414170935 | 0 |
| 2.41441452 | 0 |
| 2.414658105 | 0 |
| 2.41490169 | 0 |
| 2.415145275 | 0 |
| 2.41538886 | 0 |
| 2.415632445 | 0 |
| 2.41587603 | 0 |
| 2.416119615 | 0 |
| 2.4163632 | 0 |
| 2.416606785 | 0 |
| 2.41685037 | 0 |
| 2.417093955 | 0 |
| 2.41733754 | 0 |
| 2.417581125 | 0 |
| 2.41782471 | 0 |
| 2.418068295 | 0 |
| 2.41831188 | 0 |
| 2.418555465 | 0 |
| 2.41879905 | 0 |
| 2.419042635 | 0 |
| 2.41928622 | 0 |
| 2.419529805 | 0 |
| 2.41977339 | 0 |
| 2.420016975 | 0 |
| 2.42026056 | 0 |
| 2.420504145 | 0 |
| 2.42074773 | 0 |
| 2.420991315 | 0 |
| 2.4212349 | 0 |
| 2.421478485 | 0 |
| 2.42172207 | 0 |
| 2.421965655 | 0 |
| 2.42220924 | 0 |
| 2.422452825 | 0 |
| 2.42269641 | 0 |
| 2.422939995 | 0 |
| 2.42318358 | 0 |
| 2.423427165 | 0 |
| 2.42367075 | 0 |
| 2.423914335 | 0 |
| 2.42415792 | 0 |
| 2.424401505 | 0 |
| 2.42464509 | 0 |
| 2.424888675 | 0 |
| 2.42513226 | 0 |
| 2.425375845 | 0 |
| 2.42561943 | 0 |
| 2.425863015 | 0 |
| 2.4261066 | 0 |
| 2.426350185 | 0 |
| 2.42659377 | 0 |
| 2.426837355 | 0 |
| 2.42708094 | 0 |
| 2.427324525 | 0 |
| 2.42756811 | 0 |
| 2.427811695 | 0 |
| 2.42805528 | 0 |
| 2.428298865 | 0 |
| 2.42854245 | 0 |
| 2.428786035 | 0 |
| 2.42902962 | 0 |
| 2.429273205 | 0 |
| 2.42951679 | −0.05 |
| 2.429760375 | −0.141 |
| 2.43000396 | −0.231 |
| 2.430247545 | −0.593 |
| 2.43049113 | −0.955 |
| 2.430734715 | −1.357 |
| 2.4309783 | −1.81 |
| 2.431221885 | −2.262 |
| 2.43146547 | −3.115 |
| 2.431709055 | −4.021 |
| 2.43195264 | −3.451 |
| 2.432196225 | 0.171 |
| 2.43243981 | 3.794 |
| 2.432683395 | 4.287 |
| 2.43292698 | 3.834 |
| 2.433170565 | 3.477 |
| 2.43341415 | 3.478 |
| 2.433657735 | 3.479 |
| 2.43390132 | 3.184 |
| 2.434144905 | 2.731 |
| 2.43438849 | 2.32 |
| 2.434632075 | 2.321 |
| 2.43487566 | 2.321 |
| 2.435119245 | 2.078 |
| 2.43536283 | 1.625 |
| 2.435606415 | 1.171 |
| 2.43585 | 1.161 |
| 2.436093585 | 1.162 |
| 2.43633717 | 0.971 |
| 2.436580755 | 0.517 |
| 2.43682434 | 0.063 |
| 2.437067925 | 0 |
| 2.43731151 | 0 |
| 2.437555095 | 0 |
| 2.43779868 | 0 |
| 2.438042265 | 0 |
| 2.43828585 | 0 |
| 2.438529435 | 0 |
| 2.43877302 | 0 |
| 2.439016605 | 0 |
| 2.43926019 | 0 |
| 2.439503775 | 0 |
| 2.43974736 | 0 |
| 2.439990945 | 0 |
| 2.44023453 | 0 |
| 2.440478115 | 0 |
| 2.4407217 | 0 |
| 2.440965285 | 0 |
| 2.44120887 | 0 |
| 2.441452455 | 0 |
| 2.44169604 | 0 |
| 2.441939625 | 0.726 |
| 2.44218321 | 2.551 |
| 2.442426795 | 4.376 |
| 2.44267038 | 4.668 |
| 2.442913965 | 4.669 |
| 2.44315755 | 4.155 |
| 2.443401135 | 2.329 |
| 2.44364472 | 0.504 |
| 2.443888305 | 0 |
| 2.44413189 | 0 |
| 2.444375475 | 0 |
| 2.44461906 | 0 |
| 2.444862645 | 0 |
| 2.44510623 | −0.056 |
| 2.445349815 | −0.147 |
| 2.4455934 | −0.252 |
| 2.445836985 | −0.618 |
| 2.44608057 | −0.984 |
| 2.446324155 | −1.395 |
| 2.44656774 | −1.853 |
| 2.446811325 | −2.31 |
| 2.44705491 | −3.196 |
| 2.447298495 | −4.112 |
| 2.44754208 | −3.306 |
| 2.447785665 | 0.356 |
| 2.44802925 | 4.018 |
| 2.448272835 | 4.311 |
| 2.44851642 | 3.853 |
| 2.448760005 | 3.515 |
| 2.44900359 | 3.516 |
| 2.449247175 | 3.516 |
| 2.44949076 | 3.195 |
| 2.449734345 | 2.737 |
| 2.44997793 | 2.345 |
| 2.450221515 | 2.345 |
| 2.4504651 | 2.346 |
| 2.450708685 | 2.077 |
| 2.45095227 | 1.619 |
| 2.451195855 | 1.173 |
| 2.45143944 | 1.174 |
| 2.451683025 | 1.174 |
| 2.45192661 | 0.958 |
| 2.452170195 | 0.499 |
| 2.45241378 | 0.04 |
| 2.452657365 | 0 |
| 2.45290095 | 0 |
| 2.453144535 | 0 |
| 2.45338812 | 0 |
| 2.453631705 | 0 |
| 2.45387529 | 0 |
| 2.454118875 | 0 |
| 2.45436246 | 0 |
| 2.454606045 | 0 |
| 2.45484963 | 0 |
| 2.455093215 | 0 |
| 2.4553368 | 0 |
| 2.455580385 | 0 |
| 2.45582397 | 0 |
| 2.456067555 | 0 |
| 2.45631114 | 0 |
| 2.456554725 | 0 |
| 2.45679831 | 0 |
| 2.457041895 | 0 |
| 2.45728548 | 0 |
| 2.457529065 | 0.825 |
| 2.45777265 | 2.667 |
| 2.458016235 | 4.509 |
| 2.45825982 | 4.712 |
| 2.458503405 | 4.712 |
| 2.45874699 | 4.101 |
| 2.458990575 | 2.259 |
| 2.45923416 | 0.416 |
| 2.459477745 | 0 |
| 2.45972133 | 0 |
| 2.459964915 | 0 |
| 2.4602085 | 0 |
| 2.460452085 | 0 |
| 2.46069567 | −0.061 |
| 2.460939255 | −0.153 |
| 2.46118284 | −0.273 |
| 2.461426425 | −0.642 |
| 2.46167001 | −1.011 |
| 2.461913595 | −1.43 |
| 2.46215718 | −1.892 |
| 2.462400765 | −2.353 |
| 2.46264435 | −3.269 |
| 2.462887935 | −4.193 |
| 2.46313152 | −3.149 |
| 2.463375105 | 0.544 |
| 2.46361869 | 4.236 |
| 2.463862275 | 4.323 |
| 2.46410586 | 3.862 |
| 2.464349445 | 3.544 |
| 2.46459303 | 3.544 |
| 2.464836615 | 3.545 |
| 2.4650802 | 3.198 |
| 2.465323785 | 2.736 |
| 2.46556737 | 2.364 |
| 2.465810955 | 2.364 |
| 2.46605454 | 2.364 |
| 2.466298125 | 2.071 |
| 2.46654171 | 1.609 |
| 2.466785295 | 1.183 |
| 2.46702888 | 1.183 |
| 2.467272465 | 1.183 |
| 2.46751605 | 0.942 |
| 2.467759635 | 0.48 |
| 2.46800322 | 0.018 |
| 2.468246805 | 0 |
| 2.46849039 | 0 |
| 2.468733975 | 0 |
| 2.46897756 | 0 |
| 2.469221145 | 0 |
| 2.46946473 | 0 |
| 2.469708315 | 0 |
| 2.4699519 | 0 |
| 2.470195485 | 0 |
| 2.47043907 | 0 |
| 2.470682655 | 0 |
| 2.47092624 | 0 |
| 2.471169825 | 0 |
| 2.47141341 | 0 |
| 2.471656995 | 0 |
| 2.47190058 | 0 |
| 2.472144165 | 0 |
| 2.47238775 | 0 |
| 2.472631335 | 0 |
| 2.47287492 | 0 |
| 2.473118505 | 0.923 |
| 2.47336209 | 2.777 |
| 2.473605675 | 4.631 |
| 2.47384926 | 4.742 |
| 2.474092845 | 4.743 |
| 2.47433643 | 4.035 |
| 2.474580015 | 2.18 |
| 2.4748236 | 0.326 |
| 2.475067185 | 0 |
| 2.47531077 | 0 |
| 2.475554355 | 0 |
| 2.47579794 | 0 |
| 2.476041525 | 0 |
| 2.47628511 | −0.066 |
| 2.476528695 | −0.158 |
| 2.47677228 | −0.293 |
| 2.477015865 | −0.664 |
| 2.47725945 | −1.035 |
| 2.477503035 | −1.462 |
| 2.47774662 | −1.926 |
| 2.477990205 | −2.406 |
| 2.47823379 | −3.334 |
| 2.478477375 | −4.262 |
| 2.47872096 | −2.981 |
| 2.478964545 | 0.732 |
| 2.47920813 | 4.445 |
| 2.479451715 | 4.324 |
| 2.4796953 | 3.86 |
| 2.479938885 | 3.563 |
| 2.48018247 | 3.563 |
| 2.480426055 | 3.563 |
| 2.48066964 | 3.191 |
| 2.480913225 | 2.727 |
| 2.48115681 | 2.376 |
| 2.481400395 | 2.376 |
| 2.48164398 | 2.376 |
| 2.481887565 | 2.058 |
| 2.48213115 | 1.593 |
| 2.482374735 | 1.188 |
| 2.48261832 | 1.188 |
| 2.482861905 | 1.188 |
| 2.48310549 | 0.924 |
| 2.483349075 | 0.459 |
| 2.48359266 | 0 |
| 2.483836245 | 0 |
| 2.48407983 | 0 |
| 2.484323415 | 0 |
| 2.484567 | 0 |
| 2.484810585 | 0 |
| 2.48505417 | 0 |
| 2.485297755 | 0 |
| 2.48554134 | 0 |
| 2.485784925 | 0 |
| 2.48602851 | 0 |
| 2.486272095 | 0 |
| 2.48651568 | 0 |
| 2.486759265 | 0 |
| 2.48700285 | 0 |
| 2.487246435 | 0 |
| 2.48749002 | 0 |
| 2.487733605 | 0 |
| 2.48797719 | 0 |
| 2.488220775 | 0 |
| 2.48846436 | 0 |
| 2.488707945 | 1.019 |
| 2.48895153 | 2.88 |
| 2.489195115 | 4.74 |
| 2.4894387 | 4.759 |
| 2.489682285 | 4.759 |
| 2.48992587 | 3.955 |
| 2.490169455 | 2.095 |
| 2.49041304 | 0.234 |
| 2.490656625 | 0 |
| 2.49090021 | 0 |
| 2.491143795 | 0 |
| 2.49138738 | 0 |
| 2.491630965 | 0 |
| 2.49187455 | 0 |
| 2.492118135 | 0 |
| 2.49236172 | 0 |
| 2.492605305 | 0 |
| 2.49284889 | 0 |
| 2.493092475 | 0 |
| 2.49333606 | 0 |
| 2.493579645 | 0 |
| 2.49382323 | 0 |
| 2.494066815 | 0 |
| 2.4943104 | 0 |
| 2.494553985 | 0 |
| 2.49479757 | 0 |
| 2.495041155 | 0 |
| 2.49528474 | 0 |
| 2.495528325 | 0 |
| 2.49577191 | 0 |
| 2.496015495 | 0 |
| 2.49625908 | 0 |
| 2.496502665 | 0 |
| 2.49674625 | 0 |
| 2.496989835 | 0 |
| 2.49723342 | 0 |
| 2.497477005 | 0 |
| 2.49772059 | 0 |
| 2.497964175 | 0 |
| 2.49820776 | 0 |
| 2.498451345 | 0 |
| 2.49869493 | 0 |
| 2.498938515 | 0 |
| 2.4991821 | 0 |
| 2.499425685 | 0 |
| 2.49966927 | 0 |
| 2.499912855 | 0 |
| 2.50015644 | 0 |
| 2.500400025 | 0 |
| 2.50064361 | 0 |
| 2.500887195 | 0 |
| 2.50113078 | 0 |
| 2.501374365 | 0 |
| 2.50161795 | 0 |
| 2.501861535 | 0 |
| 2.50210512 | 0 |
| 2.502348705 | 0 |
| 2.50259229 | 0 |
| 2.502835875 | 0 |
| 2.50307946 | 0 |
| 2.503323045 | 0 |
| 2.50356663 | 0 |
| 2.503810215 | 0 |
| 2.5040538 | 0 |
| 2.504297385 | 0 |
| 2.50454097 | 0 |
| 2.504784555 | 0 |
| 2.50502814 | 0 |
| 2.505271725 | 0 |
| 2.50551531 | 0 |
| 2.505758895 | 0 |
| 2.50600248 | 0 |
| 2.506246065 | 0 |
| 2.50648965 | 0 |
| 2.506733235 | 0 |
| 2.50697682 | 0 |
| 2.507220405 | 0 |
| 2.50746399 | 0 |
| 2.507707575 | 0 |
| 2.50795116 | 0 |
| 2.508194745 | 0 |
| 2.50843833 | 0 |
| 2.508681915 | 0 |
| 2.5089255 | 0 |
| 2.509169085 | 0 |
| 2.50941267 | 0 |
| 2.509656255 | 0 |
| 2.50989984 | 0 |
| 2.510143425 | 0 |
| 2.51038701 | 0 |
| 2.510630595 | 0 |
| 2.51087418 | 0 |
| 2.511117765 | 0 |
| 2.51136135 | 0 |
| 2.511604935 | 0 |
| 2.51184852 | 0 |
| 2.512092105 | 0 |
| 2.51233569 | 0 |
| 2.512579275 | 0 |
| 2.51282286 | 0 |
| 2.513066445 | 0 |
| 2.51331003 | 0 |
| 2.513553615 | 0 |
| 2.5137972 | 0 |
| 2.514040785 | 0 |
| 2.51428437 | 0 |
| 2.514527955 | 0 |
| 2.51477154 | 0 |
| 2.515015125 | 0 |
| 2.51525871 | 0 |
| 2.515502295 | 0 |
| 2.51574588 | 0 |
| 2.515989465 | 0 |
| 2.51623305 | 0 |
| 2.516476635 | 0 |
| 2.51672022 | 0 |
| 2.516963805 | 0 |
| 2.51720739 | 0 |
| 2.517450975 | 0 |
| 2.51769456 | 0 |
| 2.517938145 | 0 |
| 2.51818173 | 0 |
| 2.518425315 | 0 |
| 2.5186689 | 0 |
| 2.518912485 | 0 |
| 2.51915607 | 0 |
| 2.519399655 | 0 |
| 2.51964324 | 0 |
| 2.519886825 | 0 |
| 2.52013041 | 0 |
| 2.520373995 | 0 |
| 2.52061758 | 0 |
| 2.520861165 | 0 |
| 2.52110475 | 0 |
| 2.521348335 | 0 |
| 2.52159192 | 0 |
| 2.521835505 | 0 |
| 2.52207909 | 0 |
| 2.522322675 | 0 |
| 2.52256626 | 0 |
| 2.522809845 | 0 |
| 2.52305343 | 0 |
| 2.523297015 | 0 |
| 2.5235406 | 0 |
| 2.523784185 | 0 |
| 2.52402777 | 0 |
| 2.524271355 | 0 |
| 2.52451494 | 0 |
| 2.524758525 | 0 |
| 2.52500211 | 0 |
| 2.525245695 | 0 |
| 2.52548928 | 0 |
| 2.525732865 | 0 |
| 2.52597645 | 0 |
| 2.526220035 | 0 |
| 2.52646362 | 0 |
| 2.526707205 | 0 |
| 2.52695079 | 0 |
| 2.527194375 | 0 |
| 2.52743796 | 0 |
| 2.527681545 | 0 |
| 2.52792513 | 0 |
| 2.528168715 | 0 |
| 2.5284123 | 0 |
| 2.528655885 | 0 |
| 2.52889947 | 0 |
| 2.529143055 | 0 |
| 2.52938664 | 0 |
| 2.529630225 | 0 |
| 2.52987381 | 0 |
| 2.530117395 | 0 |
| 2.53036098 | 0 |
| 2.530604565 | 0 |
| 2.53084815 | 0 |
| 2.531091735 | 0 |
| 2.53133532 | 0 |
| 2.531578905 | 0 |
| 2.53182249 | 0 |
| 2.532066075 | 0 |
| 2.53230966 | 0 |
| 2.532553245 | 0 |
| 2.53279683 | 0 |
| 2.533040415 | 0 |
| 2.533284 | 0 |
| 2.533527585 | 0 |
| 2.53377117 | 0 |
| 2.534014755 | 0 |
| 2.53425834 | 0 |
| 2.534501925 | 0 |
| 2.53474551 | 0 |
| 2.534989095 | 0 |
| 2.53523268 | 0 |
| 2.535476265 | 0 |
| 2.53571985 | 0 |
| 2.535963435 | 0 |
| 2.53620702 | 0 |
| 2.536450605 | 0 |
| 2.53669419 | 0 |
| 2.536937775 | 0 |
| 2.53718136 | 0 |
| 2.537424945 | 0 |
| 2.53766853 | 0 |
| 2.537912115 | 0 |
| 2.5381557 | 0 |
| 2.538399285 | 0 |
| 2.53864287 | 0 |
| 2.538886455 | 0 |
| 2.53913004 | 0 |
| 2.539373625 | 0 |
| 2.53961721 | 0 |
| 2.539860795 | 0 |
| 2.54010438 | 0 |
| 2.540347965 | 0 |
| 2.54059155 | 0 |
| 2.540835135 | 0 |
| 2.54107872 | 0 |
| 2.541322305 | 0 |
| 2.54156589 | 0 |
| 2.541809475 | 0 |
| 2.54205306 | 0 |
| 2.542296645 | 0 |
| 2.54254023 | 0 |
| 2.542783815 | 0 |
| 2.5430274 | 0 |
| 2.543270985 | 0 |
| 2.54351457 | 0 |
| 2.543758155 | 0 |
| 2.54400174 | 0 |
| 2.544245325 | 0 |
| 2.54448891 | 0 |
| 2.544732495 | 0 |
| 2.54497608 | 0 |
| 2.545219665 | 0 |
| 2.54546325 | 0 |
| 2.545706835 | 0 |
| 2.54595042 | 0 |
| 2.546194005 | 0 |
| 2.54643759 | 0 |
| 2.546681175 | 0 |
| 2.54692476 | 0 |
| 2.547168345 | 0 |
| 2.54741193 | 0 |
| 2.547655515 | 0 |
| 2.5478991 | 0 |
| 2.548142685 | 0 |
| 2.54838627 | 0 |
| 2.548629855 | 0 |
| 2.54887344 | 0 |
| 2.549117025 | 0 |
| 2.54936061 | 0 |
| 2.549604195 | 0 |
| 2.54984778 | 0 |
| 2.550091365 | 0 |
| 2.55033495 | 0 |
| 2.550578535 | 0 |
| 2.55082212 | 0 |
| 2.551065705 | 0 |
| 2.55130929 | 0 |
| 2.551552875 | 0 |
| 2.55179646 | 0 |
| 2.552040045 | 0 |
| 2.55228363 | 0 |
| 2.552527215 | 0 |
| 2.5527708 | 0 |
| 2.553014385 | 0 |
| 2.55325797 | 0 |
| 2.553501555 | 0 |
| 2.55374514 | 0 |
| 2.553988725 | 0 |
| 2.55423231 | 0 |
| 2.554475895 | 0 |
| 2.55471948 | 0 |
| 2.554963065 | 0 |
| 2.55520665 | 0 |
| 2.555450235 | 0 |
| 2.55569382 | 0 |
| 2.555937405 | 0 |
| 2.55618099 | 0 |
| 2.556424575 | 0 |
| 2.55666816 | 0 |
| 2.556911745 | 0 |
| 2.55715533 | 0 |
| 2.557398915 | 0 |
| 2.5576425 | 0 |
| 2.557886085 | 0 |
| 2.55812967 | 0 |
| 2.558373255 | 0 |
| 2.55861684 | 0 |
| 2.558860425 | 0 |
| 2.55910401 | 0 |
| 2.559347595 | 0 |
| 2.55959118 | 0 |
| 2.559834765 | 0 |
| 2.56007835 | 0 |
| 2.560321935 | 0 |
| 2.56056552 | 0 |
| 2.560809105 | 0 |
| 2.56105269 | 0 |
| 2.561296275 | 0 |
| 2.56153986 | 0 |
| 2.561783445 | 0 |
| 2.56202703 | 0 |
| 2.562270615 | 0 |
| 2.5625142 | 0 |
| 2.562757785 | 0 |
| 2.56300137 | 0 |
| 2.563244955 | 0 |
| 2.56348854 | 0 |
| 2.563732125 | 0 |
| 2.56397571 | 0 |
| 2.564219295 | 0 |
| 2.56446288 | 0 |
| 2.564706465 | 0 |
| 2.56495005 | 0 |
| 2.565193635 | 0 |
| 2.56543722 | 0 |
| 2.565680805 | 0 |
| 2.56592439 | 0 |
| 2.566167975 | 0 |
| 2.56641156 | 0 |
| 2.566655145 | 0 |
| 2.56689873 | 0 |
| 2.567142315 | 0 |
| 2.5673859 | 0 |
| 2.567629485 | 0 |
| 2.56787307 | 0 |
| 2.568116655 | 0 |
| 2.56836024 | 0 |
| 2.568603825 | 0 |
| 2.56884741 | 0 |
| 2.569090995 | 0 |
| 2.56933458 | 0 |
| 2.569578165 | 0 |
| 2.56982175 | 0 |
| 2.570065335 | 0 |
| 2.57030892 | 0 |
| 2.570552505 | 0 |
| 2.57079609 | 0 |
| 2.571039675 | 0 |
| 2.57128326 | 0 |
| 2.571526845 | 0 |
| 2.57177043 | 0 |
| 2.572014015 | 0 |
| 2.5722576 | 0 |
| 2.572501185 | 0 |
| 2.57274477 | 0 |
| 2.572988355 | 0 |
| 2.57323194 | 0 |
| 2.573475525 | 0 |
| 2.57371911 | 0 |
| 2.573962695 | 0 |
| 2.57420628 | 0 |
| 2.574449865 | 0 |
| 2.57469345 | 0 |
| 2.574937035 | 0 |
| 2.57518062 | 0 |
| 2.575424205 | 0 |
| 2.57566779 | 0 |
| 2.575911375 | 0 |
| 2.57615496 | 0 |
| 2.576398545 | 0 |
| 2.57664213 | 0 |
| 2.576885715 | 0 |
| 2.5771293 | 0 |
| 2.577372885 | 0 |
| 2.57761647 | 0 |
| 2.577860055 | 0 |
| 2.57810364 | 0 |
| 2.578347225 | 0 |
| 2.57859081 | 0 |
| 2.578834395 | 0 |
| 2.57907798 | 0 |
| 2.579321565 | 0 |
| 2.57956515 | 0 |
| 2.579808735 | 0 |
| 2.58005232 | 0 |
| 2.580295905 | 0 |
| 2.58053949 | 0 |
| 2.580783075 | 0 |
| 2.58102666 | 0 |
| 2.581270245 | 0 |
| 2.58151383 | 0 |
| 2.581757415 | 0 |
| 2.582001 | 0 |
| 2.582244585 | 0 |
| 2.58248817 | 0 |
| 2.582731755 | 0 |
| 2.58297534 | 0 |
| 2.583218925 | 0 |
| 2.58346251 | 0 |
| 2.583706095 | 0 |
| 2.58394968 | 0 |
| 2.584193265 | 0 |
| 2.58443685 | 0 |
| 2.584680435 | 0 |
| 2.58492402 | 0 |
| 2.585167605 | 0 |
| 2.58541119 | 0 |
| 2.585654775 | 0 |
| 2.58589836 | 0 |
| 2.586141945 | 0 |
| 2.58638553 | 0 |
| 2.586629115 | 0 |
| 2.5868727 | 0 |
| 2.587116285 | 0 |
| 2.58735987 | 0 |
| 2.587603455 | 0 |
| 2.58784704 | 0 |
| 2.588090625 | 0 |
| 2.58833421 | 0 |
| 2.588577795 | 0 |
| 2.58882138 | 0 |
| 2.589064965 | 0 |
| 2.58930855 | 0 |
| 2.589552135 | 0 |
| 2.58979572 | 0 |
| 2.590039305 | 0 |
| 2.59028289 | 0 |
| 2.590526475 | 0 |
| 2.59077006 | 0 |
| 2.591013645 | 0 |
| 2.59125723 | 0 |
| 2.591500815 | 0 |
| 2.5917444 | 0 |
| 2.591987985 | 0 |
| 2.59223157 | 0 |
| 2.592475155 | 0 |
| 2.59271874 | 0 |
| 2.592962325 | 0 |
| 2.59320591 | 0 |
| 2.593449495 | 0 |
| 2.59369308 | 0 |
| 2.593936665 | 0 |
| 2.59418025 | 0 |
| 2.594423835 | 0 |
| 2.59466742 | 0 |
| 2.594911005 | 0 |
| 2.59515459 | 0 |
| 2.595398175 | 0 |
| 2.59564176 | 0 |
| 2.595885345 | 0 |
| 2.59612893 | 0 |
| 2.596372515 | 0 |
| 2.5966161 | 0 |
| 2.596859685 | 0 |
| 2.59710327 | 0 |
| 2.597346855 | 0 |
| 2.59759044 | 0 |
| 2.597834025 | 0 |
| 2.59807761 | 0 |
| 2.598321195 | 0 |
| 2.59856478 | 0 |
| 2.598808365 | 0 |
| 2.59905195 | 0 |
| 2.599295535 | 0 |
| 2.59953912 | 0 |
| 2.599782705 | 0 |
| 2.60002629 | 0 |
| 2.600269875 | 0 |
| 2.60051346 | 0 |
| 2.600757045 | 0 |
| 2.60100063 | 0 |
| 2.601244215 | 0 |
| 2.6014878 | 0 |
| 2.601731385 | 0 |
| 2.60197497 | 0 |
| 2.602218555 | 0 |
| 2.60246214 | 0 |
| 2.602705725 | 0 |
| 2.60294931 | 0 |
| 2.603192895 | 0 |
| 2.60343648 | 0 |
| 2.603680065 | 0 |
| 2.60392365 | 0 |
| 2.604167235 | 0 |
| 2.60441082 | 0 |
| 2.604654405 | 0 |
| 2.60489799 | 0 |
| 2.605141575 | 0 |
| 2.60538516 | 0 |
| 2.605628745 | 0 |
| 2.60587233 | 0 |
| 2.606115915 | 0 |
| 2.6063595 | 0 |
| 2.606603085 | 0 |
| 2.60684667 | 0 |
| 2.607090255 | 0 |
| 2.60733384 | 0 |
| 2.607577425 | 0 |
| 2.60782101 | 0 |
| 2.608064595 | 0 |
| 2.60830818 | 0 |
| 2.608551765 | 0 |
| 2.60879535 | 0 |
| 2.609038935 | 0 |
| 2.60928252 | 0 |
| 2.609526105 | 0 |
| 2.60976969 | 0 |
| 2.610013275 | 0 |
| 2.61025686 | 0 |
| 2.610500445 | 0 |
| 2.61074403 | 0 |
| 2.610987615 | 0 |
| 2.6112312 | 0 |
| 2.611474785 | 0 |
| 2.61171837 | 0 |
| 2.611961955 | 0 |
| 2.61220554 | 0 |
| 2.612449125 | 0 |
| 2.61269271 | 0 |
| 2.612936295 | 0 |
| 2.61317988 | 0 |
| 2.613423465 | 0 |
| 2.61366705 | 0 |
| 2.613910635 | 0 |
| 2.61415422 | 0 |
| 2.614397805 | 0 |
| 2.61464139 | 0 |
| 2.614884975 | 0 |
| 2.61512856 | 0 |
| 2.615372145 | 0 |
| 2.61561573 | 0 |
| 2.615859315 | 0 |
| 2.6161029 | 0 |
| 2.616346485 | −0.013 |
| 2.61659007 | −0.098 |
| 2.616833655 | −0.183 |
| 2.61707724 | −0.419 |
| 2.617320825 | −0.757 |
| 2.61756441 | −1.098 |
| 2.617807995 | −1.52 |
| 2.61805158 | −1.942 |
| 2.618295165 | −2.567 |
| 2.61853875 | −3.409 |
| 2.618782335 | −4.251 |
| 2.61902592 | −1.192 |
| 2.619269505 | 2.179 |
| 2.61951309 | 4.154 |
| 2.619756675 | 3.732 |
| 2.62000026 | 3.309 |
| 2.620243845 | 3.228 |
| 2.62048743 | 3.227 |
| 2.620731015 | 3.119 |
| 2.6209746 | 2.698 |
| 2.621218185 | 2.277 |
| 2.62146177 | 2.148 |
| 2.621705355 | 2.147 |
| 2.62194894 | 2.089 |
| 2.622192525 | 1.669 |
| 2.62243611 | 1.249 |
| 2.622679695 | 1.072 |
| 2.62292328 | 1.071 |
| 2.623166865 | 1.062 |
| 2.62341045 | 0.644 |
| 2.623654035 | 0.225 |
| 2.62389762 | 0 |
| 2.624141205 | 0 |
| 2.62438479 | 0 |
| 2.624628375 | 0 |
| 2.62487196 | 0 |
| 2.625115545 | 0 |
| 2.62535913 | 0 |
| 2.625602715 | 0 |
| 2.6258463 | 0 |
| 2.626089885 | 0 |
| 2.62633347 | 0 |
| 2.626577055 | 0 |
| 2.62682064 | 0 |
| 2.627064225 | 0 |
| 2.62730781 | 0 |
| 2.627551395 | 0 |
| 2.62779498 | 0 |
| 2.628038565 | 0 |
| 2.62828215 | 0 |
| 2.628525735 | 0 |
| 2.62876932 | 0 |
| 2.629012905 | 1.653 |
| 2.62925649 | 3.31 |
| 2.629500075 | 4.237 |
| 2.62974366 | 4.235 |
| 2.629987245 | 4.234 |
| 2.63023083 | 2.774 |
| 2.630474415 | 1.119 |
| 2.630718 | 0 |
| 2.630961585 | 0 |
| 2.63120517 | 0 |
| 2.631448755 | 0 |
| 2.63169234 | 0 |
| 2.631935925 | −0.017 |
| 2.63217951 | −0.1 |
| 2.632423095 | −0.182 |
| 2.63266668 | −0.425 |
| 2.632910265 | −0.754 |
| 2.63315385 | −1.09 |
| 2.633397435 | −1.501 |
| 2.63364102 | −1.912 |
| 2.633884605 | −2.541 |
| 2.63412819 | −3.361 |
| 2.634371775 | −4.181 |
| 2.63461536 | −0.997 |
| 2.634858945 | 2.286 |
| 2.63510253 | 4.024 |
| 2.635346115 | 3.613 |
| 2.6355897 | 3.201 |
| 2.635833285 | 3.143 |
| 2.63607687 | 3.141 |
| 2.636320455 | 3.016 |
| 2.63656404 | 2.606 |
| 2.636807625 | 2.196 |
| 2.63705121 | 2.09 |
| 2.637294795 | 2.09 |
| 2.63753838 | 2.013 |
| 2.637781965 | 1.604 |
| 2.63802555 | 1.195 |
| 2.638269135 | 1.043 |
| 2.63851272 | 1.042 |
| 2.638756305 | 1.014 |
| 2.63899989 | 0.606 |
| 2.639243475 | 0.199 |
| 2.63948706 | 0 |
| 2.639730645 | 0 |
| 2.63997423 | 0 |
| 2.640217815 | 0 |
| 2.6404614 | 0 |
| 2.640704985 | 0 |
| 2.64094857 | 0 |
| 2.641192155 | 0 |
| 2.64143574 | 0 |
| 2.641679325 | 0 |
| 2.64192291 | 0 |
| 2.642166495 | 0 |
| 2.64241008 | 0 |
| 2.642653665 | 0 |
| 2.64289725 | 0 |
| 2.643140835 | 0 |
| 2.64338442 | 0 |
| 2.643628005 | 0 |
| 2.64387159 | 0 |
| 2.644115175 | 0 |
| 2.64435876 | 0.077 |
| 2.644602345 | 1.688 |
| 2.64484593 | 3.299 |
| 2.645089515 | 4.119 |
| 2.6453331 | 4.117 |
| 2.645576685 | 4.115 |
| 2.64582027 | 2.616 |
| 2.646063855 | 1.007 |
| 2.64630744 | 0 |
| 2.646551025 | 0 |
| 2.64679461 | 0 |
| 2.647038195 | 0 |
| 2.64728178 | 0 |
| 2.647525365 | −0.021 |
| 2.64776895 | −0.101 |
| 2.648012535 | −0.181 |
| 2.64825612 | −0.429 |
| 2.648499705 | −0.748 |
| 2.64874329 | −1.079 |
| 2.648986875 | −1.478 |
| 2.64923046 | −1.877 |
| 2.649474045 | −2.508 |
| 2.64971763 | −3.305 |
| 2.649961215 | −3.998 |
| 2.6502048 | −0.809 |
| 2.650448385 | 2.379 |
| 2.65069197 | 3.889 |
| 2.650935555 | 3.489 |
| 2.65117914 | 3.089 |
| 2.651422725 | 3.052 |
| 2.65166631 | 3.05 |
| 2.651909895 | 2.909 |
| 2.65215348 | 2.511 |
| 2.652397065 | 2.112 |
| 2.65264065 | 2.03 |
| 2.652884235 | 2.029 |
| 2.65312782 | 1.934 |
| 2.653371405 | 1.537 |
| 2.65361499 | 1.141 |
| 2.653858575 | 1.012 |
| 2.65410216 | 1.012 |
| 2.654345745 | 0.964 |
| 2.65458933 | 0.569 |
| 2.654832915 | 0.173 |
| 2.6550765 | 0 |
| 2.655320085 | 0 |
| 2.65556367 | 0 |
| 2.655807255 | 0 |
| 2.65605084 | 0 |
| 2.656294425 | 0 |
| 2.65653801 | 0 |
| 2.656781595 | 0 |
| 2.65702518 | 0 |
| 2.657268765 | 0 |
| 2.65751235 | 0 |
| 2.657755935 | 0 |
| 2.65799952 | 0 |
| 2.658243105 | 0 |
| 2.65848669 | 0 |
| 2.658730275 | 0 |
| 2.65897386 | 0 |
| 2.659217445 | 0 |
| 2.65946103 | 0 |
| 2.659704615 | 0 |
| 2.6599482 | 0.153 |
| 2.660191785 | 1.715 |
| 2.66043537 | 3.277 |
| 2.660678955 | 3.994 |
| 2.66092254 | 3.992 |
| 2.661166125 | 3.99 |
| 2.66140971 | 2.458 |
| 2.661653295 | 0.899 |
| 2.66189688 | 0 |
| 2.662140465 | 0 |
| 2.66238405 | 0 |
| 2.662627635 | 0 |
| 2.66287122 | 0 |
| 2.663114805 | −0.024 |
| 2.66335839 | −0.102 |
| 2.663601975 | −0.179 |
| 2.66384556 | −0.431 |
| 2.664089145 | −0.741 |
| 2.66433273 | −1.065 |
| 2.664576315 | −1.452 |
| 2.6648199 | −1.838 |
| 2.665063485 | −2.469 |
| 2.66530707 | −3.241 |
| 2.665550655 | −3.72 |
| 2.66579424 | −0.63 |
| 2.666037825 | 2.46 |
| 2.66628141 | 3.749 |
| 2.666524995 | 3.361 |
| 2.66676858 | 2.974 |
| 2.667012165 | 2.957 |
| 2.66725575 | 2.955 |
| 2.667499335 | 2.799 |
| 2.66774292 | 2.413 |
| 2.667986505 | 2.027 |
| 2.66823009 | 1.966 |
| 2.668473675 | 1.965 |
| 2.66871726 | 1.855 |
| 2.668960845 | 1.47 |
| 2.66920443 | 1.086 |
| 2.669448015 | 0.981 |
| 2.6696916 | 0.98 |
| 2.669935185 | 0.915 |
| 2.67017877 | 0.532 |
| 2.670422355 | 0.149 |
| 2.67066594 | 0 |
| 2.670909525 | 0 |
| 2.67115311 | 0 |
| 2.671396695 | 0 |
| 2.67164028 | 0 |
| 2.671883865 | 0 |
| 2.67212745 | 0 |
| 2.672371035 | 0 |
| 2.67261462 | 0 |
| 2.672858205 | 0 |
| 2.67310179 | 0 |
| 2.673345375 | 0 |
| 2.67358896 | 0 |
| 2.673832545 | 0 |
| 2.67407613 | 0 |
| 2.674319715 | 0 |
| 2.6745633 | 0 |
| 2.674806885 | 0 |
| 2.67505047 | 0 |
| 2.675294055 | 0 |
| 2.67553764 | 0.224 |
| 2.675781225 | 1.735 |
| 2.67602481 | 3.247 |
| 2.676268395 | 3.866 |
| 2.67651198 | 3.864 |
| 2.676755565 | 3.849 |
| 2.67699915 | 3.47 |
| 2.677242735 | 3.092 |
| 2.67748632 | 2.892 |
| 2.677729905 | 2.89 |
| 2.67797349 | 2.888 |
| 2.678217075 | 2.543 |
| 2.67846066 | 2.165 |
| 2.678704245 | 1.923 |
| 2.67894783 | 1.922 |
| 2.679191415 | 1.921 |
| 2.679435 | 1.62 |
| 2.679678585 | 1.244 |
| 2.67992217 | 0.959 |
| 2.680165755 | 0.958 |
| 2.68040934 | 0.958 |
| 2.680652925 | 0.702 |
| 2.68089651 | 0.327 |
| 2.681140095 | 0 |
| 2.68138368 | 0 |
| 2.681627265 | 0 |
| 2.68187085 | 0 |
| 2.682114435 | 0 |
| 2.68235802 | 0 |
| 2.682601605 | 0 |
| 2.68284519 | 0 |
| 2.683088775 | 0 |
| 2.68333236 | 0 |
| 2.683575945 | 0 |
| 2.68381953 | 0 |
| 2.684063115 | 0 |
| 2.6843067 | 0 |
| 2.684550285 | 0 |
| 2.68479387 | 0 |
| 2.685037455 | 0 |
| 2.68528104 | 0 |
| 2.685524625 | 0 |
| 2.68576821 | 0 |
| 2.686011795 | 0 |
| 2.68625538 | 0 |
| 2.686498965 | 0 |
| 2.68674255 | 0 |
| 2.686986135 | 0 |
| 2.68722972 | 0 |
| 2.687473305 | 0 |
| 2.68771689 | 0 |
| 2.687960475 | 0 |
| 2.68820406 | 0 |
| 2.688447645 | 0 |
| 2.68869123 | 0 |
| 2.688934815 | 0 |
| 2.6891784 | 0 |
| 2.689421985 | 0 |
| 2.68966557 | 0 |
| 2.689909155 | 0 |
| 2.69015274 | 0 |
| 2.690396325 | 0 |
| 2.69063991 | 0 |
| 2.690883495 | 0 |
| 2.69112708 | 0 |
| 2.691370665 | 0 |
| 2.69161425 | 0 |
| 2.691857835 | 0 |
| 2.69210142 | 0 |
| 2.692345005 | 0 |
| 2.69258859 | 0 |
| 2.692832175 | 0 |
| 2.69307576 | 0 |
| 2.693319345 | 0 |
| 2.69356293 | 0 |
| 2.693806515 | 0 |
| 2.6940501 | 0 |
| 2.694293685 | 0 |
| 2.69453727 | 0 |
| 2.694780855 | 0 |
| 2.69502444 | 0 |
| 2.695268025 | 0 |
| 2.69551161 | 0 |
| 2.695755195 | 0 |
| 2.69599878 | 0 |
| 2.696242365 | 0 |
| 2.69648595 | 0 |
| 2.696729535 | 0 |
| 2.69697312 | 0 |
| 2.697216705 | 0 |
| 2.69746029 | 0 |
| 2.697703875 | 0 |
| 2.69794746 | 0 |
| 2.698191045 | 0 |
| 2.69843463 | 0 |
| 2.698678215 | 0 |
| 2.6989218 | 0 |
| 2.699165385 | 0 |
| 2.69940897 | 0 |
| 2.699652555 | 0 |
| 2.69989614 | 0 |
| 2.700139725 | 0 |
| 2.70038331 | 0 |
| 2.700626895 | 0 |
| 2.70087048 | 0 |
| 2.701114065 | 0 |
| 2.70135765 | 0 |
| 2.701601235 | 0 |
| 2.70184482 | 0 |
| 2.702088405 | 0 |
| 2.70233199 | 0 |
| 2.702575575 | 0 |
| 2.70281916 | 0 |
| 2.703062745 | 0 |
| 2.70330633 | 0 |
| 2.703549915 | 0 |
| 2.7037935 | 0 |
| 2.704037085 | 0 |
| 2.70428067 | 0 |
| 2.704524255 | 0 |
| 2.70476784 | 0 |
| 2.705011425 | 0 |
| 2.70525501 | 0 |
| 2.705498595 | 0 |
| 2.70574218 | 0 |
| 2.705985765 | 0 |
| 2.70622935 | 0 |
| 2.706472935 | 0 |
| 2.70671652 | 0 |
| 2.706960105 | 0 |
| 2.70720369 | 0 |
| 2.707447275 | 0 |
| 2.70769086 | 0 |
| 2.707934445 | 0 |
| 2.70817803 | 0 |
| 2.708421615 | 0 |
| 2.7086652 | 0 |
| 2.708908785 | 0 |
| 2.70915237 | 0 |
| 2.709395955 | 0 |
| 2.70963954 | 0 |
| 2.709883125 | 0 |
| 2.71012671 | 0 |
| 2.710370295 | 0 |
| 2.71061388 | 0 |
| 2.710857465 | 0 |
| 2.71110105 | 0 |
| 2.711344635 | 0 |
| 2.71158822 | 0 |
| 2.711831805 | 0 |
| 2.71207539 | 0 |
| 2.712318975 | 0 |
| 2.71256256 | 0 |
| 2.712806145 | 0 |
| 2.71304973 | 0 |
| 2.713293315 | 0 |
| 2.7135369 | 0 |
| 2.713780485 | 0 |
| 2.71402407 | 0 |
| 2.714267655 | 0 |
| 2.71451124 | 0 |
| 2.714754825 | 0 |
| 2.71499841 | 0 |
| 2.715241995 | 0 |
| 2.71548558 | 0 |
| 2.715729165 | 0 |
| 2.71597275 | 0 |
| 2.716216335 | 0 |
| 2.71645992 | 0 |
| 2.716703505 | 0 |
| 2.71694709 | 0 |
| 2.717190675 | 0 |
| 2.71743426 | 0 |
| 2.717677845 | 0 |
| 2.71792143 | 0 |
| 2.718165015 | 0 |
| 2.7184086 | 0 |
| 2.718652185 | 0 |
| 2.71889577 | 0 |
| 2.719139355 | 0 |
| 2.71938294 | 0 |
| 2.719626525 | 0 |
| 2.71987011 | 0 |
| 2.720113695 | 0 |
| 2.72035728 | 0 |
| 2.720600865 | 0 |
| 2.72084445 | 0 |
| 2.721088035 | 0 |
| 2.72133162 | 0 |
| 2.721575205 | 0 |
| 2.72181879 | 0 |
| 2.722062375 | 0 |
| 2.72230596 | 0 |
| 2.722549545 | 0 |
| 2.72279313 | 0 |
| 2.723036715 | 0 |
| 2.7232803 | 0 |
| 2.723523885 | 0 |
| 2.72376747 | 0 |
| 2.724011055 | 0 |
| 2.72425464 | 0 |
| 2.724498225 | 0 |
| 2.72474181 | 0 |
| 2.724985395 | 0 |
| 2.72522898 | 0 |
| 2.725472565 | 0 |
| 2.72571615 | 0 |
| 2.725959735 | 0 |
| 2.72620332 | 0 |
| 2.726446905 | 0 |
| 2.72669049 | 0 |
| 2.726934075 | 0 |
| 2.72717766 | 0 |
| 2.727421245 | 0 |
| 2.72766483 | 0 |
| 2.727908415 | 0 |
| 2.728152 | 0 |
| 2.728395585 | 0 |
| 2.72863917 | 0 |
| 2.728882755 | 0 |
| 2.72912634 | 0 |
| 2.729369925 | 0 |
| 2.72961351 | 0 |
| 2.729857095 | 0 |
| 2.73010068 | 0 |
| 2.730344265 | 0 |
| 2.73058785 | 0 |
| 2.730831435 | 0 |
| 2.73107502 | 0 |
| 2.731318605 | 0 |
| 2.73156219 | 0 |
| 2.731805775 | 0 |
| 2.73204936 | 0 |
| 2.732292945 | 0 |
| 2.73253653 | 0 |
| 2.732780115 | 0 |
| 2.7330237 | 0 |
| 2.733267285 | 0 |
| 2.73351087 | 0 |
| 2.733754455 | 0 |
| 2.73399804 | 0 |
| 2.734241625 | 0 |
| 2.73448521 | 0 |
| 2.734728795 | 0 |
| 2.73497238 | 0 |
| 2.735215965 | 0 |
| 2.73545955 | 0 |
| 2.735703135 | 0 |
| 2.73594672 | 0 |
| 2.736190305 | 0 |
| 2.73643389 | 0 |
| 2.736677475 | 0 |
| 2.73692106 | 0 |
| 2.737164645 | 0 |
| 2.73740823 | 0 |
| 2.737651815 | 0 |
| 2.7378954 | 0 |
| 2.738138985 | 0 |
| 2.73838257 | 0 |
| 2.738626155 | 0 |
| 2.73886974 | 0 |
| 2.739113325 | 0 |
| 2.73935691 | 0 |
| 2.739600495 | 0 |
| 2.73984408 | 0 |
| 2.740087665 | 0 |
| 2.74033125 | 0 |
| 2.740574835 | 0 |
| 2.74081842 | 0 |
| 2.741062005 | 0 |
| 2.74130559 | 0 |
| 2.741549175 | 0 |
| 2.74179276 | 0 |
| 2.742036345 | 0 |
| 2.74227993 | 0 |
| 2.742523515 | 0 |
| 2.7427671 | 0 |
| 2.743010685 | 0 |
| 2.74325427 | 0 |
| 2.743497855 | 0 |
| 2.74374144 | 0 |
| 2.743985025 | 0 |
| 2.74422861 | 0 |
| 2.744472195 | 0 |
| 2.74471578 | 0 |
| 2.744959365 | 0 |
| 2.74520295 | 0 |
| 2.745446535 | 0 |
| 2.74569012 | 0 |
| 2.745933705 | 0 |
| 2.74617729 | 0 |
| 2.746420875 | 0 |
| 2.74666446 | 0 |
| 2.746908045 | 0 |
| 2.74715163 | 0 |
| 2.747395215 | 0 |
| 2.7476388 | 0 |
| 2.747882385 | 0 |
| 2.74812597 | 0 |
| 2.748369555 | 0 |
| 2.74861314 | 0 |
| 2.748856725 | 0 |
| 2.74910031 | 0 |
| 2.749343895 | 0 |
| 2.74958748 | 0 |
| 2.749831065 | 0 |
| 2.75007465 | 0 |
| 2.750318235 | 0 |
| 2.75056182 | 0 |
| 2.750805405 | 0 |
| 2.75104899 | 0 |
| 2.751292575 | 0 |
| 2.75153616 | 0 |
| 2.751779745 | 0 |
| 2.75202333 | 0 |
| 2.752266915 | 0 |
| 2.7525105 | 0 |
| 2.752754085 | 0 |
| 2.75299767 | 0 |
| 2.753241255 | 0 |
| 2.75348484 | 0 |
| 2.753728425 | 0 |
| 2.75397201 | 0 |
| 2.754215595 | 0 |
| 2.75445918 | 0 |
| 2.754702765 | 0 |
| 2.75494635 | 0 |
| 2.755189935 | 0 |
| 2.75543352 | 0 |
| 2.755677105 | 0 |
| 2.75592069 | 0 |
| 2.756164275 | 0 |
| 2.75640786 | 0 |
| 2.756651445 | 0 |
| 2.75689503 | 0 |
| 2.757138615 | 0 |
| 2.7573822 | 0 |
| 2.757625785 | 0 |
| 2.75786937 | 0 |
| 2.758112955 | 0 |
| 2.75835654 | 0 |
| 2.758600125 | 0 |
| 2.75884371 | 0 |
| 2.759087295 | 0 |
| 2.75933088 | 0 |
| 2.759574465 | 0 |
| 2.75981805 | 0 |
| 2.760061635 | 0 |
| 2.76030522 | 0 |
| 2.760548805 | 0 |
| 2.76079239 | 0 |
| 2.761035975 | 0 |
| 2.76127956 | 0 |
| 2.761523145 | 0 |
| 2.76176673 | 0 |
| 2.762010315 | 0 |
| 2.7622539 | 0 |
| 2.762497485 | 0 |
| 2.76274107 | 0 |
| 2.762984655 | 0 |
| 2.76322824 | 0 |
| 2.763471825 | 0 |
| 2.76371541 | 0 |
| 2.763958995 | 0 |
| 2.76420258 | 0 |
| 2.764446165 | 0 |
| 2.76468975 | 0 |
| 2.764933335 | 0 |
| 2.76517692 | 0 |
| 2.765420505 | 0 |
| 2.76566409 | 0 |
| 2.765907675 | 0 |
| 2.76615126 | 0 |
| 2.766394845 | 0 |
| 2.76663843 | 0 |
| 2.766882015 | 0 |
| 2.7671256 | 0 |
| 2.767369185 | 0 |
| 2.76761277 | 0 |
| 2.767856355 | 0 |
| 2.76809994 | 0 |
| 2.768343525 | 0 |
| 2.76858711 | 0 |
| 2.768830695 | 0 |
| 2.76907428 | 0 |
| 2.769317865 | 0 |
| 2.76956145 | 0 |
| 2.769805035 | 0 |
| 2.77004862 | 0 |
| 2.770292205 | 0 |
| 2.77053579 | 0 |
| 2.770779375 | 0 |
| 2.77102296 | 0 |
| 2.771266545 | 0 |
| 2.77151013 | 0 |
| 2.771753715 | 0 |
| 2.7719973 | 0 |
| 2.772240885 | 0 |
| 2.77248447 | 0 |
| 2.772728055 | 0 |
| 2.77297164 | 0 |
| 2.773215225 | 0 |
| 2.77345881 | 0 |
| 2.773702395 | 0 |
| 2.77394598 | 0 |
| 2.774189565 | 0 |
| 2.77443315 | 0 |
| 2.774676735 | 0 |
| 2.77492032 | 0 |
| 2.775163905 | 0 |
| 2.77540749 | 0 |
| 2.775651075 | 0 |
| 2.77589466 | 0 |
| 2.776138245 | 0 |
| 2.77638183 | 0 |
| 2.776625415 | 0 |
| 2.776869 | 0 |
| 2.777112585 | 0 |
| 2.77735617 | 0 |
| 2.777599755 | 0 |
| 2.77784334 | 0 |
| 2.778086925 | 0 |
| 2.77833051 | 0 |
| 2.778574095 | 0 |
| 2.77881768 | 0 |
| 2.779061265 | 0 |
| 2.77930485 | 0 |
| 2.779548435 | 0 |
| 2.77979202 | 0 |
| 2.780035605 | 0 |
| 2.78027919 | 0 |
| 2.780522775 | 0 |
| 2.78076636 | 0 |
| 2.781009945 | 0 |
| 2.78125353 | 0 |
| 2.781497115 | 0 |
| 2.7817407 | 0 |
| 2.781984285 | 0 |
| 2.78222787 | 0 |
| 2.782471455 | 0 |
| 2.78271504 | 0 |
| 2.782958625 | 0 |
| 2.78320221 | 0 |
| 2.783445795 | 0 |
| 2.78368938 | 0 |
| 2.783932965 | 0 |
| 2.78417655 | 0 |
| 2.784420135 | 0 |
| 2.78466372 | 0 |
| 2.784907305 | 0 |
| 2.78515089 | 0 |
| 2.785394475 | 0 |
| 2.78563806 | 0 |
| 2.785881645 | 0 |
| 2.78612523 | 0 |
| 2.786368815 | 0 |
| 2.7866124 | 0 |
| 2.786855985 | 0 |
| 2.78709957 | 0 |
| 2.787343155 | 0 |
| 2.78758674 | 0 |
| 2.787830325 | 0 |
| 2.78807391 | 0 |
| 2.788317495 | 0 |
| 2.78856108 | 0 |
| 2.788804665 | 0 |
| 2.78904825 | 0 |
| 2.789291835 | 0 |
| 2.78953542 | 0 |
| 2.789779005 | 0 |
| 2.79002259 | 0 |
| 2.790266175 | 0 |
| 2.79050976 | 0 |
| 2.790753345 | 0 |
| 2.79099693 | 0 |
| 2.791240515 | 0 |
| 2.7914841 | 0 |
| 2.791727685 | 0 |
| 2.79197127 | 0 |
| 2.792214855 | 0 |
| 2.79245844 | 0 |
| 2.792702025 | 0 |
| 2.79294561 | 0 |
| 2.793189195 | 0 |
| 2.79343278 | 0 |
| 2.793676365 | 0 |
| 2.79391995 | 0 |
| 2.794163535 | 0 |
| 2.79440712 | 0 |
| 2.794650705 | 0 |
| 2.79489429 | 0 |
| 2.795137875 | 0 |
| 2.79538146 | 0 |
| 2.795625045 | 0 |
| 2.79586863 | 0 |
| 2.796112215 | 0 |
| 2.7963558 | 0 |
| 2.796599385 | 0 |
| 2.79684297 | 0 |
| 2.797086555 | 0 |
| 2.79733014 | 0 |
| 2.797573725 | 0 |
| 2.79781731 | 0 |
| 2.798060895 | 0 |
| 2.79830448 | 0 |
| 2.798548065 | 0 |
| 2.79879165 | 0 |
| 2.799035235 | 0 |
| 2.79927882 | 0 |
| 2.799522405 | 0 |
| 2.79976599 | 0 |
| 2.800009575 | 0 |
| 2.80025316 | 0 |
| 2.800496745 | 0 |
| 2.80074033 | 0 |
| 2.800983915 | 0 |
| 2.8012275 | 0 |
| 2.801471085 | 0 |
| 2.80171467 | 0 |
| 2.801958255 | 0 |
| 2.80220184 | 0 |
| 2.802445425 | 0 |
| 2.80268901 | 0 |
| 2.802932595 | 0 |
| 2.80317618 | 0 |
| 2.803419765 | −0.043 |
| 2.80366335 | −0.1 |
| 2.803906935 | −0.191 |
| 2.80415052 | −0.419 |
| 2.804394105 | −0.646 |
| 2.80463769 | −0.91 |
| 2.804881275 | −1.194 |
| 2.80512486 | −1.502 |
| 2.805368445 | −2.069 |
| 2.80561203 | −2.636 |
| 2.805855615 | −1.711 |
| 2.8060992 | 0.559 |
| 2.806342785 | 2.829 |
| 2.80658637 | 2.626 |
| 2.806829955 | 2.342 |
| 2.80707354 | 2.173 |
| 2.807317125 | 2.172 |
| 2.80756071 | 2.171 |
| 2.807804295 | 1.93 |
| 2.80804788 | 1.646 |
| 2.808291465 | 1.446 |
| 2.80853505 | 1.445 |
| 2.808778635 | 1.445 |
| 2.80902222 | 1.236 |
| 2.809265805 | 0.954 |
| 2.80950939 | 0.721 |
| 2.809752975 | 0.721 |
| 2.80999656 | 0.721 |
| 2.810240145 | 0.546 |
| 2.81048373 | 0.264 |
| 2.810727315 | 0 |
| 2.8109709 | 0 |
| 2.811214485 | 0 |
| 2.81145807 | 0 |
| 2.811701655 | 0 |
| 2.81194524 | 0 |
| 2.812188825 | 0 |
| 2.81243241 | 0 |
| 2.812675995 | 0 |
| 2.81291958 | 0 |
| 2.813163165 | 0 |
| 2.81340675 | 0 |
| 2.813650335 | 0 |
| 2.81389392 | 0 |
| 2.814137505 | 0 |
| 2.81438109 | 0 |
| 2.814624675 | 0 |
| 2.81486826 | 0 |
| 2.815111845 | 0 |
| 2.81535543 | 0 |
| 2.815599015 | 0 |
| 2.8158426 | 0.667 |
| 2.816086185 | 1.782 |
| 2.81632977 | 2.852 |
| 2.816573355 | 2.851 |
| 2.81681694 | 2.85 |
| 2.817060525 | 2.313 |
| 2.81730411 | 1.199 |
| 2.817547695 | 0.085 |
| 2.81779128 | 0 |
| 2.818034865 | 0 |
| 2.81827845 | 0 |
| 2.818522035 | 0 |
| 2.81876562 | 0 |
| 2.819009205 | −0.045 |
| 2.81925279 | −0.1 |
| 2.819496375 | −0.197 |
| 2.81973996 | −0.419 |
| 2.819983545 | −0.641 |
| 2.82022713 | −0.9 |
| 2.820470715 | −1.177 |
| 2.8207143 | −1.49 |
| 2.820957885 | −2.043 |
| 2.82120147 | −2.595 |
| 2.821445055 | −1.556 |
| 2.82168864 | 0.655 |
| 2.821932225 | 2.822 |
| 2.82217581 | 2.545 |
| 2.822419395 | 2.268 |
| 2.82266298 | 2.118 |
| 2.822906565 | 2.117 |
| 2.82315015 | 2.116 |
| 2.823393735 | 1.867 |
| 2.82363732 | 1.591 |
| 2.823880905 | 1.409 |
| 2.82412449 | 1.409 |
| 2.824368075 | 1.408 |
| 2.82461166 | 1.191 |
| 2.824855245 | 0.916 |
| 2.82509883 | 0.703 |
| 2.825342415 | 0.703 |
| 2.825586 | 0.703 |
| 2.825829585 | 0.519 |
| 2.82607317 | 0.244 |
| 2.826316755 | 0 |
| 2.82656034 | 0 |
| 2.826803925 | 0 |
| 2.82704751 | 0 |
| 2.827291095 | 0 |
| 2.82753468 | 0 |
| 2.827778265 | 0 |
| 2.82802185 | 0 |
| 2.828265435 | 0 |
| 2.82850902 | 0 |
| 2.828752605 | 0 |
| 2.82899619 | 0 |
| 2.829239775 | 0 |
| 2.82948336 | 0 |
| 2.829726945 | 0 |
| 2.82997053 | 0 |
| 2.830214115 | 0 |
| 2.8304577 | 0 |
| 2.830701285 | 0 |
| 2.83094487 | 0 |
| 2.831188455 | 0 |
| 2.83143204 | 0.706 |
| 2.831675625 | 1.795 |
| 2.83191921 | 2.785 |
| 2.832162795 | 2.785 |
| 2.83240638 | 2.784 |
| 2.832649965 | 2.204 |
| 2.83289355 | 1.116 |
| 2.833137135 | 0.028 |
| 2.83338072 | 0 |
| 2.833624305 | 0 |
| 2.83386789 | 0 |
| 2.834111475 | 0 |
| 2.83435506 | 0 |
| 2.834598645 | −0.047 |
| 2.83484223 | −0.101 |
| 2.835085815 | −0.204 |
| 2.8353294 | −0.42 |
| 2.835572985 | −0.637 |
| 2.83581657 | −0.893 |
| 2.836060155 | −1.164 |
| 2.83630374 | −1.484 |
| 2.836547325 | −2.024 |
| 2.83679091 | −2.565 |
| 2.837034495 | −1.413 |
| 2.83727808 | 0.749 |
| 2.837521665 | 2.746 |
| 2.83776525 | 2.475 |
| 2.838008835 | 2.205 |
| 2.83825242 | 2.071 |
| 2.838496005 | 2.071 |
| 2.83873959 | 2.07 |
| 2.838983175 | 1.813 |
| 2.83922676 | 1.543 |
| 2.839470345 | 1.379 |
| 2.83971393 | 1.378 |
| 2.839957515 | 1.378 |
| 2.8402011 | 1.153 |
| 2.840444685 | 0.883 |
| 2.84068827 | 0.688 |
| 2.840931855 | 0.688 |
| 2.84117544 | 0.688 |
| 2.841419025 | 0.494 |
| 2.84166261 | 0.225 |
| 2.841906195 | 0 |
| 2.84214978 | 0 |
| 2.842393365 | 0 |
| 2.84263695 | 0 |
| 2.842880535 | 0 |
| 2.84312412 | 0 |
| 2.843367705 | 0 |
| 2.84361129 | 0 |
| 2.843854875 | 0 |
| 2.84409846 | 0 |
| 2.844342045 | 0 |
| 2.84458563 | 0 |
| 2.844829215 | 0 |
| 2.8450728 | 0 |
| 2.845316385 | 0 |
| 2.84555997 | 0 |
| 2.845803555 | 0 |
| 2.84604714 | 0 |
| 2.846290725 | 0 |
| 2.84653431 | 0 |
| 2.846777895 | 0 |
| 2.84702148 | 0.745 |
| 2.847265065 | 1.813 |
| 2.84750865 | 2.731 |
| 2.847752235 | 2.731 |
| 2.84799582 | 2.73 |
| 2.848239405 | 2.109 |
| 2.84848299 | 1.041 |
| 2.848726575 | 0 |
| 2.84897016 | 0 |
| 2.849213745 | 0 |
| 2.84945733 | 0 |
| 2.849700915 | 0 |
| 2.8499445 | 0 |
| 2.850188085 | −0.048 |
| 2.85043167 | −0.102 |
| 2.850675255 | −0.211 |
| 2.85091884 | −0.423 |
| 2.851162425 | −0.636 |
| 2.85140601 | −0.89 |
| 2.851649595 | −1.156 |
| 2.85189318 | −1.484 |
| 2.852136765 | −2.014 |
| 2.85238035 | −2.545 |
| 2.852623935 | −1.281 |
| 2.85286752 | 0.842 |
| 2.853111105 | 2.684 |
| 2.85335469 | 2.418 |
| 2.853598275 | 2.152 |
| 2.85384186 | 2.035 |
| 2.854085445 | 2.034 |
| 2.85432903 | 2.033 |
| 2.854572615 | 1.768 |
| 2.8548162 | 1.503 |
| 2.855059785 | 1.355 |
| 2.85530337 | 1.355 |
| 2.855546955 | 1.354 |
| 2.85579054 | 1.12 |
| 2.856034125 | 0.855 |
| 2.85627771 | 0.677 |
| 2.856521295 | 0.677 |
| 2.85676488 | 0.676 |
| 2.857008465 | 0.473 |
| 2.85725205 | 0.208 |
| 2.857495635 | 0 |
| 2.85773922 | 0 |
| 2.857982805 | 0 |
| 2.85822639 | 0 |
| 2.858469975 | 0 |
| 2.85871356 | 0 |
| 2.858957145 | 0 |
| 2.85920073 | 0 |
| 2.859444315 | 0 |
| 2.8596879 | 0 |
| 2.859931485 | 0 |
| 2.86017507 | 0 |
| 2.860418655 | 0 |
| 2.86066224 | 0 |
| 2.860905825 | 0 |
| 2.86114941 | 0 |
| 2.861392995 | 0 |
| 2.86163658 | 0 |
| 2.861880165 | 0 |
| 2.86212375 | 0 |
| 2.862367335 | 0 |
| 2.86261092 | 0.787 |
| 2.862854505 | 1.838 |
| 2.86309809 | 2.69 |
| 2.863341675 | 2.69 |
| 2.86358526 | 2.689 |
| 2.863828845 | 2.025 |
| 2.86407243 | 0.973 |
| 2.864316015 | 0 |
| 2.8645596 | 0 |
| 2.864803185 | 0 |
| 2.86504677 | 0 |
| 2.865290355 | 0 |
| 2.86553394 | 0 |
| 2.865777525 | 0 |
| 2.86602111 | 0 |
| 2.866264695 | 0 |
| 2.86650828 | 0 |
| 2.866751865 | 0 |
| 2.86699545 | 0 |
| 2.867239035 | 0 |
| 2.86748262 | 0 |
| 2.867726205 | 0 |
| 2.86796979 | 0 |
| 2.868213375 | 0 |
| 2.86845696 | 0 |
| 2.868700545 | 0 |
| 2.86894413 | 0 |
| 2.869187715 | 0 |
| 2.8694313 | 0 |
| 2.869674885 | 0 |
| 2.86991847 | 0 |
| 2.870162055 | 0 |
| 2.87040564 | 0 |
| 2.870649225 | 0 |
| 2.87089281 | 0 |
| 2.871136395 | 0 |
| 2.87137998 | 0 |
| 2.871623565 | 0 |
| 2.87186715 | 0 |
| 2.872110735 | 0 |
| 2.87235432 | 0 |
| 2.872597905 | 0 |
| 2.87284149 | 0 |
| 2.873085075 | 0 |
| 2.87332866 | 0 |
| 2.873572245 | 0 |
| 2.87381583 | 0 |
| 2.874059415 | 0 |
| 2.874303 | 0 |
| 2.874546585 | 0 |
| 2.87479017 | 0 |
| 2.875033755 | 0 |
| 2.87527734 | 0 |
| 2.875520925 | 0 |
| 2.87576451 | 0 |
| 2.876008095 | 0 |
| 2.87625168 | 0 |
| 2.876495265 | 0 |
| 2.87673885 | 0 |
| 2.876982435 | 0 |
| 2.87722602 | 0 |
| 2.877469605 | 0 |
| 2.87771319 | 0 |
| 2.877956775 | 0 |
| 2.87820036 | 0 |
| 2.878443945 | 0 |
| 2.87868753 | 0 |
| 2.878931115 | 0 |
| 2.8791747 | 0 |
| 2.879418285 | 0 |
| 2.87966187 | 0 |
| 2.879905455 | 0 |
| 2.88014904 | 0 |
| 2.880392625 | 0 |
| 2.88063621 | 0 |
| 2.880879795 | 0 |
| 2.88112338 | 0 |
| 2.881366965 | −0.052 |
| 2.88161055 | −0.104 |
| 2.881854135 | −0.226 |
| 2.88209772 | −0.434 |
| 2.882341305 | −0.642 |
| 2.88258489 | −0.896 |
| 2.882828475 | −1.155 |
| 2.88307206 | −1.501 |
| 2.883315645 | −2.02 |
| 2.88355923 | −2.539 |
| 2.883802815 | −1.045 |
| 2.8840464 | 1.031 |
| 2.884289985 | 2.598 |
| 2.88453357 | 2.339 |
| 2.884777155 | 2.079 |
| 2.88502074 | 1.99 |
| 2.885264325 | 1.99 |
| 2.88550791 | 1.964 |
| 2.885751495 | 1.704 |
| 2.88599508 | 1.445 |
| 2.886238665 | 1.326 |
| 2.88648225 | 1.326 |
| 2.886725835 | 1.326 |
| 2.88696942 | 1.07 |
| 2.887213005 | 0.811 |
| 2.88745659 | 0.663 |
| 2.887700175 | 0.663 |
| 2.88794376 | 0.663 |
| 2.888187345 | 0.437 |
| 2.88843093 | 0.178 |
| 2.888674515 | 0 |
| 2.8889181 | 0 |
| 2.889161685 | 0 |
| 2.88940527 | 0 |
| 2.889648855 | 0 |
| 2.88989244 | 0 |
| 2.890136025 | 0 |
| 2.89037961 | 0 |
| 2.890623195 | 0 |
| 2.89086678 | 0 |
| 2.891110365 | 0 |
| 2.89135395 | 0 |
| 2.891597535 | 0 |
| 2.89184112 | 0 |
| 2.892084705 | 0 |
| 2.89232829 | 0 |
| 2.892571875 | 0 |
| 2.89281546 | 0 |
| 2.893059045 | 0 |
| 2.89330263 | 0 |
| 2.893546215 | 0 |
| 2.8937898 | 0.877 |
| 2.894033385 | 1.912 |
| 2.89427697 | 2.646 |
| 2.894520555 | 2.646 |
| 2.89476414 | 2.646 |
| 2.895007725 | 1.888 |
| 2.89525131 | 0.854 |
| 2.895494895 | 0 |
| 2.89573848 | 0 |
| 2.895982065 | 0 |
| 2.89622565 | 0 |
| 2.896469235 | 0 |
| 2.89671282 | −0.003 |
| 2.896956405 | −0.055 |
| 2.89719999 | −0.106 |
| 2.897443575 | −0.236 |
| 2.89768716 | −0.442 |
| 2.897930745 | −0.649 |
| 2.89817433 | −0.904 |
| 2.898417915 | −1.163 |
| 2.8986615 | −1.52 |
| 2.898905085 | −2.037 |
| 2.89914867 | −2.553 |
| 2.899392255 | −0.936 |
| 2.89963584 | 1.13 |
| 2.899879425 | 2.574 |
| 2.90012301 | 2.315 |
| 2.900366595 | 2.057 |
| 2.90061018 | 1.982 |
| 2.900853765 | 1.982 |
| 2.90109735 | 1.943 |
| 2.901340935 | 1.684 |
| 2.90158452 | 1.426 |
| 2.901828105 | 1.321 |
| 2.90207169 | 1.321 |
| 2.902315275 | 1.312 |
| 2.90255886 | 1.054 |
| 2.902802445 | 0.795 |
| 2.90304603 | 0.661 |
| 2.903289615 | 0.66 |
| 2.9035332 | 0.66 |
| 2.903776785 | 0.423 |
| 2.90402037 | 0.165 |
| 2.904263955 | 0 |
| 2.90450754 | 0 |
| 2.904751125 | 0 |
| 2.90499471 | 0 |
| 2.905238295 | 0 |
| 2.90548188 | 0 |
| 2.905725465 | 0 |
| 2.90596905 | 0 |
| 2.906212635 | 0 |
| 2.90645622 | 0 |
| 2.906699805 | 0 |
| 2.90694339 | 0 |
| 2.907186975 | 0 |
| 2.90743056 | 0 |
| 2.907674145 | 0 |
| 2.90791773 | 0 |
| 2.908161315 | 0 |
| 2.9084049 | 0 |
| 2.908648485 | 0 |
| 2.90889207 | 0 |
| 2.909135655 | 0 |
| 2.90937924 | 0.927 |
| 2.909622825 | 1.96 |
| 2.90986641 | 2.642 |
| 2.910109995 | 2.642 |
| 2.91035358 | 2.642 |
| 2.910597165 | 1.834 |
| 2.91084075 | 0.801 |
| 2.911084335 | 0 |
| 2.91132792 | 0 |
| 2.911571505 | 0 |
| 2.91181509 | 0 |
| 2.912058675 | 0 |
| 2.91230226 | −0.006 |
| 2.912545845 | −0.057 |
| 2.91278943 | −0.109 |
| 2.913033015 | −0.246 |
| 2.9132766 | −0.452 |
| 2.913520185 | −0.659 |
| 2.91376377 | −0.917 |
| 2.914007355 | −1.175 |
| 2.91425094 | −1.545 |
| 2.914494525 | −2.062 |
| 2.91473811 | −2.578 |
| 2.914981695 | −0.833 |
| 2.91522528 | 1.234 |
| 2.915468865 | 2.56 |
| 2.91571245 | 2.302 |
| 2.915956035 | 2.044 |
| 2.91619962 | 1.982 |
| 2.916443205 | 1.982 |
| 2.91668679 | 1.93 |
| 2.916930375 | 1.672 |
| 2.91717396 | 1.414 |
| 2.917417545 | 1.322 |
| 2.91766113 | 1.322 |
| 2.917904715 | 1.3 |
| 2.9181483 | 1.041 |
| 2.918391885 | 0.783 |
| 2.91863547 | 0.661 |
| 2.918879055 | 0.661 |
| 2.91912264 | 0.661 |
| 2.919366225 | 0.41 |
| 2.91960981 | 0.152 |
| 2.919853395 | 0 |
| 2.92009698 | 0 |
| 2.920340565 | 0 |
| 2.92058415 | 0 |
| 2.920827735 | 0 |
| 2.92107132 | 0 |
| 2.921314905 | 0 |
| 2.92155849 | 0 |
| 2.921802075 | 0 |
| 2.92204566 | 0 |
| 2.922289245 | 0 |
| 2.92253283 | 0 |
| 2.922776415 | 0 |
| 2.92302 | 0 |
| 2.923263585 | 0 |
| 2.92350717 | 0 |
| 2.923750755 | 0 |
| 2.92399434 | 0 |
| 2.924237925 | 0 |
| 2.92448151 | 0 |
| 2.924725095 | 0 |
| 2.92496868 | 0.981 |
| 2.925212265 | 2.016 |
| 2.92545585 | 2.648 |
| 2.925699435 | 2.648 |
| 2.92594302 | 2.648 |
| 2.926186605 | 1.787 |
| 2.92643019 | 0.752 |
| 2.926673775 | 0 |
| 2.92691736 | 0 |
| 2.927160945 | 0 |
| 2.92740453 | 0 |
| 2.927648115 | 0 |
| 2.9278917 | 0 |
| 2.928135285 | 0 |
| 2.92837887 | 0 |
| 2.928622455 | 0 |
| 2.92886604 | 0 |
| 2.929109625 | 0 |
| 2.92935321 | 0 |
| 2.929596795 | 0 |
| 2.92984038 | 0 |
| 2.930083965 | 0 |
| 2.93032755 | 0 |
| 2.930571135 | 0 |
| 2.93081472 | 0 |
| 2.931058305 | 0 |
| 2.93130189 | 0 |
| 2.931545475 | 0 |
| 2.93178906 | 0 |
| 2.932032645 | 0 |
| 2.93227623 | 0 |
| 2.932519815 | 0 |
| 2.9327634 | 0 |
| 2.933006985 | 0 |
| 2.93325057 | 0 |
| 2.933494155 | 0 |
| 2.93373774 | 0 |
| 2.933981325 | 0 |
| 2.93422491 | 0 |
| 2.934468495 | 0 |
| 2.93471208 | 0 |
| 2.934955665 | 0 |
| 2.93519925 | 0 |
| 2.935442835 | 0 |
| 2.93568642 | 0 |
| 2.935930005 | 0 |
| 2.93617359 | 0 |
| 2.936417175 | 0 |
| 2.93666076 | 0 |
| 2.936904345 | 0 |
| 2.93714793 | 0 |
| 2.937391515 | 0 |
| 2.9376351 | 0 |
| 2.937878685 | 0 |
| 2.93812227 | 0 |
| 2.938365855 | 0 |
| 2.93860944 | 0 |
| 2.938853025 | 0 |
| 2.93909661 | 0 |
| 2.939340195 | 0 |
| 2.93958378 | 0 |
| 2.939827365 | 0 |
| 2.94007095 | 0 |
| 2.940314535 | 0 |
| 2.94055812 | 0 |
| 2.940801705 | 0 |
| 2.94104529 | 0 |
| 2.941288875 | 0 |
| 2.94153246 | 0 |
| 2.941776045 | 0 |
| 2.94201963 | 0 |
| 2.942263215 | 0 |
| 2.9425068 | 0 |
| 2.942750385 | 0 |
| 2.94299397 | 0 |
| 2.943237555 | 0 |
| 2.94348114 | 0 |
| 2.943724725 | 0 |
| 2.94396831 | 0 |
| 2.944211895 | 0 |
| 2.94445548 | 0 |
| 2.944699065 | 0 |
| 2.94494265 | 0 |
| 2.945186235 | 0 |
| 2.94542982 | 0 |
| 2.945673405 | 0 |
| 2.94591699 | 0 |
| 2.946160575 | 0 |
| 2.94640416 | 0 |
| 2.946647745 | 0 |
| 2.94689133 | 0 |
| 2.947134915 | 0 |
| 2.9473785 | 0 |
| 2.947622085 | 0 |
| 2.94786567 | 0 |
| 2.948109255 | 0 |
| 2.94835284 | 0 |
| 2.948596425 | 0 |
| 2.94884001 | 0 |
| 2.949083595 | 0 |
| 2.94932718 | 0 |
| 2.949570765 | 0 |
| 2.94981435 | 0 |
| 2.950057935 | 0 |
| 2.95030152 | 0 |
| 2.950545105 | 0 |
| 2.95078869 | 0 |
| 2.951032275 | 0 |
| 2.95127586 | 0 |
| 2.951519445 | 0 |
| 2.95176303 | 0 |
| 2.952006615 | 0 |
| 2.9522502 | 0 |
| 2.952493785 | 0 |
| 2.95273737 | 0 |
| 2.952980955 | 0 |
| 2.95322454 | 0 |
| 2.953468125 | 0 |
| 2.95371171 | 0 |
| 2.953955295 | 0 |
| 2.95419888 | 0 |
| 2.954442465 | 0 |
| 2.95468605 | 0 |
| 2.954929635 | 0 |
| 2.95517322 | 0 |
| 2.955416805 | 0 |
| 2.95566039 | 0 |
| 2.955903975 | 0 |
| 2.95614756 | 0 |
| 2.956391145 | 0 |
| 2.95663473 | 0 |
| 2.956878315 | 0 |
| 2.9571219 | 0 |
| 2.957365485 | 0 |
| 2.95760907 | 0 |
| 2.957852655 | 0 |
| 2.95809624 | 0 |
| 2.958339825 | 0 |
| 2.95858341 | 0 |
| 2.958826995 | 0 |
| 2.95907058 | −0.014 |
| 2.959314165 | −0.066 |
| 2.95955775 | −0.119 |
| 2.959801335 | −0.282 |
| 2.96094492 | −0.493 |
| 2.960288505 | −0.711 |
| 2.96053209 | −0.975 |
| 2.960775675 | −1.238 |
| 2.96101926 | −1.656 |
| 2.961262845 | −2.183 |
| 2.96150643 | −2.642 |
| 2.961750015 | −0.533 |
| 2.9619936 | 1.576 |
| 2.962237185 | 2.575 |
| 2.96248077 | 2.311 |
| 2.962724355 | 2.048 |
| 2.96296794 | 2.025 |
| 2.963211525 | 2.025 |
| 2.96345511 | 1.932 |
| 2.963698695 | 1.669 |
| 2.96394228 | 1.405 |
| 2.964185865 | 1.351 |
| 2.96442945 | 1.351 |
| 2.964673035 | 1.289 |
| 2.96491662 | 1.025 |
| 2.965160205 | 0.761 |
| 2.96540379 | 0.676 |
| 2.965647375 | 0.676 |
| 2.96589096 | 0.645 |
| 2.966134545 | 0.38 |
| 2.96637813 | 0.116 |
| 2.966621715 | 0 |
| 2.9668653 | 0 |
| 2.967108885 | 0 |
| 2.96735247 | 0 |
| 2.967596055 | 0 |
| 2.96783964 | 0 |
| 2.968083225 | 0 |
| 2.96832681 | 0 |
| 2.968570395 | 0 |
| 2.96881398 | 0 |
| 2.969057565 | 0 |
| 2.96930115 | 0 |
| 2.969544735 | 0 |
| 2.96978832 | 0 |
| 2.970031905 | 0 |
| 2.97027549 | 0 |
| 2.970519075 | 0 |
| 2.97076266 | 0 |
| 2.971006245 | 0 |
| 2.97124983 | 0 |
| 2.971493415 | 0.104 |
| 2.971737 | 1.167 |
| 2.971980585 | 2.229 |
| 2.97222417 | 2.718 |
| 2.972467755 | 2.719 |
| 2.97271134 | 2.719 |
| 2.972954925 | 1.675 |
| 2.97319851 | 0.612 |
| 2.973442095 | 0 |
| 2.97368568 | 0 |
| 2.973929265 | 0 |
| 2.97417285 | 0 |
| 2.974416435 | 0 |
| 2.97466002 | −0.016 |
| 2.974903605 | −0.07 |
| 2.97514719 | −0.123 |
| 2.975390775 | −0.296 |
| 2.97563436 | −0.509 |
| 2.975877945 | −0.733 |
| 2.97612153 | −0.999 |
| 2.976365115 | −1.266 |
| 2.9766087 | −1.702 |
| 2.976852285 | −2.236 |
| 2.97709587 | −2.567 |
| 2.977339455 | −0.433 |
| 2.97758304 | 1.702 |
| 2.977826625 | 2.592 |
| 2.97807021 | 2.326 |
| 2.978313795 | 2.06 |
| 2.97855738 | 2.049 |
| 2.978800965 | 2.05 |
| 2.97904455 | 1.943 |
| 2.979288135 | 1.676 |
| 2.97953172 | 1.409 |
| 2.979775305 | 1.368 |
| 2.98001889 | 1.368 |
| 2.980262475 | 1.292 |
| 2.98050606 | 1.024 |
| 2.980749645 | 0.757 |
| 2.98099323 | 0.684 |
| 2.981236815 | 0.685 |
| 2.9814804 | 0.639 |
| 2.981723985 | 0.372 |
| 2.98196757 | 0.104 |
| 2.982211155 | 0 |
| 2.98245474 | 0 |
| 2.982698325 | 0 |
| 2.98294191 | 0 |
| 2.983185495 | 0 |
| 2.98342908 | 0 |
| 2.983672665 | 0 |
| 2.98391625 | 0 |
| 2.984159835 | 0 |
| 2.98440342 | 0 |
| 2.984647005 | 0 |
| 2.98489059 | 0 |
| 2.985134175 | 0 |
| 2.98537776 | 0 |
| 2.985621345 | 0 |
| 2.98586493 | 0 |
| 2.986108515 | 0 |
| 2.9863521 | 0 |
| 2.986595685 | 0 |
| 2.98683927 | 0 |
| 2.987082855 | 0.159 |
| 2.98732644 | 1.236 |
| 2.987570025 | 2.312 |
| 2.98781361 | 2.754 |
| 2.988057195 | 2.755 |
| 2.98830078 | 2.721 |
| 2.988544365 | 1.644 |
| 2.98878795 | 0.567 |
| 2.989031535 | 0 |
| 2.98927512 | 0 |
| 2.989518705 | 0 |
| 2.98976229 | 0 |
| 2.990005875 | 0 |
| 2.99024946 | 0 |
| 2.990493045 | 0 |
| 2.99073663 | 0 |
| 2.990980215 | 0 |
| 2.9912238 | 0 |
| 2.991467385 | 0 |
| 2.99171097 | 0 |
| 2.991954555 | 0 |
| 2.99219814 | 0 |
| 2.992441725 | 0 |
| 2.99268531 | 0 |
| 2.992928895 | 0 |
| 2.99317248 | 0 |
| 2.993416065 | 0 |
| 2.99365965 | 0 |
| 2.993903235 | 0 |
| 2.99414682 | 0 |
| 2.994390405 | 0 |
| 2.99463399 | 0 |
| 2.994877575 | 0 |
| 2.99512116 | 0 |
| 2.995364745 | 0 |
| 2.99560833 | 0 |
| 2.995851915 | 0 |
| 2.9960955 | 0 |
| 2.996339085 | 0 |
| 2.99658267 | 0 |
| 2.996826255 | 0 |
| 2.99706984 | 0 |
| 2.997313425 | 0 |
| 2.99755701 | 0 |
| 2.997800595 | 0 |
| 2.99804418 | 0 |
| 2.998287765 | 0 |
| 2.99853135 | 0 |
| 2.998774935 | 0 |
| 2.99901852 | 0 |
| 2.999262105 | 0 |
| 2.99950569 | 0 |
| 2.999749275 | 0 |
| 2.99999286 | 0 |
| 3.000236445 | 0 |
| 3.00048003 | 0 |
| 3.000723615 | 0 |
| 3.0009672 | 0 |
| 3.001210785 | 0 |
| 3.00145437 | 0 |
| 3.001697955 | 0 |
| 3.00194154 | 0 |
| 3.002185125 | 0 |
| 3.00242871 | 0 |
| 3.002672295 | 0 |
| 3.00291588 | 0 |
| 3.003159465 | 0 |
| 3.00340305 | 0 |
| 3.003646635 | 0 |
| 3.00389022 | 0 |
| 3.004133805 | 0 |
| 3.00437739 | 0 |
| 3.004620975 | 0 |
| 3.00486456 | 0 |
| 3.005108145 | 0 |
| 3.00535173 | 0 |
| 3.005595315 | 0 |
| 3.0058389 | 0 |
| 3.006082485 | 0 |
| 3.00632607 | 0 |
| 3.006569655 | 0 |
| 3.00681324 | 0 |
| 3.007056825 | 0 |
| 3.00730041 | 0 |
| 3.007543995 | 0 |
| 3.00778758 | 0 |
| 3.008031165 | 0 |
| 3.00827475 | 0 |
| 3.008518335 | 0 |
| 3.00876192 | 0 |
| 3.009005505 | 0 |
| 3.00924909 | 0 |
| 3.009492675 | 0 |
| 3.00973626 | 0 |
| 3.009979845 | 0 |
| 3.01022343 | 0 |
| 3.010467015 | 0 |
| 3.0107106 | 0 |
| 3.010954185 | 0 |
| 3.01139777 | 0 |
| 3.011441355 | 0 |
| 3.01168494 | 0 |
| 3.011928525 | 0 |
| 3.01217211 | 0 |
| 3.012415695 | 0 |
| 3.01265928 | 0 |
| 3.012902865 | 0 |
| 3.01314645 | 0 |
| 3.013390035 | 0 |
| 3.01363362 | 0 |
| 3.013877205 | 0 |
| 3.01412079 | 0 |
| 3.014364375 | 0 |
| 3.01460796 | 0 |
| 3.014851545 | 0 |
| 3.01509513 | 0 |
| 3.015338715 | 0 |
| 3.0155823 | 0 |
| 3.015825885 | 0 |
| 3.01606947 | 0 |
| 3.016313055 | 0 |
| 3.01655664 | 0 |
| 3.016800225 | 0 |
| 3.01704381 | 0 |
| 3.017287395 | 0 |
| 3.01753098 | 0 |
| 3.017774565 | 0 |
| 3.01801815 | 0 |
| 3.018261735 | 0 |
| 3.01850532 | 0 |
| 3.018748905 | 0 |
| 3.01899249 | 0 |
| 3.019236075 | 0 |
| 3.01947966 | 0 |
| 3.019723245 | 0 |
| 3.01996683 | 0 |
| 3.020210415 | 0 |
| 3.020454 | 0 |
| 3.020697585 | 0 |
| 3.02094117 | 0 |
| 3.021184755 | 0 |
| 3.02142834 | 0 |
| 3.021671925 | 0 |
| 3.02191551 | 0 |
| 3.022159095 | 0 |
| 3.02240268 | 0 |
| 3.022646265 | 0 |
| 3.02288985 | 0 |
| 3.023133435 | 0 |
| 3.02337702 | 0 |
| 3.023620605 | 0 |
| 3.02386419 | 0 |
| 3.024107775 | 0 |
| 3.02435136 | 0 |
| 3.024594945 | 0 |
| 3.02483853 | 0 |
| 3.025082115 | 0 |
| 3.0253257 | 0 |
| 3.025569285 | 0 |
| 3.02581287 | 0 |
| 3.026056455 | 0 |
| 3.02630004 | 0 |
| 3.026543625 | 0 |
| 3.02678721 | 0 |
| 3.027030795 | 0 |
| 3.02727438 | 0 |
| 3.027517965 | 0 |
| 3.02776155 | 0 |
| 3.028005135 | 0 |
| 3.02824872 | 0 |
| 3.028492305 | 0 |
| 3.02873589 | 0 |
| 3.028979475 | 0 |
| 3.02922306 | 0 |
| 3.029466645 | 0 |
| 3.02971023 | 0 |
| 3.029953815 | 0 |
| 3.0301974 | 0 |
| 3.030440985 | 0 |
| 3.03068457 | 0 |
| 3.030928155 | 0 |
| 3.03117174 | 0 |
| 3.031415325 | 0 |
| 3.03165891 | 0 |
| 3.031902495 | 0 |
| 3.03214608 | 0 |
| 3.032389665 | 0 |
| 3.03263325 | 0 |
| 3.032876835 | 0 |
| 3.03312042 | 0 |
| 3.033364005 | 0 |
| 3.03360759 | 0 |
| 3.033851175 | 0 |
| 3.03409476 | 0 |
| 3.034338345 | 0 |
| 3.03458193 | 0 |
| 3.034825515 | 0 |
| 3.0350691 | 0 |
| 3.035312685 | 0 |
| 3.03555627 | 0 |
| 3.035799855 | 0 |
| 3.03604344 | 0 |
| 3.036287025 | 0 |
| 3.03653061 | 0 |
| 3.036774195 | 0 |
| 3.03701778 | 0 |
| 3.037261365 | 0 |
| 3.03750495 | 0 |
| 3.037748535 | 0 |
| 3.03799212 | 0 |
| 3.038235705 | 0 |
| 3.03847929 | 0 |
| 3.038722875 | 0 |
| 3.03896646 | 0 |
| 3.039210045 | 0 |
| 3.03945363 | 0 |
| 3.039697215 | 0 |
| 3.0399408 | 0 |
| 3.040184385 | 0 |
| 3.04042797 | 0 |
| 3.040671555 | 0 |
| 3.04091514 | 0 |
| 3.041158725 | 0 |
| 3.04140231 | 0 |
| 3.041645895 | 0 |
| 3.04188948 | 0 |
| 3.042133065 | 0 |
| 3.04237665 | 0 |
| 3.042620235 | 0 |
| 3.04286382 | 0 |
| 3.043107405 | 0 |
| 3.04335099 | 0 |
| 3.043594575 | 0 |
| 3.04383816 | 0 |
| 3.044081745 | 0 |
| 3.04432533 | 0 |
| 3.044568915 | 0 |
| 3.0448125 | 0 |
| 3.045056085 | 0 |
| 3.04529967 | 0 |
| 3.045543255 | 0 |
| 3.04578684 | 0 |
| 3.046030425 | 0 |
| 3.04627401 | 0 |
| 3.046517595 | 0 |
| 3.04676118 | 0 |
| 3.047004765 | 0 |
| 3.04724835 | 0 |
| 3.047491935 | 0 |
| 3.04773552 | 0 |
| 3.047979105 | 0 |
| 3.04822269 | 0 |
| 3.048466275 | 0 |
| 3.04870986 | 0 |
| 3.048953445 | 0 |
| 3.04919703 | 0 |
| 3.049440615 | 0 |
| 3.0496842 | 0 |
| 3.049927785 | 0 |
| 3.05017137 | 0 |
| 3.050414955 | 0 |
| 3.05065854 | 0 |
| 3.050902125 | 0 |
| 3.05114571 | 0 |
| 3.051389295 | 0 |
| 3.05163288 | 0 |
| 3.051876465 | 0 |
| 3.05212005 | 0 |
| 3.052363635 | 0 |
| 3.05260722 | −0.032 |
| 3.052850805 | −0.089 |
| 3.05309439 | −0.146 |
| 3.053337975 | −0.374 |
| 3.05358156 | −0.603 |
| 3.053825145 | −0.857 |
| 3.05406873 | −1.143 |
| 3.054312315 | −1.428 |
| 3.0545559 | −1.967 |
| 3.054799485 | −2.539 |
| 3.05504307 | −2.179 |
| 3.055286655 | 0.108 |
| 3.05553024 | 2.395 |
| 3.055773825 | 2.707 |
| 3.05601741 | 2.421 |
| 3.056260995 | 2.196 |
| 3.05650458 | 2.196 |
| 3.056748165 | 2.196 |
| 3.05699175 | 2.01 |
| 3.057235335 | 1.724 |
| 3.05747892 | 1.465 |
| 3.057722505 | 1.465 |
| 3.05796609 | 1.466 |
| 3.058209675 | 1.312 |
| 3.05845326 | 1.026 |
| 3.058696845 | 0.74 |
| 3.05894043 | 0.733 |
| 3.059184015 | 0.734 |
| 3.0594276 | 0.613 |
| 3.059671185 | 0.326 |
| 3.05991477 | 0.04 |
| 3.060158355 | 0 |
| 3.06040194 | 0 |
| 3.060645525 | 0 |
| 3.06088911 | 0 |
| 3.061132695 | 0 |
| 3.06137628 | 0 |
| 3.061619865 | 0 |
| 3.06186345 | 0 |
| 3.062107035 | 0 |
| 3.06235062 | 0 |
| 3.062594205 | 0 |
| 3.06283779 | 0 |
| 3.063081375 | 0 |
| 3.06332496 | 0 |
| 3.063568545 | 0 |
| 3.06381213 | 0 |
| 3.064055715 | 0 |
| 3.0642993 | 0 |
| 3.064542885 | 0 |
| 3.06478647 | 0 |
| 3.065030055 | 0.459 |
| 3.06527364 | 1.611 |
| 3.065517225 | 2.764 |
| 3.06576081 | 2.948 |
| 3.066004395 | 2.949 |
| 3.06624798 | 2.624 |
| 3.066491565 | 1.471 |
| 3.06673515 | 0.318 |
| 3.066978735 | 0 |
| 3.06722232 | 0 |
| 3.067465905 | 0 |
| 3.06770949 | 0 |
| 3.067953075 | 0 |
| 3.06819666 | 0 |
| 3.068440245 | 0 |
| 3.06868383 | 0 |
| 3.068927415 | 0 |
| 3.069171 | 0 |
| 3.069414585 | 0 |
| 3.06965817 | 0 |
| 3.069901755 | 0 |
| 3.07014534 | 0 |
| 3.070388925 | 0 |
| 3.07063251 | 0 |
| 3.070876095 | 0 |
| 3.07111968 | 0 |
| 3.071363265 | 0 |
| 3.07160685 | 0 |
| 3.071850435 | 0 |
| 3.07209402 | 0 |
| 3.072337605 | 0 |
| 3.07258119 | 0 |
| 3.072824775 | 0 |
| 3.07306836 | 0 |
| 3.073311945 | 0 |
| 3.07355553 | 0 |
| 3.073799115 | 0 |
| 3.0740427 | 0 |
| 3.074286285 | 0 |
| 3.07452987 | 0 |
| 3.074773455 | 0 |
| 3.07501704 | 0 |
| 3.075260625 | 0 |
| 3.07550421 | 0 |
| 3.075747795 | 0 |
| 3.07599138 | 0 |
| 3.076234965 | 0 |
| 3.07647855 | 0 |
| 3.076722135 | 0 |
| 3.07696572 | 0 |
| 3.077209305 | 0 |
| 3.07745289 | 0 |
| 3.077696475 | 0 |
| 3.07794006 | 0 |
| 3.078183645 | 0 |
| 3.07842723 | 0 |
| 3.078670815 | 0 |
| 3.0789144 | 0 |
| 3.079157985 | 0 |
| 3.07940157 | 0 |
| 3.079645155 | 0 |
| 3.07988874 | 0 |
| 3.080132325 | 0 |
| 3.08037591 | 0 |
| 3.080619495 | 0 |
| 3.08086308 | 0 |
| 3.081106665 | 0 |
| 3.08135025 | 0 |
| 3.081593835 | 0 |
| 3.08183742 | 0 |
| 3.082081005 | 0 |
| 3.08232459 | 0 |
| 3.082568175 | 0 |
| 3.08281176 | 0 |
| 3.083055345 | 0 |
| 3.08329893 | 0 |
| 3.083542515 | 0 |
| 3.0837861 | 0 |
| 3.084029685 | 0 |
| 3.08427327 | 0 |
| 3.084516855 | 0 |
| 3.08476044 | 0 |
| 3.085004025 | 0 |
| 3.08524761 | 0 |
| 3.085491195 | 0 |
| 3.08573478 | 0 |
| 3.085978365 | 0 |
| 3.08622195 | 0 |
| 3.086465535 | 0 |
| 3.08670912 | 0 |
| 3.086952705 | 0 |
| 3.08719629 | 0 |
| 3.087439875 | 0 |
| 3.08768346 | 0 |
| 3.087927045 | 0 |
| 3.08817063 | 0 |
| 3.088414215 | 0 |
| 3.0886578 | 0 |
| 3.088901385 | 0 |
| 3.08914497 | 0 |
| 3.089388555 | 0 |
| 3.08963214 | 0 |
| 3.089875725 | 0 |
| 3.09011931 | 0 |
| 3.090362895 | 0 |
| 3.09060648 | 0 |
| 3.090850065 | 0 |
| 3.09109365 | 0 |
| 3.091337235 | 0 |
| 3.09158082 | 0 |
| 3.091824405 | 0 |
| 3.09206799 | 0 |
| 3.092311575 | 0 |
| 3.09255516 | 0 |
| 3.092798745 | 0 |
| 3.09304233 | 0 |
| 3.093285915 | 0 |
| 3.0935295 | 0 |
| 3.093773085 | 0 |
| 3.09401667 | 0 |
| 3.094260255 | 0 |
| 3.09450384 | 0 |
| 3.094747425 | 0 |
| 3.09499101 | 0 |
| 3.095234595 | 0 |
| 3.09547818 | 0 |
| 3.095721765 | 0 |
| 3.09596535 | 0 |
| 3.096208935 | 0 |
| 3.09645252 | 0 |
| 3.096696105 | 0 |
| 3.09693969 | 0 |
| 3.097183275 | 0 |
| 3.09742686 | 0 |
| 3.097670445 | 0 |
| 3.09791403 | 0 |
| 3.098157615 | 0 |
| 3.0984012 | 0 |
| 3.098644785 | 0 |
| 3.09888837 | 0 |
| 3.099131955 | 0 |
| 3.09937554 | 0 |
| 3.099619125 | 0 |
| 3.09986271 | 0 |
| 3.100106295 | 0 |
| 3.10034988 | 0 |
| 3.100593465 | 0 |
| 3.10083705 | 0 |
| 3.101080635 | 0 |
| 3.10132422 | 0 |
| 3.101567805 | 0 |
| 3.10181139 | 0 |
| 3.102054975 | 0 |
| 3.10229856 | 0 |
| 3.102542145 | 0 |
| 3.10278573 | 0 |
| 3.103029315 | 0 |
| 3.1032729 | 0 |
| 3.103516485 | 0 |
| 3.10376007 | 0 |
| 3.104003655 | 0 |
| 3.10424724 | 0 |
| 3.104490825 | 0 |
| 3.10473441 | 0 |
| 3.104977995 | 0 |
| 3.10522158 | 0 |
| 3.105465165 | 0 |
| 3.10570875 | 0 |
| 3.105952335 | 0 |
| 3.10619592 | 0 |
| 3.106439505 | 0 |
| 3.10668309 | 0 |
| 3.106926675 | 0 |
| 3.10717026 | 0 |
| 3.107413845 | 0 |
| 3.10765743 | 0 |
| 3.107901015 | 0 |
| 3.1081446 | 0 |
| 3.108388185 | 0 |
| 3.10863177 | 0 |
| 3.108875355 | 0 |
| 3.10911894 | 0 |
| 3.109362525 | 0 |
| 3.10960611 | 0 |
| 3.109849695 | 0 |
| 3.11009328 | 0 |
| 3.110336865 | 0 |
| 3.11058045 | 0 |
| 3.110824035 | 0 |
| 3.11106762 | 0 |
| 3.111311205 | 0 |
| 3.11155479 | 0 |
| 3.111798375 | 0 |
| 3.11204196 | 0 |
| 3.112285545 | 0 |
| 3.11252913 | 0 |
| 3.112772715 | 0 |
| 3.1130163 | 0 |
| 3.113259885 | 0 |
| 3.11350347 | 0 |
| 3.113747055 | 0 |
| 3.11399064 | 0 |
| 3.114234225 | 0 |
| 3.11447781 | 0 |
| 3.114721395 | 0 |
| 3.11496498 | 0 |
| 3.115208565 | 0 |
| 3.11545215 | 0 |
| 3.115695735 | 0 |
| 3.11593932 | 0 |
| 3.116182905 | 0 |
| 3.11642649 | 0 |
| 3.116670075 | 0 |
| 3.11691366 | 0 |
| 3.117157245 | 0 |
| 3.11740083 | 0 |
| 3.117644415 | 0 |
| 3.117888 | 0 |
| 3.118131585 | 0 |
| 3.11837517 | 0 |
| 3.118618755 | 0 |
| 3.11886234 | 0 |
| 3.119105925 | 0 |
| 3.11934951 | 0 |
| 3.119593095 | 0 |
| 3.11983668 | 0 |
| 3.120080265 | 0 |
| 3.12032385 | 0 |
| 3.120567435 | 0 |
| 3.12081102 | 0 |
| 3.121054605 | 0 |
| 3.12129819 | 0 |
| 3.121541775 | 0 |
| 3.12178536 | 0 |
| 3.122028945 | 0 |
| 3.12227253 | 0 |
| 3.122516115 | 0 |
| 3.1227597 | 0 |
| 3.123003285 | 0 |
| 3.12324687 | 0 |
| 3.123490455 | 0 |
| 3.12373404 | 0 |
| 3.123977625 | 0 |
| 3.12422121 | 0 |
| 3.124464795 | 0 |
| 3.12470838 | 0 |
| 3.124951965 | 0 |
| 3.12519555 | 0 |
| 3.125439135 | 0 |
| 3.12568272 | 0 |
| 3.125926305 | 0 |
| 3.12616989 | 0 |
| 3.126413475 | 0 |
| 3.12665706 | 0 |
| 3.126900645 | 0 |
| 3.12714423 | 0 |
| 3.127387815 | 0 |
| 3.1276314 | 0 |
| 3.127874985 | 0 |
| 3.12811857 | 0 |
| 3.128362155 | 0 |
| 3.12860574 | 0 |
| 3.128849325 | 0 |
| 3.12909291 | 0 |
| 3.129336495 | 0 |
| 3.12958008 | 0 |
| 3.129823665 | 0 |
| 3.13006725 | 0 |
| 3.130310835 | 0 |
| 3.13055442 | 0 |
| 3.130798005 | 0 |
| 3.13104159 | 0 |
| 3.131285175 | 0 |
| 3.13152876 | 0 |
| 3.131772345 | 0 |
| 3.13201593 | 0 |
| 3.132259515 | 0 |
| 3.1325031 | 0 |
| 3.132746685 | 0 |
| 3.13299027 | 0 |
| 3.133233855 | 0 |
| 3.13347744 | 0 |
| 3.133721025 | 0 |
| 3.13396461 | 0 |
| 3.134208195 | 0 |
| 3.13445178 | 0 |
| 3.134695365 | 0 |
| 3.13493895 | 0 |
| 3.135182535 | 0 |
| 3.13542612 | 0 |
| 3.135669705 | 0 |
| 3.13591329 | 0 |
| 3.136156875 | 0 |
| 3.13640046 | 0 |
| 3.136644045 | 0 |
| 3.13688763 | 0 |
| 3.137131215 | 0 |
| 3.1373748 | 0 |
| 3.137618385 | 0 |
| 3.13786197 | 0 |
| 3.138105555 | 0 |
| 3.13834914 | 0 |
| 3.138592725 | 0 |
| 3.13883631 | 0 |
| 3.139079895 | 0 |
| 3.13932348 | 0 |
| 3.139567065 | 0 |
| 3.13981065 | 0 |
| 3.140054235 | 0 |
| 3.14029782 | 0 |
| 3.140541405 | 0 |
| 3.14078499 | 0 |
| 3.141028575 | 0 |
| 3.14127216 | 0 |
| 3.141515745 | 0 |
| 3.14175933 | 0 |
| 3.142002915 | 0 |
| 3.1422465 | 0 |
| 3.142490085 | 0 |
| 3.14273367 | 0 |
| 3.142977255 | 0 |
| 3.14322084 | 0 |
| 3.143464425 | 0 |
| 3.14370801 | 0 |
| 3.143951595 | 0 |
| 3.14419518 | 0 |
| 3.144438765 | 0 |
| 3.14468235 | 0 |
| 3.144925935 | 0 |
| 3.14516952 | 0 |
| 3.145413105 | 0 |
| 3.14565669 | 0 |
| 3.145900275 | 0 |
| 3.14614386 | 0 |
| 3.146387445 | 0 |
| 3.14663103 | 0 |
| 3.146874615 | 0 |
| 3.1471182 | 0 |
| 3.147361785 | 0 |
| 3.14760537 | 0 |
| 3.147848955 | 0 |
| 3.14809254 | 0 |
| 3.148336125 | 0 |
| 3.14857971 | 0 |
| 3.148823295 | 0 |
| 3.14906688 | 0 |
| 3.149310465 | 0 |
| 3.14955405 | 0 |
| 3.149797635 | 0 |
| 3.15004122 | 0 |
| 3.150284805 | 0 |
| 3.15052839 | 0 |
| 3.150771975 | 0 |
| 3.15101556 | 0 |
| 3.151259145 | 0 |
| 3.15150273 | 0 |
| 3.151746315 | 0 |
| 3.1519899 | 0 |
| 3.152233485 | 0 |
| 3.15247707 | 0 |
| 3.152720655 | 0 |
| 3.15296424 | 0 |
| 3.153207825 | 0 |
| 3.15345141 | 0 |
| 3.153694995 | 0 |
| 3.15393858 | 0 |
| 3.154182165 | 0 |
| 3.15442575 | 0 |
| 3.154669335 | 0 |
| 3.15491292 | 0 |
| 3.155156505 | 0 |
| 3.15540009 | 0 |
| 3.155643675 | 0 |
| 3.15588726 | 0 |
| 3.156130845 | 0 |
| 3.15637443 | 0 |
| 3.156618015 | 0 |
| 3.1568616 | 0 |
| 3.157105185 | 0 |
| 3.15734877 | 0 |
| 3.157592355 | 0 |
| 3.15783594 | 0 |
| 3.158079525 | 0 |
| 3.15832311 | 0 |
| 3.158566695 | 0 |
| 3.15881028 | 0 |
| 3.159053865 | 0 |
| 3.15929745 | 0 |
| 3.159541035 | 0 |
| 3.15978462 | 0 |
| 3.160028205 | 0 |
| 3.16027179 | 0 |
| 3.160515375 | 0 |
| 3.16075896 | 0 |
| 3.161002545 | 0 |
| 3.16124613 | 0 |
| 3.161489715 | 0 |
| 3.1617333 | 0 |
| 3.161976885 | 0 |
| 3.16222047 | 0 |
| 3.162464055 | 0 |
| 3.16270764 | 0 |
| 3.162951225 | 0 |
| 3.16319481 | 0 |
| 3.163438395 | 0 |
| 3.16368198 | 0 |
| 3.163925565 | 0 |
| 3.16416915 | 0 |
| 3.164412735 | 0 |
| 3.16465632 | 0 |
| 3.164899905 | 0 |
| 3.16514349 | 0 |
| 3.165387075 | 0 |
| 3.16563066 | 0 |
| 3.165874245 | 0 |
| 3.16611783 | 0 |
| 3.166361415 | 0 |
| 3.166605 | 0 |
| 3.166848585 | 0 |
| 3.16709217 | 0 |
| 3.167335755 | 0 |
| 3.16757934 | 0 |
| 3.167822925 | 0 |
| 3.16806651 | 0 |
| 3.168310095 | 0 |
| 3.16855368 | 0 |
| 3.168797265 | 0 |
| 3.16904085 | 0 |
| 3.169284435 | 0 |
| 3.16952802 | 0 |
| 3.169771605 | 0 |
| 3.17001519 | 0 |
| 3.170258775 | 0 |
| 3.17050236 | 0 |
| 3.170745945 | 0 |
| 3.17098953 | 0 |
| 3.171233115 | 0 |
| 3.1714767 | 0 |
| 3.171720285 | 0 |
| 3.17196387 | 0 |
| 3.172207455 | 0 |
| 3.17245104 | 0 |
| 3.172694625 | 0 |
| 3.17293821 | 0 |
| 3.173181795 | 0 |
| 3.17342538 | 0 |
| 3.173668965 | 0 |
| 3.17391255 | 0 |
| 3.174156135 | 0 |
| 3.17439972 | 0 |
| 3.174643305 | 0 |
| 3.17488689 | 0 |
| 3.175130475 | 0 |
| 3.17537406 | 0 |
| 3.175617645 | 0 |
| 3.17586123 | 0 |
| 3.176104815 | 0 |
| 3.1763484 | 0 |
| 3.176591985 | 0 |
| 3.17683557 | 0 |
| 3.177079155 | 0 |
| 3.17732274 | −0.056 |
| 3.177566325 | −0.114 |
| 3.17780991 | −0.243 |
| 3.178053495 | −0.477 |
| 3.17829708 | −0.71 |
| 3.178540665 | −0.993 |
| 3.17878425 | −1.285 |
| 3.179027835 | −1.66 |
| 3.17927142 | −2.243 |
| 3.179515005 | −2.827 |
| 3.17975859 | −1.292 |
| 3.180002175 | 1.043 |
| 3.18024576 | 2.937 |
| 3.180489345 | 2.644 |
| 3.18073293 | 2.352 |
| 3.180976515 | 2.238 |
| 3.1812201 | 2.238 |
| 3.181463685 | 2.222 |
| 3.18170727 | 1.93 |
| 3.181950855 | 1.639 |
| 3.18219444 | 1.491 |
| 3.182438025 | 1.491 |
| 3.18268161 | 1.49 |
| 3.182925195 | 1.218 |
| 3.18316878 | 0.926 |
| 3.183412365 | 0.745 |
| 3.18365595 | 0.745 |
| 3.183899535 | 0.745 |
| 3.18414312 | 0.506 |
| 3.184386705 | 0.215 |
| 3.18463029 | 0 |
| 3.184873875 | 0 |
| 3.18511746 | 0 |
| 3.185361045 | 0 |
| 3.18560463 | 0 |
| 3.185848215 | 0 |
| 3.1860918 | 0 |
| 3.186335385 | 0 |
| 3.18657897 | 0 |
| 3.186822555 | 0 |
| 3.18706614 | 0 |
| 3.187309725 | 0 |
| 3.18755331 | 0 |
| 3.187796895 | 0 |
| 3.18804048 | 0 |
| 3.188284065 | 0 |
| 3.18852765 | 0 |
| 3.188771235 | 0 |
| 3.18901482 | 0 |
| 3.189258405 | 0 |
| 3.18950199 | 0 |
| 3.189745575 | 0.925 |
| 3.18998916 | 2.084 |
| 3.190232745 | 2.965 |
| 3.19047633 | 2.964 |
| 3.190719915 | 2.964 |
| 3.1909635 | 2.173 |
| 3.191207085 | 1.015 |
| 3.19145067 | 0 |
| 3.191694255 | 0 |
| 3.19193784 | 0 |
| 3.192181425 | 0 |
| 3.19242501 | 0 |
| 3.192668595 | 0 |
| 3.19291218 | −0.058 |
| 3.193155765 | −0.116 |
| 3.19339935 | −0.252 |
| 3.193642935 | −0.483 |
| 3.19388652 | −0.714 |
| 3.194130105 | −0.996 |
| 3.19437369 | −1.285 |
| 3.194617275 | −1.67 |
| 3.19486086 | −2.247 |
| 3.195104445 | −2.824 |
| 3.19534803 | −1.162 |
| 3.195591615 | 1.146 |
| 3.1958352 | 2.888 |
| 3.196078785 | 2.599 |
| 3.19632237 | 2.311 |
| 3.196565955 | 2.212 |
| 3.19680954 | 2.212 |
| 3.197053125 | 2.182 |
| 3.19729671 | 1.893 |
| 3.197540295 | 1.605 |
| 3.19778388 | 1.473 |
| 3.198027465 | 1.473 |
| 3.19827105 | 1.472 |
| 3.198514635 | 1.189 |
| 3.19875822 | 0.901 |
| 3.199001805 | 0.736 |
| 3.19924539 | 0.736 |
| 3.199488975 | 0.735 |
| 3.19973256 | 0.485 |
| 3.199976145 | 0.198 |
| 3.20021973 | 0 |
| 3.200463315 | 0 |
| 3.2007069 | 0 |
| 3.200950485 | 0 |
| 3.20119407 | 0 |
| 3.201437655 | 0 |
| 3.20168124 | 0 |
| 3.201924825 | 0 |
| 3.20216841 | 0 |
| 3.202411995 | 0 |
| 3.20265558 | 0 |
| 3.202899165 | 0 |
| 3.20314275 | 0 |
| 3.203386335 | 0 |
| 3.20362992 | 0 |
| 3.203873505 | 0 |
| 3.20411709 | 0 |
| 3.204360675 | 0 |
| 3.20460426 | 0 |
| 3.204847845 | 0 |
| 3.20509143 | 0 |
| 3.205335015 | 0.969 |
| 3.2055786 | 2.113 |
| 3.205822185 | 2.924 |
| 3.20606577 | 2.923 |
| 3.206309355 | 2.923 |
| 3.20655294 | 2.086 |
| 3.206796525 | 0.944 |
| 3.20704011 | 0 |
| 3.207283695 | 0 |
| 3.20752728 | 0 |
| 3.207770865 | 0 |
| 3.20801445 | 0 |
| 3.208258035 | −0.003 |
| 3.20850162 | −0.06 |
| 3.208745205 | −0.117 |
| 3.20898879 | −0.26 |
| 3.209232375 | −0.487 |
| 3.20947596 | −0.715 |
| 3.209719545 | −0.996 |
| 3.20996313 | −1.28 |
| 3.210206715 | −1.674 |
| 3.2104503 | −2.242 |
| 3.210693885 | −2.81 |
| 3.21093747 | −1.031 |
| 3.211181055 | 1.243 |
| 3.21142464 | 2.831 |
| 3.211668225 | 2.546 |
| 3.21191181 | 2.262 |
| 3.212155395 | 2.179 |
| 3.21239898 | 2.178 |
| 3.212642565 | 2.134 |
| 3.21288615 | 1.85 |
| 3.213129735 | 1.566 |
| 3.21337332 | 1.45 |
| 3.213616905 | 1.45 |
| 3.21386049 | 1.439 |
| 3.214104075 | 1.156 |
| 3.21434766 | 0.872 |
| 3.214591245 | 0.724 |
| 3.21483483 | 0.724 |
| 3.215078415 | 0.724 |
| 3.215322 | 0.464 |
| 3.215565585 | 0.181 |
| 3.21580917 | 0 |
| 3.216052755 | 0 |
| 3.21629634 | 0 |
| 3.216539925 | 0 |
| 3.21678351 | 0 |
| 3.217027095 | 0 |
| 3.21727068 | 0 |
| 3.217514265 | 0 |
| 3.21775785 | 0 |
| 3.218001435 | 0 |
| 3.21824502 | 0 |
| 3.218488605 | 0 |
| 3.21873219 | 0 |
| 3.218975775 | 0 |
| 3.21921936 | 0 |
| 3.219462945 | 0 |
| 3.21970653 | 0 |
| 3.219950115 | 0 |
| 3.2201937 | 0 |
| 3.220437285 | 0 |
| 3.22068087 | 0 |
| 3.220924455 | 1.009 |
| 3.22116804 | 2.133 |
| 3.221411625 | 2.874 |
| 3.22165521 | 2.873 |
| 3.221898795 | 2.872 |
| 3.22214238 | 1.994 |
| 3.222385965 | 0.871 |
| 3.22262955 | 0 |
| 3.222873135 | 0 |
| 3.22311672 | 0 |
| 3.223360305 | 0 |
| 3.22360389 | 0 |
| 3.223847475 | −0.006 |
| 3.22409106 | −0.062 |
| 3.224334645 | −0.118 |
| 3.22457823 | −0.266 |
| 3.224821815 | −0.49 |
| 3.2250654 | −0.713 |
| 3.225308985 | −0.992 |
| 3.22555257 | −1.271 |
| 3.225796155 | −1.671 |
| 3.22603974 | −2.229 |
| 3.226283325 | −2.787 |
| 3.22652691 | −0.901 |
| 3.226770495 | 1.332 |
| 3.22701408 | 2.765 |
| 3.227257665 | 2.485 |
| 3.22750125 | 2.206 |
| 3.227744835 | 2.138 |
| 3.22798842 | 2.138 |
| 3.228232005 | 2.081 |
| 3.22847559 | 1.802 |
| 3.228719175 | 1.523 |
| 3.22896276 | 1.423 |
| 3.229206345 | 1.423 |
| 3.22944993 | 1.398 |
| 3.229693515 | 1.12 |
| 3.2299371 | 0.842 |
| 3.230180685 | 0.71 |
| 3.23042427 | 0.71 |
| 3.230667855 | 0.71 |
| 3.23091144 | 0.441 |
| 3.231155025 | 0.163 |
| 3.23139861 | 0 |
| 3.231642195 | 0 |
| 3.23188578 | 0 |
| 3.232129365 | 0 |
| 3.23237295 | 0 |
| 3.232616535 | 0 |
| 3.23286012 | 0 |
| 3.233103705 | 0 |
| 3.23334729 | 0 |
| 3.233590875 | 0 |
| 3.23383446 | 0 |
| 3.234078045 | 0 |
| 3.23432163 | 0 |
| 3.234565215 | 0 |
| 3.2348088 | 0 |
| 3.235052385 | 0 |
| 3.23529597 | 0 |
| 3.235539555 | 0 |
| 3.23578314 | 0 |
| 3.236026725 | 0 |
| 3.23627031 | 0 |
| 3.236513895 | 1.043 |
| 3.23675748 | 2.144 |
| 3.237001065 | 2.815 |
| 3.23724465 | 2.814 |
| 3.237488235 | 2.813 |
| 3.23773182 | 1.898 |
| 3.237975405 | 0.798 |
| 3.23821899 | 0 |
| 3.238462575 | 0 |
| 3.23870616 | 0 |
| 3.238949745 | 0 |
| 3.23919333 | 0 |
| 3.239436915 | 0 |
| 3.2396805 | 0 |
| 3.239924085 | 0 |
| 3.24016767 | 0 |
| 3.240411255 | 0 |
| 3.24065484 | 0 |
| 3.240898425 | 0 |
| 3.24114201 | 0 |
| 3.241385595 | 0 |
| 3.24162918 | 0 |
| 3.241872765 | 0 |
| 3.24211635 | 0 |
| 3.242359935 | 0 |
| 3.24260352 | 0 |
| 3.242847105 | 0 |
| 3.24309069 | 0 |
| 3.243334275 | 0 |
| 3.24357786 | 0 |
| 3.243821445 | 0 |
| 3.24406503 | 0 |
| 3.244308615 | 0 |
| 3.2445522 | 0 |
| 3.244795785 | 0 |
| 3.24503937 | 0 |
| 3.245282955 | 0 |
| 3.24552654 | 0 |
| 3.245770125 | 0 |
| 3.24601371 | 0 |
| 3.246257295 | 0 |
| 3.24650088 | 0 |
| 3.246744465 | 0 |
| 3.24698805 | 0 |
| 3.247231635 | 0 |
| 3.24747522 | 0 |
| 3.247718805 | 0 |
| 3.24796239 | 0 |
| 3.248205975 | 0 |
| 3.24844956 | 0 |
| 3.248693145 | 0 |
| 3.24893673 | 0 |
| 3.249180315 | 0 |
| 3.2494239 | 0 |
| 3.249667485 | 0 |
| 3.24991107 | 0 |
| 3.250154655 | 0 |
| 3.25039824 | 0 |
| 3.250641825 | 0 |
| 3.25088541 | 0 |
| 3.251128995 | 0 |
| 3.25137258 | 0 |
| 3.251616165 | 0 |
| 3.25185975 | 0 |
| 3.252103335 | 0 |
| 3.25234692 | 0 |
| 3.252590505 | 0 |
| 3.25283409 | 0 |
| 3.253077675 | 0 |
| 3.25332126 | 0 |
| 3.253564845 | 0 |
| 3.25380843 | 0 |
| 3.254052015 | 0 |
| 3.2542956 | 0 |
| 3.254539185 | 0 |
| 3.25478277 | 0 |
| 3.255026355 | 0 |
| 3.25526994 | 0 |
| 3.255513525 | 0 |
| 3.25575711 | 0 |
| 3.256000695 | 0 |
| 3.25624428 | 0 |
| 3.256487865 | 0 |
| 3.25673145 | 0 |
| 3.256975035 | 0 |
| 3.25721862 | 0 |
| 3.257462205 | 0 |
| 3.25770579 | 0 |
| 3.257949375 | 0 |
| 3.25819296 | 0 |
| 3.258436545 | 0 |
| 3.25868013 | 0 |
| 3.258923715 | 0 |
| 3.2591673 | 0 |
| 3.259410885 | 0 |
| 3.25965447 | 0 |
| 3.259898055 | 0 |
| 3.26014164 | 0 |
| 3.260385225 | 0 |
| 3.26062881 | 0 |
| 3.260872395 | 0 |
| 3.26111598 | 0 |
| 3.261359565 | 0 |
| 3.26160315 | 0 |
| 3.261846735 | 0 |
| 3.26209032 | 0 |
| 3.262333905 | 0 |
| 3.26257749 | 0 |
| 3.262821075 | 0 |
| 3.26306466 | 0 |
| 3.263308245 | 0 |
| 3.26355183 | 0 |
| 3.263795415 | 0 |
| 3.264039 | 0 |
| 3.264282585 | 0 |
| 3.26452617 | 0 |
| 3.264769755 | 0 |
| 3.26501334 | 0 |
| 3.265256925 | 0 |
| 3.26550051 | 0 |
| 3.265744095 | 0 |
| 3.26598768 | 0 |
| 3.266231265 | 0 |
| 3.26647485 | 0 |
| 3.266718435 | 0 |
| 3.26696202 | 0 |
| 3.267205605 | 0 |
| 3.26744919 | 0 |
| 3.267692775 | 0 |
| 3.26793636 | 0 |
| 3.268179945 | 0 |
| 3.26842353 | 0 |
| 3.268667115 | 0 |
| 3.2689107 | 0 |
| 3.269154285 | 0 |
| 3.26939787 | 0 |
| 3.269641455 | 0 |
| 3.26988504 | 0 |
| 3.270128625 | 0 |
| 3.27037221 | 0 |
| 3.270615795 | 0 |
| 3.27085938 | 0 |
| 3.271102965 | 0 |
| 3.27134655 | 0 |
| 3.271590135 | 0 |
| 3.27183372 | 0 |
| 3.272077305 | 0 |
| 3.27232089 | 0 |
| 3.272564475 | 0 |
| 3.27280806 | 0 |
| 3.273051645 | 0 |
| 3.27329523 | 0 |
| 3.273538815 | 0 |
| 3.2737824 | 0 |
| 3.274025985 | 0 |
| 3.27426957 | 0 |
| 3.274513155 | 0 |
| 3.27475674 | 0 |
| 3.275000325 | 0 |
| 3.27524391 | 0 |
| 3.275487495 | 0 |
| 3.27573108 | 0 |
| 3.275974665 | 0 |
| 3.27621825 | 0 |
| 3.276461835 | 0 |
| 3.27670542 | 0 |
| 3.276949005 | 0 |
| 3.27719259 | 0 |
| 3.277436175 | 0 |
| 3.27767976 | 0 |
| 3.277923345 | 0 |
| 3.27816693 | 0 |
| 3.278410515 | 0 |
| 3.2786541 | 0 |
| 3.278897685 | 0 |
| 3.27914127 | 0 |
| 3.279384855 | 0 |
| 3.27962844 | 0 |
| 3.279872025 | 0 |
| 3.28011561 | 0 |
| 3.280359195 | 0 |
| 3.28060278 | 0 |
| 3.280846365 | 0 |
| 3.28108995 | 0 |
| 3.281333535 | 0 |
| 3.28157712 | 0 |
| 3.281820705 | 0 |
| 3.28206429 | 0 |
| 3.282307875 | 0 |
| 3.28255146 | 0 |
| 3.282795045 | 0 |
| 3.28303863 | 0 |
| 3.283282215 | 0 |
| 3.2835258 | 0 |
| 3.283769385 | 0 |
| 3.28401297 | 0 |
| 3.284256555 | 0 |
| 3.28450014 | 0 |
| 3.284743725 | 0 |
| 3.28498731 | 0 |
| 3.285230895 | 0 |
| 3.28547448 | 0 |
| 3.285718065 | 0 |
| 3.28596165 | 0 |
| 3.286205235 | 0 |
| 3.28644882 | 0 |
| 3.286692405 | 0 |
| 3.28693599 | 0 |
| 3.287179575 | 0 |
| 3.28742316 | 0 |
| 3.287666745 | 0 |
| 3.28791033 | 0 |
| 3.288153915 | 0 |
| 3.2883975 | 0 |
| 3.288641085 | 0 |
| 3.28888467 | 0 |
| 3.289128255 | 0 |
| 3.28937184 | 0 |
| 3.289615425 | 0 |
| 3.28985901 | 0 |
| 3.290102595 | 0 |
| 3.29034618 | 0 |
| 3.290589765 | 0 |
| 3.29083335 | 0 |
| 3.291076935 | 0 |
| 3.29132052 | 0 |
| 3.291564105 | 0 |
| 3.29180769 | 0 |
| 3.292051275 | 0 |
| 3.29229486 | 0 |
| 3.292538445 | 0 |
| 3.29278203 | 0 |
| 3.293025615 | 0 |
| 3.2932692 | 0 |
| 3.293512785 | 0 |
| 3.29375637 | 0 |
| 3.293999955 | 0 |
| 3.29424354 | 0 |
| 3.294487125 | 0 |
| 3.29473071 | 0 |
| 3.294974295 | 0 |
| 3.29521788 | 0 |
| 3.295461465 | 0 |
| 3.29570505 | 0 |
| 3.295948635 | 0 |
| 3.29619222 | 0 |
| 3.296435805 | 0 |
| 3.29667939 | 0 |
| 3.296922975 | 0 |
| 3.29716656 | 0 |
| 3.297410145 | 0 |
| 3.29765373 | 0 |
| 3.297897315 | 0 |
| 3.2981409 | 0 |
| 3.298384485 | 0 |
| 3.29862807 | 0 |
| 3.298871655 | 0 |
| 3.29911524 | 0 |
| 3.299358825 | 0 |
| 3.29960241 | 0 |
| 3.299845995 | 0 |
| 3.30008958 | 0 |
| 3.300333165 | 0 |
| 3.30057675 | 0 |
| 3.300820335 | 0 |
| 3.30106392 | 0 |
| 3.301307505 | 0 |
| 3.30155109 | 0 |
| 3.301794675 | 0 |
| 3.30203826 | 0 |
| 3.302281845 | 0 |
| 3.30252543 | 0 |
| 3.302769015 | 0 |
| 3.3030126 | 0 |
| 3.303256185 | 0 |
| 3.30349977 | 0 |
| 3.303743355 | 0 |
| 3.30398694 | 0 |
| 3.304230525 | 0 |
| 3.30447411 | 0 |
| 3.304717695 | 0 |
| 3.30496128 | 0 |
| 3.305204865 | 0 |
| 3.30544845 | 0 |
| 3.305692035 | 0 |
| 3.30593562 | 0 |
| 3.306179205 | 0 |
| 3.30642279 | 0 |
| 3.306666375 | 0 |
| 3.30690996 | 0 |
| 3.307153545 | 0 |
| 3.30739713 | 0 |
| 3.307640715 | 0 |
| 3.3078843 | 0 |
| 3.308127885 | 0 |
| 3.30837147 | 0 |
| 3.308615055 | 0 |
| 3.30885864 | 0 |
| 3.309102225 | 0 |
| 3.30934581 | 0 |
| 3.309589395 | 0 |
| 3.30983298 | 0 |
| 3.310076565 | 0 |
| 3.31032015 | 0 |
| 3.310563735 | 0 |
| 3.31080732 | 0 |
| 3.311050905 | 0 |
| 3.31129449 | 0 |
| 3.311538075 | 0 |
| 3.31178166 | 0 |
| 3.312025245 | 0 |
| 3.31226883 | 0 |
| 3.312512415 | 0 |
| 3.312756 | 0 |
| 3.312999585 | 0 |
| 3.31324317 | 0 |
| 3.313486755 | 0 |
| 3.31373034 | 0 |
| 3.313973925 | 0 |
| 3.31421751 | 0 |
| 3.314461095 | 0 |
| 3.31470468 | 0 |
| 3.314948265 | 0 |
| 3.31519185 | 0 |
| 3.315435435 | 0 |
| 3.31567902 | 0 |
| 3.315922605 | 0 |
| 3.31616619 | 0 |
| 3.316409775 | 0 |
| 3.31665336 | 0 |
| 3.316896945 | 0 |
| 3.31714053 | 0 |
| 3.317384115 | 0 |
| 3.3176277 | 0 |
| 3.317871285 | 0 |
| 3.31811487 | 0 |
| 3.318358455 | 0 |
| 3.31860204 | 0 |
| 3.318845625 | 0 |
| 3.31908921 | 0 |
| 3.319332795 | 0 |
| 3.31957638 | 0 |
| 3.319819965 | 0 |
| 3.32006355 | 0 |
| 3.320307135 | 0 |
| 3.32055072 | 0 |
| 3.320794305 | 0 |
| 3.32103789 | 0 |
| 3.321281475 | 0 |
| 3.32152506 | 0 |
| 3.321768645 | 0 |
| 3.32201223 | 0 |
| 3.322255815 | 0 |
| 3.3224994 | 0 |
| 3.322742985 | 0 |
| 3.32298657 | 0 |
| 3.323230155 | 0 |
| 3.32347374 | 0 |
| 3.323717325 | 0 |
| 3.32396091 | 0 |
| 3.324204495 | 0 |
| 3.32444808 | 0 |
| 3.324691665 | 0 |
| 3.32493525 | 0 |
| 3.325178835 | 0 |
| 3.32542242 | 0 |
| 3.325666005 | 0 |
| 3.32590959 | 0 |
| 3.326153175 | 0 |
| 3.32639676 | 0 |
| 3.326640345 | 0 |
| 3.32688393 | 0 |
| 3.327127515 | 0 |
| 3.3273711 | 0 |
| 3.327614685 | 0 |
| 3.32785827 | 0 |
| 3.328101855 | 0 |
| 3.32834544 | 0 |
| 3.328589025 | 0 |
| 3.32883261 | 0 |
| 3.329076195 | 0 |
| 3.32931978 | 0 |
| 3.329563365 | 0 |
| 3.32980695 | 0 |
| 3.330050535 | 0 |
| 3.33029412 | 0 |
| 3.330537705 | 0 |
| 3.33078129 | 0 |
| 3.331024875 | 0 |
| 3.33126846 | 0 |
| 3.331512045 | 0 |
| 3.33175563 | 0 |
| 3.331999215 | 0 |
| 3.3322428 | 0 |
| 3.332486385 | 0 |
| 3.33272997 | 0 |
| 3.332973555 | 0 |
| 3.33321714 | 0 |
| 3.333460725 | 0 |
| 3.33370431 | 0 |
| 3.333947895 | 0 |
| 3.33419148 | 0 |
| 3.334435065 | 0 |
| 3.33467865 | 0 |
| 3.334922235 | 0 |
| 3.33516582 | 0 |
| 3.335409405 | 0 |
| 3.33565299 | 0 |
| 3.335896575 | 0 |
| 3.33614016 | 0 |
| 3.336383745 | 0 |
| 3.33662733 | 0 |
| 3.336870915 | 0 |
| 3.3371145 | 0 |
| 3.337358085 | 0 |
| 3.33760167 | 0 |
| 3.337845255 | 0 |
| 3.33808884 | 0 |
| 3.338332425 | 0 |
| 3.33857601 | 0 |
| 3.338819595 | 0 |
| 3.33906318 | 0 |
| 3.339306765 | 0 |
| 3.33955035 | 0 |
| 3.339793935 | 0 |
| 3.34003752 | 0 |
| 3.340281105 | 0 |
| 3.34052469 | 0 |
| 3.340768275 | 0 |
| 3.34101186 | 0 |
| 3.341255445 | 0 |
| 3.34149903 | 0 |
| 3.341742615 | 0 |
| 3.3419862 | 0 |
| 3.342229785 | 0 |
| 3.34247337 | 0 |
| 3.342716955 | 0 |
| 3.34296054 | 0 |
| 3.343204125 | 0 |
| 3.34344771 | 0 |
| 3.343691295 | 0 |
| 3.34393488 | 0 |
| 3.344178465 | 0 |
| 3.34442205 | 0 |
| 3.344665635 | 0 |
| 3.34490922 | 0 |
| 3.345152805 | 0 |
| 3.34539639 | 0 |
| 3.345639975 | 0 |
| 3.34588356 | 0 |
| 3.346127145 | 0 |
| 3.34637073 | 0 |
| 3.346614315 | 0 |
| 3.3468579 | 0 |
| 3.347101485 | 0 |
| 3.34734507 | 0 |
| 3.347588655 | 0 |
| 3.34783224 | 0 |
| 3.348075825 | 0 |
| 3.34831941 | 0 |
| 3.348562995 | 0 |
| 3.34880658 | 0 |
| 3.349050165 | 0 |
| 3.34929375 | 0 |
| 3.349537335 | 0 |
| 3.34978092 | 0 |
| 3.350024505 | 0 |
| 3.35026809 | 0 |
| 3.350511675 | 0 |
| 3.35075526 | 0 |
| 3.350998845 | 0 |
| 3.35124243 | 0 |
| 3.351486015 | 0 |
| 3.3517296 | 0 |
| 3.351973185 | 0 |
| 3.35221677 | 0 |
| 3.352460355 | 0 |
| 3.35270394 | 0 |
| 3.352947525 | 0 |
| 3.35319111 | 0 |
| 3.353434695 | 0 |
| 3.35367828 | 0 |
| 3.353921865 | 0 |
| 3.35416545 | 0 |
| 3.354409035 | 0 |
| 3.35465262 | 0 |
| 3.354896205 | 0 |
| 3.35513979 | 0 |
| 3.355383375 | 0 |
| 3.35562696 | 0 |
| 3.355870545 | 0 |
| 3.35611413 | 0 |
| 3.356357715 | 0 |
| 3.3566013 | 0 |
| 3.356844885 | 0 |
| 3.35708847 | 0 |
| 3.357332055 | 0 |
| 3.35757564 | 0 |
| 3.357819225 | 0 |
| 3.35806281 | 0 |
| 3.358306395 | 0 |
| 3.35854998 | 0 |
| 3.358793565 | 0 |
| 3.35903715 | 0 |
| 3.359280735 | 0 |
| 3.35952432 | 0 |
| 3.359767905 | 0 |
| 3.36001149 | 0 |
| 3.360255075 | 0 |
| 3.36049866 | 0 |
| 3.360742245 | 0 |
| 3.36098583 | 0 |
| 3.361229415 | 0 |
| 3.361473 | 0 |
| 3.361716585 | 0 |
| 3.36196017 | 0 |
| 3.362203755 | 0 |
| 3.36244734 | 0 |
| 3.362690925 | 0 |
| 3.36293451 | 0 |
| 3.363178095 | 0 |
| 3.36342168 | 0 |
| 3.363665265 | 0 |
| 3.36390885 | 0 |
| 3.364152435 | −0.023 |
| 3.36439602 | −0.065 |
| 3.364639605 | −0.106 |
| 3.36488319 | −0.272 |
| 3.365126775 | −0.438 |
| 3.36537036 | −0.622 |
| 3.365613945 | −0.828 |
| 3.36585753 | −1.035 |
| 3.366101115 | −1.424 |
| 3.3663447 | −1.836 |
| 3.366588285 | −1.576 |
| 3.36683187 | 0.076 |
| 3.367075455 | 1.727 |
| 3.36731904 | 1.951 |
| 3.367562625 | 1.744 |
| 3.36780621 | 1.58 |
| 3.368049795 | 1.579 |
| 3.36829338 | 1.578 |
| 3.368536965 | 1.443 |
| 3.36878055 | 1.237 |
| 3.369024135 | 1.05 |
| 3.36926772 | 1.049 |
| 3.369511305 | 1.048 |
| 3.36975489 | 0.938 |
| 3.369998475 | 0.733 |
| 3.37024206 | 0.528 |
| 3.370485645 | 0.523 |
| 3.37072923 | 0.522 |
| 3.370972815 | 0.436 |
| 3.3712164 | 0.232 |
| 3.371459985 | 0.028 |
| 3.37170357 | 0 |
| 3.371947155 | 0 |
| 3.37219074 | 0 |
| 3.372434325 | 0 |
| 3.37267791 | 0 |
| 3.372921495 | 0 |
| 3.37316508 | 0 |
| 3.373408665 | 0 |
| 3.37365225 | 0 |
| 3.373895835 | 0 |
| 3.37413942 | 0 |
| 3.374383005 | 0 |
| 3.37462659 | 0 |
| 3.374870175 | 0 |
| 3.37511376 | 0 |
| 3.375357345 | 0 |
| 3.37560093 | 0 |
| 3.375844515 | 0 |
| 3.3760881 | 0 |
| 3.376331685 | 0 |
| 3.37657527 | 0.319 |
| 3.376818855 | 1.122 |
| 3.37706244 | 1.925 |
| 3.377306025 | 2.052 |
| 3.37754961 | 2.05 |
| 3.377793195 | 1.823 |
| 3.37803678 | 1.022 |
| 3.378280365 | 0.221 |
| 3.37852395 | 0 |
| 3.378767535 | 0 |
| 3.37901112 | 0 |
| 3.379254705 | 0 |
| 3.37949829 | 0 |
| 3.379741875 | −0.024 |
| 3.37998546 | −0.064 |
| 3.380229045 | −0.11 |
| 3.38047263 | −0.268 |
| 3.380716215 | −0.427 |
| 3.3809598 | −0.605 |
| 3.381203385 | −0.803 |
| 3.38144697 | −1.001 |
| 3.381690555 | −1.383 |
| 3.38193414 | −1.778 |
| 3.382177725 | −1.43 |
| 3.38242131 | 0.152 |
| 3.382664895 | 1.733 |
| 3.38290848 | 1.859 |
| 3.383152065 | 1.66 |
| 3.38339565 | 1.513 |
| 3.383639235 | 1.512 |
| 3.38388282 | 1.511 |
| 3.384126405 | 1.372 |
| 3.38436999 | 1.174 |
| 3.384613575 | 1.005 |
| 3.38485716 | 1.004 |
| 3.385100745 | 1.004 |
| 3.38534433 | 0.888 |
| 3.385587915 | 0.692 |
| 3.3858315 | 0.501 |
| 3.386075085 | 0.501 |
| 3.38631867 | 0.5 |
| 3.386562255 | 0.408 |
| 3.38680584 | 0.213 |
| 3.387049425 | 0.017 |
| 3.38729301 | 0 |
| 3.387536595 | 0 |
| 3.38778018 | 0 |
| 3.388023765 | 0 |
| 3.38826735 | 0 |
| 3.388510935 | 0 |
| 3.38875452 | 0 |
| 3.388998105 | 0 |
| 3.38924169 | 0 |
| 3.389485275 | 0 |
| 3.38972886 | 0 |
| 3.389972445 | 0 |
| 3.39021603 | 0 |
| 3.390459615 | 0 |
| 3.3907032 | 0 |
| 3.390946785 | 0 |
| 3.39119037 | 0 |
| 3.391433955 | 0 |
| 3.39167754 | 0 |
| 3.391921125 | 0 |
| 3.39216471 | 0.344 |
| 3.392408295 | 1.113 |
| 3.39265188 | 1.881 |
| 3.392895465 | 1.964 |
| 3.39313905 | 1.963 |
| 3.393382635 | 1.707 |
| 3.39362622 | 0.94 |
| 3.393869805 | 0.173 |
| 3.39411339 | 0 |
| 3.394356975 | 0 |
| 3.39460056 | 0 |
| 3.394844145 | 0 |
| 3.39508773 | 0 |
| 3.395331315 | −0.025 |
| 3.3955749 | −0.063 |
| 3.395818485 | −0.113 |
| 3.39606207 | −0.265 |
| 3.396305655 | −0.417 |
| 3.39654924 | −0.589 |
| 3.396792825 | −0.778 |
| 3.39703641 | −0.968 |
| 3.397279995 | −1.343 |
| 3.39752358 | −1.721 |
| 3.397767165 | −1.293 |
| 3.39801075 | 0.221 |
| 3.398254335 | 1.735 |
| 3.39849792 | 1.77 |
| 3.398741505 | 1.58 |
| 3.39898509 | 1.448 |
| 3.399228675 | 1.447 |
| 3.39947226 | 1.446 |
| 3.399715845 | 1.304 |
| 3.39995943 | 1.115 |
| 3.400203015 | 0.962 |
| 3.4004466 | 0.962 |
| 3.400690185 | 0.961 |
| 3.40093377 | 0.841 |
| 3.401177355 | 0.653 |
| 3.40142094 | 0.479 |
| 3.401664525 | 0.479 |
| 3.40190811 | 0.479 |
| 3.402151695 | 0.381 |
| 3.40239528 | 0.194 |
| 3.402638865 | 0.007 |
| 3.40288245 | 0 |
| 3.403126035 | 0 |
| 3.40336962 | 0 |
| 3.403613205 | 0 |
| 3.40385679 | 0 |
| 3.404100375 | 0 |
| 3.40434396 | 0 |
| 3.404587545 | 0 |
| 3.40483113 | 0 |
| 3.405074715 | 0 |
| 3.4053183 | 0 |
| 3.405561885 | 0 |
| 3.40580547 | 0 |
| 3.406049055 | 0 |
| 3.40629264 | 0 |
| 3.406536225 | 0 |
| 3.40677981 | 0 |
| 3.407023395 | 0 |
| 3.40726698 | 0 |
| 3.407510565 | 0 |
| 3.40775415 | 0.366 |
| 3.407997735 | 1.102 |
| 3.40824132 | 1.837 |
| 3.408484905 | 1.88 |
| 3.40872849 | 1.879 |
| 3.408972075 | 1.598 |
| 3.40921566 | 0.864 |
| 3.409459245 | 0.129 |
| 3.40970283 | 0 |
| 3.409946415 | 0 |
| 3.41019 | 0 |
| 3.410433585 | 0 |
| 3.41067717 | 0 |
| 3.410920755 | −0.026 |
| 3.41116434 | −0.062 |
| 3.411407925 | −0.115 |
| 3.41165151 | −0.261 |
| 3.411895095 | −0.406 |
| 3.41213868 | −0.573 |
| 3.412382265 | −0.754 |
| 3.41262585 | −0.942 |
| 3.412869435 | −1.304 |
| 3.41311302 | −1.666 |
| 3.413356605 | −1.166 |
| 3.41360019 | 0.284 |
| 3.413843775 | 1.734 |
| 3.41408736 | 1.686 |
| 3.414330945 | 1.504 |
| 3.41457453 | 1.387 |
| 3.414818115 | 1.386 |
| 3.4150617 | 1.385 |
| 3.415305285 | 1.239 |
| 3.41554887 | 1.058 |
| 3.415792455 | 0.921 |
| 3.41603604 | 0.921 |
| 3.416279625 | 0.92 |
| 3.41652321 | 0.796 |
| 3.416766795 | 0.616 |
| 3.41701038 | 0.459 |
| 3.417253965 | 0.459 |
| 3.41749755 | 0.459 |
| 3.417741135 | 0.356 |
| 3.41798472 | 0.177 |
| 3.418228305 | 0 |
| 3.41847189 | 0 |
| 3.418715475 | 0 |
| 3.41895906 | 0 |
| 3.419202645 | 0 |
| 3.41944623 | 0 |
| 3.419689815 | 0 |
| 3.4199334 | 0 |
| 3.420176985 | 0 |
| 3.42042057 | 0 |
| 3.420664155 | 0 |
| 3.42090774 | 0 |
| 3.421151325 | 0 |
| 3.42139491 | 0 |
| 3.421638495 | 0 |
| 3.42188208 | 0 |
| 3.422125665 | 0 |
| 3.42236925 | 0 |
| 3.422612835 | 0 |
| 3.42285642 | 0 |
| 3.423100005 | 0 |
| 3.42334359 | 0.386 |
| 3.423587175 | 1.091 |
| 3.42383076 | 1.796 |
| 3.424074345 | 1.802 |
| 3.42431793 | 1.8 |
| 3.424561515 | 1.723 |
| 3.4248051 | 1.547 |
| 3.425048685 | 1.37 |
| 3.42529227 | 1.347 |
| 3.425535855 | 1.346 |
| 3.42577944 | 1.29 |
| 3.426023025 | 1.114 |
| 3.42626661 | 0.938 |
| 3.426510195 | 0.895 |
| 3.42675378 | 0.894 |
| 3.426997365 | 0.859 |
| 3.42724095 | 0.684 |
| 3.427484535 | 0.509 |
| 3.42772812 | 0.446 |
| 3.427971705 | 0.446 |
| 3.42821529 | 0.431 |
| 3.428458875 | 0.257 |
| 3.42870246 | 0.083 |
| 3.428946045 | 0 |
| 3.42918963 | 0 |
| 3.429433215 | 0 |
| 3.4296768 | 0 |
| 3.429920385 | 0 |
| 3.43016397 | 0 |
| 3.430407555 | 0 |
| 3.43065114 | 0 |
| 3.430894725 | 0 |
| 3.43113831 | 0 |
| 3.431381895 | 0 |
| 3.43162548 | 0 |
| 3.431869065 | 0 |
| 3.43211265 | 0 |
| 3.432356235 | 0 |
| 3.43259982 | 0 |
| 3.432843405 | 0 |
| 3.43308699 | 0 |
| 3.433330575 | 0 |
| 3.43357416 | 0 |
| 3.433817745 | 0 |
| 3.43406133 | 0 |
| 3.434304915 | 0 |
| 3.4345485 | 0 |
| 3.434792085 | 0 |
| 3.43503567 | 0 |
| 3.435279255 | 0 |
| 3.43552284 | 0 |
| 3.435766425 | 0 |
| 3.43601001 | 0 |
| 3.436253595 | 0 |
| 3.43649718 | 0 |
| 3.436740765 | 0 |
| 3.43698435 | 0 |
| 3.437227935 | 0 |
| 3.43747152 | 0 |
| 3.437715105 | 0 |
| 3.43795869 | 0 |
| 3.438202275 | 0 |
| 3.43844586 | 0 |
| 3.438689445 | 0 |
| 3.43893303 | 0 |
| 3.439176615 | 0 |
| 3.4394202 | 0 |
| 3.439663785 | 0 |
| 3.43990737 | 0 |
| 3.440150955 | 0 |
| 3.44039454 | 0 |
| 3.440638125 | 0 |
| 3.44088171 | 0 |
| 3.441125295 | 0 |
| 3.44136888 | 0 |
| 3.441612465 | 0 |
| 3.44185605 | 0 |
| 3.442099635 | 0 |
| 3.44234322 | 0 |
| 3.442586805 | 0 |
| 3.44283039 | 0 |
| 3.443073975 | 0 |
| 3.44331756 | 0 |
| 3.443561145 | 0 |
| 3.44380473 | 0 |
| 3.444048315 | 0 |
| 3.4442919 | 0 |
| 3.444535485 | 0 |
| 3.44477907 | 0 |
| 3.445022655 | 0 |
| 3.44526624 | 0 |
| 3.445509825 | 0 |
| 3.44575341 | 0 |
| 3.445996995 | 0 |
| 3.44624058 | 0 |
| 3.446484165 | 0 |
| 3.44672775 | 0 |
| 3.446971335 | 0 |
| 3.44721492 | 0 |
| 3.447458505 | 0 |
| 3.44770209 | 0 |
| 3.447945675 | 0 |
| 3.44818926 | 0 |
| 3.448432845 | 0 |
| 3.44867643 | 0 |
| 3.448920015 | 0 |
| 3.4491636 | 0 |
| 3.449407185 | 0 |
| 3.44965077 | 0 |
| 3.449894355 | 0 |
| 3.45013794 | 0 |
| 3.450381525 | 0 |
| 3.45062511 | 0 |
| 3.450868695 | 0 |
| 3.45111228 | 0 |
| 3.451355865 | 0 |
| 3.45159945 | 0 |
| 3.451843035 | 0 |
| 3.45208662 | 0 |
| 3.452330205 | 0 |
| 3.45257379 | 0 |
| 3.452817375 | 0 |
| 3.45306096 | 0 |
| 3.453304545 | 0 |
| 3.45354813 | 0 |
| 3.453791715 | 0 |
| 3.4540353 | 0 |
| 3.454278885 | 0 |
| 3.45452247 | 0 |
| 3.454766055 | 0 |
| 3.45500964 | 0 |
| 3.455253225 | 0 |
| 3.45549681 | 0 |
| 3.455740395 | 0 |
| 3.45598398 | 0 |
| 3.456227565 | 0 |
| 3.45647115 | 0 |
| 3.456714735 | 0 |
| 3.45695832 | 0 |
| 3.457201905 | 0 |
| 3.45744549 | 0 |
| 3.457689075 | 0 |
| 3.45793266 | 0 |
| 3.458176245 | 0 |
| 3.45841983 | 0 |
| 3.458663415 | 0 |
| 3.458907 | 0 |
| 3.459150585 | 0 |
| 3.45939417 | 0 |
| 3.459637755 | 0 |
| 3.45988134 | 0 |
| 3.460124925 | 0 |
| 3.46036851 | 0 |
| 3.460612095 | 0 |
| 3.46085568 | 0 |
| 3.461099265 | 0 |
| 3.46134285 | 0 |
| 3.461586435 | 0 |
| 3.46183002 | 0 |
| 3.462073605 | 0 |
| 3.44231719 | 0 |
| 3.462560775 | 0 |
| 3.46280436 | 0 |
| 3.463047945 | 0 |
| 3.46329153 | 0 |
| 3.463535115 | 0 |
| 3.4637787 | 0 |
| 3.464022285 | 0 |
| 3.46426587 | 0 |
| 3.464509455 | 0 |
| 3.46475304 | 0 |
| 3.464996625 | 0 |
| 3.46524021 | 0 |
| 3.465483795 | 0 |
| 3.46572738 | 0 |
| 3.465970965 | 0 |
| 3.46621455 | 0 |
| 3.466458135 | 0 |
| 3.46670172 | 0 |
| 3.466945305 | 0 |
| 3.46718889 | 0 |
| 3.467432475 | 0 |
| 3.46767606 | 0 |
| 3.467919645 | 0 |
| 3.46816323 | 0 |
| 3.468406815 | 0 |
| 3.4686504 | 0 |
| 3.468893985 | 0 |
| 3.46913757 | 0 |
| 3.469381155 | 0 |
| 3.46962474 | 0 |
| 3.469868325 | 0 |
| 3.47011191 | 0 |
| 3.470355495 | 0 |
| 3.47059908 | 0 |
| 3.470842665 | 0 |
| 3.47108625 | 0 |
| 3.471329835 | 0 |
| 3.47157342 | 0 |
| 3.471817005 | 0 |
| 3.47206059 | 0 |
| 3.472304175 | 0 |
| 3.47254776 | 0 |
| 3.472791345 | 0 |
| 3.47303493 | 0 |
| 3.473278515 | 0 |
| 3.4735221 | 0 |
| 3.473765685 | 0 |
| 3.47400927 | 0 |
| 3.474252855 | 0 |
| 3.47449644 | 0 |
| 3.474740025 | 0 |
| 3.47498361 | 0 |
| 3.475227195 | 0 |
| 3.47547078 | 0 |
| 3.475714365 | 0 |
| 3.47595795 | 0 |
| 3.476201535 | 0 |
| 3.47644512 | 0 |
| 3.476688705 | 0 |
| 3.47693229 | 0 |
| 3.477175875 | 0 |
| 3.47741946 | 0 |
| 3.477663045 | 0 |
| 3.47790663 | 0 |
| 3.478150215 | 0 |
| 3.4783938 | 0 |
| 3.478637385 | 0 |
| 3.47888097 | 0 |
| 3.479124555 | 0 |
| 3.47936814 | 0 |
| 3.479611725 | 0 |
| 3.47985531 | 0 |
| 3.480098895 | 0 |
| 3.48034248 | 0 |
| 3.480586065 | 0 |
| 3.48082965 | 0 |
| 3.481073235 | 0 |
| 3.48131682 | 0 |
| 3.481560405 | 0 |
| 3.48180399 | 0 |
| 3.482047575 | 0 |
| 3.48229116 | 0 |
| 3.482534745 | 0 |
| 3.48277833 | 0 |
| 3.483021915 | 0 |
| 3.4832655 | 0 |
| 3.483509085 | 0 |
| 3.48375267 | 0 |
| 3.483996255 | 0 |
| 3.48423984 | 0 |
| 3.484483425 | 0 |
| 3.48472701 | 0 |
| 3.484970595 | 0 |
| 3.48521418 | 0 |
| 3.485457765 | 0 |
| 3.48570135 | 0 |
| 3.485944935 | 0 |
| 3.48618852 | 0 |
| 3.486432105 | 0 |
| 3.48667569 | 0 |
| 3.486919275 | 0 |
| 3.48716286 | 0 |
| 3.487406445 | 0 |
| 3.48765003 | 0 |
| 3.487893615 | 0 |
| 3.4881372 | 0 |
| 3.488380785 | 0 |
| 3.48862437 | 0 |
| 3.488867955 | 0 |
| 3.48911154 | 0 |
| 3.489355125 | 0 |
| 3.48959871 | 0 |
| 3.489842295 | 0 |
| 3.49008588 | 0 |
| 3.490329465 | 0 |
| 3.49057305 | 0 |
| 3.490816635 | 0 |
| 3.49106022 | 0 |
| 3.491303805 | 0 |
| 3.49154739 | 0 |
| 3.491790975 | 0 |
| 3.49203456 | 0 |
| 3.492278145 | 0 |
| 3.49252173 | 0 |
| 3.492765315 | 0 |
| 3.4930089 | 0 |
| 3.493252485 | 0 |
| 3.49349607 | 0 |
| 3.493739655 | 0 |
| 3.49398324 | 0 |
| 3.494226825 | 0 |
| 3.49447041 | 0 |
| 3.494713995 | 0 |
| 3.49495758 | 0 |
| 3.495201165 | 0 |
| 3.49544475 | 0 |
| 3.495688335 | 0 |
| 3.49593192 | 0 |
| 3.496175505 | 0 |
| 3.49641909 | 0 |
| 3.496662675 | 0 |
| 3.49690626 | 0 |
| 3.497149845 | 0 |
| 3.49739343 | 0 |
| 3.497637015 | 0 |
| 3.4978806 | 0 |
| 3.498124185 | 0 |
| 3.49836777 | 0 |
| 3.498611355 | 0 |
| 3.49885494 | 0 |
| 3.499098525 | 0 |
| 3.49934211 | 0 |
| 3.499585695 | 0 |
| 3.49982928 | 0 |
| 3.500072865 | 0 |
| 3.50031645 | 0 |
| 3.500560035 | 0 |
| 3.50080362 | 0 |
| 3.501047205 | 0 |
| 3.50129079 | 0 |
| 3.501534375 | 0 |
| 3.50177796 | 0 |
| 3.502021545 | 0 |
| 3.50226513 | 0 |
| 3.502508715 | 0 |
| 3.5027523 | 0 |
| 3.502995885 | 0 |
| 3.50323947 | 0 |
| 3.503483055 | 0 |
| 3.50372664 | 0 |
| 3.503970225 | 0 |
| 3.50421381 | 0 |
| 3.504457395 | 0 |
| 3.50470098 | 0 |
| 3.504944565 | 0 |
| 3.50518815 | 0 |
| 3.505431735 | 0 |
| 3.50567532 | 0 |
| 3.505918905 | 0 |
| 3.50616249 | 0 |
| 3.506406075 | 0 |
| 3.50664966 | 0 |
| 3.506893245 | 0 |
| 3.50713683 | 0 |
| 3.507380415 | 0 |
| 3.507624 | 0 |
| 3.507867585 | 0 |
| 3.50811117 | 0 |
| 3.508354755 | 0 |
| 3.50859834 | 0 |
| 3.508841925 | 0 |
| 3.50908551 | 0 |
| 3.509329095 | 0 |
| 3.50957268 | 0 |
| 3.509816265 | 0 |
| 3.51005985 | 0 |
| 3.510303435 | 0 |
| 3.51054702 | 0 |
| 3.510790605 | 0 |
| 3.51103419 | 0 |
| 3.511277775 | 0 |
| 3.51152136 | 0 |
| 3.511764945 | 0 |
| 3.51200853 | 0 |
| 3.512252115 | 0 |
| 3.5124957 | 0 |
| 3.512739285 | 0 |
| 3.51298287 | 0 |
| 3.513226455 | 0 |
| 3.51347004 | 0 |
| 3.513713625 | 0 |
| 3.51395721 | 0 |
| 3.514200795 | 0 |
| 3.51444438 | 0 |
| 3.514687965 | 0 |
| 3.51493155 | 0 |
| 3.515175135 | 0 |
| 3.51541872 | 0 |
| 3.515662305 | 0 |
| 3.51590589 | 0 |
| 3.516149475 | 0 |
| 3.51639306 | 0 |
| 3.516636645 | 0 |
| 3.51688023 | 0 |
| 3.517123815 | 0 |
| 3.5173674 | 0 |
| 3.517610985 | 0 |
| 3.51785457 | 0 |
| 3.518098155 | 0 |
| 3.51834174 | 0 |
| 3.518585325 | 0 |
| 3.51882891 | 0 |
| 3.519072495 | 0 |
| 3.51931608 | 0 |
| 3.519559665 | 0 |
| 3.51980325 | 0 |
| 3.520046835 | 0 |
| 3.52029042 | 0 |
| 3.520534005 | 0 |
| 3.52077759 | 0 |
| 3.521021175 | 0 |
| 3.52126476 | 0 |
| 3.521508345 | 0 |
| 3.52175193 | 0 |
| 3.521995515 | 0 |
| 3.5222391 | 0 |
| 3.522482685 | 0 |
| 3.52272627 | 0 |
| 3.522969855 | 0 |
| 3.52321344 | 0 |
| 3.523457025 | 0 |
| 3.52370061 | 0 |
| 3.523944195 | 0 |
| 3.52418778 | 0 |
| 3.524431365 | 0 |
| 3.52467495 | 0 |
| 3.524918535 | 0 |
| 3.52516212 | 0 |
| 3.525405705 | 0 |
| 3.52564929 | 0 |
| 3.525892875 | 0 |
| 3.52613646 | 0 |
| 3.526380045 | 0 |
| 3.52662363 | 0 |
| 3.526867215 | 0 |
| 3.5271108 | 0 |
| 3.527354385 | 0 |
| 3.52759797 | 0 |
| 3.527841555 | 0 |
| 3.52808514 | 0 |
| 3.528328725 | 0 |
| 3.52857231 | 0 |
| 3.528815895 | 0 |
| 3.52905948 | 0 |
| 3.529303065 | 0 |
| 3.52954665 | 0 |
| 3.529790235 | 0 |
| 3.53003382 | 0 |
| 3.530277405 | 0 |
| 3.53052099 | 0 |
| 3.530764575 | 0 |
| 3.53100816 | 0 |
| 3.531251745 | 0 |
| 3.53149533 | 0 |
| 3.531738915 | 0 |
| 3.5319825 | 0 |
| 3.532226085 | 0 |
| 3.53246967 | 0 |
| 3.532713255 | 0 |
| 3.53295684 | 0 |
| 3.533200425 | 0 |
| 3.53344401 | 0 |
| 3.533687595 | 0 |
| 3.53393118 | 0 |
| 3.534174765 | 0 |
| 3.53441835 | 0 |
| 3.534661935 | 0 |
| 3.53490552 | 0 |
| 3.535149105 | 0 |
| 3.53539269 | 0 |
| 3.535636275 | 0 |
| 3.53587986 | 0 |
| 3.536123445 | 0 |
| 3.53636703 | 0 |
| 3.536610615 | 0 |
| 3.5368542 | 0 |
| 3.537097785 | 0 |
| 3.53734137 | 0 |
| 3.537584955 | 0 |
| 3.53782854 | 0 |
| 3.538072125 | 0 |
| 3.53831571 | 0 |
| 3.538559295 | 0 |
| 3.53880288 | 0 |
| 3.539046465 | 0 |
| 3.53929005 | 0 |
| 3.539533635 | 0 |
| 3.53977722 | 0 |
| 3.540020805 | 0 |
| 3.54026439 | 0 |
| 3.540507975 | 0 |
| 3.54075156 | 0 |
| 3.540995145 | 0 |
| 3.54123873 | 0 |
| 3.541482315 | 0 |
| 3.5417259 | 0 |
| 3.541969485 | 0 |
| 3.54221307 | 0 |
| 3.542456655 | 0 |
| 3.54270024 | 0 |
| 3.542943825 | 0 |
| 3.54318741 | 0 |
| 3.543430995 | 0 |
| 3.54367458 | 0 |
| 3.543918165 | 0 |
| 3.54416175 | 0 |
| 3.544405335 | 0 |
| 3.54464892 | 0 |
| 3.544892505 | 0 |
| 3.54513609 | 0 |
| 3.545379675 | 0 |
| 3.54562326 | 0 |
| 3.545866845 | 0 |
| 3.54611043 | 0 |
| 3.546354015 | 0 |
| 3.5465976 | 0 |
| 3.546841185 | 0 |
| 3.54708477 | 0 |
| 3.547328355 | 0 |
| 3.54757194 | 0 |
| 3.547815525 | 0 |
| 3.54805911 | 0 |
| 3.548302695 | 0 |
| 3.54854628 | 0 |
| 3.548789865 | 0 |
| 3.54903345 | 0 |
| 3.549277035 | 0 |
| 3.54952062 | 0 |
| 3.549764205 | 0 |
| 3.55000779 | 0 |
| 3.550251375 | 0 |
| 3.55049496 | 0 |
| 3.550738545 | 0 |
| 3.55098213 | −0.004 |
| 3.551225715 | −0.032 |
| 3.5514693 | −0.059 |
| 3.551712885 | −0.136 |
| 3.55195647 | −0.246 |
| 3.552200055 | −0.357 |
| 3.55244364 | −0.494 |
| 3.552687225 | −0.632 |
| 3.55293081 | −0.835 |
| 3.553174395 | −1.11 |
| 3.55341798 | −1.384 |
| 3.553661565 | −0.388 |
| 3.55390515 | 0.71 |
| 3.554148735 | 1.353 |
| 3.55439232 | 1.215 |
| 3.554635905 | 1.078 |
| 3.55487949 | 1.052 |
| 3.555123075 | 1.052 |
| 3.55536666 | 1.017 |
| 3.555610245 | 0.88 |
| 3.55585383 | 0.743 |
| 3.556097415 | 0.7 |
| 3.556341 | 0.7 |
| 3.556584585 | 0.682 |
| 3.55682817 | 0.545 |
| 3.557071755 | 0.408 |
| 3.55731534 | 0.35 |
| 3.557558925 | 0.35 |
| 3.55780251 | 0.347 |
| 3.558046095 | 0.21 |
| 3.55828968 | 0.074 |
| 3.558533265 | 0 |
| 3.55877685 | 0 |
| 3.559020435 | 0 |
| 3.55926402 | 0 |
| 3.559507605 | 0 |
| 3.55975119 | 0 |
| 3.559994775 | 0 |
| 3.56023836 | 0 |
| 3.560481945 | 0 |
| 3.56072553 | 0 |
| 3.560969115 | 0 |
| 3.5612127 | 0 |
| 3.561456285 | 0 |
| 3.56169987 | 0 |
| 3.561943455 | 0 |
| 3.56218704 | 0 |
| 3.562430625 | 0 |
| 3.56267421 | 0 |
| 3.562917795 | 0 |
| 3.56316138 | 0 |
| 3.563404965 | 0 |
| 3.56364855 | 0.543 |
| 3.563892135 | 1.088 |
| 3.56413572 | 1.392 |
| 3.564379305 | 1.392 |
| 3.56462289 | 1.392 |
| 3.564866475 | 0.912 |
| 3.56511006 | 0.368 |
| 3.565353645 | 0 |
| 3.56559723 | 0 |
| 3.565840815 | 0 |
| 3.5660844 | 0 |
| 3.566327985 | 0 |
| 3.56657157 | −0.006 |
| 3.566815155 | −0.033 |
| 3.56705874 | −0.06 |
| 3.567302325 | −0.14 |
| 3.56754591 | −0.249 |
| 3.567789495 | −0.36 |
| 3.56803308 | −0.495 |
| 3.568276665 | −0.631 |
| 3.56852025 | −0.839 |
| 3.568763835 | −1.11 |
| 3.56900742 | −1.381 |
| 3.569251005 | −0.329 |
| 3.56949459 | 0.756 |
| 3.569738175 | 1.33 |
| 3.56998176 | 1.195 |
| 3.570225345 | 1.059 |
| 3.57046893 | 1.04 |
| 3.570712515 | 1.04 |
| 3.5709561 | 0.999 |
| 3.571199685 | 0.863 |
| 3.57144327 | 0.728 |
| 3.571686855 | 0.693 |
| 3.57193044 | 0.693 |
| 3.572174025 | 0.667 |
| 3.57241761 | 0.532 |
| 3.572661195 | 0.397 |
| 3.57290478 | 0.346 |
| 3.573148365 | 0.346 |
| 3.57339195 | 0.337 |
| 3.573635535 | 0.201 |
| 3.57387912 | 0.066 |
| 3.574122705 | 0 |
| 3.57436629 | 0 |
| 3.574609875 | 0 |
| 3.57485346 | 0 |
| 3.575097045 | 0 |
| 3.57534063 | 0 |
| 3.575584215 | 0 |
| 3.5758278 | 0 |
| 3.576071385 | 0 |
| 3.57631497 | 0 |
| 3.576558555 | 0 |
| 3.57680214 | 0 |
| 3.577045725 | 0 |
| 3.57728931 | 0 |
| 3.577532895 | 0 |
| 3.57777648 | 0 |
| 3.578020065 | 0 |
| 3.57826365 | 0 |
| 3.578507235 | 0 |
| 3.57875082 | 0 |
| 3.578994405 | 0.026 |
| 3.57923799 | 0.565 |
| 3.579481575 | 1.105 |
| 3.57972516 | 1.38 |
| 3.579968745 | 1.379 |
| 3.58021233 | 1.379 |
| 3.580455915 | 0.877 |
| 3.5806995 | 0.338 |
| 3.580943085 | 0 |
| 3.58118667 | 0 |
| 3.581430255 | 0 |
| 3.58167384 | 0 |
| 3.581917425 | 0 |
| 3.58216101 | −0.007 |
| 3.582404595 | −0.034 |
| 3.58264818 | −0.061 |
| 3.582891765 | −0.144 |
| 3.58313535 | −0.252 |
| 3.583378935 | −0.363 |
| 3.58362252 | −0.498 |
| 3.583866105 | −0.633 |
| 3.58410969 | −0.846 |
| 3.584353275 | −1.115 |
| 3.58459686 | −1.349 |
| 3.584840445 | −0.272 |
| 3.58508403 | 0.804 |
| 3.585327615 | 1.313 |
| 3.5855712 | 1.179 |
| 3.585814785 | 1.044 |
| 3.58605837 | 1.032 |
| 3.586301955 | 1.032 |
| 3.58654554 | 0.984 |
| 3.586789125 | 0.85 |
| 3.58703271 | 0.715 |
| 3.587276295 | 0.688 |
| 3.58751988 | 0.688 |
| 3.587763465 | 0.656 |
| 3.58800705 | 0.521 |
| 3.588250635 | 0.387 |
| 3.58849422 | 0.344 |
| 3.588737805 | 0.344 |
| 3.58898139 | 0.327 |
| 3.589224975 | 0.193 |
| 3.58946856 | 0.059 |
| 3.589712145 | 0 |
| 3.58995573 | 0 |
| 3.590199315 | 0 |
| 3.5904429 | 0 |
| 3.590686485 | 0 |
| 3.59093007 | 0 |
| 3.591173655 | 0 |
| 3.59141724 | 0 |
| 3.591660825 | 0 |
| 3.59190441 | 0 |
| 3.592147995 | 0 |
| 3.59239158 | 0 |
| 3.592635165 | 0 |
| 3.59287875 | 0 |
| 3.593122335 | 0 |
| 3.59336592 | 0 |
| 3.593609505 | 0 |
| 3.59385309 | 0 |
| 3.594096675 | 0 |
| 3.59434026 | 0 |
| 3.594583845 | 0.053 |
| 3.59482743 | 0.589 |
| 3.595071015 | 1.125 |
| 3.5953146 | 1.372 |
| 3.595558185 | 1.372 |
| 3.59580177 | 1.371 |
| 3.596045355 | 0.845 |
| 3.59628894 | 0.309 |
| 3.596532525 | 0 |
| 3.59677611 | 0 |
| 3.597019695 | 0 |
| 3.59726328 | 0 |
| 3.597506865 | 0 |
| 3.59775045 | −0.008 |
| 3.597994035 | −0.035 |
| 3.59823762 | −0.062 |
| 3.598481205 | −0.149 |
| 3.59872479 | −0.256 |
| 3.598968375 | −0.368 |
| 3.59921196 | −0.502 |
| 3.599455545 | −0.636 |
| 3.59969913 | −0.855 |
| 3.599942715 | −1.123 |
| 3.6001863 | −1.289 |
| 3.600429885 | −0.218 |
| 3.60067347 | 0.854 |
| 3.600917055 | 1.3 |
| 3.60116064 | 1.167 |
| 3.601404225 | 1.033 |
| 3.60164781 | 1.027 |
| 3.601891395 | 1.027 |
| 3.60213498 | 0.973 |
| 3.602378565 | 0.839 |
| 3.60262215 | 0.706 |
| 3.602865735 | 0.685 |
| 3.60310932 | 0.685 |
| 3.603352905 | 0.646 |
| 3.60359649 | 0.512 |
| 3.603840075 | 0.379 |
| 3.60408366 | 0.342 |
| 3.604327245 | 0.342 |
| 3.60457083 | 0.319 |
| 3.604814415 | 0.186 |
| 3.605058 | 0.052 |
| 3.605301585 | 0 |
| 3.60554517 | 0 |
| 3.605788755 | 0 |
| 3.60603234 | 0 |
| 3.606275925 | 0 |
| 3.60651951 | 0 |
| 3.606763095 | 0 |
| 3.60700668 | 0 |
| 3.607250265 | 0 |
| 3.60749385 | 0 |
| 3.607737435 | 0 |
| 3.60798102 | 0 |
| 3.608224605 | 0 |
| 3.60846819 | 0 |
| 3.608711775 | 0 |
| 3.60895536 | 0 |
| 3.609198945 | 0 |
| 3.60944253 | 0 |
| 3.609686115 | 0 |
| 3.6099297 | 0 |
| 3.610173285 | 0.079 |
| 3.61041687 | 0.614 |
| 3.610660455 | 1.148 |
| 3.61090404 | 1.368 |
| 3.611147625 | 1.368 |
| 3.61139121 | 1.35 |
| 3.611634795 | 0.816 |
| 3.61187838 | 0.281 |
| 3.612121965 | 0 |
| 3.61236555 | 0 |
| 3.612609135 | 0 |
| 3.61285272 | 0 |
| 3.613096305 | 0 |
| 3.61333989 | 0 |
| 3.613583475 | 0 |
| 3.61382706 | 0 |
| 3.614070645 | 0 |
| 3.61431423 | 0 |
| 3.614557815 | 0 |
| 3.6148014 | 0 |
| 3.615044985 | 0 |
| 3.61528857 | 0 |
| 3.615532155 | 0 |
| 3.61577574 | 0 |
| 3.616019325 | 0 |
| 3.61626291 | 0 |
| 3.616506495 | 0 |
| 3.61675008 | 0 |
| 3.616993665 | 0 |
| 3.61723725 | 0 |
| 3.617480835 | 0 |
| 3.61772442 | 0 |
| 3.617968005 | 0 |
| 3.61821159 | 0 |
| 3.618455175 | 0 |
| 3.61869876 | 0 |
| 3.618942345 | 0 |
| 3.61918593 | 0 |
| 3.619429515 | 0 |
| 3.6196731 | 0 |
| 3.619916685 | 0 |
| 3.62016027 | 0 |
| 3.620403855 | 0 |
| 3.62064744 | 0 |
| 3.620891025 | 0 |
| 3.62113461 | 0 |
| 3.621378195 | 0 |
| 3.62162178 | 0 |
| 3.621865365 | 0 |
| 3.62210895 | 0 |
| 3.622352535 | 0 |
| 3.62259612 | 0 |
| 3.622839705 | 0 |
| 3.62308329 | 0 |
| 3.623326875 | 0 |
| 3.62357046 | 0 |
| 3.623814045 | 0 |
| 3.62405763 | 0 |
| 3.624301215 | 0 |
| 3.6245448 | 0 |
| 3.624788385 | 0 |
| 3.62503197 | 0 |
| 3.625275555 | 0 |
| 3.62551914 | 0 |
| 3.625762725 | 0 |
| 3.62600631 | 0 |
| 3.626249895 | 0 |
| 3.62649348 | 0 |
| 3.626737065 | 0 |
| 3.62698065 | 0 |
| 3.627224235 | 0 |
| 3.62746782 | 0 |
| 3.627711405 | 0 |
| 3.62795499 | 0 |
| 3.628198575 | 0 |
| 3.62844216 | 0 |
| 3.628685745 | 0 |
| 3.62892933 | −0.011 |
| 3.629172915 | −0.038 |
| 3.6294165 | −0.064 |
| 3.629660085 | −0.159 |
| 3.62990367 | −0.266 |
| 3.630147255 | −0.381 |
| 3.63039084 | −0.514 |
| 3.630634425 | −0.648 |
| 3.63087801 | −0.88 |
| 3.631121595 | −1.147 |
| 3.63136518 | −1.179 |
| 3.631608765 | −0.11 |
| 3.63185235 | 0.959 |
| 3.632095935 | 1.284 |
| 3.63233952 | 1.151 |
| 3.632583105 | 1.025 |
| 3.63282669 | 1.025 |
| 3.633070275 | 1.025 |
| 3.63331386 | 0.958 |
| 3.633557445 | 0.825 |
| 3.63380103 | 0.691 |
| 3.634044615 | 0.684 |
| 3.6342882 | 0.684 |
| 3.634531785 | 0.632 |
| 3.63477537 | 0.498 |
| 3.635018955 | 0.365 |
| 3.63526254 | 0.342 |
| 3.635506125 | 0.342 |
| 3.63574971 | 0.306 |
| 3.635993295 | 0.172 |
| 3.63623688 | 0.038 |
| 3.636480465 | 0 |
| 3.63672405 | 0 |
| 3.636967635 | 0 |
| 3.63721122 | 0 |
| 3.637454805 | 0 |
| 3.63769839 | 0 |
| 3.637941975 | 0 |
| 3.63818556 | 0 |
| 3.638429145 | 0 |
| 3.63867273 | 0 |
| 3.638916315 | 0 |
| 3.6391599 | 0 |
| 3.639403485 | 0 |
| 3.63964707 | 0 |
| 3.639890655 | 0 |
| 3.64013424 | 0 |
| 3.640377825 | 0 |
| 3.64062141 | 0 |
| 3.640864995 | 0 |
| 3.64110858 | 0 |
| 3.641352165 | 0.133 |
| 3.64159575 | 0.667 |
| 3.641839335 | 1.202 |
| 3.64208292 | 1.368 |
| 3.642326505 | 1.368 |
| 3.64257009 | 1.297 |
| 3.642813675 | 0.763 |
| 3.64305726 | 0.228 |
| 3.643300845 | 0 |
| 3.64354443 | 0 |
| 3.643788015 | 0 |
| 3.6440316 | 0 |
| 3.644275185 | 0 |
| 3.64451877 | −0.012 |
| 3.644762355 | −0.039 |
| 3.64500594 | −0.066 |
| 3.645249525 | −0.165 |
| 3.64549311 | −0.272 |
| 3.645736695 | −0.388 |
| 3.64598028 | −0.522 |
| 3.646223865 | −0.655 |
| 3.64646745 | −0.894 |
| 3.646711035 | −1.161 |
| 3.64695462 | −1.127 |
| 3.647198205 | −0.057 |
| 3.64744179 | 1.013 |
| 3.647685375 | 1.279 |
| 3.64792896 | 1.146 |
| 3.648172545 | 1.027 |
| 3.64841613 | 1.027 |
| 3.648659715 | 1.027 |
| 3.6489033 | 0.953 |
| 3.649146885 | 0.819 |
| 3.64939047 | 0.685 |
| 3.649634055 | 0.684 |
| 3.64987764 | 0.684 |
| 3.650121225 | 0.626 |
| 3.65036481 | 0.492 |
| 3.650608395 | 0.359 |
| 3.65085198 | 0.342 |
| 3.651095565 | 0.342 |
| 3.65133915 | 0.299 |
| 3.651582735 | 0.166 |
| 3.65182632 | 0.032 |
| 3.652069905 | 0 |
| 3.65231349 | 0 |
| 3.652557075 | 0 |
| 3.65280066 | 0 |
| 3.653044245 | 0 |
| 3.65328783 | 0 |
| 3.653531415 | 0 |
| 3.653775 | 0 |
| 3.654018585 | 0 |
| 3.65426217 | 0 |
| 3.654505755 | 0 |
| 3.65474934 | 0 |
| 3.654992925 | 0 |
| 3.65523651 | 0 |
| 3.655480095 | 0 |
| 3.65572368 | 0 |
| 3.655967265 | 0 |
| 3.65621085 | 0 |
| 3.656454435 | 0 |
| 3.65669802 | 0 |
| 3.656941605 | 0.16 |
| 3.65718519 | 0.695 |
| 3.657428775 | 1.231 |
| 3.65767236 | 1.37 |
| 3.657915945 | 1.37 |
| 3.65815953 | 1.273 |
| 3.658403115 | 0.737 |
| 3.6586467 | 0.201 |
| 3.658890285 | 0 |
| 3.65913387 | 0 |
| 3.659377455 | 0 |
| 3.65962104 | 0 |
| 3.659864625 | 0 |
| 3.66010821 | −0.014 |
| 3.660351795 | −0.04 |
| 3.66059538 | −0.067 |
| 3.660838965 | −0.17 |
| 3.66108255 | −0.278 |
| 3.661326135 | −0.395 |
| 3.66156972 | −0.529 |
| 3.661813305 | −0.663 |
| 3.66205689 | −0.909 |
| 3.662300475 | −1.177 |
| 3.66254406 | −1.075 |
| 3.662787645 | −0.003 |
| 3.66303123 | 1.069 |
| 3.663274815 | 1.275 |
| 3.6635184 | 1.141 |
| 3.663761985 | 1.028 |
| 3.66400557 | 1.028 |
| 3.664249155 | 1.029 |
| 3.66449274 | 0.948 |
| 3.664736325 | 0.814 |
| 3.66497991 | 0.686 |
| 3.665223495 | 0.686 |
| 3.66546708 | 0.686 |
| 3.665710665 | 0.621 |
| 3.66595425 | 0.487 |
| 3.666197835 | 0.353 |
| 3.66644142 | 0.343 |
| 3.666685005 | 0.343 |
| 3.66692859 | 0.293 |
| 3.667172175 | 0.159 |
| 3.66741576 | 0.025 |
| 3.667659345 | 0 |
| 3.66790293 | 0 |
| 3.668146515 | 0 |
| 3.6683901 | 0 |
| 3.668633685 | 0 |
| 3.66887727 | 0 |
| 3.669120855 | 0 |
| 3.66936444 | 0 |
| 3.669608025 | 0 |
| 3.66985161 | 0 |
| 3.670095195 | 0 |
| 3.67033878 | 0 |
| 3.670582365 | 0 |
| 3.67082595 | 0 |
| 3.671069535 | 0 |
| 3.67131312 | 0 |
| 3.671556705 | 0 |
| 3.67180029 | 0 |
| 3.672043875 | 0 |
| 3.67228746 | 0 |
| 3.672531045 | 0.187 |
| 3.67277463 | 0.723 |
| 3.673018215 | 1.26 |
| 3.6732618 | 1.373 |
| 3.673505385 | 1.373 |
| 3.67374897 | 1.248 |
| 3.673992555 | 0.712 |
| 3.67423614 | 0.175 |
| 3.674479725 | 0 |
| 3.67472331 | 0 |
| 3.674966895 | 0 |
| 3.67521048 | 0 |
| 3.675454065 | 0 |
| 3.67569765 | 0 |
| 3.675941235 | 0 |
| 3.67618482 | 0 |
| 3.676428405 | 0 |
| 3.67667199 | 0 |
| 3.676915575 | 0 |
| 3.67715916 | 0 |
| 3.677402745 | 0 |
| 3.67764633 | 0 |
| 3.677889915 | 0 |
| 3.6781335 | 0 |
| 3.678377085 | 0 |
| 3.67862067 | 0 |
| 3.678864255 | 0 |
| 3.67910784 | 0 |
| 3.679351425 | 0 |
| 3.67959501 | 0 |
| 3.679838595 | 0 |
| 3.68008218 | 0 |
| 3.680325765 | 0 |
| 3.68056935 | 0 |
| 3.680812935 | 0 |
| 3.68105652 | 0 |
| 3.681300105 | 0 |
| 3.68154369 | 0 |
| 3.681787275 | 0 |
| 3.68203086 | 0 |
| 3.682274445 | 0 |
| 3.68251803 | 0 |
| 3.682761615 | 0 |
| 3.6830052 | 0 |
| 3.683248785 | 0 |
| 3.68349237 | 0 |
| 3.683735955 | 0 |
| 3.68397954 | 0 |
| 3.684223125 | 0 |
| 3.68446671 | 0 |
| 3.684710295 | 0 |
| 3.68495388 | 0 |
| 3.685197465 | 0 |
| 3.68544105 | 0 |
| 3.685684635 | 0 |
| 3.68592822 | 0 |
| 3.686171805 | 0 |
| 3.68641539 | 0 |
| 3.686658975 | 0 |
| 3.68690256 | 0 |
| 3.687146145 | 0 |
| 3.68738973 | 0 |
| 3.687633315 | 0 |
| 3.6878769 | 0 |
| 3.688120485 | 0 |
| 3.68836407 | 0 |
| 3.688607655 | 0 |
| 3.68885124 | 0 |
| 3.689094825 | 0 |
| 3.68933841 | 0 |
| 3.689581995 | 0 |
| 3.68982558 | 0 |
| 3.690069165 | 0 |
| 3.69031275 | 0 |
| 3.690556335 | 0 |
| 3.69079992 | 0 |
| 3.691043505 | 0 |
| 3.69128709 | 0 |
| 3.691530675 | 0 |
| 3.69177426 | 0 |
| 3.692017845 | 0 |
| 3.69226143 | 0 |
| 3.692505015 | 0 |
| 3.6927486 | 0 |
| 3.692992185 | 0 |
| 3.69323577 | 0 |
| 3.693479355 | 0 |
| 3.69372294 | 0 |
| 3.693966525 | 0 |
| 3.69421011 | 0 |
| 3.694453695 | 0 |
| 3.69469728 | 0 |
| 3.694940865 | 0 |
| 3.69518445 | 0 |
| 3.695428035 | 0 |
| 3.69567162 | 0 |
| 3.695915205 | 0 |
| 3.69615879 | 0 |
| 3.696402375 | 0 |
| 3.69664596 | 0 |
| 3.696889545 | 0 |
| 3.69713313 | 0 |
| 3.697376715 | 0 |
| 3.6976203 | 0 |
| 3.697863885 | 0 |
| 3.69810747 | 0 |
| 3.698351055 | 0 |
| 3.69859464 | 0 |
| 3.698838225 | 0 |
| 3.69908181 | 0 |
| 3.699325395 | 0 |
| 3.69956898 | 0 |
| 3.699812565 | 0 |
| 3.70005615 | 0 |
| 3.700299735 | 0 |
| 3.70054332 | 0 |
| 3.700786905 | 0 |
| 3.70103049 | 0 |
| 3.701274075 | 0 |
| 3.70151766 | 0 |
| 3.701761245 | 0 |
| 3.70200483 | 0 |
| 3.702248415 | 0 |
| 3.702492 | 0 |
| 3.702735585 | 0 |
| 3.70297917 | 0 |
| 3.703222755 | 0 |
| 3.70346634 | 0 |
| 3.703709925 | 0 |
| 3.70395351 | 0 |
| 3.704197095 | 0 |
| 3.70444068 | 0 |
| 3.704684265 | 0 |
| 3.70492785 | 0 |
| 3.705171435 | 0 |
| 3.70541502 | 0 |
| 3.705658605 | 0 |
| 3.70590219 | 0 |
| 3.706145775 | 0 |
| 3.70638936 | 0 |
| 3.706632945 | 0 |
| 3.70687653 | −0.018 |
| 3.707120115 | −0.045 |
| 3.7073637 | −0.079 |
| 3.707607285 | −0.187 |
| 3.70785087 | −0.294 |
| 3.708094455 | −0.416 |
| 3.70833804 | −0.551 |
| 3.708581625 | −0.685 |
| 3.70882521 | −0.952 |
| 3.709068795 | −1.22 |
| 3.70931238 | −0.917 |
| 3.709555965 | 0.158 |
| 3.70979955 | 1.233 |
| 3.710043135 | 1.258 |
| 3.71028672 | 1.124 |
| 3.710530305 | 1.031 |
| 3.71077389 | 1.031 |
| 3.711017475 | 1.031 |
| 3.71126106 | 0.93 |
| 3.711504645 | 0.795 |
| 3.71174823 | 0.687 |
| 3.711991815 | 0.687 |
| 3.7122354 | 0.687 |
| 3.712478985 | 0.602 |
| 3.71272257 | 0.467 |
| 3.712966155 | 0.344 |
| 3.71320974 | 0.344 |
| 3.713453325 | 0.344 |
| 3.71369691 | 0.274 |
| 3.713940495 | 0.139 |
| 3.71418408 | 0.005 |
| 3.714427665 | 0 |
| 3.71467125 | 0 |
| 3.714914835 | 0 |
| 3.71515842 | 0 |
| 3.715402005 | 0 |
| 3.71564559 | 0 |
| 3.715889175 | 0 |
| 3.71613276 | 0 |
| 3.716376345 | 0 |
| 3.71661993 | 0 |
| 3.716863515 | 0 |
| 3.7171071 | 0 |
| 3.717350685 | 0 |
| 3.71759427 | 0 |
| 3.717837855 | 0 |
| 3.71808144 | 0 |
| 3.718325025 | 0 |
| 3.71856861 | 0 |
| 3.718812195 | 0 |
| 3.71905578 | 0 |
| 3.719299365 | 0.267 |
| 3.71954295 | 0.804 |
| 3.719786535 | 1.341 |
| 3.72003012 | 1.373 |
| 3.720273705 | 1.373 |
| 3.72051729 | 1.168 |
| 3.720760875 | 0.631 |
| 3.72100446 | 0.094 |
| 3.721248045 | 0 |
| 3.72149163 | 0 |
| 3.721735215 | 0 |
| 3.7219788 | 0 |
| 3.722222385 | 0 |
| 3.72246597 | −0.019 |
| 3.722709555 | −0.046 |
| 3.72295314 | −0.085 |
| 3.723196725 | −0.192 |
| 3.72344031 | −0.299 |
| 3.723683895 | −0.422 |
| 3.72392748 | −0.556 |
| 3.724171065 | −0.695 |
| 3.72441465 | −0.963 |
| 3.724658235 | −1.231 |
| 3.72490182 | −0.861 |
| 3.725145405 | 0.211 |
| 3.72538899 | 1.284 |
| 3.725632575 | 1.248 |
| 3.72587616 | 1.114 |
| 3.726119745 | 1.028 |
| 3.72636333 | 1.028 |
| 3.726606915 | 1.028 |
| 3.7268505 | 0.921 |
| 3.727094085 | 0.787 |
| 3.72733767 | 0.685 |
| 3.727581255 | 0.685 |
| 3.72782484 | 0.685 |
| 3.728068425 | 0.593 |
| 3.72831201 | 0.459 |
| 3.728555595 | 0.343 |
| 3.72879918 | 0.343 |
| 3.729042765 | 0.343 |
| 3.72928635 | 0.266 |
| 3.729529935 | 0.132 |
| 3.72977352 | 0 |
| 3.730017105 | 0 |
| 3.73026069 | 0 |
| 3.730504275 | 0 |
| 3.73074786 | 0 |
| 3.730991445 | 0 |
| 3.73123503 | 0 |
| 3.731478615 | 0 |
| 3.7317222 | 0 |
| 3.731965785 | 0 |
| 3.73220937 | 0 |
| 3.732452955 | 0 |
| 3.73269654 | 0 |
| 3.732940125 | 0 |
| 3.73318371 | 0 |
| 3.733427295 | 0 |
| 3.73367088 | 0 |
| 3.733914465 | 0 |
| 3.73415805 | 0 |
| 3.734401635 | 0 |
| 3.73464522 | 0 |
| 3.734888805 | 0.293 |
| 3.73513239 | 0.828 |
| 3.735375975 | 1.363 |
| 3.73561956 | 1.368 |
| 3.735863145 | 1.368 |
| 3.73610673 | 1.137 |
| 3.736350315 | 0.602 |
| 3.7365939 | 0.067 |
| 3.736837485 | 0 |
| 3.73708107 | 0 |
| 3.737324655 | 0 |
| 3.73756824 | 0 |
| 3.737811825 | 0 |
| 3.73805541 | 0 |
| 3.738298995 | 0 |
| 3.73854258 | 0 |
| 3.738786165 | 0 |
| 3.73902975 | 0 |
| 3.739273335 | 0 |
| 3.73951692 | 0 |
| 3.739760505 | 0 |
| 3.74000409 | 0 |
| 3.740247675 | 0 |
| 3.74049126 | 0 |
| 3.740734845 | 0 |
| 3.74097843 | 0 |
| 3.741222015 | 0 |
| 3.7414656 | 0 |
| 3.741709185 | 0 |
| 3.74195277 | 0 |
| 3.742196355 | 0 |
| 3.74243994 | 0 |
| 3.742683525 | 0 |
| 3.74292711 | 0 |
| 3.743170695 | 0 |
| 3.74341428 | 0 |
| 3.743657865 | 0 |
| 3.74390145 | 0 |
| 3.744145035 | 0 |
| 3.74438862 | 0 |
| 3.744632205 | 0 |
| 3.74487579 | 0 |
| 3.745119375 | 0 |
| 3.74536296 | 0 |
| 3.745606545 | 0 |
| 3.74585013 | 0 |
| 3.746093715 | 0 |
| 3.7463373 | 0 |
| 3.746580885 | 0 |
| 3.74682447 | 0 |
| 3.747068055 | 0 |
| 3.74731164 | 0 |
| 3.747555225 | 0 |
| 3.74779881 | 0 |
| 3.748042395 | 0 |
| 3.74828598 | 0 |
| 3.748529565 | 0 |
| 3.74877315 | 0 |
| 3.749016735 | 0 |
| 3.74926032 | 0 |
| 3.749503905 | 0 |
| 3.74974749 | 0 |
| 3.749991075 | 0 |
| 3.75023466 | 0 |
| 3.750478245 | 0 |
| 3.75072183 | 0 |
| 3.750965415 | 0 |
| 3.751209 | 0 |
| 3.751452585 | 0 |
| 3.75169617 | 0 |
| 3.751939755 | 0 |
| 3.75218334 | 0 |
| 3.752426925 | 0 |
| 3.75267051 | 0 |
| 3.752914095 | 0 |
| 3.75315768 | 0 |
| 3.753401265 | 0 |
| 3.75364485 | 0 |
| 3.753888435 | 0 |
| 3.75413202 | 0 |
| 3.754375605 | 0 |
| 3.75461919 | 0 |
| 3.754862775 | 0 |
| 3.75510636 | 0 |
| 3.755349945 | 0 |
| 3.75559353 | 0 |
| 3.755837115 | 0 |
| 3.7560807 | 0 |
| 3.756324285 | 0 |
| 3.75656787 | 0 |
| 3.756811455 | 0 |
| 3.75705504 | 0 |
| 3.757298625 | 0 |
| 3.75754221 | 0 |
| 3.757785795 | 0 |
| 3.75802938 | 0 |
| 3.758272965 | 0 |
| 3.75851655 | 0 |
| 3.758760135 | 0 |
| 3.75900372 | 0 |
| 3.759247305 | 0 |
| 3.75949089 | 0 |
| 3.759734475 | 0 |
| 3.75997806 | 0 |
| 3.760221645 | 0 |
| 3.76046523 | 0 |
| 3.760708815 | 0 |
| 3.7609524 | 0 |
| 3.761195985 | 0 |
| 3.76143957 | 0 |
| 3.761683155 | 0 |
| 3.76192674 | 0 |
| 3.762170325 | 0 |
| 3.76241391 | 0 |
| 3.762657495 | 0 |
| 3.76290108 | 0 |
| 3.763144665 | 0 |
| 3.76338825 | 0 |
| 3.763631835 | 0 |
| 3.76387542 | 0 |
| 3.764119005 | 0 |
| 3.76436259 | 0 |
| 3.764606175 | 0 |
| 3.76484976 | 0 |
| 3.765093345 | 0 |
| 3.76533693 | 0 |
| 3.765580515 | 0 |
| 3.7658241 | 0 |
| 3.766067685 | 0 |
| 3.76631127 | 0 |
| 3.766554855 | 0 |
| 3.76679844 | 0 |
| 3.767042025 | 0 |
| 3.76728561 | 0 |
| 3.767529195 | 0 |
| 3.76777278 | 0 |
| 3.768016365 | 0 |
| 3.76825995 | 0 |
| 3.768503535 | 0 |
| 3.76874712 | 0 |
| 3.768990705 | 0 |
| 3.76923429 | 0 |
| 3.769477875 | 0 |
| 3.76972146 | 0 |
| 3.769965045 | 0 |
| 3.77020863 | 0 |
| 3.770452215 | 0 |
| 3.7706958 | 0 |
| 3.770939385 | 0 |
| 3.77118297 | 0 |
| 3.771426555 | 0 |
| 3.77167014 | 0 |
| 3.771913725 | 0 |
| 3.77215731 | 0 |
| 3.772400895 | 0 |
| 3.77264448 | 0 |
| 3.772888065 | 0 |
| 3.77313165 | 0 |
| 3.773375235 | 0 |
| 3.77361882 | 0 |
| 3.773862405 | 0 |
| 3.77410599 | 0 |
| 3.774349575 | 0 |
| 3.77459316 | 0 |
| 3.774836745 | 0 |
| 3.77508033 | 0 |
| 3.775323915 | 0 |
| 3.7755675 | 0 |
| 3.775811085 | 0 |
| 3.77605467 | 0 |
| 3.776298255 | 0 |
| 3.77654184 | 0 |
| 3.776785425 | 0 |
| 3.77702901 | 0 |
| 3.777272595 | 0 |
| 3.77751618 | 0 |
| 3.777759765 | 0 |
| 3.77800335 | 0 |
| 3.778246935 | 0 |
| 3.77849052 | 0 |
| 3.778734105 | 0 |
| 3.77897769 | 0 |
| 3.779221275 | 0 |
| 3.77946486 | 0 |
| 3.779708445 | 0 |
| 3.77995203 | 0 |
| 3.780195615 | 0 |
| 3.7804392 | 0 |
| 3.780682785 | 0 |
| 3.78092637 | 0 |
| 3.781169955 | 0 |
| 3.78141354 | 0 |
| 3.781657125 | 0 |
| 3.78190071 | 0 |
| 3.782144295 | 0 |
| 3.78238788 | 0 |
| 3.782631465 | 0 |
| 3.78287505 | 0 |
| 3.783118635 | 0 |
| 3.78336222 | 0 |
| 3.783605805 | 0 |
| 3.78384939 | 0 |
| 3.784092975 | 0 |
| 3.78433656 | 0 |
| 3.784580145 | 0 |
| 3.78482373 | 0 |
| 3.785067315 | 0 |
| 3.7853109 | 0 |
| 3.785554485 | 0 |
| 3.78579807 | 0 |
| 3.786041655 | 0 |
| 3.78628524 | 0 |
| 3.786528825 | 0 |
| 3.78677241 | 0 |
| 3.787015995 | 0 |
| 3.78725958 | 0 |
| 3.787503165 | 0 |
| 3.78774675 | 0 |
| 3.787990335 | 0 |
| 3.78823392 | 0 |
| 3.788477505 | 0 |
| 3.78872109 | 0 |
| 3.788964675 | 0 |
| 3.78920826 | 0 |
| 3.789451845 | 0 |
| 3.78969543 | 0 |
| 3.789939015 | 0 |
| 3.7901826 | 0 |
| 3.790426185 | 0 |
| 3.79066977 | 0 |
| 3.790913355 | 0 |
| 3.79115694 | 0 |
| 3.791400525 | 0 |
| 3.79164411 | 0 |
| 3.791887695 | 0 |
| 3.79213128 | 0 |
| 3.792374865 | 0 |
| 3.79261845 | 0 |
| 3.792862035 | 0 |
| 3.79310562 | 0 |
| 3.793349205 | 0 |
| 3.79359279 | 0 |
| 3.793836375 | 0 |
| 3.79407996 | 0 |
| 3.794323545 | 0 |
| 3.79456713 | 0 |
| 3.794810715 | 0 |
| 3.7950543 | 0 |
| 3.795297885 | 0 |
| 3.79554147 | 0 |
| 3.795785055 | 0 |
| 3.79602864 | 0 |
| 3.796272225 | 0 |
| 3.79651581 | 0 |
| 3.796759395 | 0 |
| 3.79700298 | 0 |
| 3.797246565 | 0 |
| 3.79749015 | 0 |
| 3.797733735 | 0 |
| 3.79797732 | 0 |
| 3.798220905 | 0 |
| 3.79846449 | 0 |
| 3.798708075 | 0 |
| 3.79895166 | 0 |
| 3.799195245 | 0 |
| 3.79943883 | 0 |
| 3.799682415 | 0 |
| 3.799926 | 0 |
| 3.800169585 | 0 |
| 3.80041317 | −0.024 |
| 3.800656755 | −0.05 |
| 3.80090034 | −0.106 |
| 3.801143925 | −0.208 |
| 3.80138751 | −0.309 |
| 3.801631095 | −0.432 |
| 3.80187468 | −0.559 |
| 3.802118265 | −0.723 |
| 3.80236185 | −0.976 |
| 3.802605435 | −1.23 |
| 3.80284902 | −0.562 |
| 3.803092605 | 0.454 |
| 3.80333619 | 1.277 |
| 3.803579775 | 1.15 |
| 3.80382336 | 1.023 |
| 3.804066945 | 0.973 |
| 3.80431053 | 0.973 |
| 3.804554115 | 0.966 |
| 3.8047977 | 0.839 |
| 3.805041285 | 0.712 |
| 3.80528487 | 0.648 |
| 3.805528455 | 0.648 |
| 3.80577204 | 0.647 |
| 3.806015625 | 0.529 |
| 3.80625921 | 0.402 |
| 3.806502795 | 0.323 |
| 3.80674638 | 0.323 |
| 3.806989965 | 0.323 |
| 3.80723355 | 0.22 |
| 3.807477135 | 0.093 |
| 3.80772072 | 0 |
| 3.807964305 | 0 |
| 3.80820789 | 0 |
| 3.808451475 | 0 |
| 3.80869506 | 0 |
| 3.808938645 | 0 |
| 3.80918223 | 0 |
| 3.809425815 | 0 |
| 3.8096694 | 0 |
| 3.809912985 | 0 |
| 3.81015657 | 0 |
| 3.810400155 | 0 |
| 3.81064374 | 0 |
| 3.810887325 | 0 |
| 3.81113091 | 0 |
| 3.811374495 | 0 |
| 3.81161808 | 0 |
| 3.811861665 | 0 |
| 3.81210525 | 0 |
| 3.812348835 | 0 |
| 3.81259242 | 0 |
| 3.812836005 | 0.4 |
| 3.81307959 | 0.901 |
| 3.813323175 | 1.281 |
| 3.81356676 | 1.281 |
| 3.813810345 | 1.28 |
| 3.81405393 | 0.939 |
| 3.814297515 | 0.438 |
| 3.8145411 | 0 |
| 3.814784685 | 0 |
| 3.81502827 | 0 |
| 3.815271855 | 0 |
| 3.81551544 | 0 |
| 3.815759025 | 0 |
| 3.81600261 | 0 |
| 3.816246195 | 0 |
| 3.81648978 | 0 |
| 3.816733365 | 0 |
| 3.81697695 | 0 |
| 3.817220535 | 0 |
| 3.81746412 | 0 |
| 3.817707705 | 0 |
| 3.81795129 | 0 |
| 3.818194875 | 0 |
| 3.81843846 | 0 |
| 3.818682045 | 0 |
| 3.81892563 | 0 |
| 3.819169215 | 0 |
| 3.8194128 | 0 |
| 3.819656385 | 0 |
| 3.81989997 | 0 |
| 3.820143555 | 0 |
| 3.82038714 | 0 |
| 3.820630725 | 0 |
| 3.82087431 | 0 |
| 3.821117895 | 0 |
| 3.82136148 | 0 |
| 3.821605065 | 0 |
| 3.82184865 | 0 |
| 3.822092235 | 0 |
| 3.82233582 | 0 |
| 3.822579405 | 0 |
| 3.82282299 | 0 |
| 3.823066575 | 0 |
| 3.82331016 | 0 |
| 3.823553745 | 0 |
| 3.82379733 | 0 |
| 3.824040915 | 0 |
| 3.8242845 | 0 |
| 3.824528085 | 0 |
| 3.82477167 | 0 |
| 3.825015255 | 0 |
| 3.82525884 | 0 |
| 3.825502425 | 0 |
| 3.82574601 | 0 |
| 3.825989595 | 0 |
| 3.82623318 | 0 |
| 3.826476765 | 0 |
| 3.82672035 | 0 |
| 3.826963935 | 0 |
| 3.82720752 | 0 |
| 3.827451105 | 0 |
| 3.82769469 | 0 |
| 3.827938275 | 0 |
| 3.82818186 | 0 |
| 3.828425445 | 0 |
| 3.82866903 | 0 |
| 3.828912615 | 0 |
| 3.8291562 | 0 |
| 3.829399785 | 0 |
| 3.82964337 | 0 |
| 3.829886955 | 0 |
| 3.83013054 | 0 |
| 3.830374125 | 0 |
| 3.83061771 | 0 |
| 3.830861295 | 0 |
| 3.83110488 | 0 |
| 3.831348465 | 0 |
| 3.83159205 | 0 |
| 3.831835635 | 0 |
| 3.83207922 | 0 |
| 3.832322805 | 0 |
| 3.83256639 | 0 |
| 3.832809975 | 0 |
| 3.83305356 | 0 |
| 3.833297145 | 0 |
| 3.83354073 | 0 |
| 3.833784315 | 0 |
| 3.8340279 | 0 |
| 3.834271485 | 0 |
| 3.83451507 | 0 |
| 3.834758655 | 0 |
| 3.83500224 | 0 |
| 3.835245825 | 0 |
| 3.83548941 | 0 |
| 3.835732995 | 0 |
| 3.83597658 | 0 |
| 3.836220165 | 0 |
| 3.83646375 | 0 |
| 3.836707335 | 0 |
| 3.83695092 | 0 |
| 3.837194505 | 0 |
| 3.83743809 | 0 |
| 3.837681675 | 0 |
| 3.83792526 | 0 |
| 3.838168845 | 0 |
| 3.83841243 | 0 |
| 3.838656015 | 0 |
| 3.8388996 | 0 |
| 3.839143185 | 0 |
| 3.83938677 | 0 |
| 3.839630355 | 0 |
| 3.83987394 | 0 |
| 3.840117525 | 0 |
| 3.84036111 | 0 |
| 3.840604695 | 0 |
| 3.84084828 | 0 |
| 3.841091865 | 0 |
| 3.84133545 | 0 |
| 3.841579035 | 0 |
| 3.84182262 | 0 |
| 3.842066205 | 0 |
| 3.84230979 | 0 |
| 3.842553375 | 0 |
| 3.84279696 | 0 |
| 3.843040545 | 0 |
| 3.84328413 | 0 |
| 3.843527715 | 0 |
| 3.8437713 | 0 |
| 3.844014885 | 0 |
| 3.84425847 | 0 |
| 3.844502055 | 0 |
| 3.84474564 | 0 |
| 3.844989225 | 0 |
| 3.84523281 | 0 |
| 3.845476395 | 0 |
| 3.84571998 | 0 |
| 3.845963565 | 0 |
| 3.84620715 | 0 |
| 3.846450735 | 0 |
| 3.84669432 | 0 |
| 3.846937905 | 0 |
| 3.84718149 | 0 |
| 3.847425075 | 0 |
| 3.84766866 | 0 |
| 3.847912245 | 0 |
| 3.84815583 | 0 |
| 3.848399415 | 0 |
| 3.848643 | 0 |
| 3.848886585 | 0 |
| 3.84913017 | 0 |
| 3.849373755 | 0 |
| 3.84961734 | 0 |
| 3.849860925 | 0 |
| 3.85010451 | 0 |
| 3.850348095 | 0 |
| 3.85059168 | 0 |
| 3.850835265 | 0 |
| 3.85107885 | 0 |
| 3.851322435 | 0 |
| 3.85156602 | 0 |
| 3.851809605 | 0 |
| 3.85205319 | 0 |
| 3.852296775 | 0 |
| 3.85254036 | 0 |
| 3.852783945 | 0 |
| 3.85302753 | 0 |
| 3.853271115 | 0 |
| 3.8535147 | 0 |
| 3.853758285 | 0 |
| 3.85400187 | 0 |
| 3.854245455 | 0 |
| 3.85448904 | 0 |
| 3.854732625 | 0 |
| 3.85497621 | 0 |
| 3.855219795 | 0 |
| 3.85546338 | 0 |
| 3.855706965 | 0 |
| 3.85595055 | 0 |
| 3.856194135 | 0 |
| 3.85643772 | 0 |
| 3.856681305 | 0 |
| 3.85692489 | 0 |
| 3.857168475 | 0 |
| 3.85741206 | 0 |
| 3.857655645 | 0 |
| 3.85789923 | 0 |
| 3.858142815 | 0 |
| 3.8583864 | 0 |
| 3.858629985 | 0 |
| 3.85887357 | 0 |
| 3.859117155 | 0 |
| 3.85936074 | 0 |
| 3.859604325 | 0 |
| 3.85984791 | 0 |
| 3.860091495 | 0 |
| 3.86033508 | 0 |
| 3.860578665 | 0 |
| 3.86082225 | 0 |
| 3.861065835 | 0 |
| 3.86130942 | 0 |
| 3.861553005 | 0 |
| 3.86179659 | 0 |
| 3.862040175 | 0 |
| 3.86228376 | 0 |
| 3.862527345 | 0 |
| 3.86277093 | 0 |
| 3.863014515 | 0 |
| 3.8632581 | 0 |
| 3.863501685 | 0 |
| 3.86374527 | 0 |
| 3.863988855 | 0 |
| 3.86423244 | 0 |
| 3.864476025 | 0 |
| 3.86471961 | 0 |
| 3.864963195 | 0 |
| 3.86520678 | 0 |
| 3.865450365 | 0 |
| 3.86569395 | 0 |
| 3.865937535 | 0 |
| 3.86618112 | 0 |
| 3.866424705 | 0 |
| 3.86666829 | 0 |
| 3.866911875 | 0 |
| 3.86715546 | 0 |
| 3.867399045 | 0 |
| 3.86764263 | 0 |
| 3.867886215 | 0 |
| 3.8681298 | 0 |
| 3.868373385 | 0 |
| 3.86861697 | 0 |
| 3.868860555 | 0 |
| 3.86910414 | 0 |
| 3.869347725 | 0 |
| 3.86959131 | 0 |
| 3.869834895 | 0 |
| 3.87007848 | 0 |
| 3.870322065 | 0 |
| 3.87056565 | 0 |
| 3.870809235 | 0 |
| 3.87105282 | 0 |
| 3.871296405 | 0 |
| 3.87153999 | 0 |
| 3.871783575 | 0 |
| 3.87202716 | 0 |
| 3.872270745 | 0 |
| 3.87251433 | 0 |
| 3.872757915 | 0 |
| 3.8730015 | 0 |
| 3.873245085 | 0 |
| 3.87348867 | 0 |
| 3.873732255 | 0 |
| 3.87397584 | 0 |
| 3.874219425 | 0 |
| 3.87446301 | 0 |
| 3.874706595 | 0 |
| 3.87495018 | 0 |
| 3.875193765 | 0 |
| 3.87543735 | 0 |
| 3.875680935 | 0 |
| 3.87592452 | 0 |
| 3.876168105 | 0 |
| 3.87641169 | 0 |
| 3.876655275 | 0 |
| 3.87689886 | 0 |
| 3.877142445 | 0 |
| 3.87738603 | 0 |
| 3.877629615 | 0 |
| 3.8778732 | 0 |
| 3.878116785 | 0 |
| 3.87836037 | 0 |
| 3.878603955 | 0 |
| 3.87884754 | 0 |
| 3.879091125 | 0 |
| 3.87933471 | 0 |
| 3.879578295 | 0 |
| 3.87982188 | 0 |
| 3.880065465 | 0 |
| 3.88030905 | 0 |
| 3.880552635 | 0 |
| 3.88079622 | 0 |
| 3.881039805 | 0 |
| 3.88128339 | 0 |
| 3.881526975 | 0 |
| 3.88177056 | 0 |
| 3.882014145 | 0 |
| 3.88225773 | 0 |
| 3.882501315 | 0 |
| 3.8827449 | 0 |
| 3.882988485 | 0 |
| 3.88323207 | 0 |
| 3.883475655 | 0 |
| 3.88371924 | 0 |
| 3.883962825 | 0 |
| 3.88420641 | 0 |
| 3.884449995 | 0 |
| 3.88469358 | 0 |
| 3.884937165 | 0 |
| 3.88518075 | 0 |
| 3.885424335 | 0 |
| 3.88566792 | 0 |
| 3.885911505 | 0 |
| 3.88615509 | 0 |
| 3.886398675 | 0 |
| 3.88664226 | 0 |
| 3.886885845 | 0 |
| 3.88712943 | 0 |
| 3.887373015 | 0 |
| 3.8876166 | 0 |
| 3.887860185 | 0 |
| 3.88810377 | 0 |
| 3.888347355 | 0 |
| 3.88859094 | 0 |
| 3.888834525 | 0 |
| 3.88907811 | 0 |
| 3.889321695 | 0 |
| 3.88956528 | 0 |
| 3.889808865 | 0 |
| 3.89005245 | 0 |
| 3.890296035 | 0 |
| 3.89053962 | 0 |
| 3.890783205 | 0 |
| 3.89102679 | 0 |
| 3.891270375 | 0 |
| 3.89151396 | 0 |
| 3.891757545 | 0 |
| 3.89200113 | 0 |
| 3.892244715 | 0 |
| 3.8924883 | 0 |
| 3.892731885 | 0 |
| 3.89297547 | 0 |
| 3.893219055 | 0 |
| 3.89346264 | 0 |
| 3.893706225 | 0 |
| 3.89394981 | 0 |
| 3.894193395 | 0 |
| 3.89443698 | 0 |
| 3.894680565 | 0 |
| 3.89492415 | 0 |
| 3.895167735 | 0 |
| 3.89541132 | 0 |
| 3.895654905 | 0 |
| 3.89589849 | 0 |
| 3.896142075 | 0 |
| 3.89638566 | 0 |
| 3.896629245 | 0 |
| 3.89687283 | 0 |
| 3.897116415 | 0 |
| 3.89736 | 0 |
| 3.897603585 | 0 |
| 3.89784717 | 0 |
| 3.898090755 | 0 |
| 3.89833434 | 0 |
| 3.898577925 | 0 |
| 3.89882151 | 0 |
| 3.899065095 | 0 |
| 3.89930868 | 0 |
| 3.899552265 | 0 |
| 3.89979585 | 0 |
| 3.900039435 | 0 |
| 3.90028302 | 0 |
| 3.900526605 | 0 |
| 3.90077019 | 0 |
| 3.901013775 | 0 |
| 3.90125736 | 0 |
| 3.901500945 | 0 |
| 3.90174453 | 0 |
| 3.901988115 | 0 |
| 3.9022317 | 0 |
| 3.902475285 | 0 |
| 3.90271887 | 0 |
| 3.902962455 | 0 |
| 3.90320604 | 0 |
| 3.903449625 | 0 |
| 3.90369321 | 0 |
| 3.903936795 | 0 |
| 3.90418038 | 0 |
| 3.904423965 | 0 |
| 3.90466755 | 0 |
| 3.904911135 | 0 |
| 3.90515472 | 0 |
| 3.905398305 | 0 |
| 3.90564189 | 0 |
| 3.905885475 | 0 |
| 3.90612906 | 0 |
| 3.906372645 | 0 |
| 3.90661623 | 0 |
| 3.906859815 | 0 |
| 3.9071034 | 0 |
| 3.907346985 | 0 |
| 3.90759057 | 0 |
| 3.907834155 | 0 |
| 3.90807774 | 0 |
| 3.908321325 | 0 |
| 3.90856491 | 0 |
| 3.908808495 | 0 |
| 3.90905208 | 0 |
| 3.909295665 | 0 |
| 3.90953925 | 0 |
| 3.909782835 | 0 |
| 3.91002642 | 0 |
| 3.910270005 | 0 |
| 3.91051359 | 0 |
| 3.910757175 | 0 |
| 3.91100076 | 0 |
| 3.911244345 | 0 |
| 3.91148793 | 0 |
| 3.911731515 | 0 |
| 3.9119751 | 0 |
| 3.912218685 | 0 |
| 3.91246227 | 0 |
| 3.912705855 | 0 |
| 3.91294944 | 0 |
| 3.913193025 | 0 |
| 3.91343661 | 0 |
| 3.913680195 | 0 |
| 3.91392378 | 0 |
| 3.914167365 | 0 |
| 3.91441095 | 0 |
| 3.914654535 | 0 |
| 3.91489812 | 0 |
| 3.915141705 | 0 |
| 3.91538529 | 0 |
| 3.915628875 | 0 |
| 3.91587246 | 0 |
| 3.916116045 | 0 |
| 3.91635963 | 0 |
| 3.916603215 | 0 |
| 3.9168468 | 0 |
| 3.917090385 | 0 |
| 3.91733397 | 0 |
| 3.917577555 | 0 |
| 3.91782114 | 0 |
| 3.918064725 | 0 |
| 3.91830831 | 0 |
| 3.918551895 | 0 |
| 3.91879548 | 0 |
| 3.919039065 | 0 |
| 3.91928265 | 0 |
| 3.919526235 | 0 |
| 3.91976982 | 0 |
| 3.920013405 | 0 |
| 3.92025699 | 0 |
| 3.920500575 | 0 |
| 3.92074416 | 0 |
| 3.920987745 | 0 |
| 3.92123133 | 0 |
| 3.921474915 | 0 |
| 3.9217185 | 0 |
| 3.921962085 | 0 |
| 3.92220567 | 0 |
| 3.922449255 | 0 |
| 3.92269284 | 0 |
| 3.922936425 | 0 |
| 3.92318001 | 0 |
| 3.923423595 | 0 |
| 3.92366718 | 0 |
| 3.923910765 | 0 |
| 3.92415435 | 0 |
| 3.924397935 | 0 |
| 3.92464152 | 0 |
| 3.924885105 | −0.007 |
| 3.92512869 | −0.026 |
| 3.925372275 | −0.045 |
| 3.92561586 | −0.109 |
| 3.925859445 | −0.185 |
| 3.92610303 | −0.265 |
| 3.926346615 | −0.359 |
| 3.9265902 | −0.454 |
| 3.926833785 | −0.612 |
| 3.92707737 | −0.8 |
| 3.927320955 | −0.87 |
| 3.92756454 | −0.116 |
| 3.927808125 | 0.637 |
| 3.92805171 | 0.909 |
| 3.928295295 | 0.814 |
| 3.92853888 | 0.72 |
| 3.928782465 | 0.72 |
| 3.92902605 | 0.719 |
| 3.929269635 | 0.676 |
| 3.92951322 | 0.582 |
| 3.929756805 | 0.488 |
| 3.93000039 | 0.478 |
| 3.930243975 | 0.477 |
| 3.93048756 | 0.446 |
| 3.930731145 | 0.352 |
| 3.93097473 | 0.259 |
| 3.931218315 | 0.238 |
| 3.9314619 | 0.238 |
| 3.931705485 | 0.217 |
| 3.93194907 | 0.124 |
| 3.932192655 | 0.031 |
| 3.93243624 | 0 |
| 3.932679825 | 0 |
| 3.93292341 | 0 |
| 3.933166995 | 0 |
| 3.93341058 | 0 |
| 3.933654165 | 0 |
| 3.93389775 | 0 |
| 3.934141335 | 0 |
| 3.93438492 | 0 |
| 3.934628505 | 0 |
| 3.93487209 | 0 |
| 3.935115675 | 0 |
| 3.93535926 | 0 |
| 3.935602845 | 0 |
| 3.93584643 | 0 |
| 3.936090015 | 0 |
| 3.9363336 | 0 |
| 3.936577185 | 0 |
| 3.93682077 | 0 |
| 3.937064355 | 0 |
| 3.93730794 | 0.072 |
| 3.937551525 | 0.435 |
| 3.93779511 | 0.797 |
| 3.938038695 | 0.928 |
| 3.93828228 | 0.927 |
| 3.938525865 | 0.896 |
| 3.93876945 | 0.534 |
| 3.939013035 | 0.172 |
| 3.93925662 | 0 |
| 3.939500205 | 0 |
| 3.93974379 | 0 |
| 3.939987375 | 0 |
| 3.94023096 | 0 |
| 3.940474545 | −0.007 |
| 3.94071813 | −0.025 |
| 3.940961715 | −0.043 |
| 3.9412053 | −0.107 |
| 3.941448885 | −0.178 |
| 3.94169247 | −0.255 |
| 3.941936055 | −0.344 |
| 3.94217964 | −0.433 |
| 3.942423225 | −0.587 |
| 3.94266681 | −0.764 |
| 3.942910395 | −0.785 |
| 3.94315398 | −0.074 |
| 3.943397565 | 0.637 |
| 3.94364115 | 0.853 |
| 3.943884735 | 0.764 |
| 3.94412832 | 0.68 |
| 3.944371905 | 0.679 |
| 3.94461549 | 0.678 |
| 3.944859075 | 0.633 |
| 3.94510266 | 0.545 |
| 3.945346245 | 0.456 |
| 3.94558983 | 0.45 |
| 3.945833415 | 0.45 |
| 3.946077 | 0.416 |
| 3.946320585 | 0.328 |
| 3.94656417 | 0.239 |
| 3.946807755 | 0.224 |
| 3.94705134 | 0.224 |
| 3.947294925 | 0.2 |
| 3.94753851 | 0.113 |
| 3.947782095 | 0.025 |
| 3.94802568 | 0 |
| 3.948269265 | 0 |
| 3.94851285 | 0 |
| 3.948756435 | 0 |
| 3.94900002 | 0 |
| 3.949243605 | 0 |
| 3.94948719 | 0 |
| 3.949730775 | 0 |
| 3.94997436 | 0 |
| 3.950217945 | 0 |
| 3.95046153 | 0 |
| 3.950705115 | 0 |
| 3.9509487 | 0 |
| 3.951192285 | 0 |
| 3.95143587 | 0 |
| 3.951679455 | 0 |
| 3.95192304 | 0 |
| 3.952166625 | 0 |
| 3.95241021 | 0 |
| 3.952653795 | 0 |
| 3.95289738 | 0.085 |
| 3.953140965 | 0.426 |
| 3.95338455 | 0.767 |
| 3.953628135 | 0.872 |
| 3.95387172 | 0.872 |
| 3.954115305 | 0.826 |
| 3.95435889 | 0.485 |
| 3.954602475 | 0.145 |
| 3.95484606 | 0 |
| 3.955089645 | 0 |
| 3.95533323 | 0 |
| 3.955576815 | 0 |
| 3.9558204 | 0 |
| 3.956063985 | −0.008 |
| 3.95630757 | −0.025 |
| 3.956551155 | −0.041 |
| 3.95679474 | −0.104 |
| 3.957038325 | −0.171 |
| 3.95728191 | −0.244 |
| 3.957525495 | −0.327 |
| 3.95776908 | −0.411 |
| 3.958012665 | −0.559 |
| 3.95825625 | −0.726 |
| 3.958499835 | −0.704 |
| 3.95874342 | −0.037 |
| 3.958987005 | 0.631 |
| 3.95923059 | 0.797 |
| 3.959474175 | 0.713 |
| 3.95971776 | 0.638 |
| 3.959961345 | 0.637 |
| 3.96020493 | 0.637 |
| 3.960448515 | 0.59 |
| 3.9606921 | 0.507 |
| 3.960935685 | 0.424 |
| 3.96117927 | 0.423 |
| 3.961422855 | 0.422 |
| 3.96166644 | 0.386 |
| 3.961910025 | 0.303 |
| 3.96215361 | 0.221 |
| 3.962397195 | 0.21 |
| 3.96264078 | 0.21 |
| 3.962884365 | 0.184 |
| 3.96312795 | 0.102 |
| 3.963371535 | 0.02 |
| 3.96361512 | 0 |
| 3.963858705 | 0 |
| 3.96410229 | 0 |
| 3.964345875 | 0 |
| 3.96458946 | 0 |
| 3.964833045 | 0 |
| 3.96507663 | 0 |
| 3.965320215 | 0 |
| 3.9655638 | 0 |
| 3.965807385 | 0 |
| 3.96605097 | 0 |
| 3.966294555 | 0 |
| 3.96653814 | 0 |
| 3.966781725 | 0 |
| 3.96702531 | 0 |
| 3.967268895 | 0 |
| 3.96751248 | 0 |
| 3.967756065 | 0 |
| 3.96799965 | 0 |
| 3.968243235 | 0 |
| 3.96848682 | 0.095 |
| 3.968730405 | 0.415 |
| 3.96897399 | 0.734 |
| 3.969217575 | 0.817 |
| 3.96946116 | 0.816 |
| 3.969704745 | 0.757 |
| 3.96994833 | 0.439 |
| 3.970191915 | 0.12 |
| 3.9704355 | 0 |
| 3.970679085 | 0 |
| 3.97092267 | 0 |
| 3.971166255 | 0 |
| 3.97140984 | 0 |
| 3.971653425 | −0.008 |
| 3.97189701 | −0.024 |
| 3.972140595 | −0.04 |
| 3.97238418 | −0.1 |
| 3.972627765 | −0.163 |
| 3.97287135 | −0.232 |
| 3.973114935 | −0.31 |
| 3.97335852 | −0.388 |
| 3.973602105 | −0.531 |
| 3.97384569 | −0.687 |
| 3.974089275 | −0.627 |
| 3.97433286 | −0.003 |
| 3.974576445 | 0.621 |
| 3.97482003 | 0.741 |
| 3.975063615 | 0.662 |
| 3.9753072 | 0.596 |
| 3.975550785 | 0.596 |
| 3.97579437 | 0.595 |
| 3.976037955 | 0.548 |
| 3.97628154 | 0.47 |
| 3.976525125 | 0.395 |
| 3.97676871 | 0.395 |
| 3.977012295 | 0.395 |
| 3.97725588 | 0.357 |
| 3.977499465 | 0.279 |
| 3.97774305 | 0.202 |
| 3.977986635 | 0.196 |
| 3.97823022 | 0.196 |
| 3.978473805 | 0.168 |
| 3.97871739 | 0.091 |
| 3.978960975 | 0.014 |
| 3.97920456 | 0 |
| 3.979448145 | 0 |
| 3.97969173 | 0 |
| 3.979935315 | 0 |
| 3.9801789 | 0 |
| 3.980422485 | 0 |
| 3.98066607 | 0 |
| 3.980909655 | 0 |
| 3.98115324 | 0 |
| 3.981396825 | 0 |
| 3.98164041 | 0 |
| 3.981883995 | 0 |
| 3.98212758 | 0 |
| 3.982371165 | 0 |
| 3.98261475 | 0 |
| 3.982858335 | 0 |
| 3.98310192 | 0 |
| 3.983345505 | 0 |
| 3.98358909 | 0 |
| 3.983832675 | 0 |
| 3.98407626 | 0.104 |
| 3.984319845 | 0.402 |
| 3.98456343 | 0.7 |
| 3.984807015 | 0.761 |
| 3.9850506 | 0.761 |
| 3.985294185 | 0.691 |
| 3.98553777 | 0.394 |
| 3.985781355 | 0.097 |
| 3.98602494 | 0 |
| 3.986268525 | 0 |
| 3.98651211 | 0 |
| 3.986755695 | 0 |
| 3.98699928 | 0 |
| 3.987242865 | 0 |
| 3.98748645 | 0 |
| 3.987730035 | 0 |
| 3.98797362 | 0 |
| 3.988217205 | 0 |
| 3.98846079 | 0 |
| 3.988704375 | 0 |
| 3.98894796 | 0 |
| 3.989191545 | 0 |
| 3.98943513 | 0 |
| 3.989678715 | 0 |
| 3.9899223 | 0 |
| 3.990165885 | 0 |
| 3.99040947 | 0 |
| 3.990653055 | 0 |
| 3.99089664 | 0 |
| 3.991140225 | 0 |
| 3.99138381 | 0 |
| 3.991627395 | 0 |
| 3.99187098 | 0 |
| 3.992114565 | 0 |
| 3.99235815 | 0 |
| 3.992601735 | 0 |
| 3.99284532 | 0 |
| 3.993088905 | 0 |
| 3.99333249 | 0 |
| 3.993576075 | 0 |
| 3.99381966 | 0 |
| 3.994063245 | 0 |
| 3.99430683 | 0 |
| 3.994550415 | 0 |
| 3.994794 | 0 |
| 3.995037585 | 0 |
| 3.99528117 | 0 |
| 3.995524755 | 0 |
| 3.99576834 | 0 |
| 3.996011925 | 0 |
| 3.99625551 | 0 |
| 3.996499095 | 0 |
| 3.99674268 | 0 |
| 3.996986265 | 0 |
| 3.99722985 | 0 |
| 3.997473435 | 0 |
| 3.99771702 | 0 |
| 3.997960605 | 0 |
| 3.99820419 | 0 |
| 3.998447775 | 0 |
| 3.99869136 | 0 |
| 3.998934945 | 0 |
| 3.99917853 | 0 |
| 3.999422115 | 0 |
| 3.9996657 | 0 |
| 3.999909285 | 0 |
| 4.00015287 | 0 |
| 4.000396455 | 0 |
| 4.00064004 | 0 |
| 4.000883625 | 0 |
| 4.00112721 | 0 |
| 4.001370795 | 0 |
| 4.00161438 | 0 |
| 4.001857965 | 0 |
| 4.00210155 | 0 |
| 4.002345135 | 0 |
| 4.00258872 | 0 |
| 4.002832305 | 0 |
| 4.00307589 | 0 |
| 4.003319475 | 0 |
| 4.00356306 | 0 |
| 4.003806645 | 0 |
| 4.00405023 | 0 |
| 4.004293815 | 0 |
| 4.0045374 | 0 |
| 4.004780985 | 0 |
| 4.00502457 | 0 |
| 4.005268155 | 0 |
| 4.00551174 | 0 |
| 4.005755325 | 0 |
| 4.00599891 | 0 |
| 4.006242495 | 0 |
| 4.00648608 | 0 |
| 4.006729665 | 0 |
| 4.00697325 | 0 |
| 4.007216835 | 0 |
| 4.00746042 | 0 |
| 4.007704005 | 0 |
| 4.00794759 | 0 |
| 4.008191175 | 0 |
| 4.00843476 | 0 |
| 4.008678345 | 0 |
| 4.00892193 | 0 |
| 4.009165515 | 0 |
| 4.0094091 | 0 |
| 4.009652685 | 0 |
| 4.00989627 | 0 |
| 4.010139855 | 0 |
| 4.01038344 | 0 |
| 4.010627025 | 0 |
| 4.01087061 | 0 |
| 4.011114195 | 0 |
| 4.01135778 | 0 |
| 4.011601365 | 0 |
| 4.01184495 | 0 |
| 4.012088535 | 0 |
| 4.01233212 | 0 |
| 4.012575705 | 0 |
| 4.01281929 | 0 |
| 4.013062875 | 0 |
| 4.01330646 | 0 |
| 4.013550045 | 0 |
| 4.01379363 | 0 |
| 4.014037215 | 0 |
| 4.0142808 | 0 |
| 4.014524385 | 0 |
| 4.01476797 | 0 |
| 4.015011555 | 0 |
| 4.01525514 | 0 |
| 4.015498725 | 0 |
| 4.01574231 | 0 |
| 4.015985895 | 0 |
| 4.01622948 | 0 |
| 4.016473065 | 0 |
| 4.01671665 | 0 |
| 4.016960235 | 0 |
| 4.01720382 | 0 |
| 4.017447405 | 0 |
| 4.01769099 | 0 |
| 4.017934575 | 0 |
| 4.01817816 | 0 |
| 4.018421745 | 0 |
| 4.01866533 | 0 |
| 4.018908915 | 0 |
| 4.0191525 | 0 |
| 4.019396085 | 0 |
| 4.01963967 | 0 |
| 4.019883255 | 0 |
| 4.02012684 | 0 |
| 4.020370425 | 0 |
| 4.02061401 | 0 |
| 4.020857595 | 0 |
| 4.02110118 | 0 |
| 4.021344765 | 0 |
| 4.02158835 | 0 |
| 4.021831935 | 0 |
| 4.02207552 | 0 |
| 4.022319105 | 0 |
| 4.02256269 | 0 |
| 4.022806275 | 0 |
| 4.02304986 | 0 |
| 4.023293445 | 0 |
| 4.02353703 | 0 |
| 4.023780615 | 0 |
| 4.0240242 | 0 |
| 4.024267785 | 0 |
| 4.02451137 | 0 |
| 4.024754955 | 0 |
| 4.02499854 | 0 |
| 4.025242125 | 0 |
| 4.02548571 | 0 |
| 4.025729295 | 0 |
| 4.02597288 | 0 |
| 4.026216465 | 0 |
| 4.02646005 | 0 |
| 4.026703635 | 0 |
| 4.02694722 | 0 |
| 4.027190805 | 0 |
| 4.02743439 | 0 |
| 4.027677975 | 0 |
| 4.02792156 | 0 |
| 4.028165145 | 0 |
| 4.02840873 | 0 |
| 4.028652315 | 0 |
| 4.0288959 | 0 |
| 4.029139485 | 0 |
| 4.02938307 | 0 |
| 4.029626655 | 0 |
| 4.02987024 | 0 |
| 4.030113825 | 0 |
| 4.03035741 | 0 |
| 4.030600995 | 0 |
| 4.03084458 | 0 |
| 4.031088165 | 0 |
| 4.03133175 | 0 |
| 4.031575335 | 0 |
| 4.03181892 | 0 |
| 4.032062505 | 0 |
| 4.03239609 | 0 |
| 4.032549675 | 0 |
| 4.03279326 | 0 |
| 4.033036845 | 0 |
| 4.03328043 | 0 |
| 4.033524015 | 0 |
| 4.0337676 | 0 |
| 4.034011185 | 0 |
| 4.03425477 | 0 |
| 4.034498355 | 0 |
| 4.03474194 | 0 |
| 4.034985525 | 0 |
| 4.03522911 | 0 |
| 4.035472695 | 0 |
| 4.03571628 | 0 |
| 4.035959865 | 0 |
| 4.03620345 | 0 |
| 4.036447035 | 0 |
| 4.03669062 | 0 |
| 4.036934205 | 0 |
| 4.03717779 | 0 |
| 4.037421375 | 0 |
| 4.03766496 | 0 |
| 4.037908545 | 0 |
| 4.03815213 | 0 |
| 4.038395715 | 0 |
| 4.0386393 | 0 |
| 4.038882885 | 0 |
| 4.03912647 | 0 |
| 4.039370055 | 0 |
| 4.03961364 | 0 |
| 4.039857225 | 0 |
| 4.04010081 | 0 |
| 4.040344395 | 0 |
| 4.04058798 | 0 |
| 4.040831565 | 0 |
| 4.04107515 | 0 |
| 4.041318735 | 0 |
| 4.04156232 | 0 |
| 4.041805905 | 0 |
| 4.04204949 | 0 |
| 4.042293075 | 0 |
| 4.04253666 | 0 |
| 4.042780245 | 0 |
| 4.04302383 | 0 |
| 4.043267415 | 0 |
| 4.043511 | 0 |
| 4.043754585 | 0 |
| 4.04399817 | 0 |
| 4.044241755 | 0 |
| 4.04448534 | 0 |
| 4.044728925 | 0 |
| 4.04497251 | 0 |
| 4.045216095 | 0 |
| 4.04545968 | 0 |
| 4.045703265 | 0 |
| 4.04594685 | 0 |
| 4.046190435 | 0 |
| 4.04643402 | 0 |
| 4.046677605 | 0 |
| 4.04692119 | 0 |
| 4.047164775 | 0 |
| 4.04740836 | 0 |
| 4.047651945 | 0 |
| 4.04789553 | 0 |
| 4.048139115 | 0 |
| 4.0483827 | 0 |
| 4.048626285 | 0 |
| 4.04886987 | 0 |
| 4.049113455 | 0 |
| 4.04935704 | 0 |
| 4.049600625 | 0 |
| 4.04984421 | 0 |
| 4.050087795 | 0 |
| 4.05033138 | 0 |
| 4.050574965 | 0 |
| 4.05081855 | 0 |
| 4.051062135 | 0 |
| 4.05130572 | 0 |
| 4.051549305 | 0 |
| 4.05179289 | 0 |
| 4.052036475 | 0 |
| 4.05228006 | 0 |
| 4.052523645 | 0 |
| 4.05276723 | 0 |
| 4.053010815 | 0 |
| 4.0532544 | 0 |
| 4.053497985 | 0 |
| 4.05374157 | 0 |
| 4.053985155 | 0 |
| 4.05422874 | 0 |
| 4.054472325 | 0 |
| 4.05471591 | 0 |
| 4.054959495 | 0 |
| 4.05520308 | 0 |
| 4.055446665 | 0 |
| 4.05569025 | 0 |
| 4.055933835 | 0 |
| 4.05617742 | 0 |
| 4.056421005 | 0 |
| 4.05666459 | 0 |
| 4.056908175 | 0 |
| 4.05715176 | 0 |
| 4.057395345 | 0 |
| 4.05763893 | 0 |
| 4.057882515 | 0 |
| 4.0581261 | 0 |
| 4.058369685 | 0 |
| 4.05861327 | 0 |
| 4.058856855 | 0 |
| 4.05910044 | 0 |
| 4.059344025 | 0 |
| 4.05958761 | 0 |
| 4.059831195 | 0 |
| 4.06007478 | 0 |
| 4.060318365 | 0 |
| 4.06056195 | 0 |
| 4.060805535 | 0 |
| 4.06104912 | 0 |
| 4.061292705 | 0 |
| 4.06153629 | 0 |
| 4.061779875 | 0 |
| 4.06202346 | 0 |
| 4.062267045 | 0 |
| 4.06251063 | 0 |
| 4.062754215 | 0 |
| 4.0629978 | 0 |
| 4.063241385 | 0 |
| 4.06348497 | 0 |
| 4.063728555 | 0 |
| 4.06397214 | 0 |
| 4.064215725 | 0 |
| 4.06445931 | 0 |
| 4.064702895 | 0 |
| 4.06494648 | 0 |
| 4.065190065 | 0 |
| 4.06543365 | 0 |
| 4.065677235 | 0 |
| 4.06592082 | 0 |
| 4.066164405 | 0 |
| 4.06640799 | 0 |
| 4.066651575 | 0 |
| 4.06689516 | 0 |
| 4.067138745 | 0 |
| 4.06738233 | 0 |
| 4.067625915 | 0 |
| 4.0678695 | 0 |
| 4.068113085 | 0 |
| 4.06835667 | 0 |
| 4.068600255 | 0 |
| 4.06884384 | 0 |
| 4.069087425 | 0 |
| 4.06933101 | 0 |
| 4.069574595 | 0 |
| 4.06981818 | 0 |
| 4.070061765 | 0 |
| 4.07030535 | 0 |
| 4.070548935 | 0 |
| 4.07079252 | 0 |
| 4.071036105 | 0 |
| 4.07127969 | 0 |
| 4.071523275 | 0 |
| 4.07176686 | 0 |
| 4.072010445 | 0 |
| 4.07225403 | 0 |
| 4.072497615 | 0 |
| 4.0727412 | 0 |
| 4.072984785 | 0 |
| 4.07322837 | 0 |
| 4.073471955 | 0 |
| 4.07371554 | 0 |
| 4.073959125 | 0 |
| 4.07420271 | 0 |
| 4.074446295 | 0 |
| 4.07468988 | 0 |
| 4.074933465 | 0 |
| 4.07517705 | 0 |
| 4.075420635 | 0 |
| 4.07566422 | 0 |
| 4.075907805 | 0 |
| 4.07615139 | 0 |
| 4.076394975 | 0 |
| 4.07663856 | 0 |
| 4.076882145 | 0 |
| 4.07712573 | 0 |
| 4.077369315 | 0 |
| 4.0776129 | 0 |
| 4.077856485 | 0 |
| 4.07810007 | 0 |
| 4.078343655 | 0 |
| 4.07858724 | 0 |
| 4.078830825 | 0 |
| 4.07907441 | 0 |
| 4.079317995 | 0 |
| 4.07956158 | 0 |
| 4.079805165 | 0 |
| 4.08004875 | 0 |
| 4.080292335 | 0 |
| 4.08053592 | 0 |
| 4.080779505 | 0 |
| 4.08102309 | 0 |
| 4.081266675 | 0 |
| 4.08151026 | 0 |
| 4.081753845 | 0 |
| 4.08199743 | 0 |
| 4.082241015 | 0 |
| 4.0824846 | 0 |
| 4.082728185 | 0 |
| 4.08297177 | 0 |
| 4.083215355 | 0 |
| 4.08345894 | 0 |
| 4.083702525 | 0 |
| 4.08394611 | 0 |
| 4.084189695 | 0 |
| 4.08443328 | 0 |
| 4.084676865 | 0 |
| 4.08492045 | 0 |
| 4.085164035 | 0 |
| 4.08540762 | 0 |
| 4.085651205 | 0 |
| 4.08589479 | 0 |
| 4.086138375 | 0 |
| 4.08638196 | 0 |
| 4.086625545 | 0 |
| 4.08686913 | 0 |
| 4.087112715 | 0 |
| 4.0873563 | 0 |
| 4.087599885 | 0 |
| 4.08784347 | 0 |
| 4.088087055 | 0 |
| 4.08833064 | 0 |
| 4.088574225 | 0 |
| 4.08881781 | 0 |
| 4.089061395 | 0 |
| 4.08930498 | 0 |
| 4.089548565 | 0 |
| 4.08979215 | 0 |
| 4.090035735 | 0 |
| 4.09027932 | 0 |
| 4.090522905 | 0 |
| 4.09076649 | 0 |
| 4.091010075 | 0 |
| 4.09125366 | 0 |
| 4.091497245 | 0 |
| 4.09174083 | 0 |
| 4.091984415 | 0 |
| 4.092228 | 0 |
| 4.092471585 | 0 |
| 4.09271517 | 0 |
| 4.092958755 | 0 |
| 4.09320234 | 0 |
| 4.093445925 | 0 |
| 4.09368951 | 0 |
| 4.093933095 | 0 |
| 4.09417668 | 0 |
| 4.094420265 | 0 |
| 4.09466385 | 0 |
| 4.094907435 | 0 |
| 4.09515102 | 0 |
| 4.095394605 | 0 |
| 4.09563819 | 0 |
| 4.095881775 | 0 |
| 4.09612536 | 0 |
| 4.096368945 | 0 |
| 4.09661253 | 0 |
| 4.096856115 | 0 |
| 4.0970997 | 0 |
| 4.097343285 | 0 |
| 4.09758687 | 0 |
| 4.097830455 | 0 |
| 4.09807404 | 0 |
| 4.098317625 | 0 |
| 4.09856121 | 0 |
| 4.098804795 | 0 |
| 4.09904838 | 0 |
| 4.099291965 | 0 |
| 4.09953555 | 0 |
| 4.099779135 | 0 |
| 4.10002272 | 0 |
| 4.100266305 | 0 |
| 4.10050989 | 0 |
| 4.100753475 | 0 |
| 4.10099706 | 0 |
| 4.101240645 | 0 |
| 4.10148423 | 0 |
| 4.101727815 | 0 |
| 4.1019714 | 0 |
| 4.102214985 | 0 |
| 4.10245857 | 0 |
| 4.102702155 | 0 |
| 4.10294574 | 0 |
| 4.103189325 | 0 |
| 4.10343291 | 0 |
| 4.103676495 | 0 |
| 4.10392008 | 0 |
| 4.104163665 | 0 |
| 4.10440725 | 0 |
| 4.104650835 | 0 |
| 4.10489442 | 0 |
| 4.105138005 | 0 |
| 4.10538159 | 0 |
| 4.105625175 | 0 |
| 4.10586876 | 0 |
| 4.106112345 | 0 |
| 4.10635593 | 0 |
| 4.106599515 | 0 |
| 4.1068431 | 0 |
| 4.107086685 | 0 |
| 4.10733027 | 0 |
| 4.107573855 | 0 |
| 4.10781744 | 0 |
| 4.108061025 | 0 |
| 4.10830461 | 0 |
| 4.108548195 | 0 |
| 4.10879178 | 0 |
| 4.109035365 | 0 |
| 4.10927895 | 0 |
| 4.109522535 | 0 |
| 4.10976612 | 0 |
| 4.110009705 | 0 |
| 4.11025329 | 0 |
| 4.110496875 | 0 |
| 4.11074046 | 0 |
| 4.110984045 | 0 |
| 4.11122763 | 0 |
| 4.111471215 | 0 |
| 4.1117148 | 0 |
| 4.111958385 | −0.007 |
| 4.11220197 | −0.014 |
| 4.112445555 | −0.03 |
| 4.11268914 | −0.059 |
| 4.112932725 | −0.087 |
| 4.11317631 | −0.122 |
| 4.113419895 | −0.158 |
| 4.11366348 | −0.203 |
| 4.113907065 | −0.274 |
| 4.11415065 | −0.345 |
| 4.114394235 | −0.158 |
| 4.11463782 | 0.126 |
| 4.114881405 | 0.357 |
| 4.11512499 | 0.321 |
| 4.115368575 | 0.285 |
| 4.11561216 | 0.271 |
| 4.115855745 | 0.271 |
| 4.11609933 | 0.268 |
| 4.116342915 | 0.233 |
| 4.1165865 | 0.197 |
| 4.116830085 | 0.179 |
| 4.11707367 | 0.179 |
| 4.117317255 | 0.179 |
| 4.11756084 | 0.146 |
| 4.117804425 | 0.111 |
| 4.11804801 | 0.089 |
| 4.118291595 | 0.089 |
| 4.11853518 | 0.089 |
| 4.118778765 | 0.06 |
| 4.11902235 | 0.026 |
| 4.119265935 | 0 |
| 4.11950952 | 0 |
| 4.119753105 | 0 |
| 4.11999669 | 0 |
| 4.120240275 | 0 |
| 4.12048386 | 0 |
| 4.120727445 | 0 |
| 4.12097103 | 0 |
| 4.121214615 | 0 |
| 4.1214582 | 0 |
| 4.121701785 | 0 |
| 4.12194537 | 0 |
| 4.122188955 | 0 |
| 4.12243254 | 0 |
| 4.122676125 | 0 |
| 4.12291971 | 0 |
| 4.123163295 | 0 |
| 4.12340688 | 0 |
| 4.123650465 | 0 |
| 4.12389405 | 0 |
| 4.124137635 | 0 |
| 4.12438122 | 0.106 |
| 4.124624805 | 0.239 |
| 4.12486839 | 0.339 |
| 4.125111975 | 0.339 |
| 4.12535556 | 0.338 |
| 4.125599145 | 0.248 |
| 4.12584273 | 0.116 |
| 4.126086315 | 0 |
| 4.1263299 | 0 |
| 4.126573485 | 0 |
| 4.12681707 | 0 |
| 4.127060655 | 0 |
| 4.12730424 | 0 |
| 4.127547825 | −0.007 |
| 4.12779141 | −0.013 |
| 4.128034995 | −0.028 |
| 4.12827858 | −0.054 |
| 4.128522165 | −0.08 |
| 4.12876575 | −0.111 |
| 4.129009335 | −0.143 |
| 4.12925292 | −0.186 |
| 4.129496505 | −0.25 |
| 4.12974009 | −0.314 |
| 4.129983675 | −0.129 |
| 4.13022726 | 0.126 |
| 4.130470845 | 0.32 |
| 4.13071443 | 0.287 |
| 4.130958015 | 0.255 |
| 4.1312016 | 0.244 |
| 4.131445185 | 0.243 |
| 4.13168877 | 0.24 |
| 4.131932355 | 0.208 |
| 4.13217594 | 0.176 |
| 4.132419525 | 0.161 |
| 4.13266311 | 0.161 |
| 4.132906695 | 0.16 |
| 4.13315028 | 0.129 |
| 4.133393865 | 0.098 |
| 4.13363745 | 0.08 |
| 4.133881035 | 0.08 |
| 4.13412462 | 0.08 |
| 4.134368205 | 0.052 |
| 4.13461179 | 0.021 |
| 4.134855375 | 0 |
| 4.13509896 | 0 |
| 4.135342545 | 0 |
| 4.13558613 | 0 |
| 4.135829715 | 0 |
| 4.1360733 | 0 |
| 4.136316885 | 0 |
| 4.13656047 | 0 |
| 4.136804055 | 0 |
| 4.13704764 | 0 |
| 4.137291225 | 0 |
| 4.13753481 | 0 |
| 4.137778395 | 0 |
| 4.13802198 | 0 |
| 4.138265565 | 0 |
| 4.13850915 | 0 |
| 4.138752735 | 0 |
| 4.13899632 | 0 |
| 4.139239905 | 0 |
| 4.13948349 | 0 |
| 4.139727075 | 0 |
| 4.13997066 | 0.101 |
| 4.140214245 | 0.22 |
| 4.14045783 | 0.304 |
| 4.140701415 | 0.304 |
| 4.140945 | 0.303 |
| 4.141188585 | 0.216 |
| 4.14143217 | 0.098 |
| 4.141675755 | 0 |
| 4.14191934 | 0 |
| 4.142162925 | 0 |
| 4.14240651 | 0 |
| 4.142650095 | 0 |
| 4.14289368 | 0 |
| 4.143137265 | −0.006 |
| 4.14338085 | −0.012 |
| 4.143624435 | −0.026 |
| 4.14386802 | −0.05 |
| 4.144111605 | −0.073 |
| 4.14435519 | −0.101 |
| 4.144598775 | −0.13 |
| 4.14484236 | −0.169 |
| 4.145085945 | −0.226 |
| 4.14532953 | −0.284 |
| 4.145573115 | −0.104 |
| 4.1458167 | 0.125 |
| 4.146060285 | 0.284 |
| 4.14630387 | 0.255 |
| 4.146547455 | 0.227 |
| 4.14679104 | 0.218 |
| 4.147034625 | 0.217 |
| 4.14727821 | 0.213 |
| 4.147521795 | 0.184 |
| 4.14776538 | 0.156 |
| 4.148008965 | 0.144 |
| 4.14825255 | 0.144 |
| 4.148496135 | 0.142 |
| 4.14873972 | 0.114 |
| 4.148983305 | 0.086 |
| 4.14922689 | 0.071 |
| 4.149470475 | 0.071 |
| 4.14971406 | 0.071 |
| 4.149957645 | 0.045 |
| 4.15020123 | 0.018 |
| 4.150444815 | 0 |
| 4.1506884 | 0 |
| 4.150931985 | 0 |
| 4.15117557 | 0 |
| 4.151419155 | 0 |
| 4.15166274 | 0 |
| 4.151906325 | 0 |
| 4.15214991 | 0 |
| 4.152393495 | 0 |
| 4.15263708 | 0 |
| 4.152880665 | 0 |
| 4.15312425 | 0 |
| 4.153367835 | 0 |
| 4.15361142 | 0 |
| 4.153855005 | 0 |
| 4.15409859 | 0 |
| 4.154342175 | 0 |
| 4.15458576 | 0 |
| 4.154829345 | 0 |
| 4.15507293 | 0 |
| 4.155316515 | 0 |
| 4.1555601 | 0.095 |
| 4.155803685 | 0.201 |
| 4.15604727 | 0.271 |
| 4.156290855 | 0.271 |
| 4.15653444 | 0.27 |
| 4.156778025 | 0.187 |
| 4.15702161 | 0.082 |
| 4.157265195 | 0 |
| 4.15750878 | 0 |
| 4.157752365 | 0 |
| 4.15799595 | 0 |
| 4.158239535 | 0 |
| 4.15848312 | −0.001 |
| 4.158726705 | −0.006 |
| 4.15897029 | −0.011 |
| 4.159213875 | −0.025 |
| 4.15945746 | −0.045 |
| 4.159701045 | −0.066 |
| 4.15994463 | −0.091 |
| 4.160188215 | −0.117 |
| 4.1604318 | −0.153 |
| 4.160675385 | −0.204 |
| 4.16091897 | −0.255 |
| 4.161162555 | −0.083 |
| 4.16140614 | 0.121 |
| 4.161649725 | 0.252 |
| 4.16189331 | 0.226 |
| 4.162136895 | 0.2 |
| 4.16238048 | 0.194 |
| 4.162624065 | 0.193 |
| 4.16286765 | 0.188 |
| 4.163111235 | 0.162 |
| 4.16335482 | 0.137 |
| 4.163598405 | 0.128 |
| 4.16384199 | 0.128 |
| 4.164085575 | 0.125 |
| 4.16432916 | 0.1 |
| 4.164572745 | 0.075 |
| 4.16481633 | 0.063 |
| 4.165059915 | 0.063 |
| 4.1653035 | 0.063 |
| 4.165547085 | 0.039 |
| 4.16579067 | 0.014 |
| 4.166034255 | 0 |
| 4.16627784 | 0 |
| 4.166521425 | 0 |
| 4.16676501 | 0 |
| 4.167008595 | 0 |
| 4.16725218 | 0 |
| 4.167495765 | 0 |
| 4.16773935 | 0 |
| 4.167982935 | 0 |
| 4.16822652 | 0 |
| 4.168470105 | 0 |
| 4.16871369 | 0 |
| 4.168957275 | 0 |
| 4.16920086 | 0 |
| 4.169444445 | 0 |
| 4.16968803 | 0 |
| 4.169931615 | 0 |
| 4.1701752 | 0 |
| 4.170418785 | 0 |
| 4.17066237 | 0 |
| 4.170905955 | 0 |
| 4.17114954 | 0.089 |
| 4.171393125 | 0.183 |
| 4.17163671 | 0.24 |
| 4.171880295 | 0.239 |
| 4.17212388 | 0.239 |
| 4.172367465 | 0.219 |
| 4.17261105 | 0.195 |
| 4.172854635 | 0.178 |
| 4.17309822 | 0.178 |
| 4.173341805 | 0.177 |
| 4.17358539 | 0.16 |
| 4.173828975 | 0.137 |
| 4.17407256 | 0.117 |
| 4.174316145 | 0.117 |
| 4.17455973 | 0.117 |
| 4.174803315 | 0.103 |
| 4.1750469 | 0.08 |
| 4.175290485 | 0.058 |
| 4.17553407 | 0.058 |
| 4.175777655 | 0.058 |
| 4.17602124 | 0.047 |
| 4.176264825 | 0.024 |
| 4.17650841 | 0.002 |
| 4.176751995 | 0 |
| 4.17699558 | 0 |
| 4.177239165 | 0 |
| 4.17748275 | 0 |
| 4.177726335 | 0 |
| 4.17796992 | 0 |
| 4.178213505 | 0 |
| 4.17845709 | 0 |
| 4.178700675 | 0 |
| 4.17894426 | 0 |
| 4.179187845 | 0 |
| 4.17943143 | 0 |
| 4.179675015 | 0 |
| 4.1799186 | 0 |
| 4.180162185 | 0 |
| 4.18040577 | 0 |
| 4.180649355 | 0 |
| 4.18089294 | 0 |
| 4.181136525 | 0 |
| 4.18138011 | 0 |
| 4.181623695 | 0 |
| 4.18186728 | 0 |
| 4.182110865 | 0 |
| 4.18235445 | 0 |
| 4.182598035 | 0 |
| 4.18284162 | 0 |
| 4.183085205 | 0 |
| 4.18332879 | 0 |
| 4.183572375 | 0 |
| 4.18381596 | 0 |
| 4.184059545 | 0 |
| 4.18430313 | 0 |
| 4.184546715 | 0 |
| 4.1847903 | 0 |
| 4.185033885 | 0 |
| 4.18527747 | 0 |
| 4.185521055 | 0 |
| 4.18576464 | 0 |
| 4.186008225 | 0 |
| 4.18625181 | 0 |
| 4.186495395 | 0 |
| 4.18673898 | 0 |
| 4.186982565 | 0 |
| 4.18722615 | 0 |
| 4.187469735 | 0 |
| 4.18771332 | 0 |
| 4.187956905 | 0 |
| 4.18820049 | 0 |
| 4.188444075 | 0 |
| 4.18868766 | 0 |
| 4.188931245 | 0 |
| 4.18917483 | 0 |
| 4.189418415 | 0 |

TABLE 7

| Time (sec) | Amplitude |
|---|---|
| 1.07032475 | 0.072 |
| 1.0704045 | 0.143 |
| 1.07048425 | 0.215 |
| 1.070564 | 0.286 |
| 1.07064375 | 0.358 |
| 1.0707235 | 0.429 |
| 1.07080325 | 0.501 |
| 1.070883 | 0.572 |
| 1.07096275 | 0.644 |
| 1.0710425 | 0.715 |
| 1.07112225 | 0.787 |
| 1.071202 | 0.858 |
| 1.07128175 | 0.93 |
| 1.0713615 | 1.002 |
| 1.07144125 | 1.073 |
| 1.071521 | 1.144 |
| 1.07160075 | 1.216 |
| 1.0716805 | 1.287 |
| 1.07176025 | 1.359 |
| 1.07184 | 1.43 |
| 1.07191975 | 1.502 |
| 1.0719995 | 1.573 |
| 1.07207925 | 1.645 |
| 1.072159 | 1.716 |
| 1.07223875 | 1.788 |
| 1.0723185 | 1.859 |
| 1.07239825 | 1.931 |
| 1.072478 | 2.002 |
| 1.07255775 | 2.074 |
| 1.0726375 | 2.145 |
| 1.07271725 | 2.217 |
| 1.072797 | 2.288 |
| 1.07287675 | 2.36 |
| 1.0729565 | 2.431 |
| 1.07303625 | 2.503 |
| 1.073116 | 2.574 |
| 1.07319575 | 2.646 |
| 1.0732755 | 2.718 |
| 1.07335525 | 2.789 |
| 1.073435 | 2.861 |
| 1.07351475 | 2.932 |
| 1.0735945 | 3.004 |
| 1.07367425 | 3.075 |
| 1.073754 | 3.146 |
| 1.07383375 | 3.218 |
| 1.0739135 | 3.289 |
| 1.07399325 | 3.361 |
| 1.074073 | 3.432 |
| 1.07415275 | 3.504 |
| 1.0742325 | 3.575 |
| 1.07431225 | 3.647 |
| 1.074392 | 3.718 |
| 1.07447175 | 3.79 |
| 1.0745515 | 3.861 |
| 1.07463125 | 3.933 |
| 1.074711 | 4.004 |
| 1.07479075 | 4.076 |
| 1.0748705 | 4.147 |
| 1.07495025 | 4.219 |
| 1.07503 | 4.29 |
| 1.07510975 | 4.362 |
| 1.0751895 | 4.434 |
| 1.07526925 | 4.505 |
| 1.075349 | 4.577 |
| 1.07542875 | 4.648 |
| 1.0755085 | 4.72 |
| 1.07558825 | 4.791 |
| 1.075668 | 4.863 |
| 1.07574775 | 4.934 |
| 1.0758275 | 5.006 |
| 1.07590725 | 5.077 |
| 1.075987 | 5.148 |
| 1.07606675 | 5.22 |
| 1.0761465 | 5.291 |
| 1.07622625 | 5.363 |
| 1.076306 | 5.434 |
| 1.07638575 | 5.506 |
| 1.0764655 | 5.577 |
| 1.07654525 | 5.649 |
| 1.076625 | 5.72 |
| 1.07670475 | 5.792 |
| 1.0767845 | 5.863 |
| 1.07686425 | 5.935 |
| 1.076944 | 6.006 |
| 1.07702375 | 6.078 |
| 1.0771035 | 6.15 |
| 1.07718325 | 6.221 |
| 1.077263 | 6.293 |
| 1.07734275 | 6.364 |
| 1.0774225 | 6.436 |
| 1.07750225 | 6.507 |
| 1.077582 | 6.579 |
| 1.07766175 | 6.65 |
| 1.0777415 | 6.722 |
| 1.07782125 | 6.793 |
| 1.077901 | 6.865 |
| 1.07798075 | 6.936 |
| 1.0780605 | 7.008 |
| 1.07814025 | 7.079 |
| 1.07822 | 7.15 |
| 1.07829975 | 7.222 |
| 1.0783795 | 7.293 |
| 1.07845925 | 7.365 |
| 1.078539 | 7.436 |
| 1.07861875 | 7.508 |
| 1.0786985 | 7.579 |
| 1.07877825 | 7.651 |
| 1.078858 | 7.722 |
| 1.07893775 | 7.794 |
| 1.0790175 | 7.866 |
| 1.07909725 | 7.937 |
| 1.079177 | 8.009 |
| 1.07925675 | 8.08 |
| 1.0793365 | 8.152 |
| 1.07941625 | 8.223 |
| 1.079496 | 8.295 |
| 1.07957575 | 8.366 |
| 1.0796555 | 8.438 |
| 1.07973525 | 8.509 |
| 1.079815 | 8.581 |
| 1.07989475 | 8.652 |
| 1.0799745 | 8.724 |
| 1.08005425 | 8.795 |
| 1.080134 | 8.867 |
| 1.08021375 | 8.938 |
| 1.0802935 | 9.01 |
| 1.08037325 | 9.081 |
| 1.080453 | 9.152 |
| 1.08053275 | 9.224 |
| 1.0806125 | 9.295 |
| 1.08069225 | 9.367 |
| 1.080772 | 9.412 |
| 1.08085175 | 9.454 |
| 1.0809315 | 9.495 |
| 1.08101125 | 9.537 |
| 1.081091 | 9.579 |
| 1.08117075 | 9.621 |
| 1.0812505 | 9.662 |
| 1.08133025 | 9.704 |
| 1.08141 | 9.746 |
| 1.08148975 | 9.787 |
| 1.0815695 | 9.829 |
| 1.08164925 | 9.871 |
| 1.081729 | 9.913 |
| 1.08180875 | 9.922 |
| 1.0818885 | 9.922 |
| 1.08196825 | 9.922 |
| 1.082048 | 9.922 |
| 1.08212775 | 9.922 |
| 1.0822075 | 9.922 |
| 1.08228725 | 9.922 |
| 1.082367 | 9.922 |
| 1.08244675 | 9.922 |
| 1.0825265 | 9.922 |
| 1.08260625 | 9.922 |
| 1.082686 | 9.922 |
| 1.08276575 | 9.922 |
| 1.0828455 | 8.914 |
| 1.08292525 | 7.394 |
| 1.083005 | 5.875 |
| 1.08308475 | 4.355 |
| 1.0831645 | 2.836 |
| 1.08324425 | 1.317 |
| 1.083324 | −0.203 |
| 1.08340375 | −1.722 |
| 1.0834835 | −3.242 |
| 1.08356325 | −4.761 |
| 1.083643 | −6.281 |
| 1.08372275 | −7.8 |
| 1.0838025 | −9.32 |
| 1.08388225 | −9.961 |
| 1.083962 | −9.889 |
| 1.08404175 | −9.818 |
| 1.0841215 | −9.746 |
| 1.08420125 | −9.675 |
| 1.084281 | −9.603 |
| 1.08436075 | −9.532 |
| 1.0844405 | −9.46 |
| 1.08452025 | −9.389 |
| 1.0846 | −9.317 |
| 1.08467975 | −9.246 |
| 1.0847595 | −9.174 |
| 1.08483925 | −9.103 |
| 1.084919 | −9.031 |
| 1.08499875 | −8.96 |
| 1.0850785 | −8.888 |
| 1.08515825 | −8.817 |
| 1.085238 | −8.745 |
| 1.08531775 | −8.674 |
| 1.0853975 | −8.602 |
| 1.08547725 | −8.531 |
| 1.085557 | −8.459 |
| 1.08563675 | −8.388 |
| 1.0857165 | −8.316 |
| 1.08579625 | −8.245 |
| 1.085876 | −8.173 |
| 1.08595575 | −8.019 |
| 1.0860355 | −7.697 |
| 1.08611525 | −7.375 |
| 1.086195 | −7.054 |
| 1.08627475 | −6.732 |
| 1.0863545 | −6.41 |
| 1.08643425 | −6.088 |
| 1.086514 | −5.767 |
| 1.08659375 | −5.445 |
| 1.0866735 | −5.123 |
| 1.08675325 | −4.801 |
| 1.086833 | −4.479 |
| 1.08691275 | −4.158 |
| 1.0869925 | −3.841 |
| 1.08707225 | −3.543 |
| 1.087152 | −3.245 |
| 1.08723175 | −2.947 |
| 1.0873115 | −2.649 |
| 1.08739125 | −2.351 |
| 1.087471 | −2.054 |
| 1.08755075 | −1.756 |
| 1.0876305 | −1.458 |
| 1.08771025 | −1.16 |
| 1.08779 | −0.862 |
| 1.08786975 | −0.564 |
| 1.0879495 | −0.266 |
| 1.08802925 | 0.081 |
| 1.088109 | 0.837 |
| 1.08818875 | 1.594 |
| 1.0882685 | 2.351 |
| 1.08834825 | 3.108 |
| 1.088428 | 3.864 |
| 1.08850775 | 4.621 |
| 1.0885875 | 5.378 |
| 1.08866725 | 6.135 |
| 1.088747 | 6.891 |
| 1.08882675 | 7.648 |
| 1.0889065 | 8.405 |
| 1.08898625 | 9.162 |
| 1.089066 | 9.918 |
| 1.08914575 | 9.596 |
| 1.0892255 | 9.268 |
| 1.08930525 | 8.94 |
| 1.089385 | 8.613 |
| 1.08946475 | 8.285 |
| 1.0895445 | 7.957 |
| 1.08962425 | 7.629 |
| 1.089704 | 7.302 |
| 1.08978375 | 6.974 |
| 1.0898635 | 6.646 |
| 1.08994325 | 6.318 |
| 1.090023 | 5.991 |
| 1.09010275 | 5.663 |
| 1.0901825 | 5.625 |
| 1.09026225 | 5.625 |
| 1.090342 | 5.625 |
| 1.09042175 | 5.625 |
| 1.0905015 | 5.625 |
| 1.09058125 | 5.625 |
| 1.090661 | 5.625 |
| 1.09074075 | 5.625 |
| 1.0908205 | 5.625 |
| 1.09090025 | 5.625 |
| 1.09098 | 5.625 |
| 1.09105975 | 5.625 |
| 1.0911395 | 5.625 |
| 1.09121925 | 5.05 |
| 1.091299 | 4.305 |
| 1.09137875 | 3.56 |
| 1.0914585 | 2.815 |
| 1.09153825 | 2.07 |
| 1.091618 | 1.325 |
| 1.09169775 | 0.58 |
| 1.0917775 | −0.164 |
| 1.09185725 | −0.909 |
| 1.091937 | −1.654 |
| 1.09201675 | −2.399 |
| 1.0920965 | −3.144 |
| 1.09217625 | −3.889 |
| 1.092256 | −4.141 |
| 1.09233575 | −4.141 |
| 1.0924155 | −4.141 |
| 1.09249525 | −4.141 |
| 1.092575 | −4.141 |
| 1.09265475 | −4.141 |
| 1.0927345 | −4.141 |
| 1.09281425 | −4.141 |
| 1.092894 | −4.141 |
| 1.09297375 | −4.141 |
| 1.0930535 | −4.141 |
| 1.09313325 | −4.141 |
| 1.093213 | −4.141 |
| 1.09329275 | −4.387 |
| 1.0933725 | −4.834 |
| 1.09345225 | −5.28 |
| 1.093532 | −5.727 |
| 1.09361175 | −6.174 |
| 1.0936915 | −6.621 |
| 1.09377125 | −7.068 |
| 1.093851 | −7.515 |
| 1.09393075 | −7.962 |
| 1.0940105 | −8.408 |
| 1.09409025 | −8.855 |
| 1.09417 | −9.302 |
| 1.09424975 | −9.749 |
| 1.0943295 | −9.969 |
| 1.09440925 | −9.897 |
| 1.094489 | −9.826 |
| 1.09456875 | −9.754 |
| 1.0946485 | −9.683 |
| 1.09472825 | −9.611 |
| 1.094808 | −9.54 |
| 1.09488775 | −9.468 |
| 1.0949675 | −9.397 |
| 1.09504725 | −9.325 |
| 1.095127 | −9.254 |
| 1.09520675 | −9.183 |
| 1.0952865 | −9.111 |
| 1.09536625 | −9.016 |
| 1.095446 | −8.873 |
| 1.09552575 | −8.73 |
| 1.0956055 | −8.587 |
| 1.09568525 | −8.444 |
| 1.095765 | −8.301 |
| 1.09584475 | −8.158 |
| 1.0959245 | −8.015 |
| 1.09600425 | −7.872 |
| 1.096084 | −7.729 |
| 1.09616375 | −7.586 |
| 1.0962435 | −7.443 |
| 1.09632325 | −7.3 |
| 1.096403 | −7.142 |
| 1.09648275 | −6.927 |
| 1.0965625 | −6.713 |
| 1.09664225 | −6.498 |
| 1.096722 | −6.284 |
| 1.09680175 | −6.069 |
| 1.0968815 | −5.854 |
| 1.09696125 | −5.64 |
| 1.097041 | −5.425 |
| 1.09712075 | −5.211 |
| 1.0972005 | −4.996 |
| 1.09728025 | −4.782 |
| 1.09736 | −4.567 |
| 1.09743975 | −4.34 |
| 1.0975195 | −4.007 |
| 1.09759925 | −3.673 |
| 1.097679 | −3.339 |
| 1.09775875 | −3.005 |
| 1.0978385 | −2.672 |
| 1.09791825 | −2.338 |
| 1.097998 | −2.004 |
| 1.09807775 | −1.671 |
| 1.0981575 | −1.337 |
| 1.09823725 | −1.003 |
| 1.098317 | −0.67 |
| 1.09839675 | −0.336 |
| 1.0984765 | −0.002 |

TABLE 8

| Time (sec) | Amplitude |
|---|---|
| 3.036165725 | 0.072 |
| 3.03639195 | 0.143 |
| 3.036618175 | 0.215 |
| 3.0368444 | 0.286 |
| 3.037070625 | 0.358 |
| 3.03729685 | 0.429 |
| 3.037523075 | 0.501 |
| 3.0377493 | 0.572 |
| 3.037975525 | 0.644 |
| 3.03820175 | 0.715 |
| 3.038427975 | 0.787 |
| 3.0386542 | 0.858 |
| 3.038880425 | 0.93 |
| 3.03910665 | 1.002 |
| 3.039332875 | 1.073 |
| 3.0395591 | 1.144 |
| 3.039785325 | 1.216 |
| 3.04001155 | 1.287 |
| 3.040237775 | 1.359 |
| 3.040464 | 1.43 |
| 3.040690225 | 1.502 |
| 3.04091645 | 1.573 |
| 3.041142675 | 1.645 |
| 3.0413689 | 1.716 |
| 3.041595125 | 1.788 |
| 3.04182135 | 1.859 |
| 3.042047575 | 1.931 |
| 3.0422738 | 2.002 |
| 3.042500025 | 2.074 |
| 3.04272625 | 2.145 |
| 3.042952475 | 2.217 |
| 3.0431787 | 2.288 |
| 3.043404925 | 2.36 |
| 3.04363115 | 2.431 |
| 3.043857375 | 2.503 |
| 3.0440836 | 2.574 |
| 3.044309825 | 2.646 |
| 3.04453605 | 2.718 |
| 3.044762275 | 2.789 |
| 3.0449885 | 2.861 |
| 3.045214725 | 2.932 |
| 3.04544095 | 3.004 |
| 3.045667175 | 3.075 |
| 3.0458934 | 3.146 |
| 3.046119625 | 3.218 |
| 3.04634585 | 3.289 |
| 3.046572075 | 3.361 |
| 3.0467983 | 3.432 |
| 3.047024525 | 3.504 |
| 3.04725075 | 3.575 |
| 3.047476975 | 3.647 |
| 3.0477032 | 3.718 |
| 3.047929425 | 3.79 |
| 3.04815565 | 3.861 |
| 3.048381875 | 3.933 |
| 3.0486081 | 4.004 |
| 3.048834325 | 4.076 |
| 3.04906055 | 4.147 |
| 3.049286775 | 4.219 |
| 3.049513 | 4.29 |
| 3.049739225 | 4.362 |
| 3.04996545 | 4.434 |
| 3.050191675 | 4.505 |
| 3.0504179 | 4.577 |
| 3.050644125 | 4.648 |
| 3.05087035 | 4.72 |
| 3.051096575 | 4.791 |
| 3.0513228 | 4.863 |
| 3.051549025 | 4.934 |
| 3.05177525 | 5.006 |
| 3.052001475 | 5.077 |
| 3.0522277 | 5.148 |
| 3.052453925 | 5.22 |
| 3.05268015 | 5.291 |
| 3.052906375 | 5.363 |
| 3.0531326 | 5.434 |
| 3.053358825 | 5.506 |
| 3.05358505 | 5.577 |
| 3.053811275 | 5.649 |
| 3.0540375 | 5.72 |
| 3.054263725 | 5.792 |
| 3.05448995 | 5.863 |
| 3.054716175 | 5.935 |
| 3.0549424 | 6.006 |
| 3.055168625 | 6.078 |
| 3.05539485 | 6.15 |
| 3.055621075 | 6.221 |
| 3.0558473 | 6.293 |
| 3.056073525 | 6.364 |
| 3.05629975 | 6.436 |
| 3.056525975 | 6.507 |
| 3.0567522 | 6.579 |
| 3.056978425 | 6.65 |
| 3.05720465 | 6.722 |
| 3.057430875 | 6.793 |
| 3.0576571 | 6.865 |
| 3.057883325 | 6.936 |
| 3.05810955 | 7.008 |
| 3.058335775 | 7.079 |
| 3.058562 | 7.15 |
| 3.058788225 | 7.222 |
| 3.05901445 | 7.293 |
| 3.059240675 | 7.365 |
| 3.0594669 | 7.436 |
| 3.059693125 | 7.508 |
| 3.05991935 | 7.579 |
| 3.060145575 | 7.651 |
| 3.0603718 | 7.722 |
| 3.060598025 | 7.794 |
| 3.06082425 | 7.866 |
| 3.061050475 | 7.937 |
| 3.0612767 | 8.009 |
| 3.061502925 | 8.08 |
| 3.06172915 | 8.152 |
| 3.061955375 | 8.223 |
| 3.0621816 | 8.295 |
| 3.062407825 | 8.366 |
| 3.06263405 | 8.438 |
| 3.062860275 | 8.509 |
| 3.0630865 | 8.581 |
| 3.063312725 | 8.652 |
| 3.06353895 | 8.724 |
| 3.063765175 | 8.795 |
| 3.0639914 | 8.867 |
| 3.064217625 | 8.938 |
| 3.06444385 | 9.01 |
| 3.064670075 | 9.081 |
| 3.0648963 | 9.152 |
| 3.065122525 | 9.224 |
| 3.06534875 | 9.295 |
| 3.065574975 | 9.367 |
| 3.0658012 | 9.412 |
| 3.066027425 | 9.454 |
| 3.06625365 | 9.495 |
| 3.066479875 | 9.537 |
| 3.0667061 | 9.579 |
| 3.066932325 | 9.621 |
| 3.06715855 | 9.662 |
| 3.067384775 | 9.704 |
| 3.067611 | 9.746 |
| 3.067837225 | 9.787 |
| 3.06806345 | 9.829 |
| 3.068289675 | 9.871 |
| 3.0685159 | 9.913 |
| 3.068742125 | 9.922 |
| 3.06896835 | 9.922 |
| 3.069194575 | 9.922 |
| 3.0694208 | 9.922 |
| 3.069647025 | 9.922 |
| 3.06987325 | 9.922 |
| 3.070099475 | 9.922 |
| 3.0703257 | 9.922 |
| 3.070551925 | 9.922 |
| 3.07077815 | 9.922 |
| 3.071004375 | 9.922 |
| 3.0712306 | 9.922 |
| 3.071456825 | 9.922 |
| 3.07168305 | 8.914 |
| 3.071909275 | 7.394 |
| 3.0721355 | 5.875 |
| 3.072361725 | 4.355 |
| 3.07258795 | 2.836 |
| 3.072814175 | 1.317 |
| 3.0730404 | −0.203 |
| 3.073266625 | −1.722 |
| 3.07349285 | −3.242 |
| 3.073719075 | −4.761 |
| 3.0739453 | −6.281 |
| 3.074171525 | −7.8 |
| 3.07439775 | −9.32 |
| 3.074623975 | −9.961 |
| 3.0748502 | −9.889 |
| 3.075076425 | −9.818 |
| 3.07530265 | −9.746 |
| 3.075528875 | −9.675 |
| 3.0757551 | −9.603 |
| 3.075981325 | −9.532 |
| 3.07620755 | −9.46 |
| 3.076433775 | −9.389 |
| 3.07666 | −9.317 |
| 3.076886225 | −9.246 |
| 3.07711245 | −9.174 |
| 3.077338675 | −9.103 |
| 3.0775649 | −9.031 |
| 3.077791125 | −8.96 |
| 3.07801735 | −8.888 |
| 3.078243575 | −8.817 |
| 3.0784698 | −8.745 |
| 3.078696025 | −8.674 |
| 3.07892225 | −8.602 |
| 3.079148475 | −8.531 |
| 3.0793747 | −8.459 |
| 3.079600925 | −8.388 |
| 3.07982715 | −8.316 |
| 3.080053375 | −8.245 |
| 3.0802796 | −8.173 |
| 3.080505825 | −8.019 |
| 3.08073205 | −7.697 |
| 3.080958275 | −7.375 |
| 3.0811845 | −7.054 |
| 3.081410725 | −6.732 |
| 3.08163695 | −6.41 |
| 3.081863175 | −6.088 |
| 3.0820894 | −5.767 |
| 3.082315625 | −5.445 |
| 3.08254185 | −5.123 |
| 3.082768075 | −4.801 |
| 3.0829943 | −4.479 |
| 3.083220525 | −4.158 |
| 3.08344675 | −3.841 |
| 3.083672975 | −3.543 |
| 3.0838992 | −3.245 |
| 3.084125425 | −2.947 |
| 3.08435165 | −2.649 |
| 3.084577875 | −2.351 |
| 3.0848041 | −2.054 |
| 3.085030325 | −1.756 |
| 3.08525655 | −1.458 |
| 3.085482775 | −1.16 |
| 3.085709 | −0.862 |
| 3.085935225 | −0.564 |
| 3.08616145 | −0.266 |
| 3.086387675 | 0.081 |
| 3.0866139 | 0.837 |
| 3.086840125 | 1.594 |
| 3.08706635 | 2.351 |
| 3.087292575 | 3.108 |
| 3.0875188 | 3.864 |
| 3.087745025 | 4.621 |
| 3.08797125 | 5.378 |
| 3.088197475 | 6.135 |
| 3.0884237 | 6.891 |
| 3.088649925 | 7.648 |
| 3.08887615 | 8.405 |
| 3.089102375 | 9.162 |
| 3.0893286 | 9.918 |
| 3.089554825 | 9.596 |
| 3.08978105 | 9.268 |
| 3.090007275 | 8.94 |
| 3.0902335 | 8.613 |
| 3.090459725 | 8.285 |
| 3.09068595 | 7.957 |
| 3.090912175 | 7.629 |
| 3.0911384 | 7.302 |
| 3.091364625 | 6.974 |
| 3.09159085 | 6.646 |
| 3.091817075 | 6.318 |
| 3.0920433 | 5.991 |
| 3.092269525 | 5.663 |
| 3.09249575 | 5.625 |
| 3.092721975 | 5.625 |
| 3.0929482 | 5.625 |
| 3.093174425 | 5.625 |
| 3.09340065 | 5.625 |
| 3.093626875 | 5.625 |
| 3.0938531 | 5.625 |
| 3.094079325 | 5.625 |
| 3.09430555 | 5.625 |
| 3.094531775 | 5.625 |
| 3.094758 | 5.625 |
| 3.094984225 | 5.625 |
| 3.09521045 | 5.625 |
| 3.095436675 | 5.05 |
| 3.0956629 | 4.305 |
| 3.095889125 | 3.56 |
| 3.09611535 | 2.815 |
| 3.096341575 | 2.07 |
| 3.0965678 | 1.325 |
| 3.096794025 | 0.58 |
| 3.09702025 | −0.164 |
| 3.097246475 | −0.909 |
| 3.0974727 | −1.654 |
| 3.097698925 | −2.399 |
| 3.09792515 | −3.144 |
| 3.098151375 | −3.889 |
| 3.0983776 | −4.141 |
| 3.098603825 | −4.141 |
| 3.09883005 | −4.141 |
| 3.099056275 | −4.141 |
| 3.0992825 | −4.141 |
| 3.099508725 | −4.141 |
| 3.09973495 | −4.141 |
| 3.099961175 | −4.141 |
| 3.1001874 | −4.141 |
| 3.100413625 | −4.141 |
| 3.10063985 | −4.141 |
| 3.100866075 | −4.141 |
| 3.1010923 | −4.141 |
| 3.101318525 | −4.387 |
| 3.10154475 | −4.834 |
| 3.101770975 | −5.28 |
| 3.1019972 | −5.727 |
| 3.102223425 | −6.174 |
| 3.10244965 | −6.621 |
| 3.102675875 | −7.068 |
| 3.1029021 | −7.515 |
| 3.103128325 | −7.962 |
| 3.10335455 | −8.408 |
| 3.103580775 | −8.855 |
| 3.103807 | −9.302 |
| 3.104033225 | −9.749 |
| 3.10425945 | −9.969 |
| 3.104485675 | −9.897 |
| 3.1047119 | −9.826 |
| 3.104938125 | −9.754 |
| 3.10516435 | −9.683 |
| 3.105390575 | −9.611 |
| 3.1056168 | −9.54 |
| 3.105843025 | −9.468 |
| 3.10606925 | −9.397 |
| 3.106295475 | −9.325 |
| 3.1065217 | −9.254 |
| 3.106747925 | −9.183 |
| 3.10697415 | −9.111 |
| 3.107200375 | −9.016 |
| 3.1074266 | −8.873 |
| 3.107652825 | −8.73 |
| 3.10787905 | −8.587 |
| 3.108105275 | −8.444 |
| 3.1083315 | −8.301 |
| 3.108557725 | −8.158 |
| 3.10878395 | −8.015 |
| 3.109010175 | −7.872 |
| 3.1092364 | −7.729 |
| 3.109462625 | −7.586 |
| 3.10968885 | −7.443 |
| 3.109915075 | −7.3 |
| 3.1101413 | −7.142 |
| 3.110367525 | −6.927 |
| 3.11059375 | −6.713 |
| 3.110819975 | −6.498 |
| 3.1110462 | −6.284 |
| 3.111272425 | −6.069 |
| 3.11149865 | −5.854 |
| 3.111724875 | −5.64 |
| 3.1119511 | −5.425 |
| 3.112177325 | −5.211 |
| 3.11240355 | −4.996 |
| 3.112629775 | −4.782 |
| 3.112856 | −4.567 |
| 3.113082225 | −4.34 |
| 3.11330845 | −4.007 |
| 3.113534675 | −3.673 |
| 3.1137609 | −3.339 |
| 3.113987125 | −3.005 |
| 3.11421335 | −2.672 |
| 3.114439575 | −2.338 |
| 3.1146658 | −2.004 |
| 3.114892025 | −1.671 |
| 3.11511825 | −1.337 |
| 3.115344475 | −1.003 |
| 3.1155707 | −0.67 |
| 3.115796925 | −0.336 |
| 3.11602315 | −0.002 |

1. An electrotherapy devices comprising
a transducer; and
a controller, the controller being capable of energizing the transducer to produce a specific low frequency magnetic field pulse waveform (Cnp), the Cnp comprising a plurality of intermittent waveforms, with one or more latency periods between the waveforms.

2. The device of claim 1, wherein the waveforms are designed to initially mimic an endogenous electrical activity of target tissue of a subject.

3. The device of claim 2, wherein the latency period between the waveforms varies in a predetermined manner over time.

4. The device of claim 3, wherein the Cnp is designed to initially entrain the electrical activity of the target tissue and as a result affect endogenous electrical activity of the target tissue.

5. The device of claim 4, wherein the Cnp comprises an anti-depression Cnp (Cnp-1).

6. The device of claim 5, wherein the waveform mimics the electromagnetic waveform of: at least one of: the medial thalamus, anterior cingulated, frontal cortex and the pre-frontal cortex in an animal.

7. (canceled)

8. (canceled)

9. (canceled)

10. The device of claim 6, wherein the latency period is in the range of 1 to 200 ms milliseconds (ms).

11. (canceled)

12. The device of claim 1, wherein the Cnp comprises an analgesic Cnp (Cnp-3).

13. The device of claim 12, wherein the waveform mimics the electromagnetic waveform of at least one of: the insular cortex, Papez circuit and the anterior cingulate in an animal.

14. The device of claim 1, wherein Cnp comprises an anti-anxiety Cnp (Cnp-3).

15. (canceled)

16. (canceled)

17. The device of claim 14, wherein the latency period is in the range of 1 to 250 milliseconds (ms).

18. (canceled)

19. The device of claim 1, comprising a plurality of Cnps, the Cnps being separated by at least one refractory period therebetween, and wherein consecutive refractory periods are variable.

20. (canceled)

21. The device of claim 19, wherein the consecutive refractory periods progressively increase in duration.

22. The device of claim 21, comprising four consecutive refractory periods.

23. The device of claim 21, wherein the Cnp comprises an anti-depression Cnp (Cnp-1) and the refractory period is in the range of 100 to 4000 ms.

24. (canceled)

25. The device of claim 21, wherein the Cnp comprises an analgesic Cnp (Cnp-2) and the refractory period is in the range of 50 to 1900 ms.

26. (canceled)

27. The device of claim 21, wherein the Cnp comprises an anti-anxiety Cnp (Cnp-3) and refractory period is in the range of 200 to 7000 ms.

28. (canceled)

29. The device of claim 1, further comprising one or more additional Cnps, and wherein the controller is capable of causing the transducer to produce at least two different pluralities of Cnps in succession.

30. (canceled)

31. The device of any one of claim 29, wherein each of the at least two different pluralities of Cnps respectively comprise at least one of: an anti-depression Cnp (Cnp-1), an analgesic Cnp (Cnp-2), and an anti-anxiety Cnp (Cnp-3).

32. (canceled)

33. (canceled)

34. The device of claim 31, wherein the at least two different Cnps in succession form a sequence designed to treat eating disorders.

35. The device of claim 34, wherein the at least two different Cnps in succession respectively comprise the Cnp-1 followed by Cnp-3.

36. (canceled)

37. (canceled)

38. The device of claim 31, wherein the at least two different Cnps in succession form a sequence designed to treat chronic pain.

39. The device of claim 38, wherein the at least two different Cnps in succession comprise Cnp-1 followed by the Cnp-2 followed by the Cnp-3.

40. (canceled)

41. (canceled)

42. The device of claim 39, wherein the transducer is a wire coil.

43. The device of claim 42, wherein the wire coil is designed to target a deep brain region.

44. The device of claim 42 wherein the wire coil is elliptical in shape.

45. The device of claim 43 wherein the wire coil is selected from the group comprising: a core-less wire coil, a solid core wire coil, and a multi-layer printed circuit board wire coil.

46. A method for treating a disorder selected from the group of physiological, neurological and behavioral disorders, the method comprising: applying to a subject a specific low frequency magnetic field pulse waveform (Cnp), the Cnp comprising one or more intermittent waveforms, with a latency period between the waveforms, the waveforms being designed to initially mimic an endogenous electrical activity of target tissue of the subject, wherein application of the Cnp is for a time duration effective to produce a desired effect in the target tissue.

47. The method of claim 46, wherein the latency period between the waveforms varies in a predetermined manner over time.

48. The method of claim 46, wherein the low frequency pulsed magnetic field initially entrains the electrical activity of the target tissue and as a result affects the endogenous electrical activity of the target tissue.

49. The method of claim 48, wherein the Cnp utilized comprises a plurality of Cnps, the Cnps being separated by a plurality of refractory periods, and the refractory periods vary in a predetermined manner over time.

50. (canceled)

51. The method of claim 49, wherein at least two different pluralities of Cnps are applied in succession.

52. (canceled)

53. A low frequency magnetic field pulse (Cnp), the Cnp comprising intermittent waveforms, with a latency period between the waveforms, and the waveforms being designed to initially mimic an endogenous electrical activity of target tissue of a subject.

54. The Cnp of claim 53, wherein the Cnp is selected from the group comprising any one of an anti-depression Cnp (Cnp-1), an analgesic Cnp (Cnp-2) and an anti-anxiety Cnp (Cnp-3).

55. An array of Cnps according to claim 34.

56. An electrotherapy device comprising transducers oriented such that optimal deep tissue penetration is achieved.

57. An electrotherapy device comprising transducers oriented such that the deep tissue magnetic field is nullified without compromising the magnetic field at surface tissues.
